(19) United States
(12) Patent Application Publication
(10) Pub. No.: US 2026/0273047 A1
(43) Pub. Date: Sep. 17, 2026

(54) COMPOSITIONS AND METHODS FOR AMELIORATING ADVERSE EFFECTS OF THERAPIES

(71) Applicant: MDX Management LLC, La Jolla, CA (US)

(72) Inventors: Yuan LIU, Decatur, GA (US); Lei SHI, Decatur, GA (US); Zhen BIAN, Tucker, GA (US); Harry STYLLI, La Jolla, CA (US)

(21) Appl. No.: 19/164,937

(22) PCT Filed: Mar. 15, 2024
(86) PCT No.: PCT/US2024/020307

§ 371 Date: Sep. 12, 2025

Related U.S. Application Data
(60) Provisional application No. 63/490,995, filed on Mar. 17, 2023.
(60) Provisional application No. 63/581,184, filed on Sep. 7, 2023.

(51) Int. Cl. A61K 39/395; A61K 31/122; A61K 31/4745; A61K 31/506; A61K 31/7084; A61K 35/17; A61K 38/20; A61P 35/00

(52) U.S. Cl. CPC A61K 39/3955; A61K 31/122; A61K 31/4745; A61K 31/506; A61K 31/7084; A61K 35/17; A61K 38/2013; A61K 39/39558; A61P 35/00

(57) ABSTRACT

The present application provides a method of treating a cancer in an individual that involves administering to the individual a myeloid cell activating agent or therapy (such as a TLR agonist or a STING agonist) and a TNFα inhibitor. In some cases, the method further involves administering a SHP-1 inhibitor and/or tyrosine kinase inhibitor to the individual, optionally further administering a lymphocyte activating agent such as a cytokine (such as IL-2) and/or an immune checkpoint inhibitor (such as anti-PD-1).

### CROSS-REFERENCE TO RELATED APPLICATIONS

This application claims the benefit of, and priority to U.S. Provisional Application No. 63/490,995, filed Mar. 17, 2023, and U.S. Provisional Application No. 63/581,184, filed Sep. 7, 2023, the content of each of which is incorporated herein by reference in their entirety.

### FIELD OF THE INVENTION

The present invention relates to compositions and methods for treating a disease (e.g., cancer) involving administering a TNFα inhibitor and a myeloid cell activating agent or therapy and optionally a SHP-1 inhibitor and/or a tyrosine kinase inhibitor.

### BACKGROUND OF THE INVENTION

In cancers such as solid tumors, intratumoral myeloid leukocytes, including macrophages (i.e., tumor-associated macrophage or TAM) and myeloid-derived suppressive cells (MDSC), play critical roles in controlling the tumor microenvironment (TME) immunosuppression that supports tumor growth and also confers tumor resistance to immunotherapeutic treatments. One important mechanism for myeloid leukocytes to adapt an immunosuppressive phenotype, or to strengthen their immunosuppressive capacity following tumor therapies, is through their cell surface inhibitory receptors (iRs), which upon activation are also driven by their extracellular ligand binding that triggers multi-pathways of negative regulation via their cytoplasmic domain immunoreceptor tyrosine-based inhibitory motifs (ITIMs) that activate SHP-1, the central signal modulator, to dephosphorylate and hence deactivate a number of signal transduction molecules. This diminishes therapeutics-induced anti-cancer pro-inflammatory responses. In solid tumors, essential cell surface iRs, such as SIRPα, Siglecs, LilRBs, PirB, LAIR1, lectin receptors, SLAM family receptors, etc. (see, e.g., Kang, X. L. et al., Cell Cycle 2016; 15:25-40; and Zarrin, A. A. et al., Front Immunol. 2020; 11, each of which is hereby incorporated by reference), which also show increased expression in the TME with tumor progression to advanced stages, conduct their regulations via activation of SHP-1, which then mediates downstream inhibition.

Given these inhibitory mechanisms elucidated within previous years, pipelines of therapeutic developments aiming to blockade iRs (e.g., anti-LilRB1/2 and anti-SIRPα) and their ligands (e.g., anti-CD47) are being undertaken (see, e.g., Carosella, E. D. et al., Trends Cancer 2021; 7:389-392; Yanagita, T. Y. et al., JCI Insight 2017; 2; and Zhang, W. et al., Front Immunol. 2020; 11:18, each of which is hereby incorporated by reference). However, these efforts of targeting each iR or its ligand singularly, but not all inhibitory pathways at once, achieve weak-to-partial efficacies in controlling solid tumors.

Furthermore, the pro-inflammatory responses of anti-cancer therapeutics can lead to cytokine release syndrome (CRS) in the patient, wherein clinical manifestations include elevated circulating cytokine levels (e.g., INF-γ, CCL2, IL-10, or IL-6), acute systemic inflammatory symptoms, and secondary organ dysfunction (e.g., nephritis, hepatitis, and/or pneumonitis). CRS has been defined as a systemic inflammatory state occurring due to robust systemic immune activation induced by a cell-mediated immune response. Currently, there are several treatments for CRS. Low grade CRS is treated symptomatically with antihistamines, antipyretics, and fluids. More severe CRS can be treated, for example, with corticosteroid treatment or anti-IL-6 treatment (e.g., tocilizumab), however these treatments may negatively impact the efficacy of the anti-cancer therapy. Engaging the immune system to eliminate cancers comes with the risk of developing CRS as a serious adverse event, which can prove lethal or necessitate that the patient terminates the anti-cancer treatment. Therefore, safe and effective novel anti-cancer therapeutics are needed.

The disclosures of all publications, patents, patent applications and published patent applications referred to herein are hereby incorporated herein by reference in their entirety.

### BRIEF SUMMARY OF THE INVENTION

The present application in one aspect provides a method of treating a cancer in an individual, comprising administering to the individual a) a myeloid cell activating agent or therapy, and b) a TNFα inhibitor. In some embodiments, the method further comprises administering to the individual an inhibitor of the SHP-1 pathway.

In another aspect is provided a method of treating a cancer in an individual, comprising administering to the individual a TNFα inhibitor and an inhibitor of the SHP-1 pathway. In some embodiments, the method further comprises administering a lymphocyte activating agent.

In some embodiments according to any of the methods described above, the individual is under an inflammation reaction. In some embodiments, the inflammation reaction is characterized by a) an acute inflammation, b) a cytokine release syndrome (e.g., CRS of grade one above, two above or three above), c) an increased level of 1) at least two or three of TNFa, IL-6, IFN-g, and IFN-a, and/or 2) at least two or three of CCL2, CCL5, CXCL1, and CXCL10, further optionally wherein the inflammation reaction is characterized by an increased level of IL-2, IL-12, IL1b, and/or IL-10.

In some embodiments according to any of the methods described above, the inhibitor of the SHP-1 pathway comprises a SHP-1 inhibitor. In some embodiments, the SHP-1 inhibitor is selected from the group consisting of: a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZFN, or TALENS systems), a peptide agent, a protein agent (e.g., an antibody agent that targets SHP-1), a protein degrading or destabilizing agent, a protein modified with an unnatural amino acid, an antibody directed therapy, an antibody drug conjugate (ADC), and any combination thereof. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the SHP-1 inhibitor is TPI-1 or an analog or derivative thereof.

In some embodiments according to any of the methods described above, the inhibitor of the SHP-1 pathway comprises a tyrosine kinase inhibitor. In some embodiments, the tyrosine kinase inhibitor is selected from the group consisting of: a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZFN, or TALENS systems), a peptide agent, a protein agent (e.g., an antibody agent that targets tyrosine kinase or activated tyrosine kinase), a protein degrading or destabilizing agent, a protein modified with an unnatural amino acid, an antibody directed therapy, an antibody drug conjugate (ADC), and any combination thereof. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800.

In some embodiments according to any of the methods described above, the tyrosine kinase inhibitor inhibits any one of: Src, Syk, Hck, Lck, Lyn, JAK, and Yes. In some embodiments, the tyrosine kinase inhibitor does not or weakly inhibits one or more kinases involved in T cell activation. In some embodiments, the one or more kinases involved in T cell activation comprises any one or more of: Lck, Fyn, Zap70, Syk and Csk. In some embodiments, the tyrosine kinase inhibitor is an inhibitor of a tyrosine kinase of a Src family.

In some embodiments according to any of the methods described above, the inhibitor of the SHP-1 pathway is an antibody that blocks a cell surface inhibitory receptor. In some embodiments, the antibody that blocks cell surface inhibitory receptor is selected from any one of: LILRB1, LILRB2, LILRB3, LILRB4, LILRB5, SIRPα, PirB, gp49B1, Siglec-1, Siglec-2, Siglec-3, Siglec-5, Siglec-6, Siglec-7, Siglec-8, Siglec-9, Siglec-10, Siglec-11, Siglec-12, Siglec-14, Siglec-15, Siglec-E, Siglec-F, Siglec-G, Siglec-H, DCIR4, CD371, CD200R, SLAMF1, SLAMF3, SLAMF5, SLAMF6, SLAMF7, SLAMF8, and SLAMF9.

In some embodiments according to any of the methods described above, the pro-inflammatory agent capable of activating myeloid cells (i.e., myeloid cell activating agent or therapy) activates a cell selected from any one of: macrophages having the M1 phenotype, intratumoral dendritic cells, intratumoral B cells, antigen presenting cells, and any combination thereof. In some embodiments, the myeloid cell activating agent or therapy is selected from the group consisting of: a STING activator, a Toll-like receptor (TLR) agonist, a PAMP/DAMP activator, a chemotherapy, a pro-inflammatory cytokine, a cancer vaccine, a bacteria or component thereof, a virus or component thereof, a fungus or component thereof, an immune cell, a sound treatment, a magnetic therapy, an electrical treatment, a cryotherapy, a surgery, a thermotherapy, a radiation treatment, a radiopharmaceutical treatment, an electrostatic treatment, an antibody drug conjugate, and any combination thereof.

In some embodiments according to any of the methods described above, the myeloid cell activating agent or therapy comprises a TLR agonist. In some embodiments, the TLR agonist activates TLR2, TLR3, TLR4, TLR5, TLR7, TLR8, TLR9, and/or zymosan. In some embodiments, the TLR agonist comprises CpG, polyI:C, and/or R848.

In some embodiments according to any of the methods described above, the myeloid cell activating agent or therapy comprises a STING activator. In some embodiments, the STING activator is selected from the group consisting of: 2′3′-cGAMP, ADU-s100, G10, SR-717, Vadimezan (DMXAA; ASA-404), Sting agonist-20, MSA-2, diABZI STING agonist-1, cGAMP (Cyclic GMP-AMPP), STING agonist-3, and c-di-AMP (Cyclic diadenylate) sodium.

In some embodiments according to any of the methods described above, the myeloid cell activating agent comprises immune cells. In some embodiments, the immune cells comprise T cells. In some embodiments, the T cells express a chimeric antigen receptor (CAR) or an antigen specific TCR. In some embodiments, the immune cells comprise at least about 10⁶, 2×10⁶, 5×10⁶, 10⁷, 2×10⁷, 5×10⁷, 10⁸, 2×10⁸, 5×10⁸ T cells. In some embodiments, the method comprises administering at least two or three doses of the immune cells (i.e., two to three administration of the immune cells). In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the immune cells (e.g., each dose of immune cells).

In some embodiments according to any of the methods described above, the TNFα inhibitor is selected from the group consisting of: a small molecule inhibitor, a neutralizing antibody, a TNFα receptor blockade antibody, a soluble TNFα receptor, a TNFα-targeting short interfering RNA (siRNA), a chemical inhibitor of TNFα mRNA stability, an inhibitor of TNFα converting enzyme (TACE), and derivatives thereof. In some embodiments, the TNFα inhibitor is a TNFα neutralizing antibody. In some embodiments, the antibody is selected from the group consisting of: infliximab, adalimumab, etanercept, golimumab, and certolizumab.

In some embodiments according to any of the methods described above, the method further comprises administering to the individual an effective amount of a lymphocyte activating agent. In some embodiments, the lymphocyte is a T cell. In some embodiments, the lymphocyte activating agent is selected from the group consisting of: a cytokine, a chemokine, a metabolism-modulating drug, a metabolite antagonist, an immune checkpoint inhibitor, an immune cell, a cancer vaccine, a bacteria or component thereof, a virus or component thereof, a fungus or component thereof, a bispecific T cell engager (BiTE), an antibody drug conjugate, and any combination thereof.

In some embodiments according to any of the methods described above, the TNFα inhibitor is administered prior to the administration of the myeloid cell activating agent or therapy. In some embodiments, the TNFα inhibitor is administered after the administration of the myeloid cell activating agent or therapy. In some embodiments, the TNFα inhibitor is administered within 5, 4, 3, 2, or 1 day of the administration of the myeloid cell activating agent or therapy, or wherein the TNFα inhibitor is administered no more than four days after the administration of the myeloid cell activating agent or therapy.

In some embodiments according to any of the methods described above, the TNFα inhibitor is administered within two weeks prior to, concurrently, or within 3 hours after the administration of a) the myeloid cell activating agent or therapy and/or b) inhibitor of the SHP-1 pathway.

In some embodiments according to any of the methods described above, the TNFα inhibitor is administered within two weeks prior to, concurrently, or within 3 hours after the administration of a) the lymphocyte activating agent and/or b) inhibitor of the SHP-1 pathway.

In some embodiments according to any of the methods described above, the myeloid cell activating agent or therapy is administered systemically or locally. In some embodiments, the TNFα inhibitor is administered systemically or locally. In some embodiments, the inhibitor of the SHP-1 signaling pathway is administered systemically or locally. In some embodiments, the systemic administration comprises oral administration, intravenous administration, subcutaneous administration, or intraperitoneal administration. In some embodiments, the local administration comprises intratumoral administration.

In some embodiments according to any of the methods described above, the myeloid cell activating agent or therapy is administered daily for at least 2, 3, 4, 5, 6, or 7 days. In some embodiments, the myeloid cell activating agent or therapy is administered intermittently.

In some embodiments according to any of the methods described above, the inhibitor of the SHP-1 signaling pathway is administered daily for at least 2, 3, 4, 5, 6, or 7 days. In some embodiments, the inhibitor of the SHP-1 signaling pathway is administered intermittently.

In some embodiments according to any of the methods described above, the inhibitor of the SHP-1 pathway and the myeloid cell activating agent or therapy or the lymphocyte activating agent are administered within 24 hours of each other. In some embodiments, the inhibitor of the SHP-1 pathway and the myeloid cell activating agent or therapy or the lymphocyte activating agent are administered to the individual simultaneously or concurrently.

In some embodiments according to any of the methods described above, the TNFα inhibitor is administered at least once a week, once every five days, once every three days, or daily. In some embodiments, the TNFα inhibitor is administered intermittently. In some embodiments, the TNFα inhibitor is administered no more than about once every two weeks, no more than once a week, or no more than once every five days. In some embodiments, the TNFα inhibitor is administered to the individual for at least two cycles, wherein each cycle has about three to about seven days.

In some embodiments according to any of the methods described above, the method further comprises assessing the level of TNFα level in the individual (e.g., serum or blood TNFα level).

In some embodiments according to any of the methods described above, the method further comprises administering an IL-6 inhibitor.

In some embodiments according to any of the methods described above, the method comprises administering at least two doses of the TNFα inhibitor, optionally wherein the two doses of the TNFα inhibitor is separated a) at least by 2, 3, 4, 5, 6, or 7 days, or b) at most by 4, 3, 2 or 1 week, 6, or 5 days.

In some embodiments according to any of the methods described above, the SHP-1 pathway inhibitor comprises a tyrosine kinase inhibitor and a SHP-1 inhibitor.

In some embodiments according to any of the methods described above, the method comprises administering a) a SHP-1 inhibitor, optionally the SHP-1 inhibitor is a TPI-1 or an analog or derivative thereof, b) a TLR agonist, optionally wherein the TLR agonist activates TLR2, TLR3, TLR4, TLR5, TLR7, TLR8, TLR9, and/or zymosan, and c) an TNFα inhibitor, optionally wherein the TNFα inhibitor is an anti-TNFα antibody.

In some embodiments according to any of the methods described above, the method comprises administering a) a SHP-1 inhibitor, optionally the SHP-1 inhibitor is a TPI-1 or an analog or derivative thereof, b) a STING activator, and c) an TNFα inhibitor, optionally wherein the TNFα inhibitor is an anti-TNFα antibody.

In some embodiments according to any of the methods described above, the method comprises administering a) a SHP-1 inhibitor, optionally the SHP-1 inhibitor is a TPI-1 or an analog or derivative thereof, b) a radiotherapy, and c) an TN-Fa inhibitor, optionally wherein the TNFα inhibitor is an anti-TNFα antibody.

In some embodiments according to any of the methods described above, the inhibitor of the SHP-1 pathway is administered to the individual simultaneously with the myeloid cell activating agent or therapy. In some embodiments, the inhibitor of the SHP-1 pathway and the myeloid cell activating agent or therapy are administered sequentially. In some embodiments, the inhibitor of the SHP-1 pathway and the myeloid cell activating agent or therapy are administered to the individual until the individual undergoes tumor clearance.

In some embodiments according to any of the methods described above, the inhibitor of the SHP-1 pathway, the myeloid cell activating agent or therapy, and/or the TNFα inhibitor are further administered intermittently to the individual after tumor clearance, wherein the individual was administered a myeloid cell activating agent or therapy, an inhibitor of the SHP-1 pathway, and/or a TNFα inhibitor according to any of the methods described herein prior to tumor clearance.

In some embodiments according to any of the methods described above, the lymphocyte activating agent is a cytokine, wherein the cytokine comprises 11-2, IL-4, IL-7, IL-9, IL-21, or IL-15, or a biologically active derivative thereof. In some embodiments, the cytokine comprises IL-2 or a biologically active derivative thereof.

In some embodiments according to any of the methods described above, the myeloid cell activating agent or the lymphocyte activating agent is an immune checkpoint inhibitor, wherein the immune checkpoint inhibitor comprises an anti-PD-1 antibody.

In some embodiments according to any of the methods described above, the IL-2 or biologically active derivative thereof and/or the anti-PD-1 antibody is administered to the individual daily (e.g., for at least 2, 3, 4, 5, 6, or 7 days). In some embodiments, the IL-2 or biologically active derivative thereof and/or the anti-PD-1 antibody is administered to the individual intermittently. In some embodiments, the IL-2 or biologically active derivative thereof and/or the anti-PD-1 antibody is administered to the individual for at least two cycles, wherein each cycle has about three to about 20 days.

In some embodiments according to any of the methods described above, the individual does not develop Grade 2-4 cytokine release syndrome or pro-inflammatory organ damage. In some embodiments, administration of the TNFα inhibitor does not compromise or weakly compromises tumor clearance.

In some embodiments according to any of the methods described above, the cancer is a solid tumor. In some embodiments, the cancer is a hematological cancer. In some embodiments, the cancer is a late-stage cancer. In some embodiments, the cancer is resistant or refractory to a radiation therapy, a chemotherapeutic agent, and/or a checkpoint inhibitor.

In some embodiments, the individual is a human.

### BRIEF DESCRIPTION OF THE DRAWINGS

FIG. 1 provides a schematic overview of the LLC mouse model experimental design. Mice with multiple LLC engraftments were treated daily with a) s.c. TPI-1, 1 mg/kg, b) s.c. PolyI:C+R848, each 20 μg, and c) s.c. Dasatinib, 2 mg/kg (hereafter called KX147.AB&C) along with IL-2 and anti-PD1 mAb to promote T cell immunity. A group of mice were also given a dose of anti-TNFα mAb prior to (−1 d) the start of KX147.AB&C treatment, followed by a second dose on d5. LLC, lewis lung carcinoma; s.c., subcutaneous injection; i.p., intraperitoneal injection; d, day; tox, toxicity; mAb, monoclonal antibody.

FIG. 2 shows the luminescence images showing that KX147.AB&C, either without or with anti-TNFα mAb, induced rapid regression of LLC tumors. LLC, lewis lung carcinoma; ctl, control; CR, complete response; OS, overall survival; mAb, monoclonal antibody.

FIG. 3A shows the recorded tumor volume changes over time after treatment with vehicle (n=5 mice), with KX147.AB&C (n=5 mice), or with KX147.AB&C and anti-TNFα mAb combination treatment (n=7 mice). V, volume; d, day; ctd, control; CR, complete response; mAb, monoclonal antibody.

FIG. 3B shows the recorded animal overall survival over time after treatment with vehicle (n=5 mice), with KX147.AB&C (n=5 mice), or with KX147.AB&C and anti-TNFα mAb combination treatment (n=7 mice). Ctl, control; d, day; mAb, monoclonal antibody.

FIGS. 4A-4B show the reduction in key cytokines and chemokines in mice treated with anti-TNFα mAb and KX147.AB&C combination therapy. FIG. 4A shows the cytokine levels for TNFα, IL-6, IL-10, IFNα, IFNβ, IFNγ, IL-1β, IL-12, and GM-CSF in serum from mice treated with anti-TNFα mAb versus without anti-TNFα mAb, which include key inflammatory cytokines indicative of CRS. FIG. 4B shows the chemokine levels for CCL2, CCL5, CXCL1, and CXCL10 in serum from mice treated with anti-TNFα mAb versus without anti-TNFα mAb. H, hour(s); mAb, monoclonal antibody. ***, p<0.0001.

FIG. 5 shows the clinical scores and body weight loss for mice administered KX147.AB &C therapy and co-treated with versus without anti-TNFα mAb. D, day; mAb, monoclonal antibody.

FIGS. 6A-6B show the organ assessment post-euthanasia in KX147.AB&C-treated mice with versus without anti-TNFα mAb administration. FIG. 6A shows the organ weight for spleen, liver, kidney, and colon. FIG. 6B shows a photograph of murine colons and spleens with a ruler for size comparison. D, day; g, grams; mAb, monoclonal antibody. ***, p<0.0001.

FIG. 7 shows PMN infiltration in organs based on tissue MPO assays using tissue from spleen, liver, lung, kidney, and colon harvested from post-euthanasia KX147.AB&C-treated mice with versus without anti-TNFα mAb administration. PMN, polymorphonuclear leukocyte or neutrophil; MPO, myeloperoxidase activity assay; mAb, monoclonal antibody. ***, p<0.0001.

FIG. 8 shows tissue section staining to assess for PMN infiltration into the lungs of post-euthanasia KX147.AB&C-treated mice with versus without anti-TNFα mAb administration. PMN, polymorphonuclear leukocyte or neutrophil; d, day; mAb, monoclonal antibody.

FIG. 9 provides a schematic overview of the MC38 mouse model experimental design. Mice with single or double MC 38 colorectal carcinoma engraftments were treated daily for three days with a) s.c. TPI-1, 1 mg/kg, b) s.c. PolyI:C+R848, each 20 μg, and c) s.c. Dasatinib, 2 mg/kg (hereafter called KX147.AB&C) along with IL-2 and anti-PD1 mAb to promote T cell immunity. On d4, mice were changed to KX147.AB treatment (i.e., a) s.c. TPI-1, 1 mg/kg, and b) s.c. PolyI:C+R848, each 20 μg). A group of mice were also given a dose of anti-TNFα mAb prior to (−1d) the start of KX147.AB&C treatment, followed by a second dose on d5. MC38, C57Bl/6 murine colon adenocarcinoma cell line; s.c., subcutaneous injection; i.p., intraperitoneal injection; d, day; tox, toxicity; mAb, monoclonal antibody.

FIGS. 10A-10B show the recorded tumor volume changes over time after treatment with a) vehicle (FIG. 10A; n=3 mice per group), or b) with KX147.AB&C daily for three days followed by KX147.AB on d4, in combination with anti-TNFα mAb, IL-2, and anti-PD-1 mAb treatment (FIG. 10B; n=4 mice). Control mice tested as shown in FIG. 10A included the following groups: vehicle, IL-2 alone, anti-PD-1 mAb alone, and IL-2/anti-PD-1 mAb combination treatment. mice tested as shown in FIG. 10B included the following: mouse #1, MC38 engraftment in both flanks; mouse #2, MC38 engraftment in single flank; mouse #3, MC38 engraftment in single flank; mouse #4 MC38 engraftment in both flanks. V, volume; d, day; ctl, control; CR, complete response; OS, overall survival; mAb, monoclonal antibody.

FIG. 11 provides a schematic overview of the experimental design, wherein mice with established MC38 colorectal carcinoma (200-400 mm³) were treated with αTLR, TPI-1 and Dasatinib (s.c.), without or with additional treatment with anti-TNFα mAb or anti-IL-6 mAb (150 μg, i.p.). The treatment was repeated once (d1 and d2). In this model, KX147.AB&C treatment involved administration of a) s.c. TPI-1, 3 mg/kg, b) s.c. PolyI:C+R848, each 20 μg, and c) s.c. Dasatinib, 5 mg/kg. Mice were euthanized for analysis on d6 post-onset of treatment. αTLR, TLR agonist; s.c., subcutaneous injection; i.p. intraperitoneal injection; d, day; mAb, monoclonal antibody.

FIG. 12 shows tumor volume changes over six days following various treatments over two days of treatment administration. KX147.AB&C alone effectively controlled tumor growth and induced regression. Neither anti-TNFα mAb nor anti-IL-6 mAb treatment affected KX147.AB&C efficacy. V, volume; d, day; NT, no treatment; αTLR, TLR agonist; mAb, monoclonal antibody.

FIGS. 13A-13D show the FACS plot analysis of immune cell infiltration of multiple immune lineages (i.e., CD8 T cells, CD4 T_{H} cells, NK cells, PMNs, macrophages, and MDSCs) into the TME. FIG. 13A shows the FACS plots for the no-treatment group. FIG. 13B shows the FACS plots for αTLR/TPI-1/Dasatinib therapy (KX147.AB&C). FIG. 13C shows the FACS plots for αTLR/TPI-1/Dasatinib therapy (KX147.AB&C) in combination with anti-TNFα mAb. FIG. 13D shows the FACS plots for αTLR/TPI-1/Dasatinib therapy (KX147.AB&C) in combination with anti-IL-6 mAb. SSC, side scatter; FSC, forward scatter; T_{H}, T helper cell; NK, natural killer; PMNs, polymorphonuclear leukocytes or neutrophils; MDSCs, myeloid derived suppressor cells; αTLR, TLR agonist; mAb, monoclonal antibody; NT, no-treatment; d, day.

FIG. 14 shows as a bar graph the quantified FACS results of the TME analyses as described for FIGS. 13A-13D above. T_{c}, cytotoxic T cell; T_{H}, T helper cell; NK, natural killer; PMNs, polymorphonuclear leukocytes or neutrophils; Mac, macrophage; MDSCs, myeloid derived suppressor cells; αTLR, TLR agonist; mAb, monoclonal antibody; ns, not significant: ctl, control. **, p<0.001. ***, p<0.0001.

FIG. 15 shows the cytokine levels for TNFα, IL-6, IL-1β, IL-10, IFNα, and IFNγ as well as chemokine levels for CCL2, CCL5, CXCL1, and CXCL10 in serum from mice treated with αTLR/TPI-1/Dasatinib therapy or in combination with either anti-TNFα mAb or anti-IL-6 mAb. αTLR, TLR agonist; mAb, monoclonal antibody; Mo, monocyte; Mac, macrophage; PMN, polymorphonuclear leukocyte or neutrophil. *, p<0.05. ***, p<0.0001.

FIG. 16 shows photographs of murine colons and spleens with a ruler for size comparison from mice treated with αTLR/TPI-1/Dasatinib therapy or in combination with either anti-TNFα mAb or anti-IL-6 mAb. αTLR, TLR agonist; mAb, monoclonal antibody.

FIG. 17 provides a schematic overview of the experimental design, wherein mice were s.c. engrafted with KPC pancreatic ductal adenocarcinomas into the left and right flanks. Mice were treated with KX147.AB&C for 4 days and then switched to KX147.AB therapy until tumor clearance. IL-2 and anti-PD-1 mAb were combined to enhance T cell immunity. On one day before KX147.AB&C treatment began, and again on d5, anti-TNFα mAb was administered. D, day; s.c., subcutaneous injection; i.p., intraperitoneal injection; mAb, monoclonal antibody.

FIG. 18 shows the luminescence images showing that KX147.AB&C, either without or with anti-TNFα mAb, induced rapid regression of KPC tumors. Four treatment groups were tested: (1) no treatment (control), (2) IL-2 and anti-PD-1 mAb combination therapy, (3) KX147.AB&C in combination with IL-2 and anti-PD-1 mAb, and (4) KX147.AB&C in combination with IL-2, anti-PD-1 mAb, and anti-TNFα mAb. KPC, KPC pancreatic ductal adenocarcinomas; d, day; ctl, control; CR, complete response; OS, overall survival; mAb, monoclonal antibody.

FIG. 19 provides a schematic overview of the experimental design, wherein mice underwent i.p. orthotopic engraftment with KPC pancreatic ductal adenocarcinomas. Mice were treated with KX147.AB&C for 2 days and then switched to KX147.AB therapy until tumor clearance. IL-2 and anti-PD-1 mAb were combined to enhance T cell immunity. On one day before KX147.AB&C treatment began, and again on d5, anti-TNFα mAb was administered. D, day; s.c., subcutaneous injection; i.p., intraperitoneal injection; mAb, monoclonal antibody.

FIG. 20 shows the luminescence images showing that KX147.AB&C, either without or with anti-TNFα mAb, induced rapid regression of KPC tumors. Three treatment groups were tested: (1) IL-2 and anti-PD-1 mAb combination therapy, (2) KX147.AB&C in combination with IL-2 and anti-PD-1 mAb, and (3) KX147.AB&C in combination with IL-2, anti-PD-1 mAb, and anti-TNFα mAb. KPC, KPC pancreatic ductal adenocarcinomas; d, day; ctl, control; CR, complete response; OS, overall survival; mAb, monoclonal antibody.

FIG. 21 shows tumor volume changes from two days before treatment until nine days post-treatment in mice bearing s.c. KPC tumors. KX147.AB&C with IL-2 and anti-PD-1 mAb alone effectively controlled tumor growth and induced regression. anti-TNFα mAb did not affect KX147.AB&C efficacy. V, volume; d, day; s.c., subcutaneous injection: ctd, control; CR, complete response; mAb, monoclonal antibody.

FIG. 22 shows tumor volume changes from two days before treatment until seven days post-treatment in mice bearing i.p. orthotopic KPC tumors. KX147.AB&C with IL-2 and anti-PD-1 mAb alone effectively controlled tumor growth and induced regression. anti-TNFα mAb did not affect KX147.AB&C efficacy. V, volume: d, day; i.p., intraperitoneal injection; ctl, control; CR, complete response; mAb, monoclonal antibody.

FIG. 23 shows the survival curve of mice treated with one of the following treatments: (1) no treatment (control; n=10 mice), (2) IL-2 and anti-PD-1 mAb (n=6 mice), (3) KX147.AB&C with IL-2 and anti-PD-1 mAb (n=6 mice), and (4) KX147.AB&C in combination with IL-2, anti-PD-1 mAb, and anti-TNFα mAb (n=8 mice). Despite the anti-TNFα mAb not contributing to KX147.AB&C-induced tumor elimination, its presence ameliorated adverse effects and enabled 100% survival rates. KPC, KPC pancreatic ductal adenocarcinoma; d, day; mAb, monoclonal antibody.

FIG. 24 shows the two steps of cytokine release events associated with CAR-T therapy against cancer. In Step 1, activated CAR-T cells release cytokines (e.g., IFNγ, TNFα and IL-2) while executing effector functions including killing of cancer cells. In Step 2, cytokines released by CAR-T cells induce broad activation of macrophages and other immune and body cells, which together produce high level proinflammatory cytokines (e.g., IL-6) and chemokines. Activation in Step 2 leads to acute response, leukocyte infiltration, and immune-related adverse effects (irAEs).

FIGS. 25A-25C show that neutralization of CAR-T produced TNFα leads to curbed macrophage activation and decreased production of proinflammatory cytokines in vitro. FIG. 25A outlines the two-dish method for testing the effect of cytokines produced by activated CAR-T cells on macrophages in vitro. Dish 1 comprises co-culture of CD19 CAR-T cells and B-ALL leukemic cells, while Dish 2 comprises human macrophage cultures. Dish 2 is cultured with supernatant from Dish 1 with or without monoclonal antibody(mAb)-mediated neutralization of TNFα, IFNγ or IL-2. FIG. 25B shows quantification of CAR-T cell effector function against B-ALL cells over 24 hours of co-culture in Dish 1, as well as production of cytokines from said activated CAR-T cells. FIG. 25C shows outcome of cytokine and chemokine release from macrophages of Dish 2 after 16 hours of culture with the supernatant of Dish 1 with or without mAb-mediated neutralization of target cytokines.

FIGS. 26A-26C show that neutralization of CAR-T produced TNFα prevents surrounding macrophages from producing high levels of IL-6 and inflammatory chemokines in vitro. FIG. 26A outlines the in vitro co-culture system. CD19 CAR-T and B-ALL were co-cultured with human monocytes-derived macrophages in a single dish at a ratio of CAR-T:B-ALL:hMac=1:10:3 (1×10⁶ T cells/ml) for 24 hours with or without mAb-mediated cytokine neutralization. After culturing, cytokine and chemokine concentrations in culture medium were determined by ELISA. FIG. 26B shows that CD19 CAR-T-mediated killing of B-ALL cells was not affected by neutralization with mAb treatment. FIG. 26C shows the resulting changes on cytokine and chemokine levels after 24 hr co-culture with or without mAb-mediated cytokine neutralization.

FIGS. 27A-27E show that prophylactic anti-TNFα neutralization prevented severe Cytokine Release Syndrome (CRS) without compromising CAR-T anti-tumor efficacy in patient-derived xenograft (PDX) mouse models. FIG. 27A outlines the experimental design. The F3 B-ALL PDX mouse model was established by i.v. injection of B-ALL (1×10⁶ cells per mouse) harvested from F2 PDXs. Once B-ALL cells were detectable in peripheral blood (>10% in PBMC) of B-ALL xenograft mice, 1-2×10⁶ CD19 CAR-T cells were i.v. infused without or with prophylactic anti-TNFα mAb administration (i.p., 100 μg, 3 h prior to CAR-T infusion). The same dose of anti-TNFα mAb was then given weekly until mice reaching complete response (CR). FIG. 27B shows representative flow cytometric analyses of B-ALL cells in the peripheral blood mononuclear cells (PBMCs) of F3 B-ALL PDX model mice with or without prophylactic anti-TNFα mAb treatment. FIG. 27C establishes that CAR-T proliferation and killing of B-ALL cells in F3 B-ALL PDX models is not hindered by prophylactic anti-TNFα mAb administration. FIG. 27D shows Overall Survival (OS) of PDX mice receiving CAR-T therapy with or without prophylactic anti-TNFα mAb administration. FIG. 27E shows levels of cytokines and chemokines in the blood serum of PDX model mice following CAR-T therapy.

FIGS. 28A-28B show that prophylactic anti-TNFα mAb administration protected mice from CD3/CD28 TCR ligation-induced CRS. FIG. 28A outlines the experimental design. Mice with and without prophylactic administration of anti-TNFα mAb (100 μg, i.p., 3 h prior to CD3/CD28 Ab treatment) were given a mixture of CD3 and CD28 ligation mAbs (50 μg each, i.p.) to stimulate endogenous and systemic T cell activation. At 0 hr, 3 hr, and 16 hr post-CD3/CD28 mAb treatment blood serum from treated mice was analyzed by multiplex ELISA for cytokine and chemokine levels. FIG. 28B shows levels of cytokines and chemokines in the blood serum of mice following CD3/CD28 mAb ligation treatment.

FIGS. 29A-29D show that prophylactic anti-TNFα mAb administration ameliorated CRS induced by adoptive infusion of activated T cells. FIG. 29A outlines the experimental design. Mice with and without prophylactic administration of anti-TNFα mAb (100 μg, i.p., 3 h prior to treatment) were i.v. infused with 2×10⁷ splenocytic T cells activated by anti-CD3/CD28 mAb ligation. At 0 hr, 3 hr, and 16 hr post-infusion the blood serum from treated mice was analyzed by multiplex ELISA for cytokine and chemokine levels. FIG. 29B shows levels of cytokines and chemokines in the blood serum of mice following single infusion of activated splenocytic T cells. FIG. 29C shows body weight measurements and clinical scores of mice receiving repeated infusions of activated splenocytic T cells (3× i.v. on days 0, 2, and 4; 2×10⁷ cells each infusion) with or without prophylactic anti-TNFα mAb administration. FIG. 29D shows the effect of repeated infusions of activated splenocytic T cells with or without prophylactic anti-TNFα mAb administration on spleen weight and instances of splenomegaly in recipient mice.

FIGS. 30A-30D show topical therapy to cutaneous/subcutaneous 4T1 breast cancer. FIG. 30A shows the overall experimental design. BalbC mice were engrafted cutaneously/subcutaneously with 4T1 breast cancer cells, which were allowed to develop to tumors for 10-15 days. Following tumor growth phase, mice received prophylactic anti-TNFα intraperitoneal (i.p.) injection followed by treatment with either control conditions or experimental conditions. Control conditions included treatment with topical non-drug lotion (Johnson's lotion alone), while experimental conditions included topical lotion comprising aTLR (polyI:C and R848) alone (condition A), dTPI-1 alone (condition B) or aTLR and dTPI-1 (condition A+B). All lotion treatments were administered two times per day for nine days following prophylactic TNFα treatment, and all mice received systemic anti-PD-1 (αPD-1) therapy on days one, four, and seven of treatment. Efficacy analysis was performed at day 10 following prophylactic TNFα treatment. FIG. 30B shows quantification of 4T1 breast cancer tumor volume changes during treatment. FIG. 30C shows bioluminescence images of 4T1 breast cancer tumors in mice treated with condition A (aTLR) and condition B (dTPI-1) (A+B) plus systemic treatment with anti-PD-1 (μPD-1) therapy. FIG. 30D shows survival rates of mice receiving each treatment.

FIGS. 31A-31B show topical therapy to cutaneous/subcutaneous lung cancer (LLC). FIG. 31A shows bioluminescence images of lung cancer tumor (LLC)-engrafted mice over the course of seven days of treatment with either control topical lotion only, anti-PD-1 (αPD-1) systemic treatment only, anti-PD-1 (αPD-1) systemic treatment with topical aTLR+dTPI-1 treatment, or anti-PD-1 (αPD-1) systemic treatment with topical aSTING and dTPI-1 treatment. All topical treatments were provided two times per day, while anti-PD-1 (αPD-1) systemic treatment was provided once every three days. FIG. 31B shows quantification of tumor volume changes during treatments.

FIGS. 32A-32D show the therapeutic anti-cancer efficacy of combined SHP-1 inhibition and T cell activation. MC38 colorectal carcinoma was established in C57Bl/6 mice. After tumor formation, the tumors were treated by intratumoral (i.t.) injection with (i) anti-PD-1 Ab alone (αPD-1, 50 μg, every 3 days) or αPD-1 in combination with the SHP-1 inhibitor, TPI-1 (1 mg/kg, every 2 days) (FIG. 32A), (ii) a single dose of anti-CD3 and anti-CD28 antibodies (50 μg each) or anti-CD3/anti-CD28 antibodies in combination with TPI-1 (FIG. 32B), or (iii) IL-2 (30,000 IU, every 3 days) alone or in combination with TPI-1 or IL-2 with TPI-1 in combination with anti-TNFα and anti-IL-6 antibodies (50 μg each) (FIG. 32C). Addition of neutralizing anti-TNFα and anti-IL-6 antibodies did not affect treatment efficacy. (FIG. 32D) shows that treating mice with TPI-1 alone did not stop tumor progression. iSHP-1, SHP-1 inhibitor; V, volume.

FIGS. 33A-33C show that prophylactic anti-TNFα monoclonal antibody (mAb) treatment did not affect TLR agonist R848, TPI-1, or their combination in tumor treatment. FIG. 33A outlines the experimental design. Murine pancreatic ductal adenocarcinoma cells (KPCs) were engrafted (5×10⁵, s.c.) into the right flank of C57BL6 mice. Upon tumor formation (~200 mm³ tumor volume), mice were treated daily with R848 (20 μg or 60 μg, s.c.) or TPI-1 (1 or 3 mg/kg, s. c.), or R848 plus TPI-1. All treatment conditions were tested with or without prophylactic anti-TNFα mAb (i.p., 100 μg). FIG. 33B shows tumor burden changes following each treatment over an 8-day treatment window. FIG. 33C shows immune cell infiltrates within the tumor microenvironment (TME) on day 8 of treatment.

FIGS. 34A-34C show that prophylactic anti-TNFα monoclonal antibody (mAb) treatment ameliorated cytokine release syndrome (CRS) induced by therapies with TLR agonist R848, TPI-1, and R848 and TPI-1 in combination. Mice with and without prophylactic anti-TNFα (100 μg mAb, i.p., 3 h prior to first treatment) were treated daily with R848 (20 μg or 60 μg, s.c.), TPI-1 (1 or 3 mg/kg, s.c.), or their combination. Mice receiving each treatment regimen were assayed for cytokine and chemokine release in blood serum by multiplex ELISA (FIG. 34A). FIG. 34B shows body weight and clinical score changes in treated mice over the 8-day treatment window. FIG. 34C shows analysis of both colitis and splenomegaly, symptoms of CRS, at 8 days following treatment initiation.

FIGS. 35A-35C show that prophylactic anti-TNFα monoclonal antibody (mAb) treatment did not affect STING agonist (ADU-S100) and its combination with TPI-1 in tumor treatment. FIG. 35A shows the experimental design. C57BL6 mice were engrafted with murine pancreatic ductal adenocarcinoma (KPC; 5×10⁵, s.c.). Once tumors formed >150 mm³, mice were treated prophylactically with antiTNFα mAb (i.p., 100 μg) followed by daily treatment with ADU-S100 (100 μg/mouse, s.c.) with or without TPI-1 (1 mg/kg, s.c.). FIG. 35B shows KPC tumor burden changes over the course of 19 days following initiation of treatment. FIG. 35C shows immune cell infiltrates within the tumor microenvironment (TME) on day 8 of treatment.

FIGS. 36A-36C show that prophylactic anti-TNFα monoclonal antibody (mAb) treatment ameliorated cytokine release syndrome (CRS) associated with STING agonist therapy. Mice with and without prophylactic administration with anti-TNFα (100 μg mAb, i.p., 3 h prior to the first STING agonist treatment) were treated daily with STING agonist ADU-S100 (100 μg/mouse, s.c.). Mice receiving each treatment regimen were assayed for cytokine and chemokine release in blood serum by multiplex ELISA (FIG. 36A). FIG. 36B shows body weight and clinical score changes in treated mice over the 8-day treatment window. FIG. 36C shows analysis of both colitis and splenomegaly, symptoms of CRS, at 8 days following treatment initiation.

### DETAILED DESCRIPTION OF THE INVENTION

The present application in one aspect provides methods of treating a cancer in an individual, comprising administering to the individual a) a myeloid cell activating agent or therapy, and b) a TNFα inhibitor. The present application in some embodiments provides methods of treating a cancer in an individual that further comprises administering to the individual an inhibitor of the SHP-1 pathway. In another aspect is provided a method of treating a cancer in an individual, comprising administering to the individual a TNFα inhibitor and an inhibitor of the SHP-1 pathway, wherein the individual is under an inflammation reaction or has an ongoing infection. In some embodiments, the inhibitor of the SHP-1 pathway is a SHP-1 inhibitor, for example TPI-1 or an analog or derivative thereof. In some embodiments, the inhibitor of the SHP-1 pathway is a tyrosine kinase inhibitor, for example Dasatinib. In some embodiments, the inhibitor of the SHP-1 signaling pathway is administered systemically. In some embodiments, the method comprises administering the inhibitor of the SHP-1 signaling pathway daily for at least 2, 3, 4, 5, 6, or 7 days or intermittently. In some embodiments, the myeloid cell activating agent or therapy comprises an agent selected from the group consisting of: a STING activator, a Toll-like receptor (TLR) agonist, a PAMP/DAMP activator, a chemotherapy, a pro-inflammatory cytokine, a cancer vaccine, a bacteria or component thereof, a virus (e.g., an oncolytic virus) or component thereof, a fungus or component thereof, a sound treatment, a magnetic therapy, an electrical treatment, a radiation treatment, a radiopharmaceutical treatment, an electrostatic treatment, an antibody drug conjugate, and any combination thereof. In some embodiments, the TNFα inhibitor is selected from the group consisting of: a small molecule inhibitor, a neutralizing antibody, a TNFα receptor blockade antibody, a soluble TNFα receptor, a TNFα-targeting short interfering RNA (siRNA), a chemical inhibitor of TNFα mRNA stability, an inhibitor of TNFα converting enzyme (TACE), and derivatives thereof. In some embodiments, the myeloid cell activating agent or therapy is administered systemically. In some embodiments, the TNFα inhibitor is administered systemically. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. Further combination therapy methods are provided.

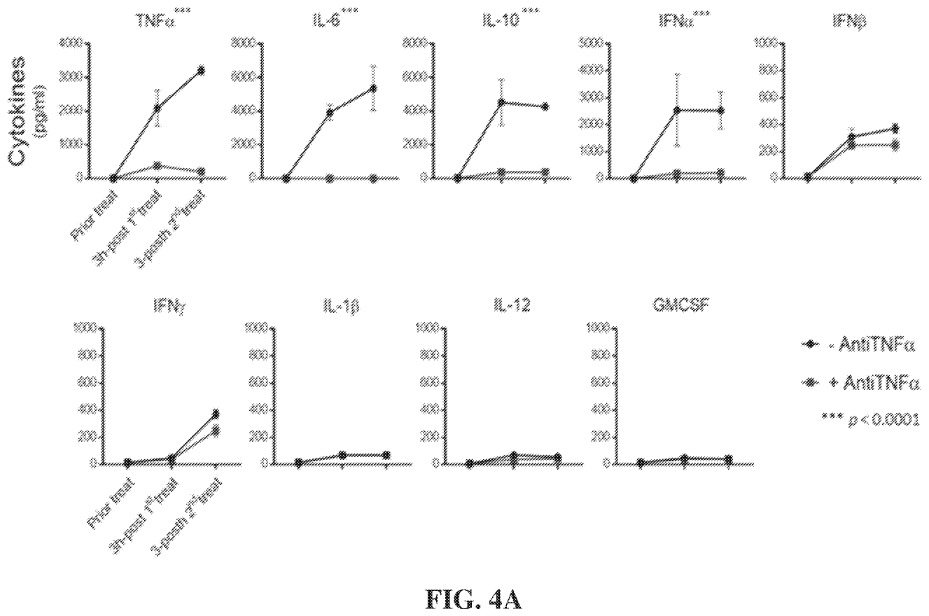

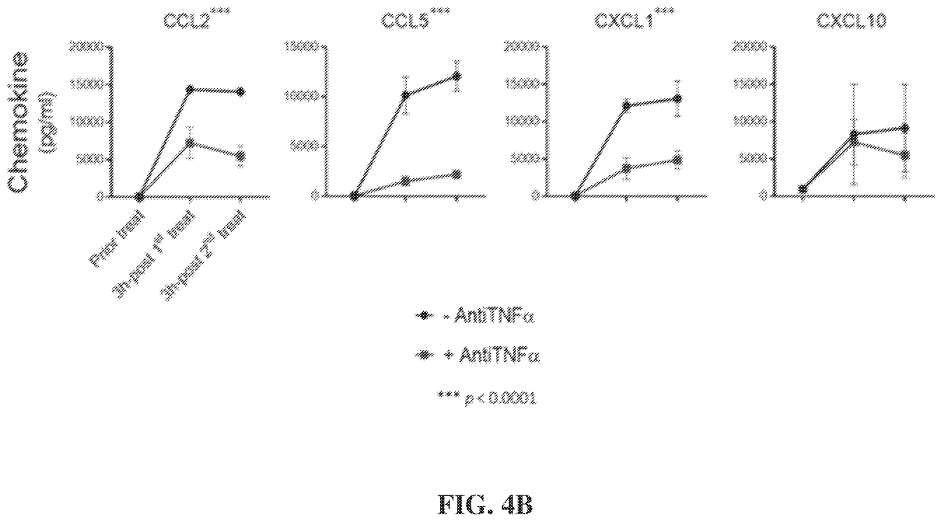

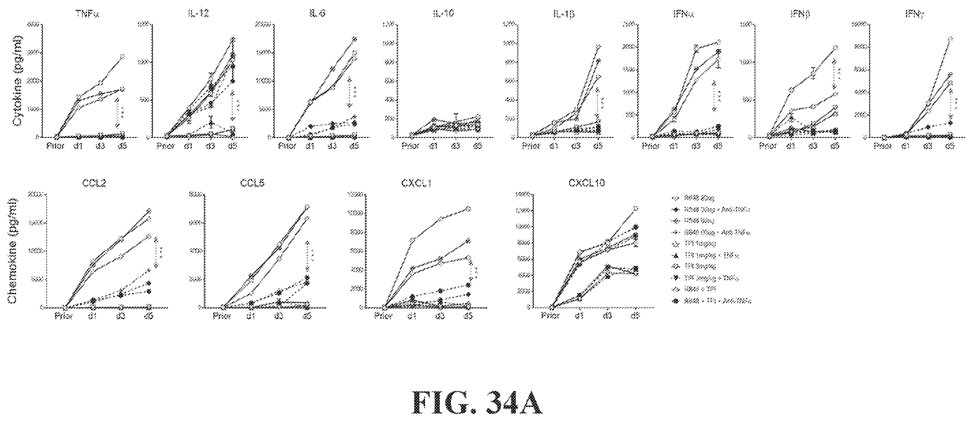

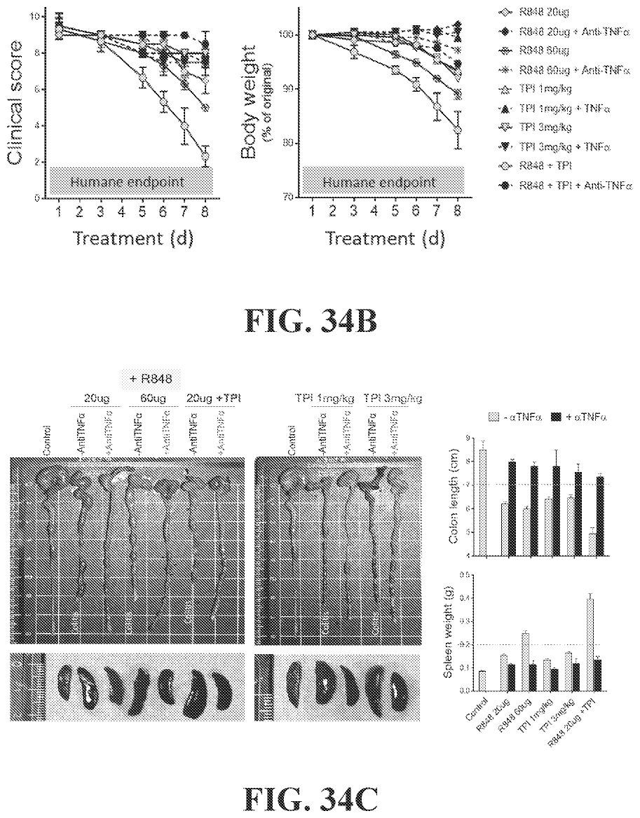

The present application is at least partly based upon a striking finding that combination of a TNFα inhibitor with a pro-inflammatory treatment that activates myeloid cells (e.g., tumor infiltrating macrophages) and one or more inhibitors of the SHP-1 signaling pathway, which potentially inhibits the activation of a “master” inhibitory executor SHP-1, leads to drastic reprogramming of the tumor microenvironment (TME) and bolstering activation of innate and adaptive immune cells to promote anti-cancer immunity with minimal or no systemic toxicity or CRS. Specifically, it was found that the administration of an anti-TNFα neutralizing antibody during the course of aggressive anti-cancer treatment in a preclinical model dramatically improved the degree of systemic toxicity and pro-inflammatory organ damage that was associated with the anti-cancer treatment without compromising treatment efficacy. This finding is particularly striking because the same effect was not seen upon administering an anti-IL-6 neutralizing antibody, wherein CRS and organ damage still occurred. See FIGS. 4A-8, 12-16, and 23. This finding underscores the potential of utilizing TNFα inhibition in order to ameliorate or eliminate adverse events (e.g., CRS or pro-inflammatory organ damage) arising from the administration of SHP-1 pathway inhibition treatment in combination with a myeloid cell (e.g., macrophages or dendritic cells) activating agent. By ameliorating or eliminating these adverse events, the potential of inhibiting SHP-1 directly or via upstream tyrosine kinases, which potentially deplete ITIM phosphorylation and SHP-1 activation, as a combination in tumor immunotherapy can be unlocked in order to achieve safe efficacies in cancer treatment. These findings were further corroborated in treatments that involve a T cell therapy such as a cell based immunotherapy (such as CAR-T cells). It was found that treatment with a TNFα inhibitor in combination with a cell-based immunotherapy either prophylactically, concurrently or within a small window shortly after (e.g., within 3 hours after) the cell therapy prevents the formation of CRS with no effect on anti-tumor activity of therapeutic intervention. See, e.g., FIGS. 34A-34C, 35A-35B, and 36A-36C.

Accordingly, this application provides novel methods that can effectively rewire tumor condition-imposed immunosuppression and license innate and adaptive immunity against cancer while significantly preventing therapeutic-induced toxicity, thereby achieving a remarkable and safe anti-tumor efficacy. This application further provides novel methods that can preserve efficacy of a T cell therapy (e.g., CAR-T therapy) against cancer while significantly preventing therapeutic-induced toxicity, thereby achieving a remarkable and safe anti-tumor efficacy.

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

The term “individual,” “subject,” or “patient” is used synonymously herein to describe an animal, for example a reptile, a bird, a fish, or a mammal, (e.g., a human). An individual includes, but is not limited to, fish, reptile, bird, human, bovine, horse, feline, canine, rodent, or primate. In some embodiments, the individual is human. In some embodiments, an individual suffers from a disease, such as cancer. In some embodiments, the individual is in need of treatment.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or individuals under treatment.

As used herein, the term “intermittent” or “intermittently” in the context of dosing refers to a non-continuous dosing. For example, in some cases, “intermittent” dosing refers to a dosing where the treatment is administered at least two times, and the two administrations are separated by at least one day (i.e., Day 1 and Day 3).

As used herein, the term “cycle” in the context of dosing refers to a time period during which there is at least one administration of a treatment. Day 1 of a cycle is defined as the day when the first administration of a treatment happens during that time period. When there are a few daily consecutive administrations of the treatment, then Day 1 of the cycle is defined as the day when first administration among the few daily consecutive administrations happens. The last day of the cycle is defined as the day before the next non-consecutive administration of the treatment happens. The cycles do not have to have the same length of time. For example, the first cycle can have five days, and the second cycle can have seven days. Each cycle may have different numbers of administrations of the treatment. For example, the first cycle, which may have five days, may have one administration of the treatment, and the second cycle, which may have seven days, may have two administrations of the treatment. Wherein the treatment involves the administration of more than one compound, then each compound can follow the same or different cycles as described above. In some examples, each compound may have cycles that are a combination of the same and different cycles as the cycles of any other compound.

As used herein the term “immunogenic” is the ability to elicit an immune response, e.g., via T-cells, B cells, or both.

As used herein, “treatment” or “treating” is an approach for obtaining beneficial or desired results including clinical results. For purposes of this invention, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread (e.g., metastasis) of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the invention contemplate any one or more of these aspects of treatment.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, by “pharmaceutically acceptable” or “pharmacologically compatible” is meant a material that is not biologically or otherwise undesirable, e.g., the material may be incorporated into a pharmaceutical composition administered to an individual without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

It is understood that embodiments of the application described herein include “consisting of” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

It should be noted that, as used in the specification and the appended claims, the singular forms “a,” “an,” and “the” include plural referents unless the context clearly dictates otherwise.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

### II. Methods of Treatment

The present application in one aspect provides methods of treating a cancer in an individual, comprising administering to the individual a) a myeloid cell activating agent or therapy, and b) a TNFα inhibitor. In some embodiments, the individual being treated has been subject to, is being subject to, or is about to be subject to one or more SHP-1 pathway inhibitor(s) such as any of those described herein. In another aspect is provided a method of treating a cancer in an individual, comprising administering to the individual a TNFα inhibitor and an inhibitor of the SHP-1 pathway, wherein the individual is under an inflammation reaction or has an ongoing infection. In some embodiments, the individual is further administered an immune checkpoint inhibitor and/or a cytokine.

In some embodiments, the method comprises administering both a TNFα inhibitor and a myeloid cell activating agent or therapy into the individual. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method comprises administering a TNFα inhibitor to an individual, wherein the individual is under an inflammation reaction or has an ongoing infection. In some embodiments, the inflammation reaction or ongoing infection promotes a pro-inflammatory immune response in the individual. In some embodiments, the method further comprises administering a SHP-1 inhibitor and/or a tyrosine kinase inhibitor. In some embodiments, the SHP-1 inhibitor and/or the tyrosine kinase inhibitor are administered intermittently. In some embodiments, the SHP-1 inhibitor and/or the tyrosine kinase inhibitor are administered daily. In some embodiments, the method comprises systemically administering the SHP-1 inhibitor and/or the tyrosine kinase inhibitor.

In some embodiments, there is provided a method of treating a cancer (e.g., a solid tumor, e.g., a hematological cancer, e.g., a late-stage cancer) in an individual, comprising administering to the individual a TNFα inhibitor (e.g., a neutralizing antibody) and a myeloid cell activating agent or therapy (e.g., a TLR agonist, e.g., a STING activator, e.g., a radiation therapy), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, and optionally wherein the SHP-1 inhibitor, tyrosine kinase inhibitor, immune checkpoint inhibitor, and/or cytokine or biologically active fragment thereof is administered systemically (e.g., intravenously or subcutaneously). In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the SHP-1 inhibitor, tyrosine kinase inhibitor, immune checkpoint inhibitor, and/or cytokine or biologically active fragment thereof is administered intermittently. In some embodiments, the SHP-4 inhibitor, tyrosine kinase inhibitor, immune checkpoint inhibitor, and/or cytokine or biologically active fragment thereof is administered at an interval of no more than once every two days. In some embodiments, the SHP-1 inhibitor and/or the tyrosine kinase inhibitor is administered no less than two times and no more than 5 times within ten consecutive days (e.g., twice in ten days, three times in ten days, four times in ten days, or five times in ten days). In some embodiments, the SHP-1 inhibitor, tyrosine kinase inhibitor, immune checkpoint inhibitor, and/or cytokine or biologically active fragment thereof is administered simultaneously. In some embodiments, the SHP-1 inhibitor, tyrosine kinase inhibitor, immune checkpoint inhibitor, and/or cytokine or biologically active fragment thereof is administered concurrently. In some embodiments, the SHP-1 inhibitor, tyrosine kinase inhibitor, immune checkpoint inhibitor, and/or cytokine or biologically active fragment thereof is administered simultaneously with the myeloid cell activating agent or therapy and/or the TNFα inhibitor. In some embodiments, the SHP-1 inhibitor, tyrosine kinase inhibitor, immune checkpoint inhibitor, and/or cytokine or biologically active fragment thereof is administered concurrently with the myeloid cell activating agent or therapy and/or the TNFα inhibitor. In some embodiments, the SHP-1 inhibitor, tyrosine kinase inhibitor, immune checkpoint inhibitor, and/or cytokine or biologically active fragment thereof and the myeloid cell activating agent or therapy and/or the TNFα inhibitor are administered sequentially and within 2 weeks (e.g., within 10 days, 7 days, 6 days, 5 days, 4 days, 3 days, 2 days, or the same day). In some embodiments, the SHP-1 inhibitor and/or the tyrosine kinase inhibitor has a half-life of no more than about 10 days (e.g., no more than about 7 days, 5 days, 4 days, or 3 days). In some embodiments, the tyrosine kinase inhibitor is effective in inhibiting more than 50% of the tyrosine kinase activity for no more than about 7 days (e.g., about 5 days, 4 days, or 3 days). In some embodiments, the SHP-1 inhibitor is effective in inhibiting more than 50% of the SHP-1 activity for no more than about 7 days (e.g., about 5 days, 4 days, or 3 days). In some embodiments, the SHP-1 inhibitor and/or the tyrosine kinase inhibitor is selected from the group consisting of: a small molecule, a nucleic acid (e.g., an siRNA, an shRNA, an antisense RNA, a microRNA), a nucleic acid base inhibitor (e.g., a circular RNA inhibitor; see, e.g., Holdt, L. M. et al., Front Physiol 2018; 9:1262), a nucleic acid editing system (e.g., CRISPR, ZFN, or TALENS systems), a peptide agent, a protein agent (e.g., an antibody agent that targets SHP-1 or tyrosine kinase or activated tyrosine kinase), a protein degrading or destabilizing agent, a protein modified with an unnatural amino acid, an antibody directed therapy, an antibody drug conjugate (ADC), and any combination thereof. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises locally (e.g., intratumorally) administering an effective amount of the myeloid cell activating agent or therapy into the individual. In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2).

In some embodiments, there is provided a method of treating a cancer (e.g., a solid tumor, e.g., a hematological cancer, e.g., a late-stage cancer) in an individual, comprising administering to the individual a TNFα inhibitor (e.g., a neutralizing antibody) and a myeloid cell activating agent or therapy (e.g., a TLR agonist, e.g., a STING activator, e.g., a radiation therapy), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, and wherein the method optionally comprises oral, intravenous or subcutaneous administration of the SHP-1 inhibitor, tyrosine kinase inhibitor, immune checkpoint inhibitor, and/or cytokine or biologically active fragment thereof. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the SHP-1 inhibitor, tyrosine kinase inhibitor, immune checkpoint inhibitor, and/or cytokine or biologically active fragment thereof is administered daily for at least 2, 3, 4, 5, 6, or 7 days. In some embodiments, the SHP-1 inhibitor, tyrosine kinase inhibitor, immune checkpoint inhibitor, and/or cytokine or biologically active fragment thereof is administered intermittently. In some embodiments, the method comprises administering the SHP-1 inhibitor and/or the tyrosine kinase inhibitor to the individual at an interval of no more than once every three days for at least twice. In some embodiments, the SHP-1 inhibitor and/or the tyrosine kinase inhibitor is administered twice (e.g., two executive days) every seven to twenty days. In some embodiments, the SHP-1 inhibitor and/or tyrosine kinase inhibitor is administered three times (e.g., three executive days) every ten to twenty days. In some embodiments, the SHP-1 inhibitor and/or the tyrosine kinase inhibitor is administered at an interval of no more than once every two days. In some embodiments, the SHP-1 inhibitor and/or the tyrosine kinase inhibitor is administered no less than two times and no more than 5 times within ten consecutive days (e.g., twice in ten days, three times in ten days, four times in ten days, or five times in ten days). In some embodiments, the SHP-1 inhibitor and/or the tyrosine kinase inhibitor is administered daily. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered simultaneously with an immune checkpoint inhibitor and/or a cytokine or biologically active fragment thereof. In some embodiments, the SHP-1 inhibitor and/or the tyrosine kinase inhibitor is administered sequentially with an immune checkpoint inhibitor and/or a cytokine or biologically active fragment thereof. In some embodiments, the SHP-1 inhibitor, tyrosine kinase inhibitor, immune checkpoint inhibitor, and/or cytokine or biologically active fragment thereof is administered simultaneously with the myeloid cell activating agent or therapy and/or the TNFα inhibitor. In some embodiments, the SHP-1 inhibitor, tyrosine kinase inhibitor, immune checkpoint inhibitor, and/or cytokine or biologically active fragment thereof is administered concurrently with the myeloid cell activating agent or therapy and/or the TNFα inhibitor. In some embodiments, the SHP-1 inhibitor, tyrosine kinase inhibitor, immune checkpoint inhibitor, and/or cytokine or biologically active fragment thereof and the myeloid cell activating agent or therapy and/or the TNFα inhibitor are administered sequentially and within 2 weeks (e.g., within 10 days, 7 days, 6 days, 5 days, 4 days, 3 days, 2 days, or the same day). In some embodiments, the SHP-1 inhibitor and/or the tyrosine kinase inhibitor has a half-life of no more than about 10 days (e.g., no more than about 7 days, 5 days, 4 days, or 3 days). In some embodiments, the SHP-1 inhibitor and/or the tyrosine kinase inhibitor is selected from the group consisting of: a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZFN, or TALENS systems), a peptide agent, a protein agent (e.g., an antibody agent that targets SHP-1 or tyrosine kinase or activated tyrosine kinase), a protein degrading or destabilizing agent, a protein modified with an unnatural amino acid, an antibody directed therapy, an antibody drug conjugate (ADC), and any combination thereof. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of Src, Syk, Hck, Lck, Lyn, and Yes. In some embodiments, the tyrosine kinase inhibitor is selected from the group consisting of: RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises locally (e.g., intratumorally) administering an effective amount of the myeloid cell activating agent or therapy into the individual. In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., a neutralizing antibody) and a myeloid cell activating agent or therapy (e.g., a TLR agonist, e.g., a STING activator, e.g., a radiation therapy), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, and wherein the method comprises orally, intravenous or subcutaneous administration of the SHP-1 inhibitor, tyrosine kinase inhibitor, immune checkpoint inhibitor, and/or cytokine or biologically active fragment thereof. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor and/or the tyrosine kinase inhibitor is administered simultaneously with the myeloid cell activating agent or therapy. In some embodiments, the SHP-1 inhibitor and/or the tyrosine kinase inhibitor is administered simultaneously with the myeloid cell activating agent or therapy and/or the TNFα inhibitor. In some embodiments, the SHP-1 inhibitor and/or the tyrosine kinase inhibitor is administered concurrently with the myeloid cell activating agent or therapy. In some embodiments, the SHP-1 inhibitor and/or the tyrosine kinase inhibitor is administered concurrently with the myeloid cell activating agent or therapy and/or the TNFα inhibitor. In some embodiments, the SHP-1 inhibitor and/or the tyrosine kinase inhibitor and the myeloid cell activating agent or therapy are administered sequentially and within 2 weeks (e.g., within 10 days, 7 days, 6 days, 5 days, 4 days, 3 days, 2 days, or the same day). In some embodiments, the SHP-1 inhibitor and/or the tyrosine kinase inhibitor has a half-life of no more than about 10 days (e.g., no more than about 7 days, 5 days, 4 days, or 3 days). In some embodiments, the SHP-1 inhibitor, the tyrosine kinase inhibitor, and the myeloid cell activating agent or therapy are administered to the individual until the individual undergoes tumor clearance. In some embodiments, the SHP-1 inhibitor, the tyrosine kinase inhibitor, the myeloid cell activating agent or therapy, and/or the TNFα inhibitor are administered intermittently to the individual after tumor clearance. In some embodiments, the SHP-1 inhibitor and/or the tyrosine kinase inhibitor is selected from the group consisting of: a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZFN, or TALENS systems), a peptide agent, a protein agent (e.g., an antibody agent that targets SHP-1 or tyrosine kinase or activated tyrosine kinase), a protein degrading or destabilizing agent, a protein modified with an unnatural amino acid, an antibody directed therapy, an antibody drug conjugate (ADC), and any combination thereof. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method further comprises locally (e.g., intratumorally) administering the myeloid cell activating agent or therapy into the individual. In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising orally, intravenously, subcutaneously, intraperitoneally, and/or intratumorally administering to the individual a SHP-1 inhibitor, a tyrosine kinase inhibitor, and a myeloid cell activating agent or therapy (e.g., a TLR agonist, e.g., a STING activator, e.g., a radiation therapy), optionally wherein the SHP-1 inhibitor and/or the tyrosine kinase inhibitor is effective in inhibiting more than 50% of the SHP-1 and/or tyrosine kinase activity for no more than about 5 days, and optionally wherein the method comprises administering the SHP-1 inhibitor and/or the tyrosine kinase inhibitor to the individual daily, wherein the individual is further administered a TNFα inhibitor and wherein the individual does not develop cytokine release syndrome or pro-inflammatory organ damage. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, there is provided a method of treating a cancer (e.g., a solid tumor, e.g., a hematological cancer, e.g., a late-stage cancer) in an individual, comprising orally, intravenously, subcutaneously, intraperitoneally, and/or intratumorally administering to the individual a SHP-1 inhibitor, a tyrosine kinase inhibitor, and a myeloid cell activating agent or therapy (e.g., a TLR agonist, e.g., a STING activator, e.g., a radiation therapy), optionally wherein the SHP-1 inhibitor and/or the tyrosine kinase inhibitor is effective in inhibiting more than 50% of the SHP-1 and/or tyrosine kinase activity for no more than about 5 days, and optionally wherein the method comprises administering the SHP-1 inhibitor and/or the tyrosine kinase inhibitor to the individual at an interval of no more than once every three days for at least twice (e.g., at least 3, 4, 5, or 6 times), wherein the individual is further administered a TNFα inhibitor and wherein the individual does not develop cytokine release syndrome or pro-inflammatory organ damage. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the SHP-1 inhibitor and/or the tyrosine kinase inhibitor is administered at an interval of no more than twice every seven to twenty days. In some embodiments, the SHP-1 inhibitor and/or the tyrosine kinase inhibitor is administered at an interval of no more than three times every seven to twenty days. In some embodiments, the SHP-1 inhibitor and/or the tyrosine kinase inhibitor is administered for a period of at least fourteen to twenty days at an interval of about 1-3 times every seven to twenty days. In some embodiments, the SHP-1 inhibitor and/or the tyrosine kinase inhibitor is administered at least about 2, 3, 4, 5, or 6 times in a period of about fourteen to about forty days (e.g., about fourteen to about twenty days). In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor and/or the tyrosine kinase inhibitor is administered simultaneously with the myeloid cell activating agent or therapy. In some embodiments, the SHP-1 inhibitor and/or the tyrosine kinase inhibitor is administered simultaneously with the myeloid cell activating agent or therapy and/or the TNFα inhibitor. In some embodiments, the SHP-1 inhibitor and/or the tyrosine kinase inhibitor is administered concurrently with the myeloid cell activating agent or therapy. In some embodiments, the SHP-1 inhibitor and/or the tyrosine kinase inhibitor is administered concurrently with the myeloid cell activating agent or therapy and/or the TNFα inhibitor. In some embodiments, the SHP-1 inhibitor and/or the tyrosine kinase inhibitor and the myeloid cell activating agent or therapy are administered sequentially and within 2 weeks (e.g., within 10 days, 7 days, 6 days, 5 days, 4 days, 3 days, 2 days, or the same day). In some embodiments, the SHP-1 inhibitor and/or the tyrosine kinase inhibitor has a half-life of no more than about 10 days (e.g., no more than about 7 days, 5 days, 4 days, or 3 days). In some embodiments, the SHP-1 inhibitor and/or the tyrosine kinase inhibitor is effective in inhibiting more than 50/6 of the SHP-1 and/or the tyrosine kinase activity for no more than about 7 days (e.g., about 5 days, 4 days, or 3 days). In some embodiments, the SHP-1 inhibitor, the tyrosine kinase inhibitor, and the myeloid cell activating agent or therapy are administered to the individual until the individual undergoes tumor clearance. In some embodiments, the SHP-1 inhibitor, the tyrosine kinase inhibitor, the myeloid cell activating agent or therapy, and/or the TNFα inhibitor are administered intermittently to the individual after tumor clearance. In some embodiments, the SHP-1 inhibitor and/or the tyrosine kinase inhibitor is selected from the group consisting of: a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZFN, or TALENS systems), a peptide agent, a protein agent (e.g., an antibody agent that targets SHP-1 or tyrosine kinase or activated tyrosine kinase), a protein degrading or destabilizing agent, a protein modified with an unnatural amino acid, an antibody directed therapy, an antibody drug conjugate (ADC), and any combination thereof. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of: RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method further comprises locally (e.g., intratumorally) administering the myeloid cell activating agent or therapy into the individual. In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the SHP-1 inhibitor and the tyrosine kinase inhibitor are administered systemically, and the myeloid cell activating agent or therapy is administered intratumorally. In some embodiments, the SHP-1 inhibitor and the tyrosine kinase inhibitor is administered systemically and intratumorally. In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., a neutralizing antibody) and a myeloid cell activating agent or therapy (e.g., a TLR agonist, e.g., a STING activator, e.g., a radiation therapy), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, and wherein the method comprises orally, intravenously, subcutaneously and/or intratumorally administering the SHP-1 inhibitor, tyrosine kinase inhibitor, immune checkpoint inhibitor, and/or cytokine or biologically active fragment thereof and the myeloid cell activating agent or therapy, wherein the SHP-1 inhibitor and/or the tyrosine kinase inhibitor is effective in inhibiting more than 50% of the SHP-1 and/or tyrosine kinase activity for no more than about 5 days (e.g., for no more than 5, 4, or 3 days). In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor and/or the tyrosine kinase inhibitor is administered simultaneously with the myeloid cell activating agent or therapy. In some embodiments, the SHP-1 inhibitor and/or the tyrosine kinase inhibitor is administered simultaneously with the myeloid cell activating agent or therapy and/or the TNFα inhibitor. In some embodiments, the SHP-1 inhibitor and/or the tyrosine kinase inhibitor is administered concurrently with the myeloid cell activating agent or therapy. In some embodiments, the SHP-1 inhibitor and/or the tyrosine kinase inhibitor is administered concurrently with the myeloid cell activating agent or therapy and/or the TNFα inhibitor. In some embodiments, the SHP-1 inhibitor and/or the tyrosine kinase inhibitor and the myeloid cell activating agent or therapy are administered sequentially and within 2 weeks (e.g., within 10 days, 7 days, 6 days, 5 days, 4 days, 3 days, 2 days, or the same day). In some embodiments, the SHP-1 inhibitor and/or the tyrosine kinase inhibitor has a half-life of no more than about 10 days (e.g., no more than about 7 days, 5 days, 4 days, or 3 days). In some embodiments, the SHP-1 inhibitor, the tyrosine kinase inhibitor, and the myeloid cell activating agent or therapy are administered to the individual until the individual undergoes tumor clearance. In some embodiments, the SHP-1 inhibitor, the tyrosine kinase inhibitor, the myeloid cell activating agent or therapy, and/or the TNFα inhibitor are administered intermittently to the individual after tumor clearance. In some embodiments, the SHP-1 inhibitor and/or the tyrosine kinase inhibitor is selected from the group consisting of: a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZFN, or TALENS systems), a peptide agent, a protein agent (e.g., an antibody agent that targets SHP-1 or tyrosine kinase or activated tyrosine kinase), a protein degrading or destabilizing agent, a protein modified with an unnatural amino acid, an antibody directed therapy, an antibody drug conjugate (ADC), and any combination thereof. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method further comprises locally (e.g., intratumorally) administering the myeloid cell activating agent or therapy into the individual. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically, and the myeloid cell activating agent or therapy is administered intratumorally. In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering (e.g., orally, intravenously, subcutaneously, and/or intratumorally) to the individual a TNFα inhibitor (e.g., a neutralizing antibody) and a myeloid cell activating agent or therapy (e.g., a TLR agonist, e.g., a STING activator, e.g., a radiation therapy), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, and wherein the method further comprises administering immune cells (such as any of the immune cells described herein). In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the individual has been subject to, is being subject to, or is about to be subject to a myeloid cell activating agent or therapy (e.g., a TLR agonist, e.g., a radiation therapy). In some embodiments, the individual is under an inflammation reaction or has an ongoing infection. In some embodiments, the immune cells are derived from the same individual. In some embodiments, the immune cells comprise monocytes or macrophages. In some embodiments, the immune cells comprise T cells (e.g., CAR-T cells). In some embodiments, the immune cells comprise NK cells (e.g., CAR-NK cells). In some embodiments, the immune cells comprise neutrophils (e.g., CAR-expressing neutrophils cells). In some embodiments, the immune cells comprise antigen presenting cells (APCs). In some embodiments, the immune cells are engineered to express a chimeric receptor that specifically binds to a tumor antigen. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the SHP-1 inhibitor, the tyrosine kinase inhibitor, the immune checkpoint inhibitor, the cytokine or biologically active fragment thereof, the immune cells, and/or the myeloid cell activating agent or therapy are administered within 7, 6, 5, 4, 3, 2 or 1 days. In some embodiments, the SHP-1 inhibitor and/or the tyrosine kinase inhibitor and the immune cells are administered within 24 hours (e.g., within 12, 8, 4, 2, or 1 hour, or within 30 minutes) of each other. In some embodiments, the SHP-1 inhibitor, the tyrosine kinase inhibitor, the immune checkpoint inhibitor, the cytokine or biologically active fragment thereof, the immune cells, and/or the myeloid cell activating agent or therapy are administered simultaneously. In some embodiments, the SHP-1 inhibitor, the tyrosine kinase inhibitor, the immune checkpoint inhibitor, the cytokine or biologically active fragment thereof, the immune cells, and/or the myeloid cell activating agent or therapy are administered concurrently. In some embodiments, the SHP-1 inhibitor, the tyrosine kinase inhibitor, the immune checkpoint inhibitor, the cytokine or biologically active fragment thereof, the immune cells, and/or the myeloid cell activating agent or therapy are administered sequentially. In some embodiments, the SHP-1 inhibitor, the tyrosine kinase inhibitor, and the myeloid cell activating agent or therapy are administered to the individual until the individual undergoes tumor clearance. In some embodiments, the SHP-1 inhibitor, the tyrosine kinase inhibitor, the myeloid cell activating agent or therapy, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method further comprises locally (e.g., intratumorally) administering the myeloid cell activating agent or therapy into the individual. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically, and the myeloid cell activating agent or therapy is administered intratumorally. In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα neutralizing antibody and a TLR agonist, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, wherein the SHP-1 inhibitor and/or the tyrosine kinase inhibitor is administered at least twice (e.g., at least 3, 4, or 5 times). In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, there is provided a method of treating a cancer (e.g., a solid tumor, e.g., a hematological cancer, e.g., a late-stage cancer) in an individual, comprising administering to the individual a TNFα neutralizing antibody and a TLR agonist, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, wherein the SHP-1 inhibitor and/or the tyrosine kinase inhibitor and the TLR agonist are administered within 24 hours (e.g., within 12, 8, 4, 2, or 1 hour, or within 30 minutes) of each other. In some embodiments, the method comprises administering the SHP-1 inhibitor and/or the tyrosine kinase inhibitor daily (e.g., every day for at least 7 days). In some embodiments, the method comprises administering the SHP-1 inhibitor and/or the tyrosine kinase inhibitor to the individual at an interval of no more than once every three days for at least twice. In some embodiments, the method comprises administering the SHP-1 inhibitor and/or the tyrosine kinase inhibitor to the individual for at least two cycles, wherein the SHP-1 inhibitor and/or the tyrosine kinase inhibitor is administered for at least once (e.g., at least twice or three time) in each cycle and wherein each cycle has about three to about twenty days. In some embodiments, the SHP-1 inhibitor and/or the tyrosine kinase inhibitor is administered systemically (e.g., intravenously or subcutaneously) and/or locally (e.g., intratumorally). In some embodiments, the TLR agonist and/or the TNFα neutralizing antibody is administered systemically (e.g., orally, intravenously, subcutaneously, or intraperitoneally) or locally (e.g., intratumorally). In some embodiments, the TLR agonist is administered daily for at least 2, 3, 4, 5, 6, or 7 days. In some embodiments, the TLR agonist is administered intermittently. In some embodiments, the TNFα neutralizing antibody is administered at least once a week, once every five days, once every three days, or daily. In some embodiments, the TNFα neutralizing antibody is administered intermittently. In some embodiments, the TNFα neutralizing antibody is administered to the individual for at least two cycles, wherein each cycle has about three to about seven days. In some embodiments, the SHP-1 inhibitor and/or the tyrosine kinase inhibitor and the TLR agonist are administered simultaneously, concurrently, or sequentially. In some embodiments, the TLR agonist activates TLR1 or TLR2, optionally wherein the TLR agonist comprises a triacylated lipoprotein, a peptidoglycan, zymosan, and/or Pam3CSK4. In some embodiments, the TLR agonist activates any one of TLR2, TLR3, TLR4, TLR5, and TLR6, optionally wherein the TLR agonist comprises a diacylated lipopeptide, a hot shock protein, HMGB1, uric acid, fibronectin, and/or ECM protein. In some embodiments, the TLR agonist activates TLR2, optionally wherein the TLR agonist comprises Pam3Cys, SMP-105, and/or CBLB612. In some embodiments, the TLR agonist activates TLR3, optionally wherein the TLR agonist comprises dsRNA, Poly I:C, PolyICIC, Poly-IC12U, IPH302, ARNAX, and/or MPLA. In some embodiments, the TLR agonist activates TLR4, optionally wherein the TLR agonist comprises LPS, lipoteichoic acid beta-defensin 2, fibronectin EDA, HMGB1, snapin, tenascin C, OK-432, AS04, and/or GLA-SE. In some embodiments, the TLR agonist activates TLR5, optionally wherein the TLR agonist comprises flagellin, CBLB502, and/or M-VM3. In some embodiments, the TLR agonist activates TLR6. In some embodiments, the TLR agonist activates TLR7 or TLR8, optionally wherein the TLR agonist comprises ssRNA, CpG-A, poly G10, and/or poly G3. In some embodiments, the TLR agonist activates TLR7, optionally wherein the TLR agonist comprises bistriazolyl and/or R848. In some embodiments, the TLR agonist activates TLR8, optionally wherein the TLR agonist comprises VTX1463 and/or R848. In some embodiments, the TLR agonist activates TLR9, optionally wherein the TLR agonist comprises unmethylated CpG DNA, CpG (e.g., CpG-7909, KSK-CpG, CpG-1826), MGN1703, dsSLIM, IMO2055, SD101, and/or ODN M362. In some embodiments, the TLR agonist activates TLR10, optionally wherein the TLR agonist comprises Pam3CSK4. In some embodiments, the TLR agonist activates TLR11, optionally wherein the TLR agonist comprises Toxoplasma gondii profilin. In some embodiments, the TLR agonist activates TLR12. In some embodiments, the TLR agonist activates TLR13, optionally wherein the TLR agonist comprises VSV. In some embodiments, the TLR agonist activates TLR1, TLR2, TLR3, TLR4, TLR7, TLR8, and/or TLR9. In some embodiments, the TLR agonist activates TLR9, TLR4 and TLR7/8. In some embodiments, the TLR agonist comprises CpG, polyI:C and/or R848. In some embodiments, the TLR agonist comprises CpG, polyI:C and R848, for example at 1:1:1 ratio. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the TLR agonist are administered to the individual until the individual undergoes tumor clearance. In some embodiments, the SHP-1 inhibitor, the tyrosine kinase inhibitor, the TLR agonist, and/or the TNFα neutralizing antibody are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα neutralizing antibody. In some embodiments, the TNFα neutralizing antibody is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα neutralizing antibody is administered simultaneously with the administration of the myeloid cell activating agent or therapy and/or the inhibitor of the SHP-1 pathway. In some embodiments, the TNFα neutralizing antibody is administered concurrently with the administration of the myeloid cell activating agent or therapy and/or the inhibitor of the SHP-1 pathway. In some embodiments, the TNFα neutralizing antibody is administered sequentially to (e.g., prior to or after) the administration of the myeloid cell activating agent or therapy and/or the inhibitor of the SHP-1 pathway. In some embodiments, the TNFα neutralizing antibody is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method further comprises locally (e.g., intratumorally) administering the TLR agonist into the individual. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically, and the TLR agonist is administered intratumorally. In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα neutralizing antibody and a TLR agonist, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the TLR agonist activates one or more TLRs selected from the group consisting of TLR9, TLR4, TLR7 and TLR8. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the TLR agonist and/or the TNFα neutralizing antibody is administered systemically (e.g., orally, intravenously, subcutaneously, or intraperitoneally) or locally (e.g., intratumorally). In some embodiments, the TLR agonist is administered daily for at least 2, 3, 4, 5, 6, or 7 days. In some embodiments, the TLR agonist is administered intermittently. In some embodiments, the TNFα neutralizing antibody is administered at least once a week, once every five days, once every three days, or daily. In some embodiments, the TNFα neutralizing antibody is administered intermittently. In some embodiments, the TNFα neutralizing antibody is administered to the individual for at least two cycles, wherein each cycle has about three to about seven days. In some embodiments, the SHP-1 inhibitor and/or the tyrosine kinase inhibitor and the TLR agonist are administered within the same day. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the SHP-1 inhibitor and/or the tyrosine kinase inhibitor and/or the TLR agonist are administered at least twice (e.g., at least three, four, five or six times). In some embodiments, the SHP-1 inhibitor and/or the tyrosine kinase inhibitor and the TLR agonist are administered at least two cycles (e.g., at least three cycles), optionally wherein the SHP-1 inhibitor and/or the tyrosine kinase inhibitor and the TLR agonist are administered within the same day for at least two consecutive days (e.g., at least three consecutive days) in each cycle. In some embodiments, the SHP-1 inhibitor and/or the tyrosine kinase inhibitor and the TLR agonist are administered simultaneously, concurrently, or sequentially. In some embodiments, each cycle has about seven to about twenty days. In some embodiments, the TLR agonist activates a TLR on a macrophage, optionally wherein the TLR comprises TLR9. In some embodiments, the TLR agonist activates at least two TLRs (e.g., TLR4, TLR7, TLR8, or TLR9). In some embodiments, the TLR agonist activates at least three TLRs (e.g., TLR9, TLR4 and TLR7/8). In some embodiments, the TLR agonist comprises CpG, polyI:C and/or R848. In some embodiments, the TLR agonist comprises CpG, polyI:C and R848, for example at 1:1:1 ratio. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the TLR agonist are administered to the individual until the individual undergoes tumor clearance. In some embodiments, the SHP-1 inhibitor, the tyrosine kinase inhibitor, the TLR agonist, and/or the TNFα neutralizing antibody are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα neutralizing antibody. In some embodiments, the TNFα neutralizing antibody is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα neutralizing antibody is administered simultaneously with the administration of the myeloid cell activating agent or therapy and/or the inhibitor of the SHP-1 pathway. In some embodiments, the TNFα neutralizing antibody is administered concurrently with the administration of the myeloid cell activating agent or therapy and/or the inhibitor of the SHP-1 pathway. In some embodiments, the TNFα neutralizing antibody is administered sequentially to (e.g., prior to or after) the administration of the myeloid cell activating agent or therapy and/or the inhibitor of the SHP-1 pathway. In some embodiments, the TNFα neutralizing antibody is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method further comprises locally (e.g., intratumorally) administering the TLR agonist into the individual. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically, and the TLR agonist is administered intratumorally. In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor and a STING activator (e.g., cGAMP), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the SHP-1 inhibitor and/or the tyrosine kinase inhibitor is administered at least twice (at least three, four, five, or six times). In some embodiments, there is provided a method of treating a cancer (e.g., a solid tumor, e.g., a hematological cancer, e.g., a late-stage cancer) in an individual, comprising administering to the individual a TNFα inhibitor and a STING activator (e.g., cGAMP), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the SHP-1 inhibitor and/or the tyrosine kinase inhibitor and the STING activator are administered within 24 hours (e.g., within 12, 8, 4, 2, or 1 hour, or within 30 minutes) of each other. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual at an interval of no more than once every three days for at least twice. In some embodiments, the method comprises administering the SHP-1 inhibitor and/or the tyrosine kinase inhibitor to the individual for at least two cycles, wherein the SHP-1 inhibitor and/or the tyrosine kinase inhibitor is administered for at least once (e.g., at least twice or three time) in each cycle and wherein each cycle has about three to about twenty days. In some embodiments, the SHP-1 inhibitor and/or the tyrosine kinase inhibitor is administered systemically (e.g., intravenously or subcutaneously) and/or locally (e.g., intratumorally). In some embodiments, the SHP-1 inhibitor and/or the tyrosine kinase inhibitor and the STING activator are administered sequentially, simultaneously, or concurrently. In some embodiments, the STING activator is a cyclic-guanosine monophosphate-adenosine monophosphate (cGAMP, e.g., 3′3′ cGAMP, e.g., 2′3′ cGAMP), a bacterial vector (e.g., SYNB1891, STACT-TREX-1), a CDN compounds (e.g., ADU-S100, BI-STING, BMS-986301, GSK532, JNJ-4412, MK-1454, SB11285, 3′3′-cyclic AIMP), a non-CDN small molecule (e.g., ALG-031048, E7755, JNJ-'6196, MK-2118, MSA-1, MSA-2, SNX281, SR-717, TAK676, TTI-10001), a nanovaccine (e.g., PC7A NP, cCAMP-NP, ONM-500) or an antibody-drug conjugate (e.g., XMT-2056, CRD-5500). In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the STING activator are administered to the individual until the individual undergoes tumor clearance. In some embodiments, the SHP-1 inhibitor, the tyrosine kinase inhibitor, the STING activator, and/or the TNFα neutralizing antibody are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα neutralizing antibody. In some embodiments, the TNFα neutralizing antibody is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα neutralizing antibody is administered simultaneously with the administration of the myeloid cell activating agent or therapy and/or the inhibitor of the SHP-1 pathway. In some embodiments, the TNFα neutralizing antibody is administered concurrently with the administration of the myeloid cell activating agent or therapy and/or the inhibitor of the SHP-1 pathway. In some embodiments, the TNFα neutralizing antibody is administered sequentially to (e.g., prior to or after) the administration of the myeloid cell activating agent or therapy and/or the inhibitor of the SHP-1 pathway. In some embodiments, the TNFα neutralizing antibody is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method further comprises locally (e.g., intratumorally) administering the STING activator into the individual. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically, and the STING activator is administered intratumorally. In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and a radiation therapy, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, and optionally wherein the method comprises administering the SHP-1 inhibitor and/or the tyrosine kinase inhibitor to the individual for at least two cycles, wherein the SHP-1 inhibitor and/or the tyrosine kinase inhibitor is administered for at least once in each cycle and wherein each cycle has about three to about twenty days. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, I hour, 30 minutes) the administration of the other one or more agents described above. In some embodiments, the radiation therapy and/or the TNFα inhibitor is administered systemically (e.g., orally, intravenously, subcutaneously, or intraperitoneally) or locally (e.g., intratumorally). In some embodiments, the radiation therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the method comprises administering the SHP-1 inhibitor and/or the tyrosine kinase inhibitor to the individual at an interval of no more than once every three days for at least twice. In some embodiments, days, the SHP-1 inhibitor and/or the tyrosine kinase inhibitor is administered at least three times. In some embodiments, the SHP-1 inhibitor and/or the tyrosine kinase inhibitor is administered systemically (e.g., intravenously, e.g., subcutaneously) and/or locally (e.g., intratumorally). In some embodiments, the SHP-1 inhibitor and/or the tyrosine kinase inhibitor and the radiation therapy are administered within 24 hours (e.g., within 12, 8, 4, 2, or 1 hour, or within 30 minutes) of each other. In some embodiments, the radiation therapy comprises irradiation at site of the cancer to be treated. In some embodiments, the radiation therapy comprises irradiation at a site that is different from the site of the cancer to be treated. In some embodiments, the dose of the radiation therapy is insufficient to kill tumor cells. In some embodiments, the radiation therapy is selected from the group consisting of external-beam radiation therapy, internal radiation therapy (brachytherapy), intraoperative radiation therapy (IORT), systemic radiation therapy, radioimmunotherapy, and administration of radiosensitizers and radioprotectors. In some embodiments, the radiation therapy is external-beam radiation therapy, optionally comprising three-dimensional conformal radiation therapy (3D-RT), intensity modulated radiation therapy (IMRT), photon beam therapy, image-guided radiation therapy (IGRT), and sterotactic radiation therapy (SRT). In some embodiments, the radiation therapy is brachytherapy, optionally comprising interstitial brachytherapy, intracavitary brachytherapy, intraluminal radiation therapy, and radioactively tagged molecules given intravenously. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the radiation therapy are administered to the individual until the individual undergoes tumor clearance. In some embodiments, the SHP-1 inhibitor, the tyrosine kinase inhibitor, the radiation therapy, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα neutralizing antibody and a radiation therapy, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, wherein the radiation therapy comprises irradiation at a site that is different from the site of the cancer to be treated. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the radiation therapy and/or the TNFα inhibitor is administered systemically (e.g., orally, intravenously, subcutaneously, or intraperitoneally) or locally (e.g., intratumorally). In some embodiments, the radiation therapy is administered intermittently. In some embodiments, the TNFα neutralizing antibody is administered at least once a week, once every five days, once every three days, or daily. In some embodiments, the TNFα neutralizing antibody is administered intermittently. In some embodiments, the TNFα neutralizing antibody is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the SHP-1 inhibitor and/or the tyrosine kinase inhibitor is administered at least twice (at least three, four, five, or six times). In some embodiments, the method comprises administering the SHP-1 inhibitor and/or the tyrosine kinase inhibitor to the individual at an interval of no more than once every three days for at least twice. In some embodiments, the method comprises administering the SHP-1 inhibitor and/or the tyrosine kinase inhibitor to the individual for at least two cycles, wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered systemically (e.g., intravenously, e.g., subcutaneously) and/or locally (e.g., intratumorally). In some embodiments, the SHP-1 inhibitor and/or the tyrosine kinase inhibitor and the radiation therapy are administered within 24 hours (e.g., within 12, 8, 4, 2, or 1 hour, or within 30 minutes) of each other. In some embodiments, the radiation therapy comprises irradiation at site of the cancer to be treated. In some embodiments, the radiation therapy comprises irradiation at a site that is different from the site of the cancer to be treated. In some embodiments, the dose of the radiation therapy is insufficient to kill tumor cells. In some embodiments, the radiation therapy is selected from the group consisting of external-beam radiation therapy, internal radiation therapy (brachytherapy), intraoperative radiation therapy (IORT), systemic radiation therapy, radioimmunotherapy, and administration of radiosensitizers and radioprotectors. In some embodiments, the radiation therapy is external-beam radiation therapy, optionally comprising three-dimensional conformal radiation therapy (3D-RT), intensity modulated radiation therapy (IMRT), photon beam therapy, image-guided radiation therapy (IGRT), and sterotactic radiation therapy (SRT). In some embodiments, the radiation therapy is brachytherapy, optionally comprising interstitial brachytherapy, intracavitary brachytherapy, intraluminal radiation therapy, and radioactively tagged molecules given intravenously. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the radiation therapy are administered to the individual until the individual undergoes tumor clearance. In some embodiments, the SHP-1 inhibitor, the tyrosine kinase inhibitor, the radiation therapy, and/or the TNFα neutralizing antibody are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα neutralizing antibody. In some embodiments, the TNFα neutralizing antibody is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα neutralizing antibody is administered simultaneously with the administration of the myeloid cell activating agent or therapy and/or the inhibitor of the SHP-1 pathway. In some embodiments, the TNFα neutralizing antibody is administered concurrently with the administration of the myeloid cell activating agent or therapy and/or the inhibitor of the SHP-1 pathway. In some embodiments, the TNFα neutralizing antibody is administered sequentially to (e.g., prior to or after) the administration of the myeloid cell activating agent or therapy and/or the inhibitor of the SHP-1 pathway. In some embodiments, the TNFα neutralizing antibody is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering a TNFα neutralizing antibody and a radiation therapy, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the SHP-1 inhibitor and/or the tyrosine kinase inhibitor and the radiation therapy are administered within the same day. In some embodiments, the SHP-1 inhibitor and/or the tyrosine kinase inhibitor and/or the radiation therapy are administered at least twice (e.g., at least three, four, five or six times). In some embodiments, the SHP-1 inhibitor and/or the tyrosine kinase inhibitor and the radiation therapy are administered at least two cycles (e.g., at least three cycles), optionally wherein the SHP-1 inhibitor and/or the tyrosine kinase inhibitor and the radiation therapy are administered within the same day for at least two consecutive days (e.g., at least three consecutive days) in each cycle. In some embodiments, each cycle has about seven to about twenty days. In some embodiments, the SHP-1 inhibitor and/or the tyrosine kinase inhibitor and the radiation therapy are administered within 24 hours (e.g., within 12, 8, 4, 2, or 1 hour, or within 30 minutes) of each other. In some embodiments, the radiation therapy comprises irradiation at site of the cancer to be treated. In some embodiments, the radiation therapy comprises irradiation at a site that is different from the site of the cancer to be treated. In some embodiments, the dose of the radiation therapy is insufficient to kill tumor cells. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the radiation therapy are administered to the individual until the individual undergoes tumor clearance. In some embodiments, the SHP-1 inhibitor, the tyrosine kinase inhibitor, the radiation therapy, and/or the TNFα neutralizing antibody are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα neutralizing antibody. In some embodiments, the TNFα neutralizing antibody is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα neutralizing antibody is administered simultaneously with the administration of the myeloid cell activating agent or therapy and/or the inhibitor of the SHP-1 pathway. In some embodiments, the TNFα neutralizing antibody is administered concurrently with the administration of the myeloid cell activating agent or therapy and/or the inhibitor of the SHP-1 pathway. In some embodiments, the TNFα neutralizing antibody is administered sequentially to (e.g., prior to or after) the administration of the myeloid cell activating agent or therapy and/or the inhibitor of the SHP-1 pathway. In some embodiments, the TNFα neutralizing antibody is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and a PAMP/DAMP activator, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the SHP-1 inhibitor and/or the tyrosine kinase inhibitor is administered at least twice (at least three, four, five, or six times). In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and a PAMP/DAMP activator, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the SHP-1 inhibitor and/or the tyrosine kinase inhibitor and the PAMP/DAMP activator are administered within 24 hours (e.g., within 12, 8, 4, 2, or 1 hour, or within 30 minutes) of each other. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the PAMP/DAMP activator and/or the TNFα inhibitor is administered systemically (e.g., orally, intravenously, subcutaneously, or intraperitoneally) or locally (e.g., intratumorally). In some embodiments, the PAMP/DAMP activator is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the method comprises administering the SHP-1 inhibitor and/or the tyrosine kinase inhibitor to the individual at an interval of no more than once every three days for at least twice. In some embodiments, the method comprises administering the SHP-1 inhibitor and/or the tyrosine kinase inhibitor to the individual for at least two cycles, wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered systemically (e.g., intravenously, e.g., subcutaneously) and/or locally (e.g., intratumorally). In some embodiments, the myeloid cell activating agent or therapy is a PAMP activator. In some embodiments, the PAMP activator is triacyl lipopeptides, LPS, lipoprotein, peptidoglycan, zymosan, lipoteichoic acid, trypanosomal phospholipids, Pam3Cys porins, lipoarabinomannan, double-stranded RNA, poly(I:C), trepanosomal lipids, taxol, Pseudomonas exoenzyme S, RSV F protein, MMTV envelope protein, flagellin, diacyl lipopeptides, single-stranded RNA, imiquimod, single-stranded RNA, resquimod, bacterial/viral DNA, CpG DNA, ureobacteria, or toxoplasma LPS. In some embodiments, the myeloid cell activating agent or therapy is a DAMP activator. In some embodiments, the DAMP activator is defensins, HSP60, HSP70, messenger RNA, low-molecular-weight hyaluronic acid, fibrinogen, fibronectin, fx1-defensin, heparan sulfate, HSP60, HSP70, HSP90, HMGB1, or unmethylated CpG DNA. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the PAMP/DAMP activator are administered to the individual until the individual undergoes tumor clearance. In some embodiments, the SHP-1 inhibitor, the tyrosine kinase inhibitor, the PAMP/DAMP activator, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method further comprises locally (e.g., intratumorally) administering the PAMP/DAMP activator into the individual. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically, and the PAMP/DAMP activator is administered intratumorally. In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., a neutralizing antibody) and a myeloid cell activating agent or therapy (e.g., a TLR agonist, e.g., a STING activator, e.g., a radiation therapy), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the SHP-1 inhibitor and/or the tyrosine kinase inhibitor is administered at least twice (at least three, four, five, or six times). In some embodiments, there is provided a method of treating a cancer (e.g., a solid tumor, e.g., a hematological cancer, e.g., a late-stage cancer) in an individual, comprising administering to the individual a TNFα inhibitor (e.g., a neutralizing antibody) and a myeloid cell activating agent or therapy (e.g., a TLR agonist, e.g., a STING activator, e.g., a radiation therapy), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, wherein the SHP-1 inhibitor and/or the tyrosine kinase inhibitor and the immune checkpoint inhibitor are administered within 24 hours (e.g., within 12, 8, 4, 2, or 1 hour, or within 30 minutes) of each other. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the checkpoint inhibitor targets LAG-3, TIM-3, B7-H3, B7-H4, A2aR, CD73, NKG2A, PVRIG/PVRL2, CEACAM1, CEACAM 5/6, FAK, CCL2/CCR2, LIF, CD47/SIRPα, CSF-1 (M-CSF)/CSF-1R, IL-1/IL-1R3 (IL-1RAP), IL-8, SEMA4D, Ang-2, CLEVER-1, Axl, or phosphatidylserine. In some embodiments, the checkpoint inhibitor comprises or is lipilimumab, Cemiplimab, Nivolumab, Pembrolizumab, Atezolizumab, Avelumab, Durvalumab, LAG525 (IMP701), REGN3767, BI 754,091, tebotelimab (MGD013), eftilagimod alpha (IMP321), FS118, MBG453, Sym023, TSR-022, MGC018, FPA150, EOS100850, AB928, CPI-006, Monalizumab, COM701, CM24, NEO-201, Defactinib, PF-04136309, MSC-1, Hu5F9-G4 (5F9), ALX148, TTI-662, RRx-001, Lanotuzumab (MCS110), LY3022855, SNDX-6352, Emactuzumab (RG7155), Pexidartinib (PLX3397), CAN04, Canakinumab (ACZ885), BMS-986253, Pepinemab (VX15/2503), Trebananib, FP-1305, Enapotamab vedotin(EnaV), or Bavituximab. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the myeloid cell activating agent or therapy are administered to the individual until the individual undergoes tumor clearance. In some embodiments, the SHP-1 inhibitor, the tyrosine kinase inhibitor, the myeloid cell activating agent or therapy, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method further comprises locally (e.g., intratumorally) administering the myeloid cell activating agent or therapy into the individual. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically, and the myeloid cell activating agent or therapy is administered intratumorally. In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., a neutralizing antibody) and a pro-inflammatory cytokine (e.g., IL-1β, IL-18, and/or IL-6), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the SHP-1 inhibitor and/or the tyrosine kinase inhibitor is administered at least twice (at least three, four, five, or six times). In some embodiments, there is provided a method of treating a cancer (e.g., a solid tumor, e.g., a hematological cancer, e.g., a late-stage cancer) in an individual, comprising administering to the individual a TNFα inhibitor (e.g., a neutralizing antibody) and a pro-inflammatory cytokine (e.g., IL-1β, IL-18, and/or IL-6), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, wherein the SHP-1 inhibitor and/or the tyrosine kinase inhibitor and the pro-inflammatory cytokine are administered within 24 hours (e.g., within 12, 8, 4, 2, or 1 hour, or within 30 minutes) of each other. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the pro-inflammatory cytokine promotes the M1 macrophages, dendritic cells (e.g., intratumoral), B cells (e.g., intratumoral), antigen presenting cells, etc. In some embodiments, the pro-inflammatory cytokine comprises or is a TNF family member, IFNγ, and/or GM-CSF. In some embodiments, the pro-inflammatory cytokine comprises IFNγ. In some embodiments, the pro-inflammatory cytokine comprises IL-1. In some embodiments, the pro-inflammatory cytokine comprises any member of the TNF family other than TNFα. In some embodiments, the pro-inflammatory cytokine comprises IL-6. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the pro-inflammatory cytokine are administered to the individual until the individual undergoes tumor clearance. In some embodiments, the SHP-1 inhibitor, the tyrosine kinase inhibitor, the pro-inflammatory cytokine, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method further comprises locally (e.g., intratumorally) administering the pro-inflammatory cytokine into the individual. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP1 inhibitor and the tyrosine kinase inhibitor is administered systemically, and the pro-inflammatory cytokine is administered intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and a chemotherapeutic agent (e.g., azathioprine), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the SHP-1 inhibitor and/or the tyrosine kinase inhibitor is administered at least twice (at least three, four, five, or six times). In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and a chemotherapeutic agent (e.g., azathioprine), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, and wherein the SHP-1 inhibitor and/or the tyrosine kinase inhibitor and the chemotherapy are administered within 24 hours (e.g., within 12, 8, 4, 2, or 1 hour, or within 30 minutes) of each other. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the chemotherapeutic agent is an alkylating agent. In some embodiments, the alkylating agent is selected from the group consisting of nitrogen mustard (e.g., endamustine, cyclophosphamide, ifosfamide), nitrosoureas (e.g., carmustine, lomustine), platinum analogs (e.g., carboplatin, cisplatin, oxaliplatin), triazenes (e.g., dacarbazine, procarbazine, temozolamide), alkyl sulfonate (e.g., busulfan), and ethyleneimine (e.g., thiotepa). In some embodiments, the chemotherapeutic agent is an antimetabolite. In some embodiments, the antimetabolite is selected from the group consisting of icytidine analogs (e.g., azacitidine, decitabine, cytarabine, gemcitabine), folate antagonists (e.g., methotrexate, pemetrexed), purine analogs (e.g., cladribine, clofarabine, nelarabine), pyrimidine analogs (e.g., fluorouracil (5-FU), capecitabine (prodrug of 5-FU)). In some embodiments, the chemotherapeutic agent is an antimicrotubular agent. In some embodiments, the antimicrotubular agent is selected from the group consisting of topoisomerase II inhibitors (e.g., anthracyclines, doxorubicin, daunorubicin, idarubicin, mitoxantrone), topoisomerase I inhibitors (e.g., irinotecan, topotecan), taxanes (e.g., paclitaxel, docetaxel, cabazitaxel), vinca alkaloids (e.g., vinblastine, vincristine, vinorelbine), antibiotics (e.g., actinomycin D, bleomycin, daunomycin). In some embodiments, the chemotherapeutic agent is hydroxyurea, tretinoin, arsenic trioxide, or a proteasome inhibitor (e.g., bortezomib). In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the chemotherapeutic agent are administered to the individual until the individual undergoes tumor clearance. In some embodiments, the SHP-1 inhibitor, the tyrosine kinase inhibitor, the chemotherapeutic agent, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method further comprises locally (e.g., intratumorally) administering the chemotherapeutic agent into the individual. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP1 inhibitor and the tyrosine kinase inhibitor is administered systemically, and the chemotherapeutic agent is administered intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and a cancer vaccine, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the SHP-1 inhibitor and/or the tyrosine kinase inhibitor is administered at least twice. In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and a cancer vaccine, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, wherein the SHP-1 inhibitor and/or the tyrosine kinase inhibitor and the cancer vaccine are administered within 24 hours (e.g., within 12, 8, 4, 2, or 1 hour, or within 30 minutes) of each other. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the cancer vaccine comprises a cell-based vaccine, a peptide-based vaccine, a viral-based vaccine, and/or a nucleic acid-based vaccine. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the cancer vaccine are administered to the individual until the individual undergoes tumor clearance. In some embodiments, the SHP-1 inhibitor, the tyrosine kinase inhibitor, the cancer vaccine, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method further comprises locally (e.g., intratumorally) administering the cancer vaccine into the individual. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP1 inhibitor and the tyrosine kinase inhibitor is administered systemically, and the cancer vaccine is administered intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and an oncolytic virus, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the SHP-1 inhibitor and/or the tyrosine kinase inhibitor is administered at least twice (at least three, four, five, or six times). In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and a oncolytic virus, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, wherein the SHP-1 inhibitor and/or the tyrosine kinase inhibitor and the oncolytic virus are administered within 24 hours (e.g., within 12, 8, 4, 2, or 1 hour, or within 30 minutes) of each other. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the oncolytic virus comprises or is an adenovirus (e.g., ONYX-15, LOAd703 virus), a protoparvovirus, a parvovirus (e.g., H-1PV), a vaccinia virus (VACV), a Reovirus (e.g., Reolysin), or a Herpes simplex virus (HSV, e.g., HSV-1, HSV-2, G207, L1BR1, HF10, T-VEC, Orien X010). In some embodiments, the oncolytic viruses comprise JX-593, Coxsackievirus A21 (CVA21), maraba virus or its MG1 variant, DNX2440 adenovirus, fowl pox virus, or Sendai virus. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the oncolytic virus are administered to the individual until the individual undergoes tumor clearance. In some embodiments, the SHP-1 inhibitor, the tyrosine kinase inhibitor, the oncolytic virus, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method further comprises locally (e.g., intratumorally) administering the oncolytic virus into the individual. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP1 inhibitor and the tyrosine kinase inhibitor is administered systemically, and the oncolytic virus is administered intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and a sound treatment (e.g., high intensity focused ultrasound (HIFU), e.g., low intensity focused ultrasound (LIPUS)), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the SHP-1 inhibitor and/or the tyrosine kinase inhibitor is administered at least twice (at least three, four, five, or six times). In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and a sound treatment (e.g., high intensity focused ultrasound (HIFU), e.g., low intensity focused ultrasound (LIPUS)), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, wherein the SHP-1 inhibitor and/or the tyrosine kinase inhibitor and the sound treatment are administered within 24 hours (e.g., within 12, 8, 4, 2, or 1 hour, or within 30 minutes) of each other. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the SHP-1 inhibitor and/or the tyrosine kinase inhibitor is administered systemically (e.g., intravenously, e.g., subcutaneously) and/or locally (e.g., intratumorally). In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the sound treatment are administered to the individual until the individual undergoes tumor clearance. In some embodiments, the SHP-1 inhibitor, the tyrosine kinase inhibitor, the sound treatment, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically, and the method comprises administering the sound treatment at the site of the cancer to be treated. In some embodiments, the SHP-1 inhibitor and/or the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and a magnetic therapy (e.g., pulsed magnetic field, e.g., static magnetic field), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the SHP-1 inhibitor and/or the tyrosine kinase inhibitor is administered at least twice (at least three, four, five, or six times). In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and a magnetic therapy (e.g., pulsed magnetic field, e.g., static magnetic field), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, wherein the SHP-1 inhibitor and/or the tyrosine kinase inhibitor and the magnetic therapy are administered within 24 hours (e.g., within 12, 8, 4, 2, or 1 hour, or within 30 minutes) of each other. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the SHP-1 inhibitor and/or the tyrosine kinase inhibitor is administered systemically (e.g., intravenously, e.g., subcutaneously) and/or locally (e.g., intratumorally). In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the magnetic treatment are administered to the individual until the individual undergoes tumor clearance. In some embodiments, the SHP-1 inhibitor, the tyrosine kinase inhibitor, the magnetic treatment, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically, and the method comprises administering the magnetic treatment at the site of the cancer to be treated. In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and an electrical treatment or electrochemical treatment, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the SHP-1 inhibitor and/or the tyrosine kinase inhibitor is administered at least twice (at least three, four, five, or six times). In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and an electrical or electrochemical treatment, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, and wherein the SHP-1 inhibitor and/or the tyrosine kinase inhibitor and the electrical treatment or electrochemical treatment are administered within 24 hours (e.g., within 12, 8, 4, 2, or 1 hour, or within 30 minutes) of each other. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the SHP-1 inhibitor and/or the tyrosine kinase inhibitor is administered systemically (e.g., intravenously, e.g., subcutaneously) and/or locally (e.g., intratumorally). In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the electrical treatment or electrochemical treatment are administered to the individual until the individual undergoes tumor clearance. In some embodiments, the SHP-1 inhibitor, the tyrosine kinase inhibitor, the electrical treatment or electrochemical treatment, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically, and the method comprises administering the electrical treatment or electrochemical treatment at the site of the cancer to be treated. In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and an electrostatic treatment, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the SHP-1 inhibitor and/or the tyrosine kinase inhibitor is administered at least twice (at least three, four, five, or six times). In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and an electrostatic treatment, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, and wherein the SHP-1 inhibitor and/or the tyrosine kinase inhibitor and the electrostatic treatment are administered within 24 hours (e.g., within 12, 8, 4, 2, or 1 hour, or within 30 minutes) of each other. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the SHP-1 inhibitor and/or the tyrosine kinase inhibitor is administered systemically (e.g., intravenously, e.g., subcutaneously) and/or locally (e.g., intratumorally). In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the electrostatic treatment are administered to the individual until the individual undergoes tumor clearance. In some embodiments, the SHP-1 inhibitor, the tyrosine kinase inhibitor, the electrostatic treatment, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically, and the method comprises administering the electrostatic treatment at the site of the cancer to be treated. In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and an antibody drug conjugate, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, optionally wherein the SHP-1 inhibitor and/or the tyrosine kinase inhibitor is administered at least twice (at least three, four, five, or six times). In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody) and an antibody drug conjugate, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, and wherein the SHP-1 inhibitor and/or the tyrosine kinase inhibitor and the antibody drug conjugate are administered within 24 hours (e.g., within 12, 8, 4, 2, or 1 hour, or within 30 minutes) of each other. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the SHP-1 inhibitor and/or the tyrosine kinase inhibitor is administered systemically (e.g., intravenously, e.g., subcutaneously) and/or locally (e.g., intratumorally). In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the antibody drug conjugate are administered to the individual until the individual undergoes tumor clearance. In some embodiments, the SHP-1 inhibitor, the tyrosine kinase inhibitor, the antibody drug conjugate, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically, and the method comprises administering the antibody drug conjugate at the site of the cancer to be treated. In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, a lymphocyte activating agent described herein is administered to the individual. For example, in some embodiments, there is provided a method of treating a cancer (e.g., a solid tumor, e.g., a hematological cancer, e.g., a late-stage cancer) in an individual, comprising administering a TNFα inhibitor (e.g., an anti-TNFα neutralizing antibody), a TLR agonist, and a myeloid cell activating agent or therapy, wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the TLR agonist activates one or more TLRs selected from the group consisting of TLR9, TLR4, TLR7 and TLR8. In some embodiments, the immune checkpoint inhibitor is an anti-PD-1 agent (such as an anti-PD-1 antibody), an anti-PD-L1 agent (such as an anti-PD-L1 antibody), or an anti-CTLA-4 agent (such as an anti-CTLA-4 antibody). In some embodiments, the tyrosine kinase inhibitor, the TLR agonist, and the immune checkpoint inhibitor are administered within the same day. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the TLR agonist activates a TLR on a macrophage, optionally wherein the TLR comprises TLR9. In some embodiments, the TLR agonist activates at least two TLRs (e.g., TLR4, TLR7, TLR8, or TLR9). In some embodiments, the TLR agonist activates at least three TLRs (e.g., TLR9, TLR4 and TLR7/8). In some embodiments, the TLR agonist comprises CpG, polyI:C and/or R848. In some embodiments, the TLR agonist comprises CpG, polyI:C and R848, for example at 1:1:1 ratio. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the TLR agonist are administered to the individual until the individual undergoes tumor clearance. In some embodiments, the SHP-1 inhibitor, the tyrosine kinase inhibitor, the TLR agonist, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically, and the TLR agonist is administered intratumorally. In some embodiments, the SHP-1 inhibitor and the tyrosine kinase inhibitor is administered systemically and intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody), wherein the individual is selected for treatment based upon the individual having an ongoing inflammation reaction, and wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the individual has an acute inflammation reaction. In some embodiments, the inflammation reaction is in the tumor. In some embodiments, the inflammation reaction is at a site distinct from the tumor. In some embodiments, the individual has an inflammation reaction when an inflammation reaction where there are at least two (e.g., two, three, four or five) events selected from the group consisting of a) an increase in one or more (e.g., at least one, two, three, four, five) inflammatory cytokines (such as IFNγ, IL-12β, TNFα, IL-6, IL-1β, IFN-α1, IFN-α2, IFN-β1), b) a decrease in one or more (e.g., at least one, two or three) anti-inflammatory cytokine (such as TGFβ1, TGFβ2, TGFβ3), c) an increase in the infiltrating immune cells (such as T cells, NK cells, macrophages, neutrophils), d) a decrease in suppressive immune cells (such as MDSCs), and/or e) an increase in one or more (e.g., at least one, two, three, four, or five) immunogenic co-stimulatory molecules (such as CD80, CD86, OX40L, CD40, ICOS-L, PD-L1, GITRL) in the tissue (e.g., tumor tissue) or immune cells (such as macrophages). In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the SHP-1 inhibitor and/or the tyrosine kinase inhibitor is selected from the group consisting of: a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZFN, or TALENS systems), a peptide agent, a protein agent (e.g., an antibody agent that targets SHP-1 or tyrosine kinase or activated tyrosine kinase), a protein degrading or destabilizing agent, a protein modified with an unnatural amino acid, an antibody directed therapy, an antibody drug conjugate (ADC), and any combination thereof. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the myeloid cell activating agent or therapy are administered to the individual until the individual undergoes tumor clearance. In some embodiments, the SHP-1 inhibitor, the tyrosine kinase inhibitor, the myeloid cell activating agent or therapy, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method further comprises locally (e.g., intratumorally) administering the myeloid cell activating agent or therapy into the individual. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP1 inhibitor and the tyrosine kinase inhibitor is administered systemically, and the myeloid cell activating agent or therapy is administered intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a TNFα inhibitor (e.g., an anti-TNFα neutralizing antibody), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof, and wherein the individual is selected for treatment based upon the individual having an ongoing immunogenic cell death (ICD). In some embodiments, the individual has ICD when a sample from the cancer has a higher level of one or more (e.g., at least 10%, 20%, 30%, 40%, 50%, 60%, 70%, 80%, 90/6 more) DAMPs than a reference sample (e.g., a corresponding sample in a healthy control, e.g., a sample from the cancer prior to the administration of a therapy that induces ICD). In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the DAMPs are selected from the group consisting of endoplasmic reticulum (ER) chaperones (e.g., calreticulin (CALR), e.g., heat-shock proteins (HSPs)), the non-histone chromatin-binding protein high-mobility group box 1 (HMGB1), the cytoplasmic protein annexin A1 (ANXA1), and the small metabolite ATP, and type I interferons (IFNs). In some embodiments, the SHP-1 inhibitor and/or the tyrosine kinase inhibitor is selected from the group consisting of: a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZFN, or TALENS systems), a peptide agent, a protein agent (e.g., an antibody agent that targets SHP-1 or tyrosine kinase or activated tyrosine kinase), a protein degrading or destabilizing agent, a protein modified with an unnatural amino acid, an antibody directed therapy, an antibody drug conjugate (ADC), and any combination thereof. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the myeloid cell activating agent or therapy are administered to the individual until the individual undergoes tumor clearance. In some embodiments, the SHP-1 inhibitor, the tyrosine kinase inhibitor, the myeloid cell activating agent or therapy, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method further comprises locally (e.g., intratumorally) administering the myeloid cell activating agent or therapy into the individual. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP-1 inhibitor and the tyrosine kinase inhibitor is administered systemically, and the myeloid cell activating agent or therapy is administered intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, the present application provides a method of treating a cancer (e.g., a solid tumor, e.g., a hematological cancer, e.g., a late-stage cancer) in an individual, comprising administering to the individual a) TNFα inhibitor (e.g., an anti-TNFα neutralizing antibody), b) monocytes or macrophages deficient in tyrosine kinase expression or activation, and c) a myeloid cell activating agent or therapy (e.g., a TLR agonist, e.g., a STING activator, e.g., a radiation therapy), wherein the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the other one or more agents described above. In some embodiments, the monocytes or macrophages are derived from the same individual. In some embodiments, the monocytes or macrophages are engineered to express a chimeric receptor targeting a tumor antigen. In some embodiments, the SHP-1 inhibitor and/or the tyrosine kinase inhibitor, the monocytes or macrophages, and the myeloid cell activating agent or therapy are administered within 24 hours (e.g., within 12, 8, 4, 2, or 1 hour, or within 30 minutes) of each other. In some embodiments, the SHP-1 inhibitor and/or the tyrosine kinase inhibitor, the monocytes or macrophages, and the myeloid cell activating agent or therapy are administered simultaneously, concurrently, or sequentially. In some embodiments, the monocytes or macrophages are administered prior to the myeloid cell activating agent or therapy. In some embodiments, the monocytes or macrophages are administered after administration of the myeloid cell activating agent or therapy. In some embodiments, the monocytes or macrophages are administered prior to the SHP-1 inhibitor and/or the tyrosine kinase inhibitor. In some embodiments, the monocytes or macrophages are administered after administration of the SHP-1 inhibitor and/or the tyrosine kinase inhibitor. In some embodiments, the immune checkpoint inhibitor and/or the cytokine or biologically active fragment thereof is administered daily. In some embodiments, the immune checkpoint inhibitor and/or the cytokine or biologically active fragment thereof is administered intermittently. In some embodiments, the immune checkpoint inhibitor and/or the cytokine or biologically active fragment thereof is administered to the individual for at least two cycles, further optionally wherein the immune checkpoint inhibitor and/or the cytokine or biologically active fragment thereof is administered for at least once in each cycle and wherein each cycle has about three to about twenty days. In some embodiments, the SHP-1 inhibitor, the tyrosine kinase inhibitor, and the myeloid cell activating agent or therapy are administered to the individual until the individual undergoes tumor clearance. In some embodiments, the SHP-1 inhibitor, the tyrosine kinase inhibitor, the myeloid cell activating agent or therapy, and/or the TNFα inhibitor are administered intermittently to the individual after tumor clearance. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the inhibitor of the SHP-1 pathway. In some embodiments, the TNFα inhibitor is administered simultaneously with the administration of the myeloid cell activating agent or therapy and/or the inhibitor of the SHP-1 pathway. In some embodiments, the TNFα inhibitor is administered concurrently with the administration of the myeloid cell activating agent or therapy and/or the inhibitor of the SHP-1 pathway. In some embodiments, the TNFα inhibitor is administered sequentially to (e.g., prior to or after) the administration of the myeloid cell activating agent or therapy and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the inhibitor of the SHP-1 pathway. In some embodiments, the method further comprises locally (e.g., intratumorally) administering the myeloid cell activating agent or therapy into the individual. In some embodiments, the method comprises administering (e.g., locally or systemically) to the individual an effective amount of both a SHP-1 inhibitor (e.g., TPI-1 or an analog or derivative thereof) and a tyrosine kinase inhibitor (e.g., Dasatinib). In some embodiments, the SHP1 inhibitor and the tyrosine kinase inhibitor is administered systemically, and the myeloid cell activating agent or therapy is administered intratumorally. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering (e.g., orally, intravenously, subcutaneously, and/or intratumorally) to the individual a TNFα inhibitor (e.g., a neutralizing antibody) and immune cells (e.g., T cells, e.g., CAR-T cells, e.g., antigen-specific T cells). In some embodiments, the immune cells comprise T cells. In some embodiments, the immune cells are derived from the same individual. In some embodiments, the immune cells are derived from a donor distinct from the individual. In some embodiments, the T cells are engineered to express a chimeric receptor that specifically binds to a tumor antigen (e.g., a CAR). In some embodiments, the CAR specifically binds to an antigen selected from the group consisting of CD19, CD20, CD22, HER2, IL13Ra2, MUC1, PSMA, EGFR, MSLN, CEA, and BMCA. In some embodiments, the CAR specifically binds to CD19. In some embodiments, the CAR specifically binds to CD22. In some embodiments, the CAR-T cells are administered to the individual in a dose that effectively treats the cancer. In some embodiments, the immune cells comprise at least about 0.1×10⁶, 0.5×10⁶, 1×10⁶, 2×10⁶, 2.5×10⁶, 3×10⁶, 4×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, 10⁷ T cells (e.g., CAR T cells or antigen-specific T cells) per kg for an individual (e.g., a human individual). In some embodiments, the immune cells comprise at least about 10⁶, 2×10⁶, 5×10⁶, 10⁷, 2×10⁷, 5×10⁷, 10⁸, 2×10⁸, 2.5×10⁸, 3×10⁸, 4×10⁸, 5×10⁸, 6×10⁸, 7×10⁸, 7.5×10⁸, 8×10⁸, 9×10⁸, or 1×10⁹ T cells (e.g., CAR T cells or antigen-specific T cells In some embodiments, the CAR-T cells are administered systemically (e.g., intravenously, subcutaneously, or intraperitoneally) or locally (e.g., intratumorally). In some embodiments, the T cells (e.g., CAR-T cells) are administered to the individual in a single dose. In some embodiments, the T cells (e.g., CAR-T cells) are administered to the individual until the individual undergoes tumor clearance. In some embodiments, the TNFα inhibitor is administered systemically (e.g., orally, intravenously, subcutaneously, or intraperitoneally) or locally (e.g., intratumorally). In some embodiments, the TNFα inhibitor is administered at least once a week, once every five days, once every three days, or daily. In some embodiments, the TNFα inhibitor is administered to the individual for at least two cycles, wherein each cycle has about three to about seven days. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the immune cells (e.g., each dose of immune cells). In some embodiments, the TNFα inhibitor is administered at least 24 hours prior to the administration of the CAR-T cells. In some embodiments, the TNFα inhibitor is administered at least 3 hours prior to the administration of the CAR-T cells. In some embodiments, the TNFα inhibitor is administered simultaneously with the administration of the CAR-T cells. In some embodiments, the TNFα inhibitor is administered concurrently with the administration of the CAR-T cells. In some embodiments, the TNFα inhibitor is administered prior to the administration of the CAR-T cells and again concurrently and/or following the administration of the CAR-T cells. In some embodiments, the TNFα inhibitor is administered prior to (e.g., within 2 weeks), concurrently with, or shortly before (e.g., within 6, 3, 2, or 1 hour) each administration of the CAR-T cells. In some embodiments, the individual does not develop cytokine release syndrome (e.g., CRS of grade 2, 3 and/or 4) or pro-inflammatory organ damage with the administration of the TNFα inhibitor. The four grading systems currently used for cytokine release syndrome are shown in Table 1. In some embodiments, administration of the TNFα inhibitor is able to lower levels of IL-6, IL-10, and IFNγ (i.e., cytokines associated with CRS) in individuals. In some embodiments, administration of the TNFα inhibitor is able to reduce flu-like symptoms associated with CRS (e.g., fever, general malaise, and fatigue). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering (e.g., orally, intravenously, subcutaneously, and/or intratumorally) to the individual a TNFα inhibitor (e.g., a neutralizing antibody) and immune cells wherein the immune cells comprise NK cells. In some embodiments, the NK cells are derived from the same individual. In some embodiments, the NK cells are derived from a donor distinct from the individual. In some embodiments, the NK cells are able to infiltrate tumor microenvironments. In some embodiments, the NK cells are able to lyse tumor cells without the need for prior sensitization. In some embodiments, the NK cells trigger cell death of tumor cells by releasing cytotoxic granules comprising granzymes and perforin. In some embodiments, the NK cells trigger cell death of tumor cells through death receptor-mediated pathways (e.g., FasL/Fas pathway). In some embodiments, the NK cells play immunomodulatory functions (e.g., by secreting chemokines and cytokines). In some embodiments, the NK cells are engineered to express a chimeric receptor that specifically binds to a tumor antigen (CAR). In some embodiments, the CAR specifically binds to an antigen selected from the group consisting of CD19, CD20, CD22, HER2, IL13Ra2, MUC1, PSMA, EGFR, MSLN, CEA, and BMCA. In some embodiments, the CAR specifically binds to CD19. In some embodiments, the CAR specifically binds to CD22. In some embodiments, the NK cells are CAR-NK cells. In some embodiments, the NK cells are administered to the individual in a dose that effectively treats the cancer. In some embodiments, the CAR-T cells are administered to the individual in a dose that effectively treats the cancer. In some embodiments, the immune cells comprise at least about 0.1×10⁶, 0.5×10⁶, 1×10⁶, 2×10⁶, 2.5×10⁶, 3×10⁶, 4×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, 10⁷ NK cells (e.g., CAR NK cells) per kg for an individual (e.g., a human individual). In some embodiments, the immune cells comprise at least about 10⁶, 2×10⁶, 5×10⁶, 10⁷, 2×10⁷, 5×10⁷, 10⁸, 2×10⁸, 2.5×10⁸, 3×10⁸, 4×10, 5×10⁸, 6×10⁸, 7×10⁸, 7.5×10⁸, 8×10⁸, 9×10⁸, or 1×10⁹ NK cells (e.g., CAR-NK cells). In some embodiments, the NK cells (e.g., CAR-NK cells) are administered systemically (e.g., intravenously, subcutaneously, or intraperitoneally) or locally (e.g., intratumorally). In some embodiments, the NK cells (e.g., CAR-NK cells) are administered to the individual in a single dose. In some embodiments, the T cells (e.g., CAR-T cells) are administered to the individual until the individual undergoes tumor clearance. In some embodiments, the TNFα inhibitor is administered systemically (e.g., orally, intravenously, subcutaneously, or intraperitoneally) or locally (e.g., intratumorally). In some embodiments, the TNFα inhibitor is administered at least once a week, once every five days, once every three days, or daily. In some embodiments, the TNFα inhibitor is administered to the individual for at least two cycles, wherein each cycle has about three to about seven days. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the immune cells (e.g., each dose of immune cells). In some embodiments, the TNFα inhibitor is administered at least 24 hours prior to the administration of the NK cells. In some embodiments, the TNFα inhibitor is administered at least 3 hours prior to the administration of the NK cells. In some embodiments, the TNFα inhibitor is administered simultaneously with the administration of the NK cells. In some embodiments, the TNFα inhibitor is administered concurrently with the administration of the NK cells. In some embodiments, the TNFα inhibitor is administered prior to the administration of the NK cells and again concurrently and/or following the administration of the NK cells. In some embodiments, the TNFα inhibitor is administered prior to (e.g., within 2 weeks), concurrently with, or shortly before (e.g., within 6, 3, 2, or 1 hour) each administration of the NK cells. In some embodiments, the individual does not develop cytokine release syndrome (e.g., CRS of grade 2, 3 and/or 4) or pro-inflammatory organ damage with the administration of the TNFα inhibitor. The four grading systems currently used for cytokine release syndrome are shown in Table 1. In some embodiments, administration of the TNFα inhibitor is able to lower levels of IL-6, IL-10, and IFNγ (i.e., cytokines associated with CRS) in individuals. In some embodiments, administration of the TNFα inhibitor is able to reduce flu-like symptoms associated with CRS (e.g., fever, general malaise, and fatigue). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering (e.g., orally, intravenously, subcutaneously, and/or intratumorally) to the individual a TNFα inhibitor (e.g., a neutralizing antibody) and immune cells, wherein the immune cells are antigen presenting cells (APCs) (e.g., APCs disclosed in WO 2023/192542, the content of which is hereby incorporated by reference in its entirety). In some embodiments, the APCs are derived from the same individual. In some embodiments, the APCs are derived from a donor distinct from the individual. In some embodiments, the APCs are capable of performing phagocytosis. In some embodiments, the APCs perform phagocytosis on debris from cells (e.g., cancer cells). In some embodiments, the APCs present antigens and/or epitopes to immune cells. In some embodiments, the APCs cross-present various epitopes (e.g., epitopes associated with cancer) or present several antigens (e.g., antigens associated with cancer) simultaneously. In some embodiments, the APCs comprise one or more tumor-associated antigen peptides (e.g., neoantigen peptides). In some embodiments, the APCs present antigens to activate tumor-specific adaptive immunity (e.g., tumoricidal T cells and long-lasting anti-cancer antibodies). In some embodiments, the APCs express a high level of one or more antigen presentation molecule, wherein the antigen presentation molecule is selected from the group consisting of: MHCI, MHCII, CD86, CD80, OX40L, ICAML, ICOSL, and CD40. In some embodiments, the APCs express a low level of an inhibitory signaling molecule, wherein the inhibitory signaling molecule is selected from the group consisting of: TGFβR, SIRPα, LILRB (LILRB1 and/or LILRB2) and Siglec 10. In some embodiments, the APCs are derived from monocytes. In some embodiments, the APCs are monocytes. In some embodiments, the APCs are macrophages. In some embodiments, the APCs are dendritic cells. In some embodiments, the APCs are engineered to express a chimeric receptor that specifically binds to a tumor antigen (CAR). In some embodiments, the CAR specifically binds to an antigen selected from the group consisting of CD19, CD20, CD22, HER2, IL13Ra2, MUC1, PSMA, EGFR, MSLN, CEA, and BMCA. In some embodiments, the CAR specifically binds to CD19. In some embodiments, the CAR specifically binds to CD22. In some embodiments, the APCs are CAR-macrophages. In some embodiments, the APCs are CAR-monocytes. In some embodiments, the APCs are administered to the individual in a dose that effectively treats the cancer. In some embodiments, the APC cells are administered systemically (e.g., intravenously, subcutaneously, or intraperitoneally) or locally (e.g., intratumorally). In some embodiments, the APC cells are administered to the individual in a single dose. In some embodiments, the APC cells are administered to the individual until the individual undergoes tumor clearance. In some embodiments, the TNFα inhibitor is administered systemically (e.g., orally, intravenously, subcutaneously, or intraperitoneally) or locally (e.g., intratumorally). In some embodiments, the TNFα inhibitor is administered at least once a week, once every five days, once every three days, or daily. In some embodiments, the TNFα inhibitor is administered to the individual for at least two cycles, wherein each cycle has about three to about seven days. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the immune cells (e.g., each dose of immune cells). In some embodiments, the TNFα inhibitor is administered at least 24 hours prior to the administration of the APC cells. In some embodiments, the TNFα inhibitor is administered at least 3 hours prior to the administration of the APC cells. In some embodiments, the TNFα inhibitor is administered simultaneously with the administration of the APC cells. In some embodiments, the TNFα inhibitor is administered concurrently with the administration of the APC cells. In some embodiments, the TNFα inhibitor is administered prior to the administration of the APC cells and again concurrently and/or following the administration of the APC cells. In some embodiments, the TNFα inhibitor is administered prior to (e.g., within 2 weeks), concurrently with, or shortly before (e.g., within 6, 3, 2, or 1 hour) each administration of the APC cells. In some embodiments, the individual does not develop cytokine release syndrome (e.g., CRS of grade 2, 3 and/or 4) or pro-inflammatory organ damage with the administration of the TNFα inhibitor. The four grading systems currently used for cytokine release syndrome are shown in Table 1. In some embodiments, administration of the TNFα inhibitor is able to lower levels of IL-6, IL-10, and IFNγ (i.e., cytokines associated with CRS) in individuals. In some embodiments, administration of the TNFα inhibitor is able to reduce flu-like symptoms associated with CRS (e.g., fever, general malaise, and fatigue). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering (e.g., orally, intravenously, subcutaneously, and/or intratumorally) to the individual a TNFα inhibitor (e.g., a neutralizing antibody) and anti-CD3/anti-CD28 monoclonal antibodies (mAbs). In some embodiments, the anti-CD3/anti-CD28 mAbs leads to ligation of TCR comprised on T cells in vivo. In some embodiments, the anti-CD3/anti-CD28 mAbs lead to activation of T cells in vivo. In some embodiments, the anti-CD3/anti-CD28 mAbs lead to proliferation of T cells in vivo. In some embodiments, the anti-CD3/anti-CD28 mAbs are administered to the individual in a dose that effectively treats the cancer. In some embodiments, the anti-CD3/anti-CD28 mAbs are administered systemically (e.g., intravenously, subcutaneously, or intraperitoneally) or locally (e.g., intratumorally). In some embodiments, the anti-CD3/anti-CD28 mAbs are administered in a single dose. In some embodiments, the anti-CD3/anti-CD28 mAbs are administered intermittently. In some embodiments, the anti-CD3/anti-CD28 mAbs are administered to the individual until the individual undergoes tumor clearance. In some embodiments, the TNFα inhibitor is administered systemically (e.g., orally, intravenously, subcutaneously, or intraperitoneally) or locally (e.g., intratumorally). In some embodiments, the TNFα inhibitor is administered systemically (e.g., orally, intravenously, subcutaneously, or intraperitoneally) or locally (e.g., intratumorally). In some embodiments, the TNFα inhibitor is administered at least once a week, once every five days, once every three days, or daily. In some embodiments, the TNFα inhibitor is administered to the individual for at least two cycles, wherein each cycle has about three to about seven days. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the anti-CD3/anti-CD28 monoclonal antibodies (mAbs). In some embodiments, the TNFα inhibitor is administered at least 24 hours prior to the administration of the anti-CD3/anti-CD28 monoclonal antibodies (mAbs). In some embodiments, the TNFα inhibitor is administered at least 3 hours prior to the administration of the anti-CD3/anti-CD28 monoclonal antibodies (mAbs). In some embodiments, the TNFα inhibitor is administered simultaneously with the administration of the anti-CD3/anti-CD28 monoclonal antibodies (mAbs). In some embodiments, the TNFα inhibitor is administered concurrently with the administration of the anti-CD3/anti-CD28 monoclonal antibodies (mAbs). In some embodiments, the TNFα inhibitor is administered prior to the administration of the anti-CD3/anti-CD28 monoclonal antibodies (mAbs) and again concurrently and/or following the administration of the anti-CD3/anti-CD28 monoclonal antibodies (mAbs). In some embodiments, the TNFα inhibitor is administered prior to (e.g., within 2 weeks), concurrently with, or shortly before (e.g., within 6, 3, 2, or 1 hour) each administration of the anti-CD3/anti-CD28 monoclonal antibodies (mAbs). In some embodiments, the individual does not develop cytokine release syndrome (e.g., CRS of grade 2, 3 and/or 4) or pro-inflammatory organ damage with the administration of the TNFα inhibitor. The four grading systems currently used for cytokine release syndrome are shown in Table 1. In some embodiments, administration of the TNFα inhibitor is able to lower levels of IL-6, IL-10, and IFNγ (i.e., cytokines associated with CRS) in individuals. In some embodiments, administration of the TNFα inhibitor is able to reduce flu-like symptoms associated with CRS (e.g., fever, general malaise, and fatigue). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering (e.g., orally, intravenously, subcutaneously, and/or intratumorally) to the individual a TNFα inhibitor (e.g., a neutralizing antibody) and a bispecific T cell engagers (BiTe). In some embodiments, the BiTe redirects T cells to target cancer cells. In some embodiments, the BiTe binds to T cells and cancer cells simultaneously. In some embodiments, the BiTe is selected from the group consisting of Blinatumomab, Pasotuxizumab, Cibisatamab, AMV564, AMG 160, AMG 330, AMG 673, AMG 420, AMG 701, AMG 596, AMG 757, AMG 199, AMG 910, HPN424, M701, M802, and ERY974. In some embodiments, the BiTe is administered to the individual in a dose that effectively treats the cancer. In some embodiments, the TNFα inhibitor is administered systemically (e.g., orally, intravenously, subcutaneously, or intraperitoneally) or locally (e.g., intratumorally). In some embodiments, the TNFα inhibitor is administered systemically (e.g., orally, intravenously, subcutaneously, or intraperitoneally) or locally (e.g., intratumorally). In some embodiments, the TNFα inhibitor is administered at least once a week, once every five days, once every three days, or daily. In some embodiments, the TNFα inhibitor is administered to the individual for at least two cycles, wherein each cycle has about three to about seven days. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of the bispecific T cell engagers (BiTe) (e.g., each dose of bispecific T cell engagers (BiTe)). In some embodiments, the TNFα inhibitor is administered at least 24 hours prior to the administration of the bispecific T cell engagers (BiTe). In some embodiments, the TNFα inhibitor is administered at least 3 hours prior to the administration of the BiTe. In some embodiments, the TNFα inhibitor is administered simultaneously with the administration of the BiTe. In some embodiments, the TNFα inhibitor is administered concurrently with the administration of the BiTe. In some embodiments, the TNFα inhibitor is administered prior to the administration of the BiTe and again concurrently and/or following the administration of the BiTe. In some embodiments, the TNFα inhibitor is administered prior to (e.g., within 2 weeks), concurrently with, or shortly before (e.g., within 6, 3, 2, or 1 hour) each administration of the BiTe. In some embodiments, the individual does not develop cytokine release syndrome (e.g., CRS of grade 2, 3 and/or 4) or pro-inflammatory organ damage with the administration of the TNFα inhibitor. The four grading systems currently used for cytokine release syndrome are shown in Table 1. In some embodiments, administration of the TNFα inhibitor is able to lower levels of IL-6, IL-10, and IFNγ (i.e., cytokines associated with CRS) in individuals. In some embodiments, administration of the TNFα inhibitor is able to reduce flu-like symptoms associated with CRS (e.g., fever, general malaise, and fatigue). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering (e.g., orally, intravenously, subcutaneously, and/or intratumorally) to the individual a TNFα inhibitor (e.g., a neutralizing antibody), a SHP-1 inhibitor (e.g., TPI-1, a analog or a derivative thereof) and a TLR agonist. In some embodiments, there is provided a method of treating a cancer (e.g., a solid tumor, e.g., a hematological cancer, e.g., a late-stage cancer) in an individual, comprising administering (e.g., orally, intravenously, subcutaneously, and/or intratumorally) to the individual a TNFα inhibitor (e.g., a neutralizing antibody), a SHP-1 inhibitor (e.g., TPI-1, a analog or a derivative thereof) and a STING activator. In some embodiments, there is provided a method of treating a cancer (e.g., a solid tumor, e.g., a hematological cancer, e.g., a late-stage cancer) in an individual, comprising administering (e.g., orally, intravenously, subcutaneously, and/or intratumorally) to the individual a TNFα inhibitor (e.g., a neutralizing antibody), a SHP-1 inhibitor (e.g., TPI-1, a analog or a derivative thereof) and a radiotherapy. In some embodiments, the TNFα inhibitor is administered systemically (e.g., orally, intravenously, subcutaneously, or intraperitoneally) or locally (e.g., intratumorally). In some embodiments, the TNFα inhibitor is administered systemically (e.g., orally, intravenously, subcutaneously, or intraperitoneally) or locally (e.g., intratumorally). In some embodiments, the TNFα inhibitor is administered at least once a week, once every five days, once every three days, or daily. In some embodiments, the TNFα inhibitor is administered to the individual for at least two cycles, wherein each cycle has about three to about seven days. In some embodiments, the TNFα inhibitor is administered prior to (e.g., within 2 weeks, 1 week, 5 days, 3 days, 2 days, or 1 day), concurrently with, or shortly after (e.g., within 6 hours, 5 hours, 4 hours, 3 hours, 2 hours, 1 hour, 30 minutes) the administration of one or more of the other agent (e.g., SHP-1 inhibitor, TLR agonist, STING activator, and/or radiation therapy). In some embodiments, the TNFα inhibitor is administered at least 24 hours prior to the administration of one or more of the other agent. In some embodiments, the TNFα inhibitor is administered at least 3 hours prior to the administration of one or more of the other agent. In some embodiments, the TNFα inhibitor is administered simultaneously with the administration of one or more of the other agent. In some embodiments, the TNFα inhibitor is administered concurrently with the administration of one or more of the other agent. In some embodiments, the TNFα inhibitor is administered prior to the administration of one or more of the other agent and again concurrently and/or following the administration of one or more of the other agent. In some embodiments, the TNFα inhibitor is administered prior to (e.g., within 2 weeks), concurrently with, or shortly before (e.g., within 6, 3, 2, or 1 hour) each administration of the other agent. In some embodiments, the individual does not develop cytokine release syndrome (e.g., CRS of grade 2, 3 and/or 4) or pro-inflammatory organ damage with the administration of the TNFα inhibitor. The four grading systems currently used for cytokine release syndrome are shown in Table 1. In some embodiments, administration of the TNFα inhibitor is able to lower levels of IL-6, IL-10, and IFNγ (i.e., cytokines associated with CRS) in individuals. In some embodiments, administration of the TNFα inhibitor is able to reduce flu-like symptoms associated with CRS (e.g., fever, general malaise, and fatigue). In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

In some embodiments, there is provided a method of treating a cancer (e.g., a solid tumor, e.g., a hematological cancer, e.g., a late-stage cancer) in an individual, comprising administering to the individual a) a TNFα inhibitor (e.g., a neutralizing antibody), b) a myeloid cell activating agent such as a TLR agonist (e.g., Poly I:C or R848) or a STING activator (e.g., ADU-s100), c) a SHP-1 inhibitor (e.g., TPI-1 or an analog or a derivative thereof), and d) a tyrosine kinase inhibitor (e.g., dasatinib). In some embodiments, the TNFα inhibitor is administered prior to (e.g., within 2 weeks), concurrently with, or shortly before (e.g., within 6, 3, 2, or 1 hour) each administration of one or more the other agent. In some embodiments, the method further comprises administering an immune checkpoint inhibitor (e.g., an anti-PD-1 antibody) and/or a cytokine (IL-2) or biologically active fragment thereof. In some embodiments, the SHP-1 inhibitor, tyrosine kinase inhibitor, and/or the myeloid cell activating agent are comprised in a topical treatment vehicle. In some embodiments, the topical treatment vehicle is a lotion. In some embodiments, the topical treatment vehicle is Johnson's lotion. In some embodiments, the topical treatment vehicle comprises one or more of the agents selected from the group consisting of a myeloid cell activating agent, a SHP-1 inhibitor, a tyrosine kinase inhibitor, or any combination thereof. In some embodiments, the topical treatment vehicle is administered to a region comprising a cutaneous or subcutaneous cancer (e.g., cutaneous or subcutaneous breast cancer). In some embodiments, the topical treatment vehicle is administered to a superficial cancer lesion. In some embodiments, the topical treatment vehicle triggers a local anti-cancer response. In some embodiments, the topical treatment vehicle triggers a systemic anti-cancer response. In some embodiments, the method further comprises administering the immune checkpoint inhibitor, and/or cytokine or biologically active fragment thereof systemically (e.g., through intraperitoneal injection or intravenous injection). In some embodiments, the topical treatment vehicle is administered intermittently. In some embodiments, the topical treatment vehicle is administered at an interval of no more than once every two days. In some embodiments, the SHP-1 inhibitor and/or the tyrosine kinase inhibitor is administered no less than two times and no more than 5 times within ten consecutive days (e.g., twice in ten days, three times in ten days, four times in ten days, or five times in ten days). In some embodiments, the topical treatment vehicle is administered concurrently with the immune checkpoint inhibitor and/or the cytokine or biologically active fragment thereof. In some embodiments, the topical treatment vehicle is administered concurrently with the TNFα inhibitor. In some embodiments, the topical treatment vehicle and the TNFα inhibitor are administered sequentially and within 2 weeks (e.g., within 10 days, 7 days, 6 days, 5 days, 4 days, 3 days, 2 days, or the same day). In some embodiments, the SHP-1 inhibitor and/or the tyrosine kinase inhibitor has a half-life of no more than about 10 days (e.g., no more than about 7 days, 5 days, 4 days, or 3 days). In some embodiments, the tyrosine kinase inhibitor is effective in inhibiting more than 50% of the tyrosine kinase activity for no more than about 7 days (e.g., about 5 days, 4 days, or 3 days). In some embodiments, the SHP-1 inhibitor is effective in inhibiting more than 50% of the SHP-1 activity for no more than about 7 days (e.g., about 5 days, 4 days, or 3 days). In some embodiments, the SHP-1 inhibitor and/or the tyrosine kinase inhibitor is selected from the group consisting of: a small molecule, a nucleic acid (e.g., an siRNA, an shRNA, an antisense RNA, a microRNA), a nucleic acid base inhibitor (e.g., a circular RNA inhibitor; see, e.g., Holdt, L. M. et al., Front Physiol 2018; 9:1262), a nucleic acid editing system (e.g., CRISPR, ZFN, or TALENS systems), a peptide agent, a protein agent (e.g., an antibody agent that targets SHP-1 or tyrosine kinase or activated tyrosine kinase), a protein degrading or destabilizing agent, a protein modified with an unnatural amino acid, an antibody directed therapy, an antibody drug conjugate (ADC), and any combination thereof. In some embodiments, the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator. In some embodiments, the tyrosine kinase inhibitor specifically inhibits SHP-1 signaling. In some embodiments, the tyrosine kinase inhibitor is a Src inhibitor. In some embodiments, the tyrosine kinase is a Syk inhibitor. In some embodiments, the tyrosine kinase inhibitor is an Hck inhibitor. In some embodiments, the tyrosine kinase inhibitor inhibits any one or more of: Src, Syk, Hck, Lck, Lyn, JAK, and Yes. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. In some embodiments, the tyrosine kinase inhibitor does not or weakly inhibits one or more kinases involved in T cell activation (for example, Lck, Fyn, Zap70, Syk and Csk). In some embodiments, the method comprises locally (e.g., intratumorally) administering an effective amount of the myeloid cell activating agent into the individual. In some embodiments, the method comprises administering (e.g., locally or systemically) to the individual an effective amount of a TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent and/or the inhibitor of the SHP-1 pathway. In some embodiments, the TNFα inhibitor is administered simultaneously with the administration of the topical treatment vehicle. In some embodiments, the TNFα inhibitor is administered concurrently with the administration of the topical treatment vehicle. In some embodiments, the TNFα inhibitor is administered sequentially to (e.g., prior to or after) the administration of the myeloid cell activating agent and/or the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the topical treatment vehicle. In some embodiments, the method further comprises administering (e.g., locally or systemically) to the individual an effective amount of both an immune checkpoint inhibitor (e.g., anti-PD-1 antibody) and a cytokine or biologically active fragment thereof (e.g., IL-2).

The present application also provides a method of modulating monocytes or macrophages derived from an individual having a cancer, comprising contacting the monocytes or macrophages with a SHP-1 inhibitor and/or a tyrosine kinase inhibitor as described above, a myeloid cell activating agent or therapy as described above, and TNFα inhibitor as described above. In some embodiments, the monocytes or macrophages are derived from the same individual. In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

The present application also provides methods of activating phagocytosis against tumor cells in an individual having a tumor, comprising administering to the individual a TNFα inhibitor (e.g., an anti-TNFα neutralizing antibody), wherein the individual a) has been subject to, is being subject to, or is about to be subject to a myeloid cell activating agent or therapy, or b) is under an inflammation reaction or has an ongoing infection, and wherein further the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof. In some embodiments, the SHP-1 inhibitor and/or the tyrosine kinase inhibitor is administered systemically (e.g., orally, intravenously, subcutaneously, or intraperitoneally). The present application also provides a method of activating tumor infiltrating T cells in an individual having a tumor comprising administering to the individual a TNFα inhibitor (e.g., an anti-TNFα neutralizing antibody), wherein the individual a) has been subject to, is being subject to, or is about to be subject to a myeloid cell activating agent or therapy, or b) is under an inflammation reaction or has an ongoing infection, and wherein further the individual a) has been subject to, is being subject to, or is about to be subject to administration of a SHP-1 inhibitor and/or a tyrosine kinase inhibitor, and/or b) has been subject to, is being subject to, or is about to be subject to an immune checkpoint inhibitor and/or a cytokine or biologically active fragment thereof. In some embodiments, the myeloid cell activating agent or therapy and/or the TNFα inhibitor is administered systemically (e.g., orally, intravenously, subcutaneously, or intraperitoneally) or locally (e.g., intratumorally). In some embodiments, the myeloid cell activating agent or therapy is administered daily for at least 2, 3, 4, 5, 6, or 7 days. In some embodiments, the myeloid cell activating agent or therapy is administered intermittently. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the SHP-1 inhibitor and/or the tyrosine kinase inhibitor is administered daily for at least 2, 3, 4, 5, 6, or 7 days. In some embodiments, the SHP-1 inhibitor and/or the tyrosine kinase inhibitor is administered intermittently. In some embodiments, the method comprises administering the SHP-1 inhibitor and/or the tyrosine kinase inhibitor to the individual for at least two cycles, further optionally wherein the SHP-1 inhibitor and/or the tyrosine kinase inhibitor is administered for at least once in each cycle and wherein each cycle has about three to about twenty days. In some embodiments, the SHP-1 inhibitor and/or the tyrosine kinase inhibitor is administered for at least twice (e.g., at least two consecutive days) in each cycle. In some embodiments, the SHP-1 inhibitor and/or the tyrosine kinase inhibitor is administered for at least three times (e.g., at least three consecutive days) in each cycle. In some embodiments, the myeloid cell activating agent or therapy and the SHP-1 inhibitor and/or the tyrosine kinase inhibitor are administered within 24 hours of each other. In some embodiments, the myeloid cell activating agent or therapy comprises an agent selected from the group consisting of a TLR agonist, a STING activator, a radiation therapy, a PAMP/DAMP activator, a pro-inflammatory cytokine, a chemotherapeutic agent, a bacterium or component thereof, a fungus or component thereof, a cancer vaccine, a virus or component thereof, an antibody drug conjugate, and any combination thereof. In some embodiments, the method further comprises administering to the individual an agent that reduces systemic inflammation and/or reduces inflammatory cytokine cascade or cytokine storm (e.g., an anti-IL-6 antibody or an anti-IL-1 antibody).

The cancer treatment methods described above are useful for preventing, mitigating, and/or ameliorating side effects such as treatment toxicity due to CRS and/or pro-inflammatory organ damage without decreasing or only minimally decreasing the efficacy of an anti-cancer treatment that can be useful for 1) activating SHP-1 signaling pathway in an individual; 2) depleting the tyrosine kinase-iR-SHP-1 axis immunosuppression in the individual; 3) activating intratumoral anti-cancer innate and/or adaptive immunity in the individual; 4) unleashing TLR-induced pro-inflammatory response; and/or 5) increasing antigen presentation by tumor-relevance macrophages (TAM). Thus, in some embodiments, there is provided a method of mitigating side effects (e.g., treatment toxicity, CRS, and/or pro-inflammatory organ damage) in an individual, comprising administering to an individual in need thereof a TNFα inhibitor, wherein the individual has been administered, or is about to be administered, an anti-cancer treatment comprising a myeloid cell activating agent or therapy and an inhibitor of the SHP-1 pathway. In some embodiments, there is provided a method of mitigating side effects (e.g., treatment toxicity, CRS, and/or pro-inflammatory organ damage) in an individual, comprising administering to an individual in need thereof a TNFα inhibitor, wherein the individual has been administered, or is about to be administered, an anti-cancer treatment comprising a myeloid cell activating agent or therapy and a SHP-1 inhibitor and/or tyrosine kinase inhibitor. In some embodiments, there is provided a method of preventing side effects (e.g., treatment toxicity, CRS, and/or pro-inflammatory organ damage) in an individual, comprising administering to an individual in need thereof a TNFα inhibitor, wherein the individual has been administered, or is about to be administered, an anti-cancer treatment comprising a myeloid cell activating agent or therapy and an inhibitor of the SHP-1 pathway. In some embodiments, there is provided a method of preventing side effects (e.g., treatment toxicity, CRS, and/or pro-inflammatory organ damage) in an individual, comprising administering to an individual in need thereof a TNFα inhibitor, wherein the individual has been administered, or is about to be administered, an anti-cancer treatment comprising a myeloid cell activating agent or therapy and a SHP-1 inhibitor and/or tyrosine kinase inhibitor. In some embodiments, there is provided a method of ameliorating side effects (e.g., treatment toxicity, CRS, and/or pro-inflammatory organ damage) in an individual, comprising administering to an individual in need thereof a TNFα inhibitor, wherein the individual has been administered, or is about to be administered, an anti-cancer treatment comprising a myeloid cell activating agent or therapy and an inhibitor of the SHP-1 pathway. In some embodiments, there is provided a method of ameliorating side effects (e.g., treatment toxicity, CRS, and/or pro-inflammatory organ damage) in an individual, comprising administering to an individual in need thereof a TNFα inhibitor, wherein the individual has been administered, or is about to be administered, an anti-cancer treatment comprising a myeloid cell activating agent or therapy and a SHP-1 inhibitor and/or tyrosine kinase inhibitor. The present application thus also provides methods for any one or more of these purposes. It is to be understood that, while the administration of the TNFα inhibitor is provided as part of a cancer treatment methods described above, it is not necessary that the TNFα inhibitor itself has any anti-cancer effect. The TNFα inhibitor can be provided, for example, for the purpose of ameliorating side effects caused by the administration of other agents (e.g., the myeloid cell activating agent or therapy or the inhibitor of the SHP-1 pathway). Thus, for example, in some embodiments, there is provided a method of reducing side effects in an individual undergoing anti-cancer treatment (including, for example, treatment methods comprising any of the non-TNFα administrations described above), wherein the method comprises administering to the individual an effective amount of a TNFα inhibitor. In some embodiments, the individual is administered with a myeloid cell activating agent or therapy. In some embodiments, the individual is administered with an inhibitor of the SHP-1 pathway. The various dosing regimens described in the paragraphs above apply equally well in this context and are herein specifically incorporated by reference.

### Tumor Microenvironment (TME) Immunosuppression and SHP-1 Signaling

Src homology region 2 (SH-2) domain-containing phosphatase 1 (SHP-1) is a non-receptor tyrosine phosphatase encoded by the PTPN6 gene that is located on human chromosome 12p13 and contains two promoter regions (within exon 1 and 2), giving rise to two forms of SHP-1 which differ in their N-terminal amino acid sequences but have a similar phosphatase activity. Promoter I is active in non-hematopoietic cells, while promoter II is active in hematopoietic-derived cells; in some epithelial cancer cells, both promoters may function and generate various SHP-1-alternative transcripts. The two SHP-1 isoforms show different subcellular localizations: form I is mainly located in the nucleus, while form II is in the cytoplasm, suggesting that they have different targets.

SHP-1 is a 595 amino acid protein composed of two tandem N-terminal SH2 domains (N-SH2 and C-SH2), a classic catalytic protein tyrosine phosphatase (PTP) domain, and a C-terminal tail containing several phosphorylation sites. Its crystal revealed a structure in which the N-SH2 is bound to the catalytic site of the protein through charge-charge interaction. In this auto-inhibited inactive state, the access of substrates to the active site is prevented, but binding of phosphotyrosine residues to the SH2 domains causes a conformational change that impairs the interaction between the N-SH2 and the catalytic domains. This opens the conformation to allow the access of substrate and is further stabilized by new interactions between SH2 domains and the catalytic domain. These molecular rearrangements determine a sophisticated regulatory mechanism controlled by substrate recruitment.

An additional mechanism of activation is mediated by the phosphorylation of amino acids within the C-terminal tail. So far, three phosphorylation sites have been found, two tyrosine (Tyr536 and Tyr564) residues and a serine (Ser591) residue. Tyr536 and Tyr564 become phosphorylated upon various stimuli (i.e., insulin stimulation or apoptosis inducers), giving rise to increased SHP-1 activity. The molecular mechanism is not clear, although it has been proposed that Tyr phosphorylations could lead to interaction with the N-SH2 domain, releasing the inhibitory effect of this domain on the PTPase activity. SHP-1 activity can also be negatively regulated by protein kinase C (PKC) or mitogen-activated protein kinases (MAPKs) through phosphorylation at Ser591, whose mechanism of inhibition has not been well-characterized.

Protein-tyrosine phosphorylation is a reversible post-translational modification, tightly regulated by both kinases and phosphatases. Any deviation in the phosphorylation/dephosphorylation balance can promote the intracellular accumulation of tyrosine-phosphorylated proteins, which cause an altered regulation of cellular processes including cell growth, migration, invasion, differentiation, survival, and cellular trafficking. In this scenario, SHP-1 acts as a classical tumor suppressor, mainly involved in the homeostatic maintenance of potentially all these processes. SHP-1 function is indeed altered in both solid and hematological human cancers through somatic mutations or epigenetic mechanisms. Besides its well-documented role in the regulation of hematopoietic cell biology, SHP-1 has now been correlated to a number of signal transduction pathways relevant to cancer pathogenesis and progression. Indeed, intratumoral iRs-SHP-1 mediated inhibitory regulations are particularly strong under tumor therapies, as these treatments often induce ITIMs to be hyper-phosphorylated, thereby spurring ‘hyper-activation’ of SHP-1, a feedback loop safeguarding tumors from therapeutic damage and inflammatory afront, while also eliciting a wound healing response that promotes tumor progression.

However, inhibition of SHP-1 risks severe adverse effects. Studies of SHP-1 genetically deficient mice, the motheaten mice (me/me or me^{v}/me^{v}), reveal critical immunological abnormalities and hyperactivation of immune cells associated with the global loss of the tyrosine kinase (see, e.g., Green & Schultz, J Hered 1975; 66:250-258; and Shultz & Green, J Immunol. 1976; 116:936-943, both hereby incorporated by reference). The motheaten mice usually succumb to life-threatening autoimmune inflammatory conditions at an early age. Even partial depletion of SHP-1 in WT mice after they grew to adults led to features of inflammatory disease, causing extensive lung inflammation and splenomegaly. Like a double-edged sword, inhibition of SHP-1, despite the potential of empowering anti-cancer immunity, inevitably endangers hosts for heightened inflammatory response, cytokine storm, and autoimmunity.

Inhibitors targeting SHP-1 activity have been under development for some times, and some have now entered preclinical studies, including NSC-87877, sodium stibogluconate (SSG), tyrosine phosphatase inhibitor 1 (TPI-1), and suramine; however, only a few of them have been shown to be active in experimental tumor models. SSG has been through Phase I trials for both malignant melanoma (NCT00498979) and advanced malignancies (NCT00629200); the drug was administrated in combination with interferons followed or not by chemotherapy treatment. Unfortunately, no effect was seen against tumor development, with the most common toxic side-effects being thrombocytopenia, elevated serum lipase, fatigue, fever, chills, anemia, hypokalemia, pancreatitis, and skin rash (observed in up to 68% of patients). At present, no SHP-1 inhibitor is under Phase II trial.

The SHP-1 inhibitors described herein can be administered along with a tyrosine kinase inhibitor, both of which target proteins that act in the SHP-1 signaling pathway. In some embodiments, the method comprises administering (e.g., locally or systemically) to the individual an effective amount of a SHP-1 inhibitor (e.g., TPI-1 or an analog or derivative thereof). In some embodiments, the SHP-1 inhibitor is administered simultaneously with a tyrosine kinase inhibitor. In some embodiments, the SHP-1 inhibitor is administered sequentially (e.g., prior to or after) with a tyrosine kinase inhibitor. In some embodiments, the SHP-1 inhibitor administration follows the same dosing schedule as the tyrosine kinase inhibitor.

As shown in the Examples provided herein, the features of inflammatory disease, (e.g., lung inflammation and splenomegaly) can be prevented and/or ameliorated by the administration of a TNFα inhibitor. Exemplary TNFα inhibitors can include but are not limited to a small molecule inhibitor, a neutralizing antibody, a soluble TNFα Receptor (e.g., a fusion protein), a TNFα Receptor blockade antibody, a TNFα-targeting short interfering RNA (siRNA), chemical inhibitors of TNFα mRNA stability, and an inhibitor of TNFα converting enzyme (TACE). In some embodiments, the TNFα inhibitor is a neutralizing antibody. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy. In some embodiments, the TNFα inhibitor is administered simultaneously with the administration of the myeloid cell activating agent or therapy. In some embodiments, the TNFα inhibitor is administered concurrently with the administration of the myeloid cell activating agent or therapy. In some embodiments, the TNFα inhibitor is administered sequentially to (e.g., prior to or after) the administration of the myeloid cell activating agent or therapy. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy. In some embodiments, the TNFα inhibitor is administered no more than four days (e.g., three, two, or one day) after the administration of the myeloid cell activating agent or therapy. In some embodiments, the TNFα inhibitor is administered prior to (e.g., within about any of one week, 7 days, 6 days, 5 days, 4 days, 3 days, 2 days, 1 day, or less prior to) the administration of the inhibitor of the SHP-1 pathway. In some embodiments, the TNFα inhibitor is administered simultaneously with the administration of the inhibitor of the SHP-1 pathway. In some embodiments, the TNFα inhibitor is administered concurrently with the administration of the inhibitor of the SHP-1 pathway. In some embodiments, the TNFα inhibitor is administered sequentially to (e.g., prior to or after) the administration of the inhibitor of the SHP-1 pathway. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the inhibitor of the SHP-1 pathway. In some embodiments, the TNFα inhibitor is administered systemically. In some embodiments, administration of the TNFα inhibitor does not compromise or only moderately compromises the efficacy of the myeloid cell activating agent or therapy. In some embodiments, administration of the TNFα inhibitor does not compromise the efficacy of the inhibitor of the SHP-1 pathway.

### Cytokine Release Syndrome and the Role of TNFα

Immunotherapies (such as CAR-T therapy) to cancer is associated with risks of cytokine release syndrome (CRS), potentially leading to serious side effects and multi organ dysfunction. In some cases, severe CRS can cause further life-threatening immune-related adverse effects (irAEs) and dire pathophysiological consequences. In addition to CAR-T therapy, other immunotherapies that involve activation of T cells, NK cells and macrophages, such as TIL, TCR-T, NeoT, CAR-NK, CAR-M, NK, CAR-Mac, SIRPant-M, immune checkpoint blockades, BsAbs and other T cell activation Ab therapies, T and NK stimulating cytokine therapies (e.g. IL-2, IL-7, IL-15 IL-21, etc.), as well as neoantigen vaccines, dendritic cell vaccine, oncolytic varus, etc. also sometimes induce typical CRS episodes and irAEs.

While the rise of CRS suggests risks of patients for severe adverse effects, it ironically also indicates CAR-T therapy effectiveness, reflecting T cells identifying targets, activation, and executing cancer killing function. As part of these “on-target” effector activities, T cells release a panel of stimulating cytokines such as IFNg, TNFa and IL-2, each playing a role to further enhance and direct the anti-cancer immune response, as well as sensitizing cancer cells for T cell mediated killing. However, when T cells-produced cytokines permeate surrounding tissues and the bloodstream, they stimulate macrophages and other immune and tissue cells, and induce a systemic response and immune activation. Among these second-layer responsive cells, recent studies indicate macrophages to be the most important activating immune cells, which produce a large amount of proinflammatory cytokines and chemokines leading to a full-blown CRS. Currently, mild cases of CRS in clinical practices are managed with standard supportive therapies. For severe cases, Tocilizumab (Actemra), an IL-6 blockade antibody, and steroids are commonly used as the major managements. During CRS, IL-6 is often found in large amounts in serum and its blockade can ameliorate the CRS symptom. However, these managements sometimes fail to, or only moderately release the adverse response, especially when patients experience severe cases of CRS with persistently high cytokine and chemokine levels. Further mechanistic understanding of CRS and new clinical management innovations are needed to improve immunotherapeutic effectiveness, while avoiding therapy-associated immunological adverse effects.

TNFα, a major pro-inflammatory cytokine, is secreted by activated macrophages, monocytes, and lymphocytes. TNFα plays a role in the non-specific immune response, but it is still not completely understood what role, if any, TNFα plays in the specific, cell-mediated immune response. TNFα, as well as various other cytokines, has been identified as a potential inducer of such biological reactions as cytokine release syndrome (CRS). CRS can be induced by direct target cell lysis and the consecutive release of cytokines like TNFα or IFNγ, or by activation of T cells due to therapeutic stimuli that is followed by subsequent cytokine release. These cytokines trigger a chain reaction due to the activation of innate immune cells like macrophages and endothelial cells, which then induces further cytokine release.

IL-6, IL-10, and IFNγ are most commonly found to be elevated in patients with CRS. IFNγ causes fever, chills, headache, dizziness, and fatigue. IFNγ induces activation of macrophages, which in turn produce excessive amounts of additional cytokines such as IL-6, TNFα, and IL-10.

TNFα elicits flu-like symptoms similar to IFNγ, i.e., fever, general malaise, and fatigue. TNFα further has been linked to watery diarrhea, vascular leakage, cardiomyopathy, lung injury, and the synthesis of acute phase proteins. However, the mechanism of action and the degree to which TNFα overproduction contributes to the development and/or the severity of CRS remains elusive.

IL-6 in particular is believed to play an integral role in CRS pathophysiology and patient symptoms. The IL-6R targeting antibody, tocilizumab, has become standard care for the initial treatment of severe CRS in patients receiving CAR T cells, and additional therapies include corticosteroids. In cases where IL-6 blockage has been ineffective, some patients have responded to TNFα blockade in conjunction with IL-6 blockade and corticosteroids. See, e.g., Shimabukuro-Vornhagen, A. et al., J Immunother Cancer. 2018 Jun. 15; 6(1):56.

A TNFα Inhibitor that Reduces Systemic Inflammation

The methods described herein comprises administering to the individual a) a myeloid cell activating agent or therapy, and b) a TNFα inhibitor. The inventors surprisingly found that the administration of an anti-TNFα neutralizing antibody to an individual who has been administered, or is about to be administered, with a SHP-1 inhibitor and/or tyrosine kinase inhibitor and a myeloid cell activating agent or therapy alleviates toxicity caused by systemic inflammation without compromising the efficacy of the therapeutic agents (see Examples below).

The methods of the present application therefore in some embodiments comprises administration of a TNFα inhibitor (e.g., in the context where the myeloid cell activating agent or therapy is not TNFα). In some embodiments, the TNFα inhibitor is selected from the group consisting of: a small molecule inhibitor, a neutralizing antibody, a TNFα receptor blockade antibody, a soluble TNFα receptor, a TNFα-targeting short interfering RNA (siRNA), a chemical inhibitor of TNFα mRNA stability, an inhibitor of TNFα converting enzyme (TACE), and derivatives thereof. In some embodiments, the TNFα inhibitor is an anti-TNFα neutralizing antibody. In some embodiments, the TNFα inhibitor is an anti-TNFα receptor blockade antibody. In some embodiments, the anti-TNFα antibody is a monoclonal antibody. In some embodiments, the anti-TNFα antibody is a chimeric, humanized, and/or fully human antibody.

Suitable antibodies for use in the methods provided herein include, but are not limited to, Remicade® (Infliximab (Centocor)), and those antibodies described, for example, in U.S. Pat. Nos. 6,835,823; 6,790,444; 6,284,471; 6,277,969; 5,919,452; 5,698,195; 5,656,272; and 5,223,395 and in EP Patent No. 0610201, the contents of each of which are hereby incorporated by reference in their entirety, or antibodies that bind to the same epitope as Remicade®. Other suitable anti-TNFα antibodies for use in the methods provided herein are, by way of non-limiting example, Humira (Adalimumab (Abbott Laboratories, Esai)) as described in U.S. Pat. Nos. 6,090,382; 6,258,562; or 6,509,015 and related patents and applications, the contents of which are hereby incorporated by reference in their entirety; Simponi™ (Golimimab, CNTO 148 (Centocor)) as described in PCT Publication No. WO 02/12502 and related patents and applications, the contents of which are hereby incorporated by reference in their entirety; ART621 (Arana Therapeutics), SSS 07 (Epitopmics and 3SBio) or antibodies that bind to the same epitope as Humira, Simponi, ART621 or SSS 07.

In some embodiments, the TNFα inhibitor is a fusion protein. Suitable fusion proteins for use in the methods provided herein include, but are not limited to, Enbrel (Etanercept (Amgen)) and other fusion proteins or fragments thereof described in U.S. Pat. No. 5,712,155, PCT Publication No. WO 91/03553, and related patents and applications, the contents of which are hereby incorporated by reference in their entirety.

In some embodiments, the anti-TNFα antagonist is a modified antibody antagonist or a non-antibody-based antagonist. Such antagonists include advanced antibody therapeutics, such as antibody fragments including, but not limited to, Cimzia™ (Certolizumab pegol, CDP870 (Enzon)), bispecific antibodies, Nanobodies® such as ABX 0402 (Ablynx), immunotoxins, and radiolabeled therapeutics; peptide therapeutics; gene therapies, particularly intrabodies; oligonucleotide therapeutics such as aptamer therapeutics, antisense therapeutics, interfering RNA therapeutics; and small molecules such as LMP-420 (LeukoMed) as described in EP Patent No. 0767793, and related patents and applications, the contents of which are hereby incorporated by reference in their entirety.

In one aspect, there is provided a method of treating a cancer in an individual, comprising administering to the individual a) a myeloid cell activating agent or therapy, and b) a TNFα inhibitor. In some embodiments, the TNFα inhibitor is selected from the group consisting of: a small molecule inhibitor, a neutralizing antibody, a TNFα receptor blockade antibody, a soluble TNFα receptor, a TNFα-targeting short interfering RNA (siRNA), a chemical inhibitor of TNFα mRNA stability, an inhibitor of TNFα converting enzyme (TACE), and derivatives thereof. In some embodiments, the TNFα inhibitor is an anti-TNFα neutralizing antibody. In some embodiments, the TNFα inhibitor is an anti-TNFα receptor blockade antibody. In some embodiments, the anti-TNFα antibody is a monoclonal antibody. In some embodiments, the anti-TNFα antibody is a chimeric, humanized, and/or fully human antibody. In some embodiments, the TNFα inhibitor is an antibody, such as infliximab, adalimumab, etanercept, golimumab, and certolizumab. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy. In some embodiments, the TNFα inhibitor is administered simultaneously with the administration of the myeloid cell activating agent or therapy. In some embodiments, the TNFα inhibitor is administered concurrently with the administration of the myeloid cell activating agent or therapy. In some embodiments, the TNFα inhibitor is administered sequentially to (e.g., prior to or after) the administration of the myeloid cell activating agent or therapy. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy, for example no more than four days after the administration of the myeloid cell activating agent or therapy. In some embodiments the method further comprises administering to the individual an inhibitor of the SHP-1 pathway. In some embodiments, the inhibitor of the SHP-1 pathway is a SHP-1 inhibitor, for example TPI-1 or an analog or derivative thereof. In some embodiments, the inhibitor of the SHP-1 pathway is a tyrosine kinase inhibitor, for example Dasatinib.

In some embodiments, the TNFα inhibitor is administered systemically. In some embodiments, the TNFα inhibitor is administered at least once a week, once every five days, once every three days, or daily. In some embodiments, the TNFα inhibitor is administered more than once every day (e.g., at least twice or three times daily). In some embodiments, the TNFα inhibitor is administered intermittently. In some embodiments, the TNFα inhibitor is administered to the individual for at least two cycles, wherein each cycle has about three to about seven days. In some embodiments, the individual does not develop cytokine release syndrome or pro-inflammatory organ damage. In some embodiments, administration of the TNFα inhibitor does not compromise or weakly compromises tumor clearance.

In some embodiments, an IL-6 inhibitor (e.g., an IL-6 antibody) is administered in lieu of or in additional to the TNF-a inhibitor. In some embodiments, the IL-6 is administered prior to (e.g., within 7, 6, 5, 4, 3, 2, or 1 day, e.g., within 24 hours, 12 hours, 8 hours, 6 hours, 5 hours, 4 hours, 3 hours, 2 hour or 1 hour from) the administration of another agent (e.g., a myloid cell activating agent, e.g., a lymphocyte activating agent, e.g., a SHP-1 inhibitor, e.g., TPI-1 or an analog or derivative thereof, e.g., a tyrosine kinase inhibitor, e.g., a TLR agonist, e.g., a STING activator, e.g., an immune cell such as a CAR-T cell, e.g., a cytokine such as IL-2, e.g., an anti-PD-1 antibody). In some embodiments, the IL-6 inhibitor is administered concurrently with the agent. In some embodiments, the IL-6 inhibitor is administered shortly after (e.g., within 24 hours, 12 hours, 6 hours, or 3 hours) the administration of the agent.

### SHP-1 Inhibitors

The SHP-1 inhibitors referred herein is an agent of any kind or sort that inhibits the expression or activation of SHP-1. In some embodiments, the SHP-1 inhibitor directly targets SHP-1. In some embodiments, the SHP-1 inhibitor targets a molecule involved in SHP-1 signaling pathway in macrophages that is distinct from SHP-1. In some embodiments, the SHP-1 inhibitor is a competitive inhibitor, a partial non-competitive inhibitor, a covalent inhibitor, a noncovalent inhibitor, an allosteric inhibitor, an inhibitor that targets the catalytic site, or an inhibitor that targets a regulatory site of SHP-1. Exemplary inhibitors can be found e.g., WO 2024/054934, the content of which is hereby incorporated by reference in its entirety.

In some embodiments, the SHP-1 inhibitor is capable of inhibiting at least about 20% (e.g., at least 20%, 30%, 40%, or 50%) of the SHP-1 activity. In some embodiments, the SHP-1 inhibitor is capable of inhibiting at least about 20% (e.g., at least 20%, 30%, 40%, or 50%) of the SHP-1 expression.

In some embodiments, the SHP-1 inhibitor is selected from the group consisting of: a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZFN, or TALENS systems), a peptide agent, a protein agent (e.g., an antibody agent that targets SHP-1, e.g., an antibody agent that targets SHP-1 or activated SHP-1, e.g., a dominant negative SHP-1 or a constitutively active SHP-1 mutant), a protein agent that contains a SH2 domain (by competing for binding to ITIM motif so to inhibit SHP-1 activation), and a tyrosine kinase inhibitor that inhibits ITIM phosphorylation, a protein degrading or destabilizing agent, a protein modified with an unnatural amino acid, an antibody directed therapy, an antibody drug conjugate (ADC), and any combination thereof.

In some embodiments, the SHP-1 inhibitor does not significantly inhibit SHP-2 (e.g., does not inhibit the SHP-2 activity for more than 50%, 40%, 30%, or 20%).

In some embodiments, the SHP-1 inhibitor also inhibits SHP-2.

In some embodiments, the SHP-1 inhibitor has a half-life of no more than about 10, 9, 8, or 7 days (e.g., a half-life of no more than about 7, 6, 5, 4, 3, 2 or 1 day).

In some embodiments, the SHP-1 inhibitor is effective in inhibiting more than 50% of the SHP-1 activity for no more than about 10, 9, 8, 7, 6, or 5 days. In some embodiments, the SHP-1 inhibitor is effective in inhibiting more than 50% of the SHP-1 activity for no more than 4, 3, 2 or 1 day.

In some embodiments, the SHP-1 inhibitor is a covalent inhibitor. In some embodiments, the SHP-1 inhibitor is a noncovalent inhibitor.

In some embodiments, the SHP-1 inhibitor is a competitive inhibitor. In some embodiments, the SHP-1 inhibitor is Phomoxanthone A (PXA) or Phomoxanthone B (PXB). See e.g., Yang et al., ACS Omega. 2020 Sep. 29; 5(40):25927-25935

In some embodiments, the SHP-1 inhibitor targets the catalytic site. In some embodiments, the SHP-1 inhibitor targets the allosteric or regulatory site. See, e.g., Wang et al. J Cell Biochem. 2011 August; 112(8): 2062-2071 for the structure of SHP-1.

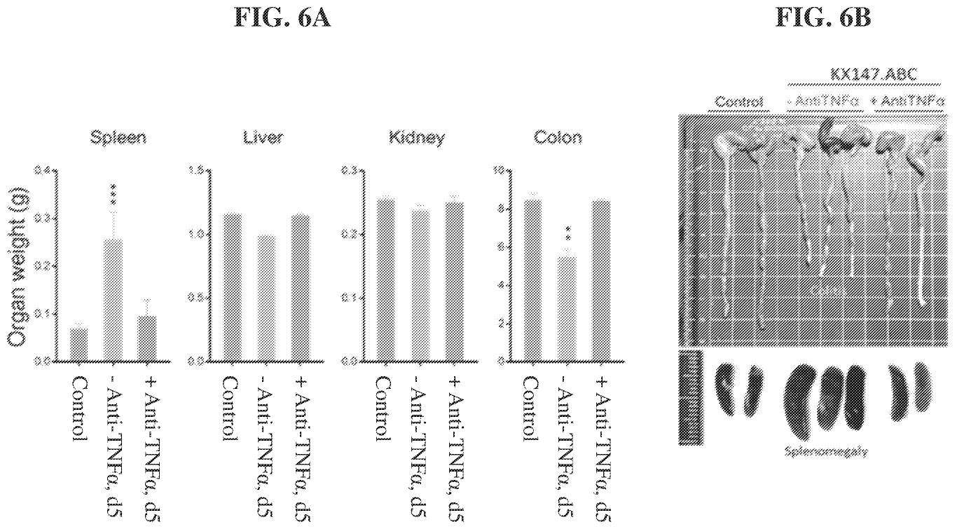

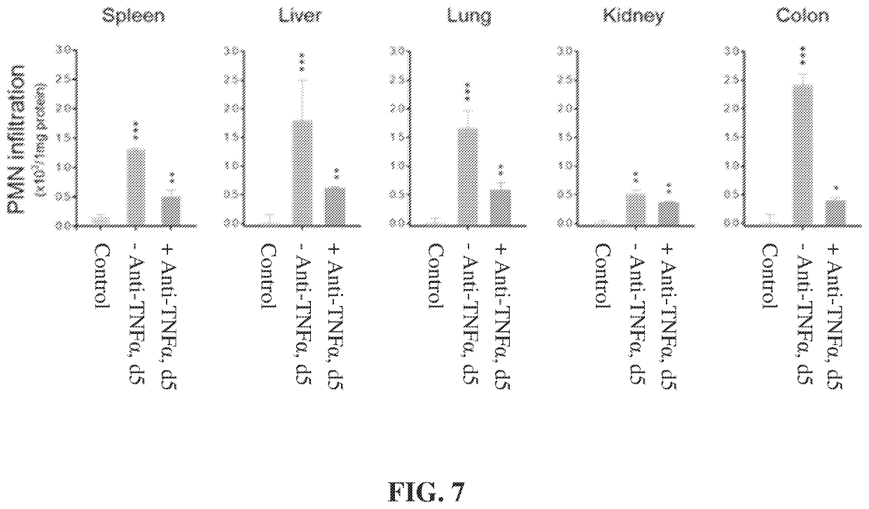

In some embodiments, the SHP-1 inhibitor is TPI-1 or an analog or derivative thereof. Exemplary analogs or derivatives include those disclosed below and in Kundu et al. (J Immunol. 2010 Jun. 1, 184(11): 6529-6536, hereby incorporated by reference in its entirety.) See, e.g., FIGS. 6-7 and Table 1 of Kundu et al.

The compound 2-(2,5-dichlorophenyl)benzoquinone (which is also referred to as 2-(2,5-dichlorophenyl)cyclohexa-2,5-diene-1,4-dione, Tyrosine Phosphatase Inhibitor 1 or TPI-1; CAS Registry No. 79756-69-7) is an inhibitor of SHP-1. TPI-1 has the following structure:

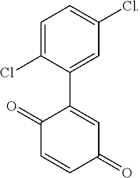 [SMILES: O=C1C=CC(=O)C(c2cc(Cl)ccc2Cl)=C1]

TPI-1 can be derivatized with a functional group for facile attachment to other compounds, for use in conjugate compounds. For example, a carboxyl group can be introduced at the 3-position of the dichlorophenyl ring to provide:

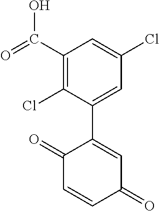

which can be readily coupled to an amino or hydroxy group on another molecule, such as a therapeutic molecule, or a linker to another molecule. Derivatives and analogs of TPI-1 include compounds of the following structure:

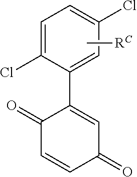

where RC is RCA or —C1-C4 alkyl-RCA, where RCA is —COOH, —NH2, or —OH; or a pharmaceutically acceptable salt thereof.

In some embodiments, the TPI-1 or an analog or derivative thereof comprises a perdeuterated TPI-1 with the following structure, where D is deuterium (i.e., 2H):

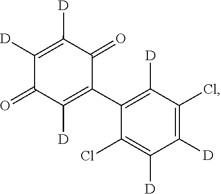 [SMILES: [2H]C1=C([2H])C(=O)C(c2c([2H])c(Cl)c([2H])c([2H])c2Cl)=C([2H])C1=O]

and can be used in non-salt form or as a pharmaceutically acceptable salt.

In some embodiments, the SHP-1 inhibitor is PTP-I.

In some embodiments, the SHP-1 inhibitor is NSC-87877. In some embodiments, the SHP-1 inhibitor is NSC-87877 disodium.

In some embodiments, the SHP-1 inhibitor is sodium stibogluconate.

In some embodiments, the SHP-1 inhibitor is vitamin E. In some embodiments, the SHP-1 inhibitor is tocofersolan (TPGS). In some embodiments, the SHP-1 inhibitor is α-tocopherol acetate (αTA). In some embodiments, the SHP-1 inhibitor is α-tocopheryl succinate (αTOS).

In some embodiments, the SHP-1 inhibitor is phomoxanthone A (PXA).

In some embodiments, the SHP-1 inhibitor is PKCθ activator (such as PMA).

In some embodiments, the SHP-1 inhibitor is phenylhydrazonopyrazolone (PHPS1) sulfonate or a derivative thereof.

In some embodiments, the SHP-1 inhibitor is oxindole or a derivative thereof, e.g., NSC-117199.

In some embodiments, the SHP-1 inhibitor is salicylic acid or a derivative thereof.

In some embodiments, the SHP-1 inhibitor is diterpenoid quinone or a derivative thereof, e.g., cryptotanshinone.

In some embodiments, the SHP-1 inhibitor is an siRNA or an shRNA that inhibits or knocks down the amount of endogenous SHP-1 protein. See, e.g., WO2009/023333.

In some embodiments, the SHP-1 inhibitor is a dominant negative SHP-1 or a constitutively active SHP-1 mutant. See, e.g., WO2009/023333.

In some embodiments, the SHP-1 inhibitor is a nucleic acid editing system (such as a CRISPR system). In some embodiments, the CRISPR components are introduced into the cell (e.g., the monocytes and the macrophages) but no DNA encoding a guide RNA or Cas9 are incorporated into the cell's genome. Under this approach, the CRISPR system only cleaves the cell's genomic DNA for a limited period of time. See, e.g., Fister et al., Front Plant Sci. 2018 Mar. 2; 9:268.

In some embodiments, the SHP-1 inhibitor is a chemical inducer of dimerization. See, e.g., Buck et. al., ACS Omega. 2022 Apr. 11; 7(16):14180-14188.

In some embodiments, the SHP-1 inhibitor (e.g., TPI-1 or an analog or derivative thereof) is administered at least two times (such as at least 3, 4, 5, or 6 times).

In some embodiments, the SHP-1 inhibitor (e.g., TPI-1 or an analog or derivative thereof) is administered at least two times (such as at least 3, 4, 5, or 6 times). In some embodiments, the method comprises administering the SHP-1 inhibitor at a daily interval for at least twice (such as at least three times, four times, five times, or six times).

In some embodiments, the method comprises administering the SHP-1 inhibitor (e.g., TPI-1 or an analog or derivative thereof) at an interval of no more than once every two days for at least twice (such as at least three times, four times, five times, or six times).

In some embodiments, the method comprises administering the SHP-1 inhibitor (e.g., TPI-1 or an analog or derivative thereof) at an interval of no more than once every three days for at least twice (such as at least three times, four times, five times, or six times).

In some embodiments, the method comprises administering the SHP-1 inhibitor (e.g., TPI-1 or an analog or derivative thereof) for at least two cycles. In some embodiments, SHP-1 inhibitor is administered for at least once (e.g., for twice, three times, four times) in each cycle. In some embodiments, each cycle has about one to about 50 days (e.g., about 1-40 days, about 1-30 days, about 1-20 days, about 1-15 days, about 1-10 days, or about 2-10 days).

In some embodiments, the SHP-1 inhibitor is administered systemically (e.g., orally, intravenously, subcutaneously, intraperitoneally). In some embodiments, the SHP-1 inhibitor is administered locally (e.g., intratumorally). In some embodiments, the SHP-1 inhibitor is administered both systemically and locally.

In some embodiments, the SHP-1 inhibitor is complexed with a delivery vehicle before being administered into the individual. In some embodiments, the delivery vehicle promotes the delivery into the tumor.

In some embodiments, the SHP-1 inhibitor modulates a monocyte or macrophage (e.g., a monocyte or macrophage derived from the individual to be treated) in vitro.

In some embodiments, the SHP-1 inhibitor, the myeloid cell activating agent or therapy, and the TNFα inhibitor above are administered within 24 hours (e.g., within 12, 8, 4, 2, or 1 hour, or within 30 minutes) of each other. In some embodiments, the SHP-1 inhibitor, the myeloid cell activating agent or therapy, and the TNFα inhibitor are administered simultaneously, concurrently, or sequentially. In some embodiments, the SHP-1 inhibitor is administered prior to the myeloid cell activating agent or therapy and/or the TNFα inhibitor. In some embodiments, the SHP-1 inhibitor is administered following the myeloid cell activating agent or therapy and/or TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the SHP-1 inhibitor. In some embodiments, the TNFα inhibitor is administered simultaneously with the administration of the myeloid cell activating agent or therapy and/or the SHP-1 inhibitor. In some embodiments, the TNFα inhibitor is administered concurrently with the administration of the myeloid cell activating agent or therapy and/or the SHP-1 inhibitor. In some embodiments, the TNFα inhibitor is administered sequentially to (e.g., prior to or after) the administration of the myeloid cell activating agent or therapy and/or the SHP-1 inhibitor. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the SHP-1 inhibitor.

In some embodiments, the SHP-1 inhibitor, the tyrosine kinase inhibitor below, and the myeloid cell activating agent or therapy below are administered within 24 hours (e.g., within 12, 8, 4, 2, or 1 hour, or within 30 minutes) of each other. In some embodiments, the SHP-1 inhibitor, the tyrosine kinase inhibitor, and the myeloid cell activating agent or therapy are administered simultaneously, concurrently, or sequentially. In some embodiments, the SHP-1 inhibitor and the tyrosine kinase inhibitor are administered prior to the myeloid cell activating agent or therapy. In some embodiments, the SHP-1 inhibitor and the tyrosine kinase inhibitor are administered following the myeloid cell activating agent or therapy.

In some embodiments, the SHP-1 inhibitor, the tyrosine kinase inhibitor below, the myeloid cell activating agent or therapy, and the TNFα inhibitor above are administered within 24 hours (e.g., within 12, 8, 4, 2, or 1 hour, or within 30 minutes) of each other. In some embodiments, the SHP-1 inhibitor, the tyrosine kinase inhibitor, and the myeloid cell activating agent or therapy are administered simultaneously, concurrently, or sequentially. In some embodiments, the SHP-1 inhibitor and the tyrosine kinase inhibitor are administered prior to the myeloid cell activating agent or therapy and/or the TNFα inhibitor. In some embodiments, the SHP-1 inhibitor and the tyrosine kinase inhibitor are administered following the myeloid cell activating agent or therapy and/or the TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the SHP-1 inhibitor and the tyrosine kinase inhibitor. In some embodiments, the TNFα inhibitor is administered simultaneously with the administration of the myeloid cell activating agent or therapy and/or the SHP-1 inhibitor and the tyrosine kinase inhibitor. In some embodiments, the TNFα inhibitor is administered concurrently with the administration of the myeloid cell activating agent or therapy and/or the SHP-1 inhibitor and the tyrosine kinase inhibitor. In some embodiments, the TNFα inhibitor is administered sequentially to (e.g., prior to or after) the administration of the myeloid cell activating agent or therapy and/or the SHP-1 inhibitor and the tyrosine kinase inhibitor. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the SHP-1 inhibitor and the tyrosine kinase inhibitor.

### Cell Surface Inhibitory Receptors

The SHP-1 pathway can also be inhibited by blockade of cell surface inhibitory receptors. Such receptors act to signal through immunoreceptor tyrosine based inhibitory motifs (ITIMs), and recruit phosphatases such as SHP-1, which transduce the intracellular inhibitory signaling cascade. These receptors can dampen inflammation and auto-immune responses in vivo, and these actions can be targeted for novel anti-cancer treatments that engage the immune system in tumor clearance.

Leukocyte immunoglobulin-like (LIL) receptor family comprises a set of paired immunomodulatory receptors that are expressed on human myeloid and lymphoid cells. In particular, LILR subfamily B (LILRB) members signal via multiple cytoplasmic immunoreceptor tyrosine-based inhibitory motifs (ITIMs). The mouse ortholog, PirB, has been shown to regulate functional development of myeloid-derived suppressor cell and the formation of a tumor-permissive microenvironment (see, e.g., van der Touw, W. et al. Cancer Immunol Immunother. 2017 Sep. 9; 66(8): 1079-1087). An ortholog to PirB in mice, gp49B1, has been seen to support leukemia development such that gp49B1 deficiency delayed the development of leukemia (see, e.g., Pend, H. & Zou, Y., Biochem Biophys Res Commun 2021; 565:72-78). Furthermore, the T cells and NK cells of gp49BI deficient mice produced greater levels of IFNγ after vaccinia virus infection compared to wild-type controls (see, e.g., Gu, X. et al., J Immunol 2003; 170(8):4095-4101). LILRB1 is expressed on myeloid cells, B cells, and subsets of NK cells and T cells. LILRB2-5 are restricted to cells of myeloid origin and DCs.

Signal-regulatory proteins (SIRPs) are involved in protein phosphatase binding activity and protein phosphorylated amino acid binding activity. SIRPs, such as SIRPα, are negative regulators of cytokine production. SIRPα is a known myeloid-inhibitory receptor that binds to CD47 and signals through ITIMs.

Siglecs are sialic-acid-binding immunoglobulin-like lectins that are predominantly expressed by cells of the immune system and can be divided into two subsets. Many Siglec sequences include ITIM domains and act in a cell inhibitory capacity when bound by their ligands. Examples of Siglecs that are implicated in myeloid cell signaling include: Siglec-1, Siglec-2, Siglec-3, Siglec-5, Siglec-6, Siglec-7, Siglec-8, Siglec-9, Siglec-10, Siglec-11, Siglec-12, Siglec-14, Siglec-15, Siglec-E, Siglec-F, Siglec-G, and Siglec-H. See, e.g., Crocker, P. R. et al. Nat Rev Immunol. 2007 April; 7:255-266.

Dendritic cell inhibitory receptor (DCIR4, Clec4a1) is a lectin receptor expressed on the cell surface of cells of the myeloid lineage. For example, monocytes that differentiated into dendritic cells showed greatly diminished expression of DCIR4, while monocytes differentiating into macrophages did not show a significantly affected DCIR4 expression level (see, e.g., Kameda, Y. et al., Biochem Biophys Res Commun 2016; 480(2):215-221). DCIR4 has been found to be expressed at high levels in patrolling monocytes and low levels in inflammatory monocytes (see, e.g., Hsu, Y. et al., Biochem Biophys Res Commun 2017; 494(3-4):440-445). The intracellular domain of DCIR4 and other members of the DCIR family contains ITIM domains. Inhibitory DCIRs are found on DCs, B cells, monocytes, macrophages, and neutrophils, but not T or NK cells and relay signal through Src homology region 2 domain-containing phosphatases 1 and 2 (SHP-1/2). CD371 is also known as CLL-1, Clec12A, DCAL-2, or Myeloid Inhibitory C-type Lectin-like receptor (MICL). CD371 is a highly glycosylated transmembrane receptor with a cytoplasmic ITIM that associates with SHP1 and SHP2. CD371 is expressed mainly on granulocytes, monocytes, macrophages, and dendritic cells, including plasmacytoid dendritic cells (see, e.g., Ma, H. et al., J Hematol Oncol 2019; 12:41).

CD200R binds to its ligand, CD200, and inhibits the activation of myeloid cells. Unlike other inhibitors described herein, CD200R intracellular domain does not comprise ITIM domains but does interact with tyrosine kinases, likely at the CD200R NP×Y phosphotyrosine binding domain (PTB). See, e.g., Zhang, S. et al., J Immunol 2004; 173(11):6786-6793.

The SLAM family (i.e., SLAMF1-9) is a group of type I transmembrane receptors, wherein SLAM-related receptors typically act as self-ligands (e.g., SLAMF1, 3, 5, 6, 7, 8, and 9), the exception being 2B4 (i.e., SLAMF4, CD244), which interacts with CD48 (i.e., SLAMF2). SLAM receptors associate with SAP-family adaptors, which contain SH2 domains. In the absence of SAP adaptors, SLAM receptors associate with inhibitor effectors, e.g., SHP-1, SHP-2, SHIP-1, or CSK. See, e.g., Veillette, A. Cold Spring Harb Perspect Biol 2012; 2(3):a002469.

In some embodiments, the inhibitor of the SHP-1 pathway is an inhibitor of one or more cell surface inhibitory receptors. In some embodiments, the inhibitor of one or more cell surface inhibitory receptors is selected from the group consisting of: a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZFN, or TALENS systems), a peptide agent, a protein agent (e.g., an antibody agent that targets cell surface inhibitory receptors), a protein degrading or destabilizing agent, a protein modified with an unnatural amino acid, an antibody directed therapy, an antibody drug conjugate (ADC), and any combination thereof. In some embodiments, the inhibitor of one or more cell surface inhibitory receptors is a cell surface inhibitory receptor blockade antibody.

In some embodiments, the inhibitor of the SHP-1 pathway is one or more antibodies that blockade cell surface inhibitory receptors. In some embodiments, the one or more antibodies that blockade cell surface inhibitory receptors are selected from any one of: LILRB1, LILRB2, LILRB3, LILRB4, LILRB5, SIRPα, PirB, gp49B1, Siglec-1, Siglec-2, Siglec-3, Siglec-5, Siglec-6, Siglec-7, Siglec-8, Siglec-9, Siglec-10, Siglec-11, Siglec-12, Siglec-14, Siglec-15, Siglec-E, Siglec-F, Siglec-G, Siglec-H, DCIR4, CD371, CD200R, SLAMF1, SLAMF3, SLAMF5, SLAMF6, SLAMF7, SLAMF8, and SLAMF9.

### Tyrosine Kinase Inhibitors (TKIs)

The tyrosine kinase inhibitors referred to herein are an agent of any kind or sort that inhibits the expression or activation of tyrosine kinase. In some embodiments, the tyrosine kinase inhibitor is a competitive inhibitor, a partial non-competitive inhibitor, a covalent inhibitor, a noncovalent inhibitor, an allosteric inhibitor, an inhibitor that targets the catalytic site, or an inhibitor that targets a regulatory site of tyrosine kinase. Exemplary TKIs can be found e.g., PCT/US2023/078419, the content of which is hereby incorporated by reference in its entirety.

In some embodiments, the tyrosine kinase inhibitor is capable of inhibiting at least about 20% (e.g., at least 20%, 30%, 40%, or 50%) of the tyrosine kinase activity. In some embodiments, the tyrosine kinase inhibitor is capable of inhibiting at least about 20% (e.g., at least any of 20%, 30%, 40%, or 50%) of the tyrosine kinase expression.

In some embodiments, the tyrosine kinase inhibitor specifically inhibits SHP-1 signaling.

In some embodiments, the tyrosine kinase inhibitor is selected from the group consisting of a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZFN, or TALENS systems), a peptide agent, a protein agent (e.g., an antibody agent that targets tyrosine kinase or activated tyrosine kinase, e.g., an antibody agent that targets tyrosine kinase or activated tyrosine kinase, e.g., a dominant negative tyrosine kinase or a constitutively active tyrosine kinase mutant), a protein degrading or destabilizing agent, a protein modified with an unnatural amino acid, an antibody directed therapy, an antibody drug conjugate (ADC), and any combination thereof.

In some embodiments, the tyrosine kinase inhibitor has a half-life of no more than about 10, 9, 8, or 7 days (e.g., a half-life of no more than about 7, 6, 5, 4, 3, 2 or 1 day).

In some embodiments, the tyrosine kinase inhibitor is effective in inhibiting more than 50% of the tyrosine kinase activity for no more than about 10, 9, 8, 7, 6, or 5 days. In some embodiments, the tyrosine kinase inhibitor is effective in inhibiting more than 50% of the tyrosine kinase activity for no more than 4, 3, 2 or 1 day.

In some embodiments, the tyrosine kinase inhibitor is a covalent inhibitor. In some embodiments, the tyrosine kinase inhibitor is a noncovalent inhibitor.

In some embodiments, the tyrosine kinase inhibitor is a competitive inhibitor.

In some embodiments, the tyrosine kinase inhibitor is a nucleic acid editing system (such as a CRISPR system). In some embodiments, the CRISPR components are introduced into the cell (e.g., the monocytes and the macrophages) but no DNA encoding a guide RNA or Cas9 are incorporated into the cell's genome. Under this approach, the CRISPR system only cleaves the cell's genomic DNA for a limited period of time. See, e.g., Fister et al., Front Plant Sci. 2018 Mar. 2; 9:268.

In some embodiments, the tyrosine kinase inhibitor is administered at least two times (such as at least 3, 4, 5, or 6 times).

In some embodiments, the tyrosine kinase inhibitor is administered at least two times (such as at least 3, 4, 5, or 6 times). In some embodiments, the method comprises administering the tyrosine kinase inhibitor at a daily interval for at least twice (such as at least three times, four times, five times, or six times).

In some embodiments, the tyrosine kinase inhibitor is administered at least two times (such as at least 3, 4, 5, or 6 times). In some embodiments, the method comprises administering the tyrosine kinase inhibitor at an interval of no more than once every two days for at least twice (such as at least three times, four times, five times, or six times).

In some embodiments, the tyrosine kinase inhibitor is administered at least two times (such as at least 3, 4, 5, or 6 times). In some embodiments, the method comprises administering the tyrosine kinase inhibitor at an interval of no more than once every three days for at least twice (such as at least three times, four times, five times, or six times).

In some embodiments, the method comprises administering the tyrosine kinase inhibitor for at least two cycles. In some embodiments, the tyrosine kinase inhibitor is administered for at least once (e.g., for twice, three times, four times) in each cycle. In some embodiments, each cycle has about one to about 50 days (e.g., about 1-40 days, about 1-30 days, about 1-20 days, about 1-15 days, about 1-10 days, or about 2-10 days).

In some embodiments, the tyrosine kinase inhibitor is administered systemically (e.g., orally, intravenously, subcutaneously, intraperitoneally). In some embodiments, the tyrosine kinase inhibitor is administered locally (e.g., intratumorally). In some embodiments, the tyrosine kinase inhibitor is administered both systemically and locally.

In some embodiments, the tyrosine kinase inhibitor is complexed with a delivery vehicle before being administered into the individual. In some embodiments, the delivery vehicle promotes the delivery into the tumor.

In some embodiments, the tyrosine kinase inhibitor modulates a monocyte or macrophage (e.g., a monocyte or macrophage derived from the individual to be treated) in vitro.

In some embodiments, the tyrosine kinase inhibitor, the myeloid cell activating agent or therapy below, and the TNFα inhibitor above are administered within 24 hours (e.g., within 12, 8, 4, 2, or 1 hour, or within 30 minutes) of each other. In some embodiments, the tyrosine kinase inhibitor, the myeloid cell activating agent or therapy, and the TNFα inhibitor are administered simultaneously, concurrently, or sequentially. In some embodiments, the tyrosine kinase inhibitor is administered prior to the myeloid cell activating agent or therapy and/or the TNFα inhibitor. In some embodiments, the tyrosine kinase inhibitor is administered following the myeloid cell activating agent or therapy and/or the TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the tyrosine kinase inhibitor. In some embodiments, the TNFα inhibitor is administered simultaneously with the administration of the myeloid cell activating agent or therapy and/or the tyrosine kinase inhibitor. In some embodiments, the TNFα inhibitor is administered concurrently with the administration of the myeloid cell activating agent or therapy and/or the tyrosine kinase inhibitor. In some embodiments, the TNFα inhibitor is administered sequentially to (e.g., prior to or after) the administration of the myeloid cell activating agent or therapy and/or the tyrosine kinase inhibitor. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the tyrosine kinase inhibitor.

In some embodiments, the tyrosine kinase inhibitor, the SHP-1 inhibitor above, and the myeloid cell activating agent or therapy below are administered within 24 hours (e.g., within 12, 8, 4, 2, or 1 hour, or within 30 minutes) of each other. In some embodiments, the tyrosine kinase inhibitor, the SHP-1 inhibitor, and the myeloid cell activating agent or therapy are administered simultaneously, concurrently, or sequentially. In some embodiments, the tyrosine kinase inhibitor and the SHP-1 inhibitor are administered prior to the myeloid cell activating agent or therapy. In some embodiments, the tyrosine kinase inhibitor and the SHP-1 inhibitor are administered following the myeloid cell activating agent or therapy.

In some embodiments, the tyrosine kinase inhibitor, the SHP-1 inhibitor above, the myeloid cell activating agent or therapy below, and the TNFα inhibitor above are administered within 24 hours (e.g., within 12, 8, 4, 2, or 1 hour, or within 30 minutes) of each other. In some embodiments, the tyrosine kinase inhibitor, the SHP-1 inhibitor, and the myeloid cell activating agent or therapy are administered simultaneously, concurrently, or sequentially. In some embodiments, the tyrosine kinase inhibitor and the SHP-1 inhibitor are administered prior to the myeloid cell activating agent or therapy and/or the TNFα inhibitor. In some embodiments, the tyrosine kinase inhibitor and the SHP-1 inhibitor are administered following the myeloid cell activating agent or therapy and/or the TNFα inhibitor. In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and/or the tyrosine kinase inhibitor and the SHP-1 inhibitor. In some embodiments, the TNFα inhibitor is administered simultaneously with the administration of the myeloid cell activating agent or therapy and/or the tyrosine kinase inhibitor and the SHP-1 inhibitor. In some embodiments, the TNFα inhibitor is administered concurrently with the administration of the myeloid cell activating agent or therapy and/or the tyrosine kinase inhibitor and the SHP-1 inhibitor. In some embodiments, the TNFα inhibitor is administered sequentially to (e.g., prior to or after) the administration of the myeloid cell activating agent or therapy and/or the tyrosine kinase inhibitor and the SHP-1 inhibitor. In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and/or the tyrosine kinase inhibitor and the SHP-1 inhibitor.

In some embodiments, the tyrosine kinase is a tyrosine kinase of the Src family. Src-family kinases have a similar structure, comprised of an N-terminal Src-homology (“SH”) 4 (“SH4”) domain, a “unique” domain, an SH3 domain, an SH2 domain, a catalytic domain (also known as the SH1 domain or the kinase domain) and a short C-terminal tail. Activity is regulated by tyrosine phosphorylation at two sites. Phosphorylation of a tyrosine (Tyr-505, Src numbering) in the C-terminal tail leads to down-regulation by promoting an intramolecular interaction between the tail and the SH2 domain. The eight known mammalian members of the Src-family break down into two sub-families. Lck is most similar to Hck, Lyn, and Blk (identities greater than 65% between any two members). The other sub-family consists of Src, Yes, Fyn, and Fgr (identities greater than 70/6 between any two members). Residues that are important for Src-family kinase activity and/or substrate specificity have been identified by X-ray crystal structures and by structural modeling studies, and are highly conserved among family members.

In some embodiments, the tyrosine kinase inhibitor is a Src inhibitor. In some embodiments, the tyrosine kinase inhibitor is a Syk inhibitor. In some embodiments, the tyrosine kinase inhibitor is an Hck inhibitor. In some embodiments, the tyrosine kinase inhibitor inhibits any one or more (such as any of 2, 3, 4, 5, or 6) of: Src, Syk, Hck, Lck, Lyn, JAK, and Yes. In some embodiments, the tyrosine kinase inhibitor does not or weakly inhibits one or more kinases involved in T cell activation. In some embodiments, the one or more kinases involved in T cell activation comprises any one or more of: Lck, Fyn, Zap70, Syk and Csk. In some embodiments, the tyrosine kinase inhibitor inhibits Bcr-Abl. In some embodiments, the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800. These tyrosine kinase inhibitors are further discussed below.

### Src Inhibitors

Src is a member of non-receptor protein tyrosine kinases, and has an activity that phosphorylates a specific tyrosine residue in a target protein. The Src may be originated from any species of animals (e.g., mammals), and for example may be at least one selected from the group consisting of primate Src including human Src (e.g., Accession No. NP_005408), monkey Src (e.g., Accession No. XP_002830325), and the like, and rodent Src including mouse Src (e.g., Accession No. NP_001020566), rat Src (e.g., Accession No. NP_114183), and the like, but not be limited thereto.

In some embodiments, the Src inhibitor (SRCi) may be an inhibitor of Src gene or Src protein expression; or an inhibitor of Src protein activity. The Src gene or Src protein expression inhibitor may be one or more selected from the group consisting of antisense nucleotides complementarily binding to mRNA of the gene, short interfering RNA (siRNA), short hairpin RNA (shRNA) and ribozyme, but not limited thereto. Further, the Src protein activity inhibitor may be one or more selected from the group consisting of a compound, a peptide, peptide mimetics, aptamers, antibodies, and natural products that specifically bind to the protein, but not limited thereto. The antibody includes a monoclonal antibody, a polyclonal antibody, or a recombinant antibody capable of specifically binding to the Src protein, and can be constructed by methods known to those skilled in the art or purchased and used. According to the present disclosure, the compound may be one or more selected from the group consisting of dasatinib, bosutinib, ponatinib, saracatinib, WH-4-023, KX2-391, and WZ3105.

In one embodiment, the Src inhibitor may be at least one selected from the group consisting of dasatinib, saracatinib, and bosutinib, or any combination thereof.

KX2-391 (Tirbanibulin), which is also called N-benzyl-2-(5-(4-(2-morpholinoethoxy)phenyl)pyridin-2-yl)acetamide, has the following structure:

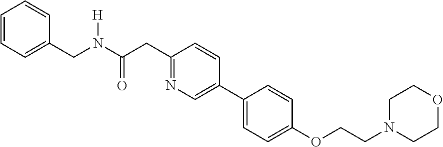 [SMILES: O=C(Cc1ccc(-c2ccc(OCCN3CCOCC3)cc2)cn1)NCc1ccccc1]

Dasatinib, which is also called N-(2-chloro-6-methylphenyl)-2-[[6-[4-(2-hydroxyethyl)-1-piperazinyl]-2-methyl-4-pyrimidinyl]amino]-5-thiazole carboxamide monohydrate, has the following structure:

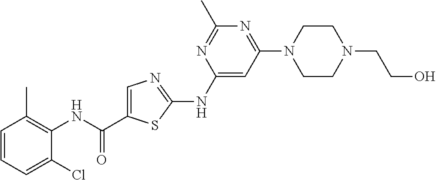 [SMILES: Cc1nc(Nc2ncc(C(=O)Nc3c(C)cccc3Cl)s2)cc(N2CCN(CCO)CC2)n1]

Saracatinib, which is also called AZD0530 (4-Quinazolinamine, N-(5-Chloro-1,3-benzodioxol-4-yl)-7-[2-(4-methyl-1-piperazinyl)ethoxy]-5-[(tetrahydro-2H-pyran-4-yl)oxy]-4-quinazolinamine), has the following structure:

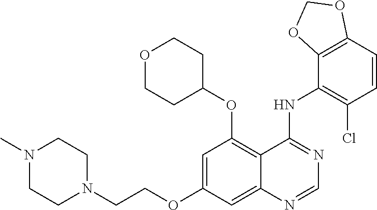 [SMILES: CN1CCN(CCOc2cc(OC3CCOCC3)c3c(Nc4c(Cl)ccc5c4OCO5)ncnc3c2)CC1]

Bosutinib, which is also called 4-[(2,4-dichloro-5-methoxyphenyl)amino]-6-methoxy-7-[3-(4-methylpiperazin-1-yl)propoxy]quinoline-3-carbonitrile, has the following structure:

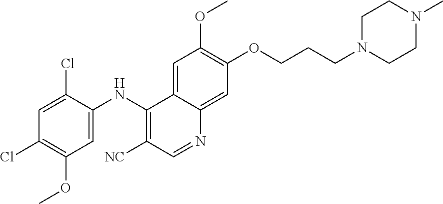 [SMILES: COc1cc(Nc2c(C#N)cnc3cc(OCCCN4CCN(C)CC4)c(OC)cc23)c(Cl)cc1Cl]

### Syk Inhibitors

Spleen tyrosine kinase (Syk) is a cytosolic non-receptor protein tyrosine kinase (PTK). The human SYK gene is located in the region of chromosome 9 q22. Syk, along with ZAP70, is a member of the Syk family of tyrosine kinases. These cytoplasmic non-receptor tyrosine kinases share a characteristic dual SH2 domain separated by a linker domain.

In some embodiments, the Syk inhibitor may be an inhibitor of Syk gene or Syk protein expression; or an inhibitor of Syk protein activity. The Syk gene or Syk protein expression inhibitor may be one or more selected from the group consisting of antisense nucleotides complementarily binding to mRNA of the gene, short interfering RNA (siRNA), short hairpin RNA (shRNA) and ribozyme, but not limited thereto. Further, the Syk protein activity inhibitor may be one or more selected from the group consisting of a compound, a peptide, peptide mimetics, aptamers, antibodies, and natural products that specifically bind to the protein, but not limited thereto. The antibody includes a monoclonal antibody, a polyclonal antibody, or a recombinant antibody capable of specifically binding to the Syk protein, and can be constructed by methods known to those skilled in the art or purchased and used.

In some embodiments, the Syk inhibitor is a small molecule inhibitor. In some embodiments, the Syk inhibitor is selected from the group consisting of Entospletinib (GS-9973), Fostamatinib (R788), R406, Cerdulatinib (PRT0626070), and TAK-659.

In some embodiments, the Syk inhibitor is R406 having the formula as follows:

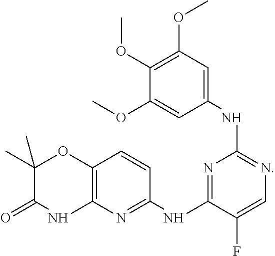 [SMILES: COc1cc(Nc2ncc(F)c(Nc3ccc4c(n3)NC(=O)C(C)(C)O4)n2)cc(OC)c1OC]

### Hck Inhibitors

Hck is a member of the Src-family of non-receptor tyrosine kinases, which plays many roles in signaling pathways involved in the regulation of cell processes. Hck is expressed in cells of hematopoietic origin, specifically myelomonocytic cells and B lymphocytes. It participates in phagocytosis, adhesion, migration, regulation of protrusion formation on cell membrane, lysosome exocytosis, podosome formation and actin polymerization. High levels of Hck are present in chronic myeloid leukemia and other hematologic tumors. Hck could also play a role in the genesis of acute myeloid leukemia.

In some embodiments, the Hck inhibitor may be an inhibitor of Hck gene or Hck protein expression; or an inhibitor of Hck protein activity. The Hck gene or Hck protein expression inhibitor may be one or more selected from the group consisting of antisense nucleotides complementarily binding to mRNA of the gene, short interfering RNA (siRNA), short hairpin RNA (shRNA) and ribozyme, but not limited thereto. Further, the Hck protein activity inhibitor may be one or more selected from the group consisting of a compound, a peptide, peptide mimetics, aptamers, antibodies, and natural products that specifically bind to the protein, but not limited thereto. The antibody includes a monoclonal antibody, a polyclonal antibody, or a recombinant antibody capable of specifically binding to the Hck protein, and can be constructed by methods known to those skilled in the art or purchased and used.

In some embodiments, the Hck inhibitor is a small molecule inhibitor. In some embodiments, the Hck inhibitor is selected from the group consisting of RK-20449, RK-20693, RK-24466, RK-20444, RK-20445, and RK-20466. In other embodiments, the HCK inhibitor is selected from RK-20449, RK-20693, RK-24466, RK-20444, RK-20445, RK-20466, RK-20730, RK-20690, RK-20781, RK-20786, RK-20888, RK-20658, RK-20686, RK-20696, RK-20709, RK-20721, RK-20694, RK-20703, RK-20718, RK-20744, and compounds having Hck inhibitory activity disclosed in WO2014/017659, incorporated herein by reference. Hck inhibitors are also disclosed in WO2018/052120, which are incorporated herein by reference.

RK-20449 (also known as A 419259). 7-((1R,4R)-4-(4-methylpiperazin-1-yl)cyclohexyl)-5-(4-phenoxyphenyl)-7H-pyrrolo[2,3-d]pyrimidin-4-amine has a structure as follows:

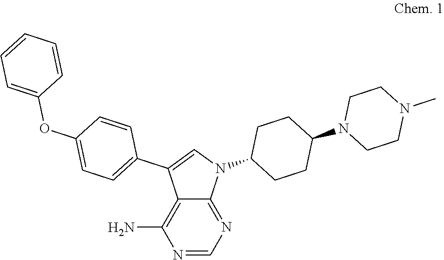 [SMILES: CN1CCN([C@H]2CC[C@H](n3cc(-c4ccc(Oc5ccccc5)cc4)c4c(N)ncnc43)CC2)CC1]

### Lck Inhibitors

Lck (or lymphocyte-specific protein tyrosine kinase) is a member of Src kinase family important for the activation of the T-cell receptor signaling in both naive T cells and effector T cells. The N-terminal tail of Lck is myristoylated and palmitoylated, which tethers the protein to the plasma membrane of the cell. The protein furthermore contains a SH3 domain, a SH2 domain and in the C-terminal part the tyrosine kinase domain.

In some embodiments, the Lck inhibitor may be an inhibitor of Lck gene or Lck protein expression; or an inhibitor of Lck protein activity. The Lck gene or Lck protein expression inhibitor may be one or more selected from the group consisting of antisense nucleotides complementarily binding to mRNA of the gene, short interfering RNA (siRNA), short hairpin RNA (shRNA) and ribozyme, but not limited thereto. Further, the Lck protein activity inhibitor may be one or more selected from the group consisting of a compound, a peptide, peptide mimetics, aptamers, antibodies, and natural products that specifically bind to the protein, but not limited thereto. The antibody includes a monoclonal antibody, a polyclonal antibody, or a recombinant antibody capable of specifically binding to the Lck protein, and can be constructed by methods known to those skilled in the art or purchased and used.

In some embodiments, the Lck inhibitor is a small molecule inhibitor. In some embodiments, the Lck inhibitor is selected from the group consisting of Saractinib, Masitinib, and NVP-BEP800.

### BCR-ABL Inhibitor

BCR-ABL, a fusion gene created as a consequence of a reciprocal translocation mutation in the long arms of Chromosome 9 and 12, encodes the BCR-ABL protein, a constitutively active cytoplasmic tyrosine kinase present in >90% of all patients with chronic myelogenous leukemia (CML) and in 15-30% of adult patients with acute lymphoblastic leukemia (ALL). Exemplary Bcr-Abl inhibitors include, but are not limited to, imatinib, nilotinib, dasatinib, bosutinib, ponatinib, bafetinib, rebastinib, tozasertib, danusertib, HG-7-85-01, GNF-2, and 1,3,4-thiadiazole derivatives. Additional Bcr-Abl inhibitors can be found, for example, at WO2006/052810, specifically incorporated herein by reference.

Ponatinib (AP24534) is a dual Src/Abl inhibitor having the following structure.

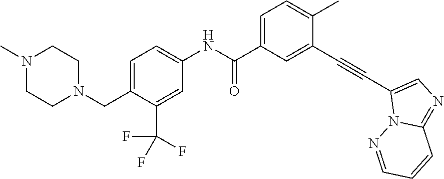 [SMILES: Cc1ccc(C(=O)Nc2ccc(CN3CCN(C)CC3)c(C(F)(F)F)c2)cc1C#Cc1cnc2cccnn12]

### Myeloid Cell Activating Agent or Therapys

Infection and tissue injury are the two classic instigators of inflammation. See e.g., Medzhitov, Nature. 2008 Jul. 24; 454(7203):428-35. Myeloid cell activating agent or therapys described herein include at least two overlapping categories: 1) an agent or therapy of any kind or sort that can promote inflammation (e.g., by promoting one or more pro-inflammatory cytokines or chemokines, inhibiting one or more anti-inflammatory cytokines or chemokines, recruiting macrophages, NK cells, neutrophils, effector T cells, or B cells to the tissue or activating any of these cells, or suppressing regulatory/suppressive immune cells such as regulatory T cells or MDSC), and 2) an agent or therapy that can cause damage of cancer cells (e.g., necrosis of cancer cells).

In some embodiments, the myeloid cell activating agent or therapy activates cells derived from the myeloid immune cell lineage. In some embodiments, the myeloid cell activating agent or therapy activates myeloid lineage antigen presenting cells, for example dendritic cells, macrophages, and normal/classic fibrocytes (e.g., intratumoral). In some embodiments, the myeloid cell activating agent or therapy activates non-myeloid lineage antigen presenting cells, such as B lymphocytes (e.g., intratumoral B cells). Professional antigen presenting cells typically are derived from the myeloid cell lineage, however some lymphocytes, e.g., B cells, may further act as an antigen presenting cell to immune cells that mediate the adaptive immune response, e.g., T cells. In such cases, these antigen presenting cells are included herein as a cell that is activated by the myeloid cell activating agent or therapy. For example, in some embodiments, the antigen presenting cell may be a B cell that presents an antigen to, e.g., a T cell.

In some embodiments, the myeloid cell activating agent or therapy triggers a pro-inflammatory signal on macrophages. In some embodiments, the myeloid cell activating agent or therapy activates a TLR, a TNFR, or ITAM-R. See Lionel et al., Eur J Immunol. 2011 September: 41(9): 2477-2481. The myeloid cell activating agent or therapy can activate a pro-inflammatory signal on macrophages via a direct manner or indirect manner. For example, a TLR agonist, which directly activates TLR on macrophages, or a radiotherapy which indirectly activates a pro-inflammatory signal on macrophages, when used with a SHP-1 inhibitor and/or a tyrosine kinase inhibitor both demonstrated remarkable anti-tumor effects. See the Examples.

Exemplary myeloid cell activating agent or therapys include TLR agonists, STING activators, radiation therapies, PAMP/DAMP activators, pro-inflammatory cytokines chemotherapies, cancer vaccines, bacteria, fungi, and viruses and components or combinations thereof. Other exemplary myeloid cell activating agent or therapys include sound treatments (e.g., high intensity focused ultrasound), magnetic therapies, electrical treatments, and electrostatic treatments that can kill cancer cells. See e.g., Naud et al., Nanoscale Adv., 2020, 2, 3632-3655; Rominiyi et al., Br J Cancer. 2021 February; 124(4):697-709; Zandi et al., Cancer Med. 2021 November; 10(21): 7475-7491.

In some embodiments, the myeloid cell activating agent or therapy comprises an agent selected from the group consisting of TLR agonists, STING activators, radiation therapies, PAMP/DAMP activators, pro-inflammatory cytokines chemotherapies, cancer vaccines, bacteria or bacterial components, fungi or fungal components, viruses or viral components, sound treatments (e.g., high intensity focused ultrasound), magnetic therapies, electrical treatments, electrostatic treatments, and any combination thereof. In some embodiments, the myeloid cell activating agent or therapy comprises two or more agents described herein.

In some embodiments, the myeloid cell activating agent or therapy is a sound treatment (e.g., high intensity focused ultrasound (HIFU), e.g., low intensity focused ultrasound (LIPUS)). See e.g., Wood et al., Ultrasound Med Biol. 2015 April; 41(4): 905-928; Sengupta et al., J Adv Res. 2018 November; 14: 97-111.

In some embodiments, the myeloid cell activating agent or therapy is a magnetic therapy (e.g., pulsed magnetic field, e.g., static magnetic field). See e.g., Tatarov et al., Comp Med. 2011 August; 61(4): 339-345; Sengupta et al., J Adv Res. 2018 November; 14: 97-111.

In some embodiments, the myeloid cell activating agent or therapy is an electrical treatment or electrochemical treatment. See e.g., Ciria et al., Chin J Cancer Res. 2013 April; 25(2): 223-234; Das et al., Front Bioeng Biotechnol. 2021; 9: 795300.

In some embodiments, the myeloid cell activating agent or therapy is an electrostatic treatment. See e.g., Zandi et al., Cancer Med. 2021 November; 10(21): 7475-7491.

In some embodiments, the myeloid cell activating agent or therapy is a thermoacoustic treatment. See e.g., Wen et al., Theranostics. 2017; 7(7): 1976-1989.

In some embodiments, the myeloid cell activating agent or therapy comprises a microbe or component thereof (e.g., a fragment or lysate of a microbe). Examples of microbes include bacteria, fungi, and viruses.

In some embodiments, the myeloid cell activating agent or therapy is an antibody drug conjugate (ADC) or derivative thereof. See, e.g., Baah, S. et al., Molecules 2021; 26(10):2943.

In some embodiments, myeloid cells are engineered by any method known in the art (e.g., CRISPR, TALENS, ZFN, BAC recombineering) to produce SHP-1 and/or tyrosine kinase inhibitory molecules (e.g., inhibitory nucleic acid molecules or inhibitory peptides). In some embodiments, the engineered myeloid cells can be further engineered to produce any of the myeloid cell activating agent or therapys, e.g., pro-inflammatory molecules, described herein.

### TLR Agonists

In some embodiments, the myeloid cell activating agent or therapy comprises or is a TLR agonist.

TLRs play a vital role in activating immune responses. TLRs recognize conserved pathogen-associated molecular patterns (PAMPs) expressed on a wide array of microbes, as well as endogenous DAMPs released from stressed or dying cells. TLR1, -2, -4, -5, -6, and -10 are expressed on the cell surface, whereas TLR3, -7, -8, and -9 are situated on endosomal membranes within the cell. TLR1 and TLR2 can heterodimerize to recognize a variety of bacterial lipid structures and cell wall components, such as triacylated lipoproteins, lipoteichoic acid, and p-glucans. TLR2 also heterodimerizes with TLR6 to bind diacylated lipopeptides. Additionally, TLR2 can bind various endogenous DAMPs, such as HSPs, HMGB1, uric acid, fibronectin, and other extracellular matrix proteins. It has also been suggested that TLR1 and TLR6 can heterodimerize with TLR10; however, the TLR agonist recognized by this dimer remains to be identified. TLR3 recognizes viral dsRNA, as well as synthetic analogs of dsRNA, such as ligand Poly I:C. TLR4 binds LPS in complex with lipid A binding protein, CD14, and myeloid differentiation protein 2 (MD2) as well as recognizing various DAMPs. Endogenous TLR4 ligands, which have been described, include β-defensin 2, fibronectin extra domain A EDA, HMGB1, Snapin, and tenascin C. TLR5 recognizes bacterial flagellin; TLR7 and TLR8 bind viral ssRNA: TLR9 interacts with unmethylated CpG DNA from bacteria and some viruses. Additional TLRs have been identified more recently in mice based on sequence homology of the highly conserved TIR domain. TLR10 is a surface receptor whose natural ligand remains unknown. TLR11, -12, and -13 are present in mice but not in humans. TLR11 was shown to bind a T. gondii profilin and uropathogenic Escherichia coli. The ligand for TLR12 has not yet been identified, whereas TLR13 is an endosomal receptor that recognizes VSV. See e.g., Kaczanowska et al., J Leukoc Biol. 2013 June; 93(6):847-63.

TLR signaling can act as a double-edged sword in cancer. It was found that TLR stimulation of cancer cells can lead to either tumor progression or inhibition. For example, stimulation of TLR2, -4, and -7/-8 was found to lead to tumor progression via production of immunosuppressive cytokines, increased cell proliferation, and resistance to apoptosis. R848-stimulation of a TLR7/8-overexpressing pancreatic cancer cell line resulted in increased cell proliferation and reduced chemosensitivity. However, stimulation of TLR2, -3, -4, -5, -7/-8, and -9, often combined with chemo- or immunotherapy, can lead to tumor inhibition via different pathways. See e.g., Grimmig et al., Int J Oncol. (2015) 47:857-66; Urban-Wojciuk et al., Front Immunol. 2019; 10: 2388.

In some embodiments, the TLR agonist activates any of the TLRs.

In some embodiments, the TLR agonist activates TLR1 or TLR2, optionally wherein the TLR agonist comprises a triacylated lipoprotein, a peptidoglycan, zymosan, and/or Pam₃CSK₄.

In some embodiments, the TLR agonist activates any one of TLR2, TLR3, TLR4, TLR5, and TLR6, optionally wherein the TLR agonist comprises a diacylated lipopeptide, a hot shock protein, HMGB1, uric acid, fibronectin, and/or ECM protein.

In some embodiments, the TLR agonist activates TLR2, optionally wherein the TLR agonist comprises Pam3Cys, SMP-105, and/or CBLB612.

In some embodiments, the TLR agonist activates TLR3, optionally wherein the TLR agonist comprises dsRNA, Poly I:C, PolyICIC, Poly-IC12U, IPH302, ARNAX, and/or MPLA.

In some embodiments, the TLR agonist activates TLR4, optionally wherein the TLR agonist comprises LPS, lipoteichoic acid beta-defensin 2, fibronectin EDA, HMGB1, snapin, tenascin C, OK-432, AS04, and/or GLA-SE.

In some embodiments, the TLR agonist activates TLR5, optionally wherein the TLR agonist comprises flagellin, CBLB502, and/or M-VM3.

In some embodiments, the TLR agonist activates TLR6.

In some embodiments, the TLR agonist activates TLR7 or TLR8, optionally wherein the TLR agonist comprises ssRNA, CpG-A, poly G10, and/or poly G3.

In some embodiments, the TLR agonist activates TLR7, optionally wherein the TLR agonist comprises bistriazolyl and/or R848.

In some embodiments, the TLR agonist activates TLR8, optionally wherein the TLR agonist comprises VTX1463 and/or R848.

In some embodiments, the TLR agonist activates TLR9, optionally wherein the TLR agonist comprises unmethylated CpG DNA, CpG (e.g., CpG-7909, KSK-CpG, CpG-1826), MGN1703, dsSLIM, IMO2055, SD101, and/or ODN M362.

In some embodiments, the TLR agonist activates TLR10, optionally wherein the TLR agonist comprises Pam₃CSK₄.

In some embodiments, the TLR agonist activates TLR11, optionally wherein the TLR agonist comprises Toxoplasma gondii profilin.

In some embodiments, the TLR agonist activates TLR12.

In some embodiments, the TLR agonist activates TLR13, optionally wherein the TLR agonist comprises VSV.

In some embodiments, the TLR agonist activates a TLR on a macrophage.

In some embodiments, the TLR agonist activates TLR1, TLR2, TLR3, TLR4, TLR7, TLR8, and/or TLR9.

In some embodiments, the TLR comprises TLR1, TLR4, and/or TLR9. In some embodiments, the TLR comprises TLR9.

In some embodiments, the TLR comprises TLR2, TLR4, TLR7, and/or TLR8.

In some embodiments, the TLR agonist comprises CpG. In some embodiments, the TLR agonist comprises polyI:C. In some embodiments, the TLR agonist comprises CpG and/or polyI:C. In some embodiments, the TLR agonist comprises CpG, polyI:C, and/or R848. In some embodiments, the TLR agonist comprises CpG, polyI:C, and R848, for example at 1:1:1 ratio.

In some embodiments, the method described herein further comprises assessing whether the individual has an ongoing infection. In some embodiments, a reduced amount of the TLR agonist is administered when the individual has an ongoing infection. In some embodiments, the administration of TLR agonist can be avoided when the individual has an ongoing infection.

### Radiation Therapy

In some embodiments, the myeloid cell activating agent or therapy comprises or is a radiation therapy. Radiation activates the interconnected network of cytokines, adhesion molecule, ROS/RNS, and DAMPs, leading to a self-amplified cascade, which generates pro-inflammatory, pro-oxidant tumor microenvironment and ultimately tumor cell death. See e.g., McKelvey et al., Mamm Genome. 2018; 29(11): 843-865.

In some embodiments, the radiation therapy comprises irradiation at the site of the cancer to be treated.

In some embodiments, the radiation therapy comprises irradiation at a site that is different from the site of the cancer to be treated.

In some embodiments, the radiation therapy is intraoperative radiation therapy (“IORT”). In particular embodiments, the radiation is localized to a tumor site. The patient may be subjected to intraoperative radiation prior to resection of the tumor or following resection of the tumor. The tumor site may comprise different types of cells including cancerous and benign cells. In certain embodiments, the radiation therapy is stereotactic body radiotherapy (“SBRT”) or stereotactic radiosurgery (“SRS”).

In some embodiments, the radiation is ionizing radiation such as particle beam radiation. The particle beam radiation may be selected from any of electrons, protons, neutrons, heavy ions such as carbon ions, or pions. The ionizing radiation may be selected from x-rays, UV-light, γ-rays, or microwaves. In some embodiments, the radiation therapy may comprise subjecting the patient to one or more types of radiation therapy.

In some embodiments, a radio sensitizer is used to sensitize the tumor cells to radiation. The use of such pharmaceuticals, called radiosensitizers, provides a method of increasing the radiosensitivity of tumors to radiation therapy, avoiding the need to increase radiation dosages to levels that are harmful to surrounding organs and tissues. See e.g., U.S. Pat. No. 9,656,098B2.

In some embodiments, the dose of the radiation therapy is non-ablative, and therefore insufficient to eliminate the tumor (i.e., to kill all tumor cells). In some embodiments, the radiation therapy is selected from the group consisting of external-beam radiation therapy, internal radiation therapy (brachytherapy), intraoperative radiation therapy (IORT), systemic radiation therapy, radioimmunotherapy, and administration of radiosensitizers and radioprotectors.

In some embodiments, the radiation therapy is external-beam radiation therapy, optionally comprising three-dimensional conformal radiation therapy (3D-RT), intensity modulated radiation therapy (IMRT), photon beam therapy, image-guided radiation therapy (IGRT), and sterotactic radiation therapy (SRT).

In some embodiments, the radiation therapy comprises administering a radiopharmaceutical. The radiopharmaceuticals can be delivered via any vehicle such as a cell, a protein, or a small molecule complex. In some embodiments, the radiopharmaceutical is administered to the tumor tissue. See e.g., Sgouros et al., Nat Rev Drug Discov 19, 589-608 (2020).

In some embodiments, the radiation therapy is brachytherapy, optionally comprising interstitial brachytherapy, intracavitary brachytherapy, intraluminal radiation therapy, and radioactively tagged molecules given intravenously.

### STING Activator

In some embodiments, the myeloid cell activating agent or therapy comprises or is a STING activator.

Stimulator of LFN genes (STING, also known as TMEM173, MITA, MPYS, or ERIS) is a pattern recognition receptor (PRR) that recognizes cytosolic DNA in the form of cyclic dinucleotides (CDNs), such as the bacterial product cyclic-guanosine monophosphate-adenosine monophosphate (3′3′ cGAMP). In addition to bacterial components, other forms of DNA from viruses, or the host cell, that find their way into the cytosol are recognized by an enzyme c-GMP-AMP (cGAMP) synthase (cGAS). Upon cytosolic DNA binding, cGAS converts ATP and GTP into the metazoan-specific CDN 2′3′-cGAMP for STING recognition and activation. STING is a transmembrane protein that exists as dimers anchored within the endoplasmic reticulum membrane and forms a V-shaped pocket that enables cytosolic CDN binding. Ligand binding results in significant conformational changes in the C-terminal domain of STING, mediating its transport to Golgi compartments. At the Golgi, STING recruits TANK-binding kinase 1 (TBK1), which facilitates IRF3 phosphorylation, nuclear translocation and the strong induction of transcription of type I IFNs (e.g., IFN-p). STING also triggers a robust pro-inflammatory cytokine response (e.g., tumor necrosis factor (TNF)) by activating Nuclear Factor-kappa B (NF-κB) and this part of the pathway can be mediated independent of TBK1 via a closely related homologue protein, IKKϵ. See e.g., Peng et al., Front Immunol. 2022 Feb. 25; 13:794776; Amougezar et al., Cancers (Basel). 2021 May 30; 13(11):2695.

In some embodiments, the STING activator is a cyclic-guanosine monophosphate-adenosine monophosphate (cGAMP, e.g., 3′3′ cGAMP, e.g., 2′3′ cGAMP).

In some embodiments, the STING activator is a bacterial vector (e.g., SYNB1891, STACT-TREX-1).

In some embodiments, the STING activator is a CDN compound (e.g., ADU-S100, BI-STING, BMS-986301, GSK532, JNJ-4412, MK-1454, SB11285, 3′3′-cyclic AIMP).

In some embodiments, the STING activator is a non-CDN small molecule (e.g., ALG-031048, E7755, JNJ-'6196, MK-2118, MSA-1, MSA-2, SNX281, SR-717, TAK676, TTI-10001).

In some embodiments, the STING activator is a nanovaccine (e.g., PC7A NP, cCAMP-NP, ONM-500).

In some embodiments, the STING activator is an ATR inhibitor (e.g., berzosertib). See e.g., Cancer Commun (Lond). 2023 April; 43(4): 435-454. In some embodiments, the ATR inhibitor is used in combination with a radiation therapy.

In some embodiments, the STING activator is an antibody-drug conjugate (e.g., XMT-2056, CRD-5500).

Other exemplary STING activators can be found in Amougezar et al., Cancers (Basel). 2021 May 30; 13(11):2695, which is incorporated by reference here by its entirety.

### PAMP/DAMP Activators

In some embodiments, the myeloid cell activating agent or therapy comprises or is a PAMP/DAMP activator.

The organism senses microbial infection through innate receptors encoded in the genome, called pattern-recognition receptors, including the Toll-like receptors (TLRs), the nucleotide-binding and oligomerization domain (NOD)-like receptors, and retinoic acid-inducible gene I (RIG-I)-like receptors. These receptors recognize pathogen-associated molecular patterns (PAMPs) expressed by bacteria, fungi, and viruses, but also bind damage-associated molecular patterns (DAMPs), which are molecules released by sterile injury. Thus, PAMPs and DAMPs that bind to the same type of receptors initiate identical intracellular pathways terminating in identical effector functions. See e.g., Alisi et al., Hepatology. 2011 November; 54(5):1500-2.

In some embodiments, the myeloid cell activating agent or therapy is a PAMP activator. Exemplary PAMP activator includes triacyl lipopeptides, LPS, lipoprotein, peptidoglycan, zymosan, lipoteichoic acid, trypanosomal phospholipids, Pam3Cys porins, lipoarabinomannan, double-stranded RNA, poly(I:C), trepanosomal lipids, taxol, Pseudomonas exoenzyme S, RSV F protein, MMTV envelope protein, flagellin, diacyl lipopeptides, single-stranded RNA, imiquimod, single-stranded RNA, resquimod, bacterial/viral DNA, CpG DNA, ureobacteria, and toxoplasma LPS.

In some embodiments, the myeloid cell activating agent or therapy is a DAMP activator. Exemplary DAMP activator includes defensins, HSP60, HSP70, messenger RNA, low-molecular-weight hyaluronic acid, fibrinogen, fibronectin, fx1-defensin, heparan sulfate, HSP60, HSP70, HSP90, HMGB1, and unmethylated CpG DNA.

### Chemotherapeutic Agent

In some embodiments, the myeloid cell activating agent or therapy comprises or is a chemotherapeutic agent.

In some embodiments, the chemotherapeutic agent is an alkylating agent. Exemplary alkylating agents include nitrogen mustard (e.g., endamustine, cyclophosphamide, ifosfamide), nitrosoureas (e.g., carmustine, lomustine), platinum analogs (e.g., carboplatin, cisplatin, oxaliplatin), triazenes (e.g., dacarbazine, procarbazine, temozolamide), alkyl sulfonate (e.g., busulfan), and ethyleneimine (e.g., thiotepa).

In some embodiments, the chemotherapeutic agent is an antimetabolite. Exemplary antimetabolites include cytidine analogs (e.g., azacitidine, decitabine, cytarabine, gemcitabine), folate antagonists (e.g., methotrexate, pemetrexed), purine analogs (e.g., cladribine, clofarabine, nelarabine), pyrimidine analogs (e.g., fluorouracil (5-FU), and capecitabine (prodrug of 5-FU)).

In some embodiments, the chemotherapeutic agent is an antimicrotubular agent. Exemplary antimicrotubular agents include topoisomerase II inhibitors (e.g., anthracyclines, doxorubicin, daunorubicin, idarubicin, mitoxantrone), topoisomerase I inhibitors (e.g., irinotecan, topotecan), taxanes (e.g., paclitaxel, docetaxel, cabazitaxel), vinca alkaloids (e.g., vinblastine, vincristine, vinorelbine), and antibiotics (e.g., actinomycin D, bleomycin, daunomycin).

Other exemplary chemotherapeutic agents include hydroxyurea, tretinoin, arsenic trioxide, and proteasome inhibitors (e.g., bortezomib).

### Pro-Inflammatory Cytokines

In some embodiments, the myeloid cell activating agent or therapy is a pro-inflammatory cytokine.

In some embodiments, the pro-inflammatory cytokine promotes the M1 macrophages. In some embodiments, the pro-inflammatory cytokine promotes dendritic cells (e.g., intratumoral). In some embodiments, the pro-inflammatory cytokine promotes B cells (e.g., intatumoral). In some embodiments, the pro-inflammatory cytokine promotes antigen presenting cells. See e.g., Duque et al., Front Immunol. 2014; 5: 491. In some embodiments, the pro-inflammatory cytokine comprises or is IFNγ and/or GM-CSF.

In some embodiments, the pro-inflammatory cytokine comprises IL-6, a cytokine from IL-1 family (e.g., IL-1α, IL-1β, IL-18, IL-33, and IL-36), and/or IFNγ.

In some embodiments, the pro-inflammatory cytokine comprises a cytokine from IL-1 family. In some embodiments, the pro-inflammatory cytokine comprises any one or more of IL-1α, IL-1β, IL-18, IL-33, and IL-36. See e.g., Sims, J., Smith, D. The IL-1 family: regulators of immunity. Nat Rev Immunol 10, 89-102 (2010).

In some embodiments, the myeloid cell activating agent or therapy comprises a cytokine that triggers inflammation. In some embodiments, the cytokine can include, but are not limited to, any one of: TNF family member, IL-1β, IL-6, IL-10, IL-12, IFNα, IFNβ, IFNγ, TGFβ, and GM-CSF.

### Cancer Vaccine

In some embodiments, the myeloid cell activating agent or therapy comprises or is a cancer vaccine. Cancer vaccines stimulate anti-tumor immunity with tumor antigens, which could be delivered in the form of whole cells, peptides, nucleic acids, etc. Ideal cancer vaccines could overcome the immune suppression in tumors and induce both humoral immunity and cellular immunity.

In some embodiments, the cancer vaccine comprises a cell-based vaccine, a peptide-based vaccine, a viral-based vaccine, and/or a nucleic acid-based vaccine. See e.g., Liu et al., J Hematol Oncol 15, 28 (2022).

Cell-based vaccines were the initial form of cancer vaccines. Cell-based cancer vaccines are often prepared from whole cells or cell fragments, containing most tumor antigens, inducing a broader antigen immune response. The DC cell vaccine is an important branch of cell-based vaccines. Personalized neoantigen cancer vaccines based on DCs have shown promising anti-tumor effects in clinical trials. Viruses are naturally immunogenic, and their genetic material can be engineered to contain sequences encoding tumor antigens. Several recombinant viruses, such as adenovirus, can infect immune cells as vectors. The engineered virus vaccines can present tumor antigens in large quantities to the immune system in order to produce anti-tumor immunity. Furthermore, the oncolytic virus can be used as a vectors. Except for providing tumor antigens, the virus itself can also lyse the tumor, release tumor antigens, further increase the vaccine's effectiveness, and produce long-term immune memory.

Peptide-based subunit vaccines, including chemical and biosynthetic preparations of predicted or known specific tumor antigens, induce a robust immune response against the particular tumor antigen site. Peptide-based subunit vaccines combined with adjuvants can efficiently provoke humoral immune response, suitable for preventing and treating viral infectious diseases.

HBV and HPV vaccines for liver and cervical cancers were primarily peptide-based subunit vaccines. Especially, virus-like particles (VLP)-based subunit vaccines that can activate cellular immune responses have shown good anti-tumor activity in recent years.

The nucleic acid vaccine induces strong MHC I mediated CD8+ T cell responses; thus, it is a desirable cancer vaccine platform. Nucleic acid vaccines can simultaneously deliver multiple antigens to trigger humoral and cellular immunity. Additionally, nucleic acid vaccines can encode full-length tumor antigens, allowing APCs to cross-present various epitopes or present several antigens simultaneously. Finally, the nucleic acid vaccine preparation is simple and fast, which is suitable for developing personalized neoantigen cancer vaccines.

### Bacteria, Viruses, and Fungi

In some embodiments, the myeloid cell activating agent or therapy is a bacterium or a component thereof. For example, in some embodiments, the myeloid cell activating agent or therapy is a dead or otherwise inactivated bacterium. In some embodiments, the myeloid cell activating agent or therapy is a weakened bacterium. In some embodiments, the myeloid cell activating agent or therapy is a bacterial protein or a nucleic acid sequence encoding a bacterial protein. In some embodiments, the myeloid cell activating agent or therapy is a bacterial epitope or antigenic region of a bacterial protein or nucleic acid sequence thereof.

In some embodiments, the myeloid cell activating agent or therapy is a virus or a component thereof. For example, in some embodiments, the myeloid cell activating agent or therapy is a dead or otherwise inactivated virus. In some embodiments, the myeloid cell activating agent or therapy is a weakened virus. In some embodiments, the myeloid cell activating agent or therapy is a viral protein or a nucleic acid sequence encoding a viral protein. In some embodiments, the myeloid cell activating agent or therapy is a viral epitope or antigenic region of a viral protein or nucleic acid sequence thereof.

In some embodiments, the myeloid cell activating agent or therapy is a fungus or a component thereof. For example, in some embodiments, the myeloid cell activating agent or therapy is a dead or otherwise inactivated fungus. In some embodiments, the myeloid cell activating agent or therapy is a weakened fungus. In some embodiments, the myeloid cell activating agent or therapy is a fungal protein or a nucleic acid sequence encoding a fungal protein. In some embodiments, the myeloid cell activating agent or therapy is a fungal epitope or antigenic region of a fungal protein or nucleic acid sequence thereof.

### Oncolytic Virus

In some embodiments, the myeloid cell activating agent or therapy is an oncolytic virus (OV). Oncolytic viruses (OVs) are organisms able to identify, infect, and lyse different cells in the tumor environment, aiming to stabilize and decrease the tumor progression. They can present a natural tropism to the cancer cells or be oriented genetically to identify specific targets. See e.g., Apolonio et al., World J Virol. 2021 Sep. 25; 10(5): 229-255.

Oncolytic viruses represent an exciting new avenue of cancer therapy. Such viruses have the remarkable ability to hunt and terminate cancer cells while leaving healthy cells unharmed, as well as enhancing the immune system's ability to recognize and terminate cancer cells. See e.g., Cancer Cell. 2022 Aug. 15; S1535-6108(22):00357-9.

In some embodiments, the oncolytic virus comprises or is an adenovirus (e.g., ONYX-15, LOAd703 virus), a protoparvovirus, a parvovirus (e.g., H-1PV), a vaccinia virus (VACV), a Reovirus (e.g., Reolysin), or a Herpes simplex virus (HSV, e.g., HSV-1, HSV-2, G207, L1BR1, HF10, T-VEC, Orien X010).

Other exemplary oncolytic viruses include JX-593, Coxsackievirus A21 (CVA21), marabá virus or its MG1 variant, DNX2440 adenovirus, fowl pox virus, and Sendai virus.

### Antibody Drug Conjugates

In some embodiments, the myeloid cell activating agent or therapy comprises an antibody drug conjugate (ADC). In some embodiments, the ADC presents a payload to the cancer cells. In some embodiments, the payload is a cytotoxic drug. In some embodiments, the ADC enhances cancer cell killing and thereby induces antigen spreading and activation of antigen-presenting cells. Exemplary ADCs include, but are not limited to, brentuximab vedotin, enfortumab vedotin, gemtuzumab ozogamicin, inotuzumab ozogamicin, polatuzumab vedotin, sacituzumab govitecan, trastuzumab deruxtecan, trastuzumab emtansine, and belantamab mafodotin. Any ADC known in the art may be used as described herein. See, e.g., Baah, S. et al., Molecules 2021; 26(10):2943.

### Cells

In some embodiments, the myeloid cell activating agent or therapy comprises cells that trigger inflammatory factors. In some embodiments, the cells are tumor-infiltrating lymphocytes (TILs). In some embodiments, the cells specifically recognize a tumor antigen (e.g., being engineered to express a CAR recognizing a tumor antigen). In some embodiments, the cells are T cells. In some embodiments, the cells are CAR-T cells. In some embodiments, the cells are NK cells (e.g., CAR-NK cells). In some embodiments, the immune cells are neutrophils (e.g., CAR-expressing neutrophils cells). In some embodiments, the cells are TCR-T cells. In some embodiments, the cells are APCs (e.g., macrophages or dendritic cells). In some embodiments, the cells are CAR-macrophages or CAR-monocytes. In some embodiments, the cells are SIRPant-macrophages. In some embodiments, the cells are stem cells. In some embodiments, the cells are allogenic. In some embodiments, the cells are autologous.

In some embodiments, the methods of treatment comprise administering to the individual an effective amount of tumor-infiltrating lymphocytes (TILs). TILs arise in the microenvironment of solid tumors and possess specific cytotoxicity against tumor cells, yet tend to be inhibited in situ due to immunosuppressive factors within the said microenvironment. In some embodiments, the TILs are T cells. In some embodiments, the TILs are NK cells. In some embodiments, the TILs are B cells.

Immunotherapeutic methods exist to isolate infiltrating lymphocytes (e.g., tumor-resident T cells) from a patient's tumor tissue, amplify TILs in vitro, and deliver back to the patient through transplantation to kill tumor cells specifically. In some embodiments, the method comprises obtaining fresh tumor tissue through surgery. In some embodiments, tumor tissue cells comprising TILs are separated in vitro. In some embodiments, the separation comprises collagenase-free mechanical separation the fresh tumor tissue. In some embodiments, separated tumor tissue cells are cultured in vitro. In some embodiments, separated tumor tissue cells are cultured in the presence of IL-2. In some embodiments, separated tumor tissue cells are clonally expanded in vitro. In some embodiments, separated tumor tissue cells are screened to detect TILs. In some embodiments, screening comprises exon sequencing. In some embodiments, screening of TILs comprises detection of antigen specificity to tumor cells. In some embodiments, detection of antigen specificity is conducted with methods comprising the ELISpot test.

In some embodiments, TILs are expanded in vitro following screening. In some embodiments, TILs are expanded in cultures. In some embodiments, cultures for TIL expansion comprise IL-2. In some embodiments, cultures for TIL expansion comprise one or more antibody agonists. In some embodiments, cultures for TIL expansion comprise anti-4-1BB. In some embodiments, cultures for TIL expansion comprise anti-CD3. In some embodiments, cultures for TIL expansion comprise Cd137/4-1bb and IL-2. In some embodiments, TILs are expanded in gas-permeable flasks (e.g., G-REX flasks).

In some embodiments, TILs are administered to patients following in vitro expansion. In some embodiments, TILs are administered to patients receiving chemotherapy. In some embodiments, TILs are administered to patients before receiving chemotherapy. In some embodiments, TILs are administered to patients during chemotherapy. In some embodiments, TILs are administered to patients after receiving chemotherapy.

In some embodiments, the methods of treatment comprise administering to the individual an effective amount of cells comprising a chimeric antigen receptor (CAR). CARs are engineered synthetic receptors that function to redirect lymphocytes (e.g., T cells) to recognize and eliminate cells expressing a specific target antigen. CAR binding to target antigens expressed on the cell surface is independent from the MHC receptor, and can result in vigorous lymphocytes activation (e.g., T cell activation). In some embodiments, a CAR specifically binds an antigen associated with cancer. In some embodiments, the CAR is specific against an antigen selected from the group consisting of CD19, CD20, CD22, HER2, IL13Ra2, MUC1, PSMA, EGFR, MSLN, CEA, and BMCA. In some embodiments, the cells comprising a chimeric antigen receptor (CAR) are CAR-T cells.

### Lymphocyte Activating Agents

In some embodiments, the methods of treatment described herein further comprise administering to the individual an effective amount of a lymphocyte activating agent. In some embodiments, the lymphocyte is a T cell (e.g., a CD4+ T cell, a CD8+ T cell, a regulatory T cell, a helper T cell, a cytotoxic T cell, a memory T cell, an effector T cell, a naïve T cell, a genetically engineered T cell). In some embodiments, the lymphocyte is an NKT cell (e.g., a CAR-NKT cell). In some embodiments, the lymphocyte is a B cell (e.g., an intratumoral B cell, a follicular B cell, a marginal zone B cell, a transitional B cell, a naïve B cell, a plasma cell, a memory B cell, a CAR-B cell)

In some embodiments, the lymphocyte activating agent is selected from the group consisting of: a cytokine, a chemokine, a metabolism-modulating drug, a metabolite antagonist, an immune checkpoint inhibitor, an immune cell, a cancer vaccine, a bacteria or component thereof, a virus or component thereof, a fungus or component thereof, a bispecific T cell engager (BiTE), an antibody drug conjugate, and any combination thereof.

Cancer vaccines, bacteria and components thereof, viruses (e.g., oncolytic viruses) and components thereof, fungi and components thereof, and antibody drug conjugates and derivatives thereof are described above as myeloid cell activating agent or therapys. These same components can also be capable of activating lymphocytes and are included herein by reference.

### Cytokines and Chemokines

In some embodiments, the lymphocyte activating agent is a cytokine or chemokine (e.g., a cytokine or chemokine that promotes T cells, such as CD8+ cytotoxic T cells).

In some embodiments, the cytokine promotes T cell survival and/or expansion, such as IL-2, IL-7, IL-15, and IL-21. In some embodiments, the cytokine promotes T cell stimulation, such as CD27L or 41BBL. In some embodiments, the cytokine promotes B cell proliferation and/or activation, including but not limited to IL-4, IL-5, IL-6, and IL-10. See, e.g., Zhang, Y. et al., Front Immunol. 2020 Dec. 14; 11:594609; and Vazquez, M. et al., Cytokine 2015 August; 74(2):318-326.

In some embodiments, the lymphocyte activating agent comprises a chemokine. In some embodiments, the chemokine can include chemokines that signal for cells, e.g., T cells or NK cells, to migrate into the tumor or to migrate to DCs for priming. For example, CXCL9, CXCL10, and CXCL11 can attract T cells and are produced by dendritic cells to induce T cells to migrate to dendritic cells where the T cells can be primed, e.g., against tumor cells. Additional chemokines can include, but are not limited to, CCL2, CCL3, CCL4, CCL5, CCL21, CCL27, CCL28, CXCL1, CXCL2, CXCL8, CXCL16, and LTB4.

### Checkpoint Inhibitors

In some embodiments, the lymphocyte activating agent is a checkpoint inhibitor. Immune checkpoints are pathways with inhibitory or stimulatory features that maintain self-tolerance and assist with immune response. The most well-described checkpoints are inhibitory in nature and include the cytotoxic T lymphocyte-associated molecule-4 (CTLA-4), programmed cell death receptor-1 (PD-1), and programmed cell death ligand-1 (PD-L1). See e.g., Marin-Acevedo et al., J Hematol Oncol 14, 45 (2021).

In some embodiments, the checkpoint inhibitor targets CLTA-4, PD-1 or PD-L1 (e.g., an antibody targeting CTLA-4, PD-1 or PD-L1).

In some embodiments, the checkpoint inhibitor targets LAG-3, TIM-3, B7-H3, B7-H4, A2aR, CD73, NKG2A, PVRIG/PVRL2, CEACAM1, CEACAM 5/6, FAK, CCL2/CCR2, LIF, CD47/SIRPα, CSF-1 (M-CSF)/CSF-1R, IL-1/IL-1R3 (IL-1RAP), IL-8, SEMA4D, Ang-2, CLEVER-1, Axl, or phosphatidylserine.

In some embodiments, the checkpoint inhibitor comprises or is ipilimumab, Cemiplimab, Nivolumab, Pembrolizumab, Atezolizumab, Avelumab, Durvalumab, LAG525 (IMP701), REGN3767, BI 754,091, tebotelimab (MGD013), eftilagimod alpha (IMP321), FS118, MBG453, Sym023, TSR-022, MGC018, FPA150, EOS100850, AB928, CPI-006, Monalizumab, COM701, CM24, NEO-201, Defactinib, PF-04136309, MSC-1, Hu5F9-G4 (5F9), ALX148, TTI-662, RRx-001, Lanotuzumab (MCS110), LY3022855, SNDX-6352, Emactuzumab (RG7155), Pexidartinib (PLX3397), CAN04, Canakinumab (ACZ885), BMS-986253, Pepinemab (VX15/2503), Trebananib, FP-1305, Enapotamab vedotin(EnaV), or Bavituximab.

### Metabolism-Modulating Drugs and Metabolite Antagonists

Tumors are highly metabolically active and have high metabolic demands. Similarly, immune cells become highly metabolically active once the immune cells (e.g., T cells) are activated. The metabolic demands of cancer cells and immune cells, however, are not identical, thereby creating the opportunity to target specific metabolic pathways. Similar to metabolism-modulating drugs, metabolites can be similarly targeted to promote antitumor immunity.

Examples of metabolism-modulating drugs to target cancer cells include, but are not limited to: drugs that target mutant isocitrate dehydrogenases (e.g., Enasidenib, Ivosidenib, Indoximod, Epacadostat); glutaminase inhibitor, CB-839; inhibitor of LATI-dependent neutral amino acid transport, JPH203 or KYT-0353; kidney-type glutaminase (GLS1)-specific inhibitors, such as the allosteric inhibitor BPTES or CB-839; lactate dehydrogenase A inhibitor, NCGC00420737-09; monocarboxylate transporter 1 (MCT1) inhibitor, AZ3965; RNA to DNA conversion inhibitor, hydroxyurea; purine synthesis inhibitor, 6-Mercaptopurine; etc.

Examples of metabolite antagonists include, but are not limited to: folate antagonists (e.g., aminopterin, methotrexate, pemetrexed); ASCT2/glutamine antagonist (V-9302); reactive diazo glutamine analogue, DON, DON prodrugs JHU-083 (ethyl 2-(2-amino-4-methylpentanamido)-DON) or DRP-104; 2-amido-6-benzenesulfonamide glucosamine inhibitor, GSK compound 27; nucleotide analogues (e.g., Gemcitabine, Fludarabine); etc. See, e.g., Stine, Z. E. et al. Nat Rev. Drug Discov. 2022 February; 21(2): 141-162.

### T Cell Binding Moieties

In some embodiments, the lymphocyte activating agent comprises a binding moiety that binds to a T cell. In some embodiments, the binding moiety binds to a T cell receptor (TCR). In some embodiments, the binding moiety is an antibody. In some embodiments, the antibody is a TCR activating antibody (e.g., an anti-CD3 antibody, e.g., an anti-CD28 antibody). In some embodiments, the TCR activating antibody is an anti-CD3 antibody. In some embodiments, the TCR activating antibody is an anti-CD28 antibody. In some embodiments, activation of TCR initiates T cell activation and determines the specificity of the immune response.

### Bispecific T Cell Engagers

In some embodiments, the lymphocyte activating agent comprises a bispecific T cell engager (BiTe). Bispecific T cell engagers redirect T cells to target the cancer cells for T cell-mediated cytolysis of the cancer cells by binding to T cells (e.g., through an anti-CD3 antigen binding moiety) and binding to cancer cells (e.g., through an anti-tumor antigen binding moiety, for example CD19 or CD22 to target B cell malignancies), thereby creating an immunological synapse to drive T cell-mediated cytolysis of the cancer cells. Bispecific T cell engagers generally include two single-chain variable fragments (scFvs) that are connected to each other in tandem by a short linker. In some embodiments, the bispecific T cell engager possesses TCR ligating functionality.

Exemplary BiTes include but are not limited to, Blinatumomab, Pasotuxizumab, Cibisatamab, AMV564, AMG 160, AMG 330, AMG 673, AMG 420, AMG 701, AMG 596, AMG 757, AMG 199, AMG 910, HPN424, M701, M802, and ERY974. Any BiTe known in the art may be used as described herein. See, e.g., Zhou, S. et al., Biomarker Res 2021; 9:38.

### Cells

In some embodiments, the lymphocyte activating agent comprises cells that trigger or otherwise activate lymphocytes. In some embodiments, the cells are APCs (e.g., macrophages or dendritic cells). In some embodiments, the cells are macrophages (e.g., tumor-infiltrating). In some embodiments, the cells are dendritic cells. In some embodiments, the cells are SIRPant-macrophages. In some embodiments, the immune cells comprise monocytes or macrophages described herein. In some embodiments, the macrophages are identified by F4/80 expression. In some embodiments, the macrophages have a M1 phenotype. In some embodiments, at least 50%, 60%, 70%, 80%, 90%, 95%, 98%, or 99%) of the macrophages in the immune cells have a M1 phenotype.

In some embodiments, the myeloid cells (e.g., monocytes or macrophages) are engineered to be deficient in SHP-1 expression and/or activity or tyrosine kinase inhibitor expression and/or activation. In some embodiments, the myeloid cells (e.g., monocytes or macrophages) are engineered to be deficient in SHP-1 expression and/or activity and tyrosine kinase inhibitor expression and/or activation. In some embodiments, the monocytes or macrophages express a reduced level of SHP-1 and/or tyrosine kinase for at least a period of time (e.g., for at least 1, 2, 3, 4, or 5 days) or are resistant to activation for at least a period of time (e.g., for at least 1, 2, 3, 4, or 5 days). In some embodiments, the period of time is no more than about 10, 9, 8, 7, 6, 5, 4, or 3 days. In some embodiments, the myeloid cells (e.g., monocytes or macrophages) have reduced SHP-1 and/or tyrosine kinase activity for no more than about 5 consecutive days (e.g., for no more than 5, 4, or 3 days) before the SHP-1 and/or tyrosine kinase activity level returns to normal.

Methods to engineer myeloid cells (e.g., monocytes or macrophages) to transiently express a reduced level of tyrosine kinase are well-known in the field. Exemplary methods include contacting the monocytes or macrophages with a tyrosine kinase inhibitor described herein (such as a small molecule, a nucleic acid (e.g., a siRNA, a shRNA, an antisense RNA, a microRNA), a nucleic acid editing system (e.g., a CRISPR system), and a protein agent (e.g., an antibody agent that targets SHP-1, tyrosine kinases, or activated tyrosine kinase)) in vivo or in vitro.

In some embodiments, the myeloid cells express a high level of MHC-I, MHC-II, CD80 and/or CD86. In some embodiments, the myeloid cells express a high level of MHC-I, MHC-II, CD80, and/or CD86 when the expression level of MHC-I, MHC-II, CD80 and/or CD86 on the immune cells is comparable (e.g., at least more than 50%) of that on activated antigen presenting cells (APCs). In some embodiments, the myeloid cells express a pro-inflammatory cytokine, optionally wherein the pro-inflammatory cytokine comprises IL-12. In some embodiments, the myeloid cells do not express a significant level of TGFβ and/or IL-10.

In some embodiments, the cells are tumor-infiltrating lymphocytes. In some embodiments, the cells specifically recognize a tumor antigen (e.g., being engineered to express a CAR recognizing a tumor antigen). In some embodiments, the cells are T cells. In some embodiments, the cells are CAR-T cells. In some embodiments, the cells are NK cells (e.g., CAR-NK cells). In some embodiments, the cells are neutrophils (e.g., CAR-expressing neutrophils cells). In some embodiments, the cells are TCR-T cells. In some embodiments, the cells are CAR-macrophages or CAR-monocytes.

In some embodiments, the cells are stem cells. In some embodiments, the stem cells are hematopoietic stem cells (HSC). In some embodiments, the stem cells are pluripotent stem cells. In some embodiments, the stem cells are capable of differentiating and/or producing lymphocytes. In some embodiments, the cells are progenitors. In some embodiments, the progenitors are multipotent progenitors (MPPs). In some embodiments, the progenitors are common lymphocyte progenitors (CLPs). In some embodiments, the progenitors are capable of differentiating and/or producing lymphocytes.

In some embodiments, the cells are allogenic. In some embodiments, the cells are autologous.

In some embodiments, the SHP-1 inhibitor and/or the tyrosine kinase inhibitor, the immune cells, a myeloid cell activating agent or therapy, and a TNFα inhibitor described above are administered within 24 hours (e.g., 12 hours, 8 hours, 4 hours, 3 hours, 2 hours, 1 hour, or 0.5 hour) of each other. In some embodiments, the SHP-1 inhibitor and/or the tyrosine kinase inhibitor, the immune cells, a myeloid cell activating agent or therapy, and a TNFα inhibitor described above are administered within 3 hours of each other. In some embodiments, the immune cells are administered simultaneously or concurrently with the SHP-1 inhibitor, the tyrosine kinase inhibitor, the TNFα inhibitor, and/or the myeloid cell activating agent or therapy.

### Immunogenic Cell Death

In some embodiments, the individual has immunogenic cell death when being treated with the methods described herein.

Immunogenic cell death (ICD) is a type of cancer cell death that can be induced by different stressors, including but not limited to (1) intracellular pathogens; (2) conventional chemotherapeutics such as anthracyclines, DNA-damaging agents, and proteasomal inhibitors; (3) targeted anti-cancer agents such as the tyrosine kinase inhibitor crizotinib, the epidermal growth factor receptor-specific monoclonal antibody cetuximab and poly-ADP-ribose polymerase (PARP) inhibitors; and (4) numerous physical modalities, encompassing hypericin- and redaporfin-based photodynamic therapy, extracorporeal photochemotherapy, various forms of ionizing radiation, high hydrostatic pressure, and severe heat shock. It involves the activation of the immune system against cancer in immunocompetent hosts. ICD comprises the release of damage-associated molecular patterns (DAMPs) from dying tumor cells that result in the activation of tumor-specific immune responses, thus eliciting long-term efficacy of anti-cancer drugs by combining direct cancer cell killing and antitumor immunity. DAMPs include the cell surface exposure of calreticulin (CRT) and heat-shock proteins (HSP70 and HSP90), extracellular release of adenosine triphosphate (ATP), high-mobility group box-1 (HMGB1), type I IFNs and members of the IL-1 cytokine family. See e.g., Ahmed et al., Mol Oncol. 2020 December; 14(12):2994-3006 and Fucikova et al., Cell Death Dis. 2020 Nov. 26; 11(11):1013.

Key DAMPs for cell death to be perceived as immunogenic include calreticulin, high-mobility group box 1 (HMGB1), ATP, annexin A1 (ANXA1), and type I IFN. The main hallmarks of immunogenic cell death (ICD) can be assessed by flow cytometry, (immuno)fluorescence microscopy, immunoblotting, or luminometry, based on a variety of different approaches. See e.g., Cell Death Dis. 2020 Nov. 26; 11(11):1013.

In some embodiments, the individual has ICD (e.g., in the tumor, e.g., in a site distinct from the tumor) within about one week, 6 days, 5 days, 4 days, 3 days, 2 days, or one day prior to and/or after the administration of the SHP-1 inhibitor and/or the tyrosine kinase inhibitor.

In some embodiments, the individual has ongoing ICD (e.g., in the tumor, e.g., in a site distinct from the tumor) when the SHP-1 inhibitor and/or the tyrosine kinase inhibitor are administered.

In some embodiments, the individual has ICD when a sample from the cancer has a higher level of one or more (e.g., at least 10%, 20%, 30%, 40%, 50%, 60%, 70%, 80%, 90°/more) DAMPs than a reference sample (e.g., a corresponding sample in a healthy control, e.g., a sample from the cancer prior to the administration of a therapy that induces ICD. In some embodiments, the DAMPs are selected from the group consisting of endoplasmic reticulum (ER) chaperones (e.g., calreticulin (CALR), e.g., heat-shock proteins (HSPs)), the non-histone chromatin-binding protein high-mobility group box 1 (HMGB1), the cytoplasmic protein annexin A1 (ANXA1), and the small metabolite ATP, and type I interferons (IFNs).

### Individuals

In some embodiments, the individual has a solid tumor. In some embodiments, the individual has a hematologic cancer.

In some embodiments, the individual has an advanced cancer. In some embodiments, the individual has a late-stage cancer. In some embodiments, the individual has a malignant cancer. In some embodiments, the individual has a cancer that is in stage II, III or IV. In some embodiments, the individual has an inoperable tumor and/or metastases. In some embodiments, the individual is a terminally ill individual.

In some embodiments, the individual has been subjected (e.g., within 1, 2, 4, 8, 12, 16, 20, or 24 hours, e.g., within 1, 2, 3, 4, 5, 6 or 7 days before the administration of the TNFα inhibitor and myeloid cell activating agent or therapy, and optionally before the administration of SHP-1 inhibitor and/or tyrosine kinase inhibitor) to a therapy that induces an inflammation reaction or an immunogenic cell death (e.g., radiotherapy). In some embodiments, the individual is to be subjected to (e.g., within 1, 2, 4, 8, 12, 16, 20, or 24 hours, e.g., within 1, 2, 3, 4, 5, 6 or 7 days after the administration of the TNFα inhibitor and myeloid cell activating agent or therapy, and optionally before the administration of SHP-1 inhibitor and/or tyrosine kinase inhibitor) a therapy that induces an inflammation reaction or an immunogenic cell death (e.g., radiotherapy).

In some embodiments, the individual has been subjected (e.g., within 1, 2, 4, 8, 12, 16, 20, or 24 hours, e.g., within 1, 2, 3, 4, 5, 6 or 7 days before the administration of the SHP-1 inhibitor and/or the tyrosine kinase inhibitor) to a myeloid cell activating agent or therapy (such as any of the agents described herein). In some embodiments, the individual is to be subjected to (e.g., within 1, 2, 4, 8, 12, 16, 20, or 24 hours, e.g., within 1, 2, 3, 4, 5, 6 or 7 days after the administration of the SHP-1 inhibitor and/or the tyrosine kinase inhibitor) a myeloid cell activating agent or therapy (such as any of the agents described herein).

In some embodiments, the individual has been subjected (e.g., within 1, 2, 4, 8, 12, 16, 20, or 24 hours, e.g., within 1, 2, 3, 4, 5, 6 or 7 days before the administration of the SHP-1 inhibitor and/or the tyrosine kinase inhibitor) to a TNFα inhibitor (such as any of the TNFα inhibitors described herein). In some embodiments, the individual is to be subjected to (e.g., within 1, 2, 4, 8, 12, 16, 20, or 24 hours, e.g., within 1, 2, 3, 4, 5, 6 or 7 days after the administration of the SHP-1 inhibitor and/or the tyrosine kinase inhibitor) a TNFα inhibitor (such as any of the TNFα inhibitors described herein).

In some embodiments, the individual does not have an autoimmune disease.

In some embodiments, the individual is a female. In some embodiments, the individual is a male.

In some embodiments, the individual is a human. In some embodiments, the individual is at least about 50, 55, 60, 65, 70 or 75 years old.

In some embodiments, the individual is selected for treatment based upon a high expression level and/or a high activation level of SHP-1 in the tumor tissue. In some embodiments, the individual has a high expression level and/or a high activation level of SHP-1 when the expression level and/or the activation level is at least about 20%, 30%, 40%, 50%, 60%, 70%, 80%, 90%, 100%, 125%, 150%, 175%, or 200% more than a reference expression level and/or a reference activation level of SHP-1. In some embodiments, the individual has a high expression level and/or a high activation level of SHP-1 when the expression level and/or the activation level is at least about 5-fold, 10-fold, 20-fold, 30-fold, 40-fold, 50-fold, 60-fold, 70-fold, 80-fold, 90-fold, 100-fold, 150-fold, 200-fold, 250-fold, 500-fold, or 1000-fold more than a reference expression level and/or a reference activation level of SHP-1. In some embodiments, the reference expression level or the reference activation level of SHP-1 is the corresponding expression or activation level of SHP-1 in a reference state, wherein the individual is not treated with a myeloid cell activating agent or therapy (or any immune therapy).

In some embodiments, the individual is selected for treatment based upon a high expression level and/or a high activation level of tyrosine kinases in the tumor tissue. In some embodiments, the individual has a high expression level and/or a high activation level of tyrosine kinase when the expression level and/or the activation level is at least about 20%, 30%, 40%, 50%, 60%, 70%, 80%, 90%, 100%, 125%, 150%, 175%, or 200% more than a reference expression level and/or a reference activation level of tyrosine kinase. In some embodiments, the individual has a high expression level and/or a high activation level of TYROSINE KINASE when the expression level and/or the activation level is at least about 5-fold, 10-fold, 20-fold, 30-fold, 40-fold, 50-fold, 60-fold, 70-fold, 80-fold, 90-fold, 100-fold, 150-fold, 200-fold, 250-fold, 500-fold, or 1000-fold more than a reference expression level and/or a reference activation level of tyrosine kinases. In some embodiments, the reference expression level or the reference activation level of tyrosine kinases is the corresponding expression or activation level of tyrosine kinases in a reference state, wherein the individual is not treated with a myeloid cell activating agent or therapy (or any immune therapy).

In some embodiments, the individual is selected for treatment based upon a high expression level and/or a high activation level of SHP-1 and of tyrosine kinases in the tumor tissue. In some embodiments, the individual has a high expression level and/or a high activation level of SHP-1 and of tyrosine kinase when the expression level and/or the activation level is at least about 20%, 30%, 40%, 50%, 60%, 70%, 80%, 90%, 100%, 125%, 150%, 175%, or 200%6 more than a reference expression level and/or a reference activation level of SHP-1 and of TYROSINE KINASE. In some embodiments, the individual has a high expression level and/or a high activation level of SHP-1 and of tyrosine kinase when the expression level and/or the activation level is at least about 5-fold, 10-fold, 20-fold, 30-fold, 40-fold, 50-fold, 60-fold, 70-fold, 80-fold, 90-fold, 100-fold, 150-fold, 200-fold, 250-fold, 500-fold, or 1000-fold more than a reference expression level and/or a reference activation level of SHP-1 and of tyrosine kinase. In some embodiments, the reference expression level or the reference activation level of SHP-1 and of tyrosine kinase is the corresponding expression or activation level of SHP-1 and of tyrosine kinase in a reference state, wherein the individual is not treated with a myeloid cell activating agent or therapy (or any immune therapy).

In some embodiments, the individual does not develop cytokine release syndrome or pro-inflammatory organ damage. Cytokine release syndrome can damage or cause organ failure in most organ systems. For example, organs that can become damaged due to CRS WO 2024/1%822 PCT/US2024/020307 may include, but are not limited to, the lungs, the kidneys, the liver, the brain, the heart, the spleen, or any combination thereof, for example multi-organ failure.

In some embodiments, the individual develops mild cytokine release syndrome. In some embodiments, the individual develops CRS of grade 1. Mild symptoms of CRS can include fever, fatigue, headache, rash, arthralgia, and myalgia. Mild CRS can be treated by treating the symptoms or by administration of anti-inflammatory drugs such as corticosteroids. Mild CRS can often be resolved within one to two weeks and does not require or necessitate hospitalization.

In some embodiments, the individual does not develop severe cytokine release syndrome. In some embodiments, the individual does not develop CRS of grade 2. In some embodiments, the individual does not develop CRS of grade 3. In some embodiments, the individual does not develop CRS of grade 4. More severe cases are characterized by hypotension and high fever, and severe CRS can progress to an uncontrolled systemic inflammatory response with vasopressor-requiring circulatory shock, vascular leakage, disseminated intravascular coagulation, and multi-organ system failure. More severe cases of CRS often require hospitalization of symptoms. Laboratory abnormalities that are common in patients with CRS include cytopenias, elevated creatinine and liver enzymes, deranged coagulation parameters, and a high CRP. There are four grading systems currently used for cytokine release syndrome, as shown in Table 1 below. See, e.g., Liu, D. and Zhao, J., J Hematol Oncol. 2018 Sep. 24; 11(1):121; and Shimabukuro-Vornhagen, A. et al., J Immunother Cancer. 2018 Jun. 15; 6(1):56, hereby incorporated by reference in their entirety.

In some embodiments, the individual has developed CRS prior to administration of a TNFα inhibitor. In some embodiments, the individual has developed CRS of grade 1. In some embodiments, the individual has developed CRS of grade 2. In some embodiments, the individual has developed CRS of grade 3. In some embodiments, the individual has developed CRS of grade 4. In some embodiments, the individual who has developed CRS is administered a TNFα inhibitor. In some embodiments, the TNFα inhibitor ameliorates, eliminates, or reverses the CRS, including organ damage, for example pro-inflammatory organ damage (e.g., nephritis, hepatitis, pneumonitis, myocarditis, appendicitis).

TABLE 1
Cytokine release syndrome medical grading systems.

| | Penn grading scale | CTCAE v4.0 | 2014 Lee et al. | MDACC |
|---|---|---|---|---|
| Grade | Mild reaction: treated with | Mild reaction; | Symptoms are | Temperature ≥ |
| 1 | supportive care such as | infusion | not life- | 38° C. (fever) or |
| | antipyretics and | interruption not | threatening and | grade 1 organ |
| | antiemetics | indicated; | require | toxicity |
| | | intervention not | symptomatic | |
| | | indicated | treatment only, | |
| | | | e.g., fever, | |
| | | | nausea, fatigue, | |
| | | | headache, | |
| | | | myalgias, and | |
| | | | malaise | |
| Grade | Moderate reaction: some | Therapy or | Symptoms | Systolic blood |
| 2 | signs of organ dysfunction | infusion | require and | pressure < 90 |
| | (e.g., grade 2 creatinine or | interruption | respond to | mmHg |
| | grade 3 LFTs) related to | indicated but | moderate | (hypotension) but |
| | CRS and not attributable | responds | intervention. | responds to IV |
| | to any other condition. | promptly to | Oxygen | fluids or low- |
| | Hospitalization for | symptomatic | requirement < 40% | dose vasopressors |
| | management of CRS- | treatment (e.g., | or hypotension | or needing |
| | related symptoms, | antihistamines, | responsive to | oxygen |
| | including fevers with | NSAIDS, | fluids or low dose | (FiO2 < 40%) |
| | associated neutropenia and | narcotics, IV | pressors or grade | for SaO2 > 90% |
| | need for IV therapies (not | fluids); | 2 organ toxicity | (hypoxia) |
| | including fluid | prophylactic | | or grade 2 organ |
| | resuscitation for | medications | | toxicity |
| | hypotension) | indicated | | |
| | | for ≤24 h | | |
| Grade | More severe reaction: | Prolonged | Symptoms | Systolic blood |
| 3 | hospitalization required | reaction (e.g., not | require and | pressure < 90 |
| | for management of | rapidly | respond to | mmHg |
| | symptoms related to organ | responsive to | aggressive | (hypotension) and |
| | dysfunction, including | symptomatic | intervention. | needs high-dose |
| | grade 4 LFTs or grade 3 | medication and/or | Oxygen | or multiple |
| | creatinine related to CRS | brief interruption | requirement ≥ 40 | vasopressors or |
| | and not attributable to any | of infusion); | % or | needing |
| | other conditions; this | recurrence of | hypotension | oxygen(FiO2 ≥ 40%) |
| | excludes management of | symptoms | requiring high- | for SaO2 > 90% |
| | fever or myalgias; | following initial | dose or multiple | (hypoxia) |
| | includes hypotension | improvement; | pressors or grade | or grade 3 organ |
| | treated with IV fluids | indicated for | 3 organ toxicity | toxicity or grade |
| | (defined as multiple fluid | clinical sequelae | or grade 4 | 4 transaminitis |
| | boluses for blood pressure | (e.g., renal | transaminitis | |
| | support) or low-dose | impairment, | | |
| | vasopressors, | pulmonary | | |
| | coagulopathy requiring | infiltrates) | | |
| | fresh frozen plasma or | | | |
| | cryoprecipitate or | | | |
| | fibrinogen concentrate, | | | |
| | and hypoxia requiring | | | |
| | supplemental oxygen | | | |
| | (nasal cannula oxygen, | | | |
| | high-flow oxygen, CPAP, | | | |
| | or BiPAP). Patients | | | |
| | admitted for management | | | |
| | of suspected infection due | | | |
| | to fevers and/or | | | |
| | neutropenia may have | | | |
| | grade 2 CRS | | | |
| Grade | Life-threatening | Life-threatening | Life-threatening | Life-threatening |
| 4 | complications such as | consequences; | symptoms. | hypotension or |
| | hypotension requiring | pressor or | Requirements for | needing ventilator |
| | high dose vasopressors | ventilator support | ventilator support | support or grade |
| | and a hypoxia requiring | indicated | or grade 4 oxygen | 4 oxygen toxicity |
| | mechanical ventilation | | toxicity | (excluding |
| | | | (excluding | transaminitis) |
| | | | transaminitis) | |

In some embodiments, the individual does not develop cytokine storm. In some embodiments, the individual develops mild cytokine storm. In some embodiments, the individual does not develop severe or life-threatening cytokine storm. Cytokine storm appears to be mainly a result of non-specific T cell activation, whereas CRS is more often a direct consequence of antigen-specific T cell activation. The clinical manifestations of cytokine storm and CRS can be similar (Liu, D. and Zhao, J., J Hematol Oncol. 2018 Sep. 24; 11(1):121).

In some embodiments, administration of the TNFα inhibitor to an individual in need thereof does not compromise or weakly compromises tumor clearance in the individual. In some embodiments, administration of the TNFα inhibitor to an individual in need thereof decreases serum IFNγ levels by less than about 50% (e.g., by less than about any of 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, or less). In some embodiments, administration of the TNFα inhibitor to an individual in need thereof decreases serum IFNγ levels by less than 30%. In some embodiments, administration of the TNFα inhibitor to an individual in need thereof decreases CXCL10 levels by less than about 50% (e.g., by less than about any of 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, or less). In some embodiments, administration of the TNFα inhibitor to an individual in need thereof decreases CXCL10 levels by less than 30%. In some embodiments, the tumor clearance in an individual administered the TNFα inhibitor and any of the anti-cancer therapies described herein is comparable to the tumor clearance in an individual administered any of the anti-cancer therapies described herein without a TNFα inhibitor. In some embodiments, administration of the TNFα inhibitor to an individual in need thereof reduces overall systemic pro-inflammatory cytokine levels, for example as measured from a sample of the individual's serum.

In some embodiments, the individual is an animal, for example a reptile, a bird, a fish, or a mammal. In some embodiments, the individual is a mammal. In some embodiments, the individual includes human and veterinary subjects. In some embodiments, the individual is a mammal, including, but not limited to, humans, rodents, simians, felines, canines, equines, bovines, porcines, ovines, caprines, mammalian laboratory animals, mammalian farm animals, mammalian sport animals, and mammalian pets. In some embodiments, the individual is a human.

### Cancer

Cancer described here can be any type or kind. In some embodiments, the cancer is a solid tumor. In some embodiments, the cancer is a hematologic cancer.

In some embodiments, the cancer is an advanced cancer. In some embodiments, the cancer is a late-stage cancer. In some embodiments, the cancer is a terminal cancer. In some embodiments, the cancer is in stage II, III or IV. In some embodiments, the cancer is an inoperable tumor and/or is malignant.

In some embodiments, the tumor is at least 0.2 cm, 0.4 cm, 0.6 cm, 0.8 cm, 1 cm, 2 cm, 3 cm, 4 cm or 5 cm in length.

Examples of cancers described herein include, but are not limited to, adrenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, craniopharyngioma, haemangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, melanoma, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), and Meigs' syndrome.

In some embodiments, the cancer is a virus-infection-related cancer. In some embodiments, the cancer is a human papillomavirus (HPV)-related cancer (e.g., HPV-related cervical cancer, e.g., HPV-related head and neck cancer, e.g., HPV related squamous cell carcinoma). In some embodiments, the cancer is human herpes virus 8 (HHV8) related cancer (e.g., Kaposi sarcoma). In some embodiments, the cancer is human T-lymphotrophic virus (HTLV-1)-related cancer (e.g., adult T cell leukemia or lymphoma). In some embodiments, the cancer is Epstein-Barr virus (EBV) related cancer (e.g., Burkitt lymphoma, Hodgkin's and non-Hodgkin's lymphoma, stomach cancer). In some embodiments, the cancer is hepatitis B virus (HBV) related cancer (e.g., liver cancer). In some embodiments, the cancer is hepatitis C virus) related cancer (e.g., liver cancer, non-Hodgkin's lymphoma).

In some embodiments, the cancer is a liver cancer, a kidney cancer, an endometrial cancer, a thymic epithelial neoplasma, lung cancer, spindle cell sarcoma, chondrosarcoma, uterine smooth muscle, colon cancer, or pancreatic cancer.

In some embodiments, the cancer has been subjected to and/or failed one or more prior therapy (e.g., an immune checkpoint blockage therapy (e.g., a PD-1 antibody), a chemotherapy, a surgery, a cell therapy (e.g., an allogenic NK cell infusion therapy)).

In some embodiments, the cancer is a recurrent or refractory cancer.

In some embodiments, the cancer is refractory to one or more of irradiation therapy, chemotherapy, or immunotherapy (e.g., checkpoint blockade).

### Dosing, Method of Administration, and Delivery Vehicles

The tyrosine kinase inhibitors, the SHP-1 inhibitors, the myeloid cell activating agent or therapy, the lymphocyte activating agent, the TNFα inhibitors, and/or the immune cells (e.g., monocytes/macrophages) described herein can be administered at any desired dosage. Exemplary dosing regimens are described in e.g., “SHP-1 inhibitors” and “tyrosine kinases inhibitors” sections.

In some aspects, the size of the dose of the myeloid cell activating agent or therapy, the lymphocyte activating agent, the TNFα inhibitor, the SHP-1 inhibitor, the tyrosine kinase inhibitor, and/or the immune cells (e.g., monocytes/macrophages) is determined based on one or more criteria such as disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the activated immune cells being administered. For example, in some aspects, the number of monocytes or macrophages administered in the dose is determined based on the tumor burden that is present in the subject immediately prior to administration of the initiation of the dose of cells.

The myeloid cell activating agent or therapy, the lymphocyte activating agent, the TNFα inhibitor, the SHP-1 inhibitor, the tyrosine kinase inhibitor, and/or the immune cells (e.g., monocytes/macrophages) can be administered by any suitable means, for example, by bolus infusion, by injection, e.g., intravenous or subcutaneous injections. In some embodiments, the myeloid cell activating agent or therapy, the lymphocyte activating agent, the tyrosine kinase inhibitor, and/or the immune cells (e.g., monocytes or macrophages) are administered systemically (e.g., intravenously, subcutaneously, or intraperitoneally). In some embodiments, the myeloid cell activating agent or therapy, the lymphocyte activating agent, the SHP-1 inhibitor, the tyrosine kinase inhibitor, and/or the immune cells (e.g., monocytes or macrophages) are administered locally (e.g., intratumorally).

In some embodiments, the myeloid cell activating agent or therapy, the lymphocyte activating agent, the TNFα inhibitor, the SHP-1 inhibitor, the tyrosine kinase inhibitor, and/or the immune cells (e.g., monocytes or macrophages) are administered systemically (e.g., by parenteral administration) and/or, if desired for local treatment, intralesional or intratumorally administration. Parenteral infusions include intramuscular, intravenous, intraarterial, intraperitoneal, or subcutaneous administration. In some embodiments, the myeloid cell activating agent or therapy, the lymphocyte activating agent, the TNFα inhibitor, the SHP-1 inhibitor, and/or the tyrosine kinase inhibitor are administered orally.

In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy. In some embodiments, the TNFα inhibitor and the myeloid cell activating agent or therapy are administered simultaneously. In some embodiments the TNFα inhibitor and the myeloid cell activating agent or therapy are administered concurrently. In some embodiments, the TNFα inhibitor and the myeloid cell activating agent or therapy are administered sequentially (e.g., prior to or after). In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy. In some embodiments, the TNFα inhibitor and the myeloid cell activating agent or therapy are administered within about 7, 6, 5, 4, 3, 2, or 1 day of each other. In some embodiments, the TNFα inhibitor and the myeloid cell activating agent or therapy are administered within about 24, 16, 12, 8, 4, 2, or 1 hour of each other. In some embodiments, the TNFα inhibitor and the myeloid cell activating agent or therapy are administered within 30 minutes of each other.

In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy and the immune cells (e.g., monocytes or macrophages). In some embodiments, the immune cells (e.g., monocytes or macrophages), the TNFα inhibitor, and the myeloid cell activating agent or therapy are administered simultaneously. In some embodiments the immune cells (e.g., monocytes or macrophages), the TNFα inhibitor, and the myeloid cell activating agent or therapy are administered concurrently. In some embodiments, the immune cells (e.g., monocytes or macrophages), the TNFα inhibitor, and the myeloid cell activating agent or therapy are administered sequentially (e.g., prior to or after). In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy and the immune cells (e.g., monocytes or macrophages). In some embodiments, the immune cells (e.g., monocytes or macrophages), the TNFα inhibitor, and the myeloid cell activating agent or therapy are administered within about 7, 6, 5, 4, 3, 2, or 1 day of each other. In some embodiments, the immune cells (e.g., monocytes or macrophages), the TNFα inhibitor, and the myeloid cell activating agent or therapy are administered within about 24, 16, 12, 8, 4, 2, or 1 hour of each other. In some embodiments, the immune cells (e.g., monocytes or macrophages), the TNFα inhibitor, and the myeloid cell activating agent or therapy are administered within 30 minutes of each other.

In some embodiments, the SHP-1 inhibitor and the myeloid cell activating agent or therapy are administered simultaneously. In some embodiments, the SHP-1 inhibitor and the myeloid cell activating agent or therapy are administered concurrently. In some embodiments, the SHP-1 inhibitor and the myeloid cell activating agent or therapy are administered sequentially. In some embodiments, the SHP-1 inhibitor and the myeloid cell activating agent or therapy are administered within about 7, 6, 5, 4, 3, 2, or 1 day of each other. In some embodiments, the SHP-1 inhibitor and the myeloid cell activating agent or therapy are administered within about 24, 16, 12, 8, 4, 2, or 1 hour of each other. In some embodiments, the SHP-1 inhibitor and the myeloid cell activating agent or therapy are administered within 30 minutes of each other.

In some embodiments, the tyrosine kinase inhibitor and the myeloid cell activating agent or therapy are administered simultaneously. In some embodiments, the tyrosine kinase inhibitor and the myeloid cell activating agent or therapy are administered concurrently. In some embodiments, the tyrosine kinase inhibitor and the myeloid cell activating agent or therapy are administered sequentially. In some embodiments, the tyrosine kinase inhibitor and the myeloid cell activating agent or therapy are administered within about 7, 6, 5, 4, 3, 2, or 1 day of each other. In some embodiments, the tyrosine kinase inhibitor and the myeloid cell activating agent or therapy are administered within about 24, 16, 12, 8, 4, 2, or 1 hour of each other. In some embodiments, the tyrosine kinase inhibitor and the myeloid cell activating agent or therapy are administered within 30 minutes of each other.

In some embodiments, the SHP-1 inhibitor, the tyrosine kinase inhibitor, and the myeloid cell activating agent or therapy are administered simultaneously. In some embodiments, the SHP-1 inhibitor, the tyrosine kinase inhibitor, and the myeloid cell activating agent or therapy are administered concurrently. In some embodiments, the SHP-1 inhibitor, the tyrosine kinase inhibitor, and the myeloid cell activating agent or therapy are administered sequentially. In some embodiments, the SHP-1 inhibitor, the tyrosine kinase inhibitor, and the myeloid cell activating agent or therapy are administered within about 7, 6, 5, 4, 3, 2, or 1 day of each other. In some embodiments, the SHP-1 inhibitor, the tyrosine kinase inhibitor, and the myeloid cell activating agent or therapy are administered within about 24, 16, 12, 8, 4, 2, or 1 hour of each other. In some embodiments, the SHP-1 inhibitor, the tyrosine kinase inhibitor, and the myeloid cell activating agent or therapy are administered within 30 minutes of each other.

In some embodiments, the TNFα inhibitor is administered prior to (e.g., within about any of one week, 7 days, 6 days, 5 days, 4 days, 3 days, 2 days, 1 day, or less prior to) the administration of the myeloid cell activating agent or therapy, the SHP-1 inhibitor, and the tyrosine kinase inhibitor. In some embodiments, the SHP-1 inhibitor, the tyrosine kinase inhibitor, the TNFα inhibitor, and/or the myeloid cell activating agent or therapy are administered simultaneously. In some embodiments, the SHP-1 inhibitor, the tyrosine kinase inhibitor, the TNFα inhibitor, and/or the myeloid cell activating agent or therapy are administered concurrently. In some embodiments, the SHP-1 inhibitor, the tyrosine kinase inhibitor, the TNFα inhibitor, and/or the myeloid cell activating agent or therapy are administered sequentially (e.g., prior to or after). In some embodiments, the TNFα inhibitor is administered immediately after (e.g., within about any of 5 min, 10 min, 15 min, 30 min, 45 min, 60 min, 1 hr, 2 hrs, or 3 hrs after) the administration of the myeloid cell activating agent or therapy, the SHP-1 inhibitor, and the tyrosine kinase inhibitor. In some embodiments, the SHP-1 inhibitor, the tyrosine kinase inhibitor, the TNFα inhibitor, and/or the myeloid cell activating agent or therapy are administered within about 7, 6, 5, 4, 3, 2, or 1 day of each other. In some embodiments, the SHP-1 inhibitor, the tyrosine kinase inhibitor, the TNFα inhibitor, and/or the myeloid cell activating agent or therapy are administered within about 24, 16, 12, 8, 4, 2, or 1 hour of each other. In some embodiments, the SHP-1 inhibitor, the tyrosine kinase inhibitor, the TNFα inhibitor, and/or the myeloid cell activating agent or therapy are administered within 30 minutes of each other.

In some embodiments, the TNFα inhibitor is administered prior to administration of the myeloid cell activating agent or therapy. In some embodiments, the TNFα inhibitor is administered about 7, 6, 5, 4, 3, 2, or 1 day prior to administration of the myeloid cell activating agent or therapy. In some embodiments, the TNFα inhibitor is administered about 24, 16, 12, 8, 4, 2, or 1 hour prior to administration of the myeloid cell activating agent or therapy.

In some embodiments, the TNFα inhibitor is administered prior to administration of the immune cells (e.g., monocytes/macrophages) and the myeloid cell activating agent or therapy. In some embodiments, the TNFα inhibitor is administered about 7, 6, 5, 4, 3, 2, or 1 day prior to administration of the immune cells (e.g., monocytes/macrophages) and the myeloid cell activating agent or therapy. In some embodiments, the TNFα inhibitor is administered about 24, 16, 12, 8, 4, 2, or 1 hour prior to administration of the immune cells (e.g., monocytes/macrophages) and the myeloid cell activating agent or therapy.

In some embodiments, the TNFα inhibitor is administered prior to administration of the SHP-1 inhibitor and the myeloid cell activating agent or therapy. In some embodiments, the TNFα inhibitor is administered about 7, 6, 5, 4, 3, 2, or 1 day prior to administration of the SHP-1 inhibitor and the myeloid cell activating agent or therapy. In some embodiments, the TNFα inhibitor is administered about 24, 16, 12, 8, 4, 2, or 1 hour prior to administration of the SHP-1 inhibitor and the myeloid cell activating agent or therapy.

In some embodiments, the TNFα inhibitor is administered prior to administration of the tyrosine kinase inhibitor and the myeloid cell activating agent or therapy. In some embodiments, the TNFα inhibitor is administered about 7, 6, 5, 4, 3, 2, or 1 day prior to administration of the tyrosine kinase inhibitor and the myeloid cell activating agent or therapy. In some embodiments, the TNFα inhibitor is administered about 24, 16, 12, 8, 4, 2, or 1 hour prior to administration of the tyrosine kinase inhibitor and the myeloid cell activating agent or therapy.

In some embodiments, the TNFα inhibitor is administered prior to administration of the SHP-1 inhibitor, the tyrosine kinase inhibitor, and/or and the myeloid cell activating agent or therapy. In some embodiments, the TNFα inhibitor is administered about 7, 6, 5, 4, 3, 2, or 1 day prior to administration of the SHP-1 inhibitor, the tyrosine kinase inhibitor, and/or and the myeloid cell activating agent or therapy. In some embodiments, the TNFα inhibitor is administered about 24, 16, 12, 8, 4, 2, or 1 hour prior to administration of the SHP-1 inhibitor, the tyrosine kinase inhibitor, and/or and the myeloid cell activating agent or therapy.

It is also contemplated that TNFα inhibitors, SHP-1 inhibitors, tyrosine kinase inhibitors, and/or myeloid cell activating agent or therapy described herein can be delivered via any proper vehicles or methods. In some embodiments, the TNFα inhibitor, SHP-1 inhibitor, tyrosine kinase inhibitor, and/or myeloid cell activating agent or therapy is directly delivered into the tumor tissue. Different carrier systems can be utilized for this purpose. See, e.g., Manzari et al., Nat Rev Mater 6, 351-370 (2021); and Tewabe et al., J Multidiscip Healthc. 2021; 14: 1711-1724. In some embodiments, the TNFα inhibitor, SHP-1 inhibitor, tyrosine kinase inhibitor, and/or myeloid cell activating agent or therapy is delivered via a nanoparticle. In some embodiments, the TNFα inhibitor, SHP-1 inhibitor, tyrosine kinase inhibitor, and/or myeloid cell activating agent or therapy is delivered via a controlled release system. In some embodiments, the TNFα inhibitor, SHP-1 inhibitor, the tyrosine kinase inhibitor and/or the myeloid cell activating agent or therapy is delivered via a biomaterial implant scaffold. In some embodiments, the TNFα inhibitor, SHP-1 inhibitor, tyrosine kinase inhibitor, and/or myeloid cell activating agent or therapy is delivered via an injectable biomaterial scaffold. In some embodiments, the TNFα inhibitor, SHP-1 inhibitor, tyrosine kinase inhibitor, and/or myeloid cell activating agent or therapy is delivered via a transdermal delivery system. See, e.g., Riley et al., Nat Rev Drug Discov. 2019 March; 18(3): 175-196.

In some embodiments, the TNFα inhibitor, SHP-1 inhibitor, tyrosine kinase inhibitor, and/or myeloid cell activating agent or therapy is delivered by a cell. See, e.g., Millian et al., Ther Deliv. 2012 January; 3(1):25-41. In some embodiments, the cell comprises a macrophage. See, e.g., Visser et al., Front Pharmacol. 2019 Jan. 25; 10:22. In some embodiments, the cell comprises a polymer encapsulated human retinal pigmented epithelial (aRPE) cell. See, e.g., Nash et al., Clin Cancer Res. 2022 Aug. 22; CCR-22-1493. In some embodiments, the cells are encapsulated in a biocompatible material (e.g., biocompatible alginate capsules as discussed in Nash et al.)

In some embodiments, the TNFα inhibitor, SHP-1 inhibitor, tyrosine kinase inhibitor, and/or myeloid cell activating agent or therapy is associated with an antibody construct. In some embodiments, the TNFα inhibitor, SHP-1 inhibitor, tyrosine kinase inhibitor, and/or myeloid cell activating agent or therapy is connected with an antibody construct via a linker (e.g., a cleavable linker). In some embodiments, the antibody construct specifically recognizes a tumor associated antigen. In some embodiments, the antibody construct comprises an antibody recognizing a tumor antigen. In some embodiments, the antibody construct is an antibody drug conjugate (ADC).

In some embodiments, the TNFα inhibitor, SHP-1 inhibitor, tyrosine kinase inhibitor, and/or myeloid cell activating agent or therapy is a delivered via a method or device that promotes delivery into a particular organ (e.g., the organ that has a tumor). See examples of these methods or devices in e.g., Alsaggar et al., J Drug Target. 2018 June-July; 26(5-6):385-397; Zhao et al., Cell. 2020 Apr. 2; 181(1):151-167, which are incorporated by reference in their entirety.

In embodiments, the SHP-1 inhibitor and/or the tyrosine kinase inhibitor is delivered via a controlled drug delivery system (e.g., a slow-release system or vehicle, e.g., a sustained release system or vehicle). Examples of such systems can be found in e.g., Adepu et al., Molecules. 2021 October; 26(19): 5905; Oh et al., Chem. Asian J. 2022, 17, e202200333, which are incorporated by reference in their entirety.

### VI. Compositions Comprising the SHP-1 Pathway Inhibitor

The present application also provides compositions (e.g., pharmaceutical compositions) comprising the SHP-1 inhibitor, the tyrosine kinase inhibitor, the myeloid cell activating agent or therapy, and/or the immune cells for treatment as described above.

In some embodiments, there is provided a composition (e.g., a pharmaceutical composition) comprising a SHP-1 inhibitor and a myeloid cell activating agent or therapy (such as any of the agents described here). In some embodiments, the composition further comprises a tyrosine kinase inhibitor (such as Dasatinib). In some embodiments, the composition further comprises immune cells (such as monocytes or macrophages described herein). In some embodiments, the composition further comprises IL-2 or a biologically active fragment or derivative thereof. In some embodiments, the composition further comprises an immune checkpoint inhibitor (such as an anti-PD-1 antibody). In some embodiments, the composition further comprises a pharmaceutically acceptable carrier.

In some embodiments, there is provided a composition (e.g., a pharmaceutical composition) comprising a tyrosine kinase inhibitor and a myeloid cell activating agent or therapy (such as any of the agents described here). In some embodiments, the composition further comprises a SHP-1 inhibitor (such as TPI-1 or an analog or derivative thereof). In some embodiments, the composition further comprises immune cells (such as monocytes or macrophages described herein). In some embodiments, the composition further comprises IL-2 or a biologically active fragment or derivative thereof. In some embodiments, the composition further comprises an immune checkpoint inhibitor (such as an anti-PD-1 antibody). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and a myeloid cell activating agent or therapy (such as any of the agents described here). In some embodiments, the composition further comprises immune cells (such as monocytes or macrophages described herein). In some embodiments, the composition further comprises IL-2 or a biologically active fragment or derivative thereof. In some embodiments, the composition further comprises an immune checkpoint inhibitor (such as an anti-PD-1 antibody). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and a TLR agonist (e.g., CpG, polyI:C and/or R848). In some embodiments, the composition further comprises immune cells (such as monocytes or macrophages described herein). In some embodiments, the composition further comprises IL-2 or a biologically active fragment or derivative thereof. In some embodiments, the composition further comprises an immune checkpoint inhibitor (such as an anti-PD-1 antibody). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and a STING activator (e.g., cGAMP, e.g., 2′3′-cGAMP, e.g., 3′3′-cGAMP). In some embodiments, the composition further comprises immune cells (such as monocytes or macrophages described herein). In some embodiments, the composition further comprises IL-2 or a biologically active fragment or derivative thereof. In some embodiments, the composition further comprises an immune checkpoint inhibitor (such as an anti-PD-1 antibody). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and a chemotherapeutic agent (e.g., azathioprine (AZA), e.g., gemcitabine). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and a pro-inflammatory cytokine (e.g., IL-2, IL-1β, IL-18, and/or IL-6). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and a bacteria component (e.g., LPS). In some embodiments, the composition further comprises a pharmaceutically acceptable carrier. In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and an agent that promotes immunogenic cell death (ICD). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and an agent used in a radiation therapy (such as any of the radiation therapy described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and a PAMP/DAMP activator (such as any of the PAMP/DAMP activators described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and a cancer vaccine (such as any of the cancer vaccines described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and an oncolytic virus (such as any of the oncolytic viruses described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and an agent used in a sound treatment (such as any of the sound treatments described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and an agent used in a magnetic therapy (such as any of the magnetic therapies described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and an agent used in electrical or electrochemical treatment (such as any of the electrical or electrochemical treatments described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and an agent used in an electrostatic treatment (such as any of the electrostatic treatments described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor and/or a tyrosine kinase inhibitor, and an antibody drug conjugate (such as any of the ADCs described herein). In some embodiments, the composition further comprises immune cells (such as monocytes or macrophages described herein). In some embodiments, the composition further comprises a pharmaceutically acceptable carrier.

### EXEMPLARY EMBODIMENTS

Exemplary embodiment 1. A method of treating a cancer in an individual, comprising administering to the individual a) a myeloid cell activating agent or therapy, and b) a TNFα inhibitor.

Exemplary embodiment 2. The method of embodiment 1, wherein the method further comprises administering to the individual an inhibitor of the SHP-1 pathway.

Exemplary embodiment 3. A method of treating a cancer in an individual, comprising administering to the individual a TNFα inhibitor and an inhibitor of the SHP-1 pathway, wherein the individual is under an inflammation reaction.

Exemplary embodiment 4. The method of embodiment 2 or 3, wherein the inhibitor of the SHP-1 pathway comprises a SHP-1 inhibitor.

Exemplary embodiment 5. The method of embodiment 4, wherein the SHP-1 inhibitor is selected from the group consisting of: a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZFN, or TALENS systems), a peptide agent, a protein agent (e.g., an antibody agent that targets SHP-1), a protein degrading or destabilizing agent, a protein modified with an unnatural amino acid, an antibody directed therapy, an antibody drug conjugate, and any combination thereof.

Exemplary embodiment 6. The method of embodiment 4 or 5, wherein the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator.

Exemplary embodiment 7. The method of embodiment 6, wherein the SHP-1 inhibitor is TPI-1 or an analog or derivative thereof.

Exemplary embodiment 8. The method of any one of embodiments 2-7, wherein the inhibitor of the SHP-1 pathway comprises a tyrosine kinase inhibitor.

Exemplary embodiment 9. The method of embodiment 8, wherein the tyrosine kinase inhibitor is selected from the group consisting of: a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZFN, or TALENS systems), a peptide agent, a protein agent (e.g., an antibody agent that targets tyrosine kinase or activated tyrosine kinase), a protein degrading or destabilizing agent, a protein modified with an unnatural amino acid, an antibody directed therapy, an antibody drug conjugate (ADC), and any combination thereof.

Exemplary embodiment 10. The method of embodiment 8 or embodiment 9, wherein the tyrosine kinase inhibitor is selected from the group consisting of RK-20449, Dasatinib, R406, Entospletinib, Fostamatinib, Cerdulatinib, TAK-659, bosutinib, ponatinib, saracatinib, WH-4-023, KX2-391, and WZ3105, RK-20449, RK-20693, RK-24466, RK-20444, RK-20445, RK-20466, Masitinib, Ponatinib, and NVP-BEP800.

Exemplary embodiment 11. The method of embodiment 8 or embodiment 9, wherein the tyrosine kinase inhibitor inhibits any one of: Src, Syk, Hck, Lck, Lyn, JAK, and Yes.

Exemplary embodiment 12. The method of embodiment 8 or embodiment 9, wherein the tyrosine kinase inhibitor does not or weakly inhibits one or more kinases involved in T cell activation.

Exemplary embodiment 13. The method of embodiment 12, wherein the one or more kinases involved in T cell activation comprises any one or more of: Lck, Fyn, Zap70, Syk and Csk.

Exemplary embodiment 14. The method of embodiment 11, wherein the tyrosine kinase inhibitor is an inhibitor of a tyrosine kinase of a Src family.

Exemplary embodiment 15. The method of embodiment 2, wherein the inhibitor of the SHP-1 pathway is an antibody that blocks a cell surface inhibitory receptor.

Exemplary embodiment 16. The method of embodiment 15, wherein the antibody that blocks a cell surface inhibitory receptor is selected from any one of: LILRB1, LILRB2, LILRB3, LILRB4, LILRB5, SIRPα, PirB, gp49B1, Siglec-1, Siglec-2, Siglec-3, Siglec-5, Siglec-6, Siglec-7, Siglec-8, Siglec-9, Siglec-10, Siglec-11, Siglec-12, Siglec-14, Siglec-15, Siglec-E, Siglec-F, Siglec-G, Siglec-H, DCIR4, CD371, CD200R, SLAMF1, SLAMF3, SLAMF5, SLAMF6, SLAMF7, SLAMF8, and SLAMF9.

Exemplary embodiment 17. The method of any one of embodiments 1-16, wherein the myeloid cell activating agent or therapy activates a cell selected from any one of: macrophages having the M1 phenotype, intratumoral dendritic cells, intratumoral B cells, antigen presenting cells, and any combination thereof.

Exemplary embodiment 18. The method of any one of embodiments 1-17, wherein the myeloid cell activating agent or therapy is selected from the group consisting of: a STING activator, a Toll-like receptor (TLR) agonist, a PAMP/DAMP activator, a chemotherapy, a pro-inflammatory cytokine, a cancer vaccine, a bacteria or component thereof, a virus or component thereof, a fungus or component thereof, an immune cell, a sound treatment, a magnetic therapy, an electrical treatment, a radiation treatment, a radiopharmaceutical treatment, an electrostatic treatment, an antibody drug conjugate, and any combination thereof.

Exemplary embodiment 19. The method of embodiment 18, wherein the myeloid cell activating agent or therapy comprises a TLR agonist.

Exemplary embodiment 20. The method of embodiment 19, wherein the TLR agonist activates TLR2, TLR3, TLR4, TLR5, TLR7, TLR8, TLR9, and/or zymosan.

Exemplary embodiment 21. The method of embodiment 19 or embodiment 20, wherein the TLR agonist comprises CpG, polyI:C, and/or R848.

Exemplary embodiment 22. The method of embodiment 18, wherein the myeloid cell activating agent or therapy comprises a STING activator.

Exemplary embodiment 23. The method of embodiment 22, wherein the STING activator is selected from the group consisting of: 2′3′-cGAMP, ADU-s100, G10, SR-717, Vadimezan (DMXAA; ASA-404), Sting agonist-20, MSA-2, diABZI STING agonist-1, cGAMP (Cyclic GMP-AMPP), STING agonist-3, and c-di-AMP (Cyclic diadenylate) sodium.

Exemplary embodiment 24. The method of any one of embodiments 1-23, wherein the TNFα inhibitor is selected from the group consisting of: a small molecule inhibitor, a neutralizing antibody, a TNFα receptor blockade antibody, a soluble TNFα receptor, a TNFα-targeting short interfering RNA (siRNA), a chemical inhibitor of TNFα mRNA stability, an inhibitor of TNFα converting enzyme (TACE), and derivatives thereof.

Exemplary embodiment 25. The method of embodiment 24, wherein the TNFα inhibitor is a TNFα neutralizing antibody.

Exemplary embodiment 26. The method of embodiment 25, wherein the antibody is selected from the group consisting of: infliximab, adalimumab, etanercept, golimumab, and certolizumab.

Exemplary embodiment 27. The method of any one of embodiments 1-26, wherein the method further comprises administering to the individual an effective amount of a lymphocyte activating agent.

Exemplary embodiment 28. The method of embodiment 27, wherein the lymphocyte is a T cell.

Exemplary embodiment 29. The method of embodiment 27 or embodiment 28, wherein the lymphocyte activating agent is selected from the group consisting of: a cytokine, a chemokine, a metabolism-modulating drug, a metabolite antagonist, an immune checkpoint inhibitor, an immune cell, a cancer vaccine, a bacteria or component thereof, a virus or component thereof, a fungus or component thereof, a bispecific T cell engager (BiTE), an antibody-drug conjugate, and any combination thereof.

Exemplary embodiment 30. The method of any one of embodiments 1-29, wherein the TNFα inhibitor is administered prior to the administration of the myeloid cell activating agent or therapy.

Exemplary embodiment 31. The method of any one of embodiments 1-30, wherein the TNFα inhibitor is administered after the administration of the myeloid cell activating agent or therapy.

Exemplary embodiment 32. The method of embodiment 30 or 31, wherein the TNFα inhibitor is administered within 5, 4, 3, 2, or 1 day of the administration of the myeloid cell activating agent or therapy, or wherein the TNFα inhibitor is administered no more than four days after the administration of the myeloid cell activating agent or therapy.

Exemplary embodiment 33. The method of any one of embodiments 1-32, wherein the myeloid cell activating agent or therapy is administered systemically or locally.

Exemplary embodiment 34. The method of any one of embodiments 1-33, wherein the TNFα inhibitor is administered systemically or locally.

Exemplary embodiment 35. The method of any one of embodiments 2-34, wherein the inhibitor of the SHP-1 signaling pathway is administered systemically or locally.

Exemplary embodiment 36. The method of any one of embodiments 33-35, wherein the systemic administration comprises oral administration, intravenous administration, subcutaneous administration, or intraperitoneal administration.

Exemplary embodiment 37. The method of any one of embodiments 33-35, wherein the local administration comprises intratumoral administration.

Exemplary embodiment 38. The method of any one of embodiments 1-37, wherein the myeloid cell activating agent or therapy is administered daily for at least 2, 3, 4, 5, 6, or 7 days.

Exemplary embodiment 39. The method of any one of embodiments 1-37, wherein the myeloid cell activating agent or therapy is administered intermittently.

Exemplary embodiment 40. The method of any one of embodiments 2-39, wherein the inhibitor of the SHP-1 signaling pathway is administered daily for at least 2, 3, 4, 5, 6, or 7 days.

Exemplary embodiment 41. The method of any one of embodiments 2-39, wherein the inhibitor of the SHP-1 signaling pathway is administered intermittently.

Exemplary embodiment 42. The method of any one of embodiments 1-41, wherein the TNFα inhibitor is administered at least once a week, once every five days, once every three days, or daily.

Exemplary embodiment 43. The method of any one of embodiments 1-41, wherein the TNFα inhibitor is administered intermittently.

Exemplary embodiment 44. The method of embodiment 43, wherein the TNFα inhibitor is administered to the individual for at least two cycles, wherein each cycle has about three to about seven days.

Exemplary embodiment 45. The method of any one of embodiments 2-44, wherein the SHP-1 pathway inhibitor comprises a tyrosine kinase inhibitor and a SHP-1 inhibitor.

Exemplary embodiment 46. The method of any one of embodiments 2-45, wherein the inhibitor of the SHP-1 pathway is administered to the individual simultaneously with the myeloid cell activating agent or therapy.

Exemplary embodiment 47. The method of any one of embodiments 2-45, wherein the inhibitor of the SHP-1 pathway and the myeloid cell activating agent or therapy are administered sequentially.

Exemplary embodiment 48. The method of any one of embodiments 2-47, wherein the inhibitor of the SHP-1 pathway and the myeloid cell activating agent or therapy are administered to the individual until the individual undergoes tumor clearance.

Exemplary embodiment 49. The method of any one of embodiments 2-48, wherein the inhibitor of the SHP-1 pathway, the myeloid cell activating agent or therapy, and/or the TNFα inhibitor are further administered intermittently to the individual after tumor clearance, wherein the individual was administered a myeloid cell activating agent or therapy, an inhibitor of the SHP-1 pathway, and/or a TNFα inhibitor prior to tumor clearance.

Exemplary embodiment 50. The method of any one of embodiments 27-49, wherein the lymphocyte activating agent is a cytokine, wherein the cytokine comprises IL-2 or a biologically active derivative thereof.

Exemplary embodiment 51. The method of any one of embodiments 27-49, wherein the lymphocyte activating agent is an immune checkpoint inhibitor, wherein the immune checkpoint inhibitor comprises an anti-PD-1 antibody.

Exemplary embodiment 52. The method of embodiment 50 or embodiment 51, wherein the IL-2 or biologically active derivative thereof and/or the anti-PD-1 antibody is administered to the individual daily.

Exemplary embodiment 53. The method of embodiment 50 or embodiment 51, wherein the IL-2 or biologically active derivative thereof and/or the anti-PD-1 antibody is administered to the individual intermittently.

Exemplary embodiment 54. The method of embodiment 53, wherein the IL-2 or biologically active derivative thereof and/or the anti-PD-1 antibody is administered to the individual for at least two cycles, wherein each cycle has about three to about 20 days.

Exemplary embodiment 55. The method of any one of embodiments 1-54, wherein the individual does not develop Grade 2-4 cytokine release syndrome or pro-inflammatory organ damage.

Exemplary embodiment 56. The method of any one of embodiments 1-55, wherein administration of the TNFα inhibitor does not compromise or weakly compromises tumor clearance.

Exemplary embodiment 57. The method of any one of embodiments 1-56, wherein the cancer is a solid tumor.

Exemplary embodiment 58. The method of any one of embodiments 1-57, wherein the cancer is a hematological cancer.

Exemplary embodiment 59. The method of any one of embodiments 1-58, wherein the cancer is a late-stage cancer.

Exemplary embodiment 60. The method of any one of embodiments 1-59, wherein the cancer is resistant or refractory to a radiation therapy, a chemotherapeutic agent, and/or a checkpoint inhibitor.

Exemplary embodiment 61. The method of any one of embodiments 1-60, wherein the individual is a human.

### EXAMPLES

The examples below are intended to be purely exemplary of the invention and should therefore not be considered to limit the invention in any way. The following examples and detailed description are offered by way of illustration and not by way of limitation.

### Example 1. KX147.AB&C Combination Therapy and Effects of ±Anti-TNFα to Promote the Treatment of Multi-Lesion Lewis Lung Cancer (LLC)

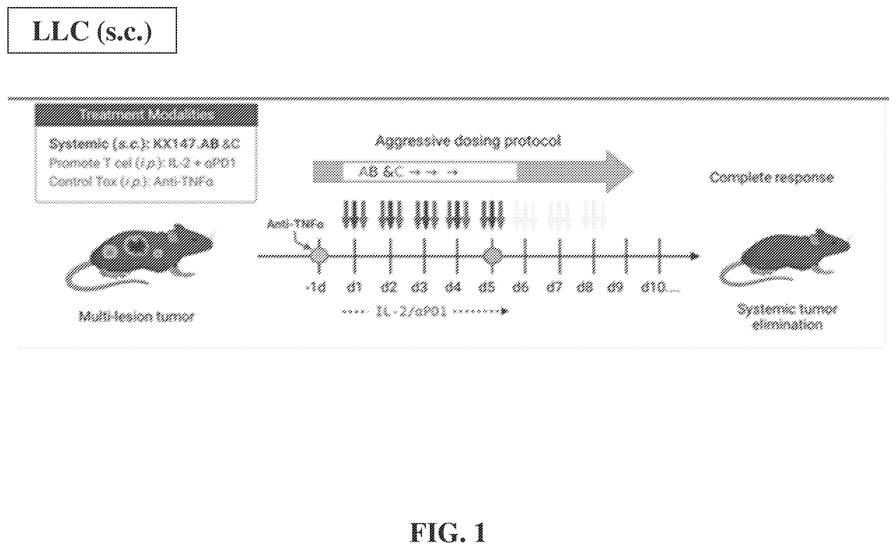

Described herein is a syngeneic Lewis lung cancer (LLC) murine model to test the efficacy of combined KX147.AB&C combination treatment of LLC, IL-2 and anti-PD-1 co-administration to promote T cell immunity, and anti-TNFα treatment to control adverse side effects, as shown in FIG. 1. Briefly, LLCs were injected subcutaneously (s.c.) into the flanks of syngeneic mice to generate multi-lesion engraftments. Then, an anti-TNFα antibody at 100 μg or vehicle was administered by intraperitoneal injection (i.p.) starting one day before KX147.AB&C treatment and again on Day 5. On Day 1, aggressive KX147.AB&C treatment was administered daily (s.c.) until mice showed complete response by Day 5. KX147.AB&C treatment comprised: A: s.c. TPI-1 at 1 mg/kg; B: s.c. PolyI:C+R848, each at 20 μg; and C: s.c. Dasatinib at 2 mg/kg. Simultaneous administration of daily IL-2 administration at 10000 IU (i.p.) and anti-PD-1 administration every three days at 100 μg (i.p.) also occurred starting on Day 1 to promote T cell immunity.

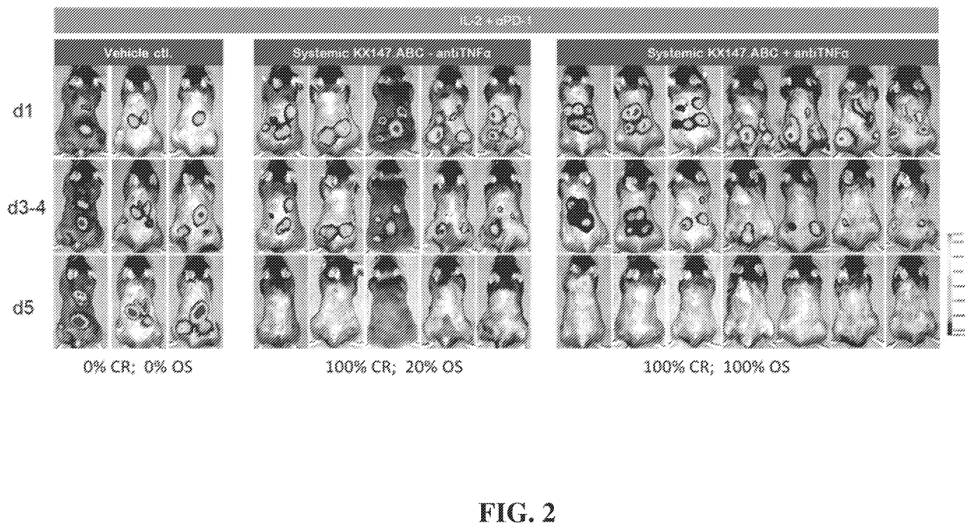

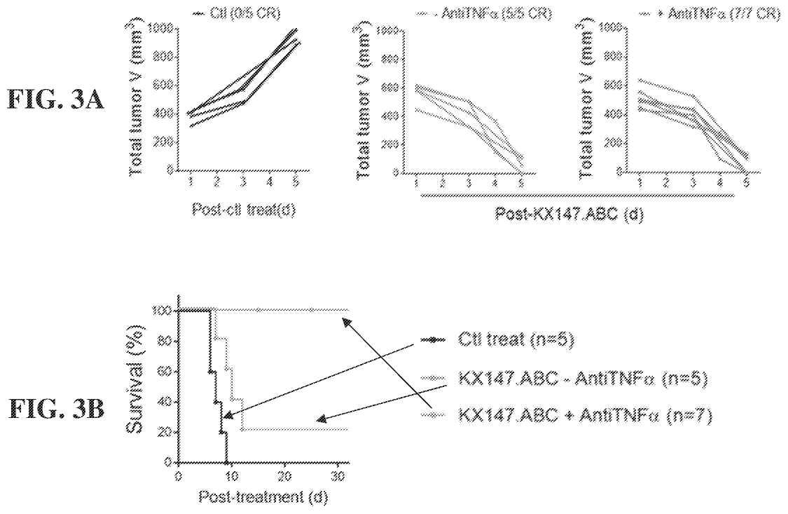

This aggressive treatment combined with IL-2/αPD1, without or with anti-TNFα antibody treatment, rapidly eliminated multiple lesions of tumors in the entire body in 5 days, as shown in FIG. 2 and FIG. 3A where the mice given the KX147.AB&C treatment displayed complete response (CR) and the mice given a vehicle showed continued LLC growth. Mice that received the anti-TNFα antibody maintained clinical health and achieved 100% overall survival (OS), whereas the group without the anti-TNFα antibody suffered high rates of post-treatment death (20% OS) due to strong adverse effects (FIG. 3B). Administration of the anti-TNFα antibody did not reduce LLC response to KX147.AB &C combination therapy, nor did administration of the anti-TNFα antibody reduce treatment efficacies.

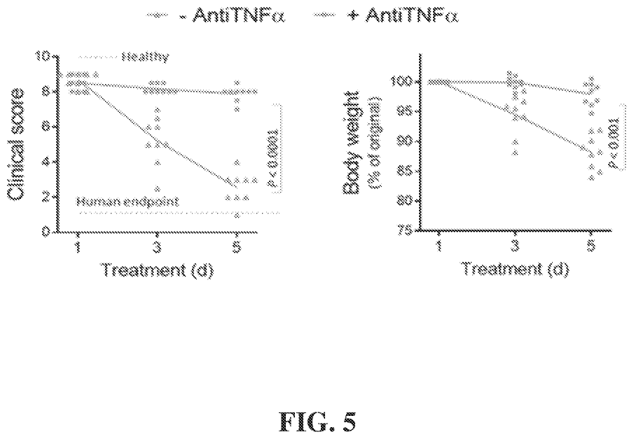

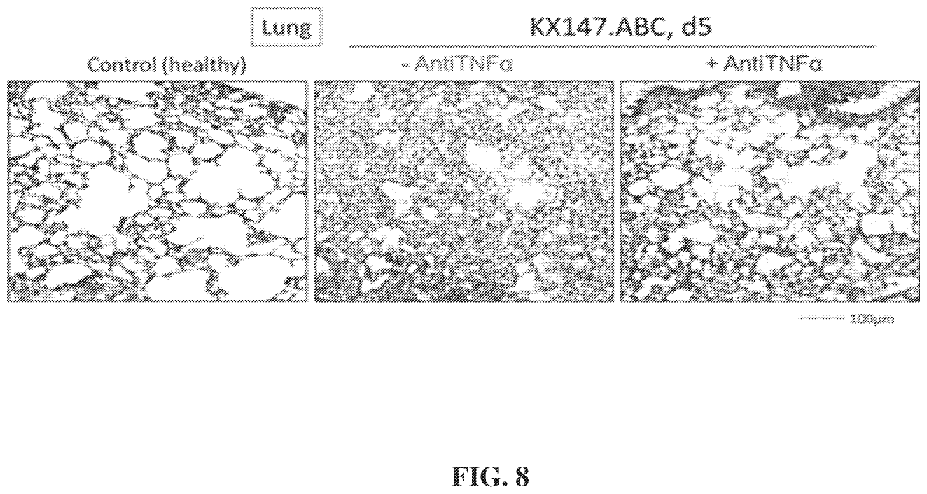

Furthermore, anti-TNFα administration was shown to ameliorate the adverse effects associated with KX147.AB&C therapy, including cytokine release syndrome (CRS; FIGS. 4A and 4B), pneumonitis (FIG. 7), nephritis (FIG. 7), and hepatitis (FIG. 7). In mice that were not given anti-TNFα therapy, nephritis and hepatitis resulted in kidney and liver dysfunction, while severe pneumonitis caused death. Lung cross sections in FIG. 8 of healthy control mice and mice administered or not administered anti-TNFα therapy show infiltration of polymorphonuclear leukocytes (PMNs) and tissue damage in the lungs of mice not administered anti-TNFα therapy compared to control and anti-TNFα therapy groups. FIG. 5 shows the marked reduction in clinical score and body weight, indicating the rapid decline in overall health in mice that were not given anti-TNFα therapy, whereas mice administered anti-TNFα therapy showed minimal impact. Additionally, mice that were not given anti-TNFα therapy displayed splenomegaly and colitis compared to mice that were administered anti-TNFα therapy (FIGS. 6A and 6B). Blood biochemistry assays also showed signs of pathology in mice that were not administered anti-TNFα therapy (Table 2 below): anti-TNFα protected from anemia and kidney and liver damage caused by KX147.AB&C treatment, whereas increases of BUN and ATL in mice not treated with anti-TNFα therapy suggested kidney and liver damage.

TABLE 2
Results of blood biochemistry assays.

| Blood Test (post- | | KX147.AB&C − | KX147.AB&C + |
|---|---|---|---|
| treat Day 5) | Ctrl | anti-TNFα | anti-TNFα |
| Hemoglobin | 8.3 ± 0.2 | 5.7 ± 0.7 ** | 7.2 ± 0.2* |
| (OD540 ± SEM) | | (Anemia) | |
| (10 μL in whole | | | |
| blood in 1 mL) | | | |
| Blood Urea Nitrogen | 39.0 ± 6.0 | 71.1 ± 10.0 ** | 46.3 ± 3.0 |
| (BUN, mg/dL) | | (Nephritis) | |
| Alanine | 28.5 ± 6.5 | 70.5 ± 18.5 *** | 37.0 ± 4.0 |
| aminotransferase | | (Liver pathology) | |
| (ATL, IU/L) | | | |

Taken together, these experiments showed that anti-TNFα therapy was key to eliminating the toxic effects of aggressive KX147.AB&C combination treatment. This finding opens up new dosing regimens for cancer treatment without toxicities.

### Example 2: Effects of KX147.AB&C Combination Therapy and Effects of ±Anti-TNFα to Promote the Treatment of Single and Multi-Lesion MC38 Colorectal Carcinoma

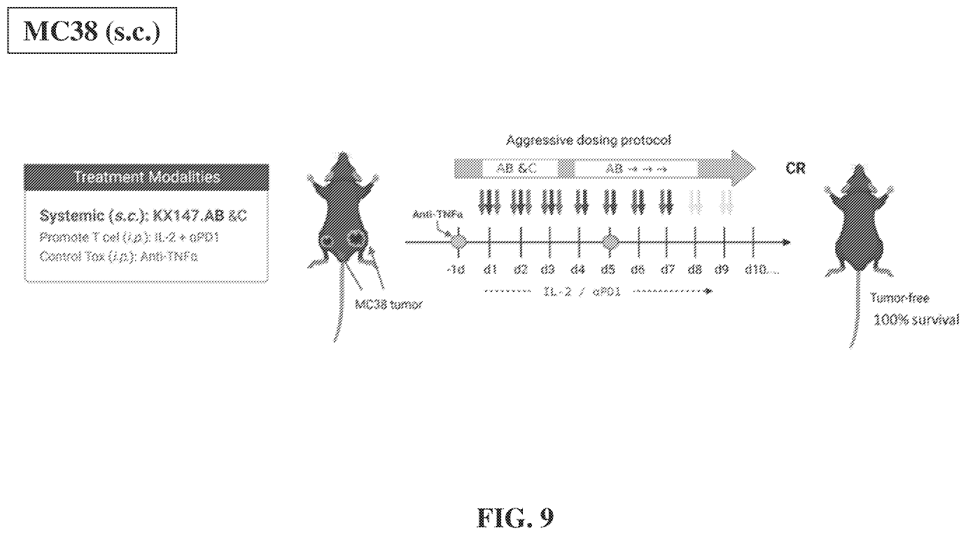

A second cancer murine model was tested to confirm and expand upon the results obtained in Example 1 above. As shown in FIG. 9, MC38 colorectal carcinoma was engrafted into one or both of the flanks of syngeneic C57Bl/6 mice. One day before KX147.AB&C combination treatment was to begin and again on Day 5, mice were administered either an anti-TNFα antibody at 100 μg or vehicle (i.p.). Then on each of Days 1-3, mice were administered KX147.AB&C combination treatment (s.c.). Starting from Day 4 until tumor elimination, mice were then switched to KX147.AB combination treatment. KX147.AB&C treatment comprised: A: s.c. TPI-1 at 1 mg/kg; B: s.c. PolyI:C+R848, each at 20 μg; and C: s.c. Dasatinib at 2 mg/kg. KX147.AB treatment comprised: A: s.c. TPI-1 at 1 mg/kg; and B: s.c. PolyI:C+R848, each at 20 μg. Simultaneous administration of daily IL-2 administration at 10000 IU (i.p.) and anti-PD-1 administration every three days at 100 μg (i.p.) also occurred starting on Day 1 to promote T cell immunity.

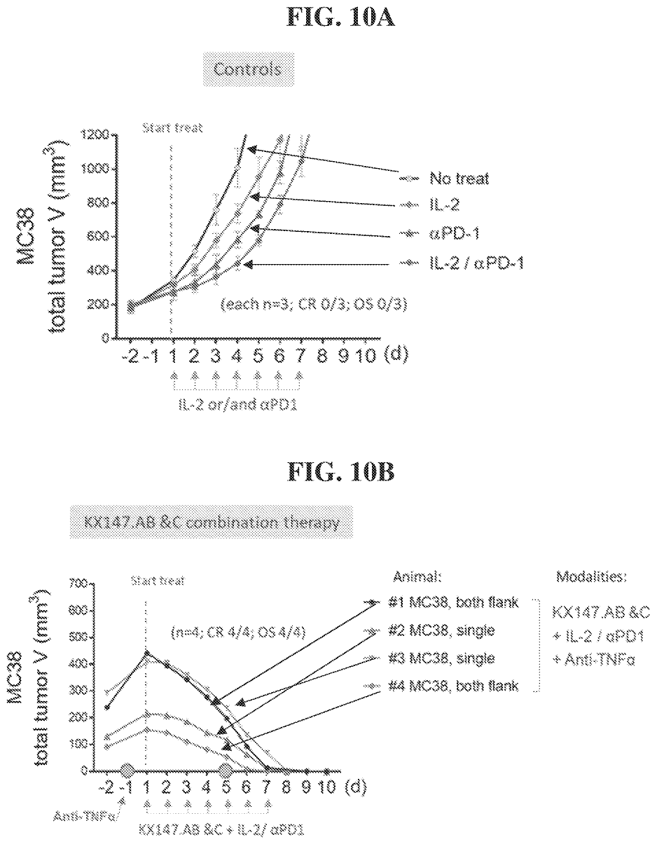

An aggressive dosing strategy was applied to administer treatment daily with KX147.AB &C for 3 days, followed by KX147.AB daily until tumor clearance. Control mice that were treated with vehicle, IL-2, anti-PD-1, or combination IL-2 and anti-PD-1 without KX147.AB&C therapy did not display a reduction in tumor burden, as seen in FIG. 10A. As shown in FIG. 10B, three days of KX147.AB&C followed by daily KX147.AB in combination with IL-2 and anti-PD-1 therapy led to rapid elimination of MC38 carcinoma in both flanks, achieving CR in 7 days. Anti-TNFα protected from adverse effects and supported animal health and survival (100% survival rate).

These results corroborated the results of Example 1 in a second tumor model.

### Example 3: Protective Effects of Anti-TNFα Treatment Against Adverse Events when Used in Combination with KX147.AB&C Combination Therapy to Treat MC38 Colorectal Carcinoma

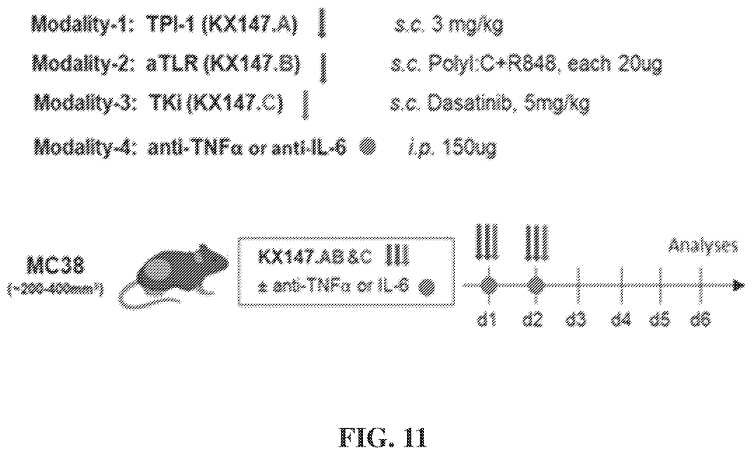

Mice with established MC38 colorectal carcinoma (200-400 mm³) were treated with KX147.AB&C alone or in combination with either anti-TNFα mAb or anti-IL-6 mAb (each at 150 μg, i.p.), as shown in FIG. 11. KX147.AB&C treatment comprised: A: s.c. TPI-1 at 3 mg/kg; B: s.c. PolyI:C+R848, each at 20 μg; and C: s.c. Dasatinib at 2 mg/kg. The treatment was administered twice, on Days 1 and 2. Tumor volume changes were recorded daily, and tumor microenvironments (TMEs) were analyzed for immune cell infiltration on Day 6 after treatment administration was started. Immune cell lineages assessed by flow cytometry included: CD8⁺ T cells, CD4⁺ T_{H} cells, NK cells, PMNs, macrophages, and myeloid derived suppressor cells (MDSCs). CRS was assessed by collecting blood serum prior to and 3 hrs after each round of KX147.AB&C treatment. Serum cytokine and chemokine levels (TNFα, IL-6, IL-1β, IL-10, IFNα, IFNγ, CCL2, CCL5, CXCL1, etc.) were assayed. Organ examination was performed after euthanasia. Various organs such as spleen, liver, kidney, and colon were resected, examined, and weighed.

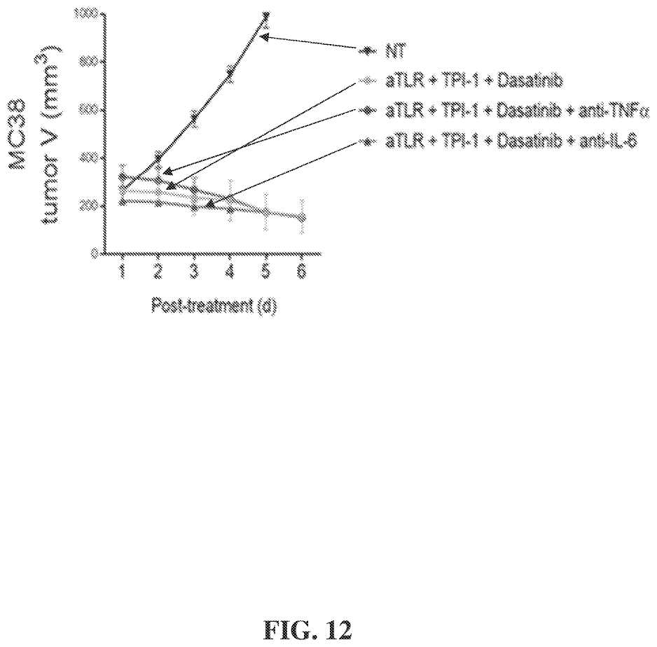

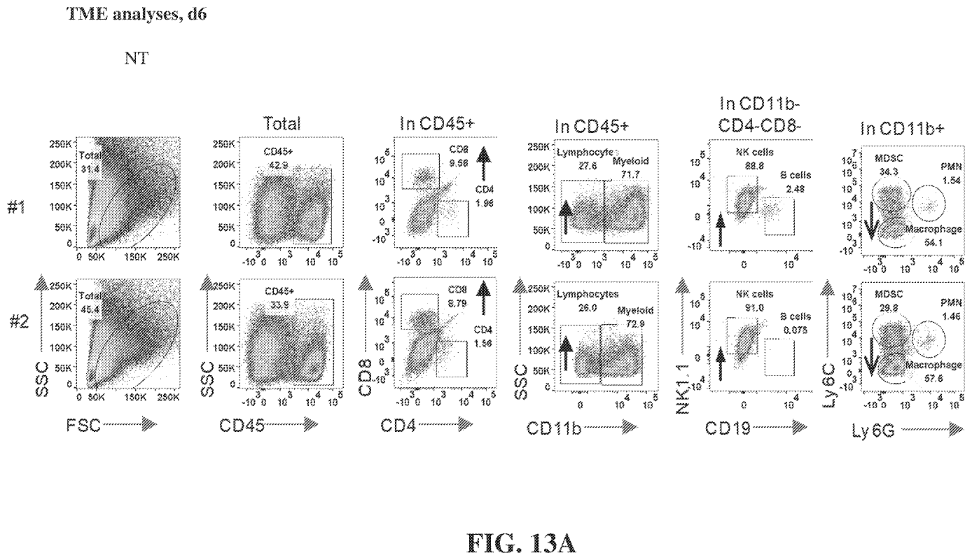

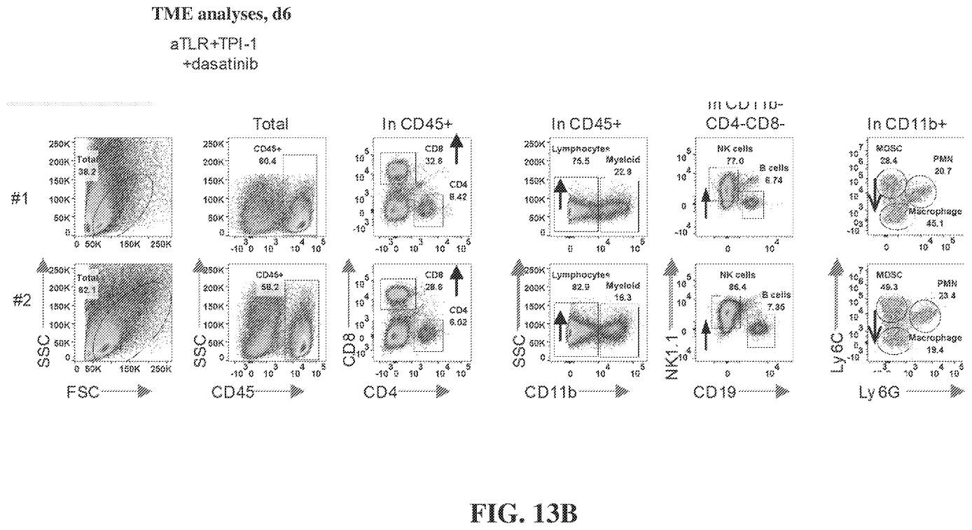

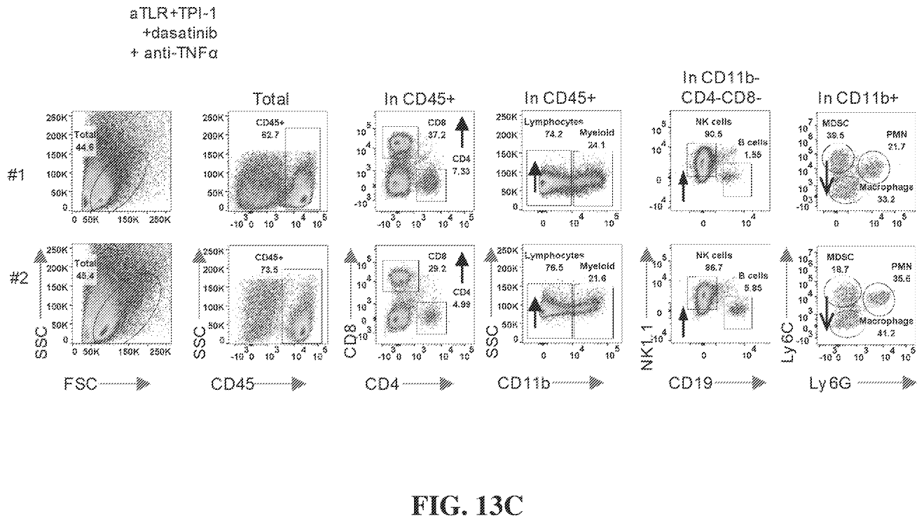

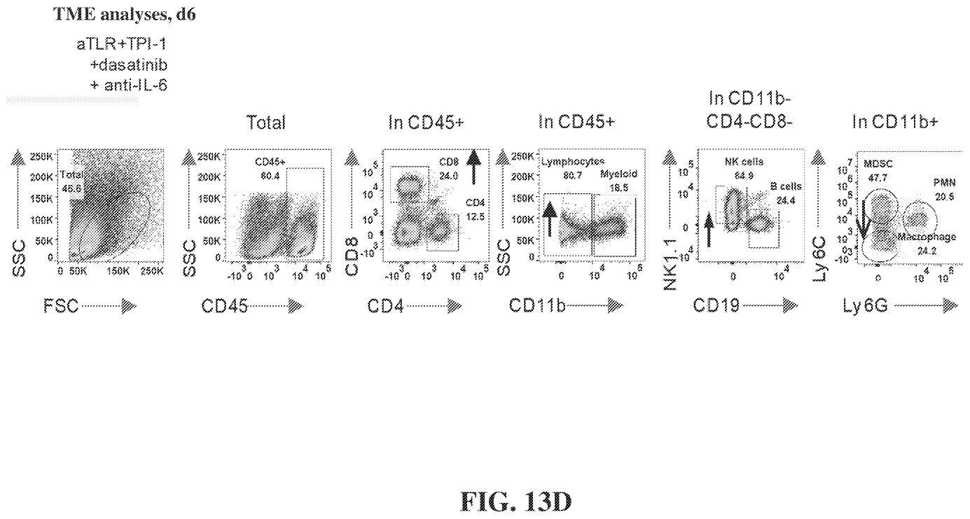

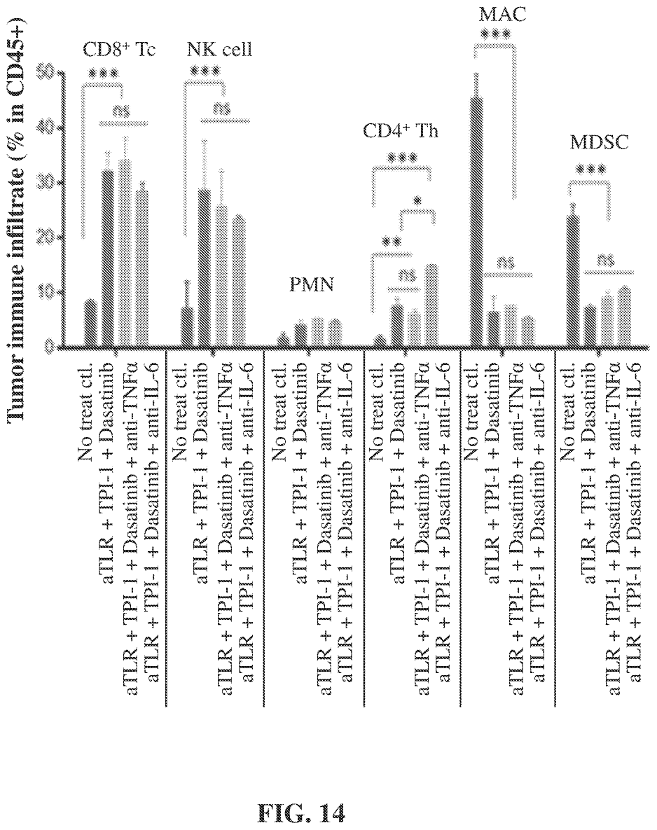

As shown in FIG. 12, all mice treated with KX147.AB&C showed tumor elimination. The immune cell lineage infiltration into the MC38 TME differed based on the treatment (FIGS. 13A-D and FIG. 14). Tumors from control mice showed poor infiltration of anti-tumor-acting T cells, NK cells, and PMNs but high levels of tumor-promoting macrophages and MDSCs (FIG. 13A and FIG. 14). Tumors from mice treated with KX147.AB&C alone (FIG. 13B and FIG. 14) or with combination KX147.AB&C and anti-TNFα mAb (FIG. 13C and FIG. 14) displayed significant increase in the infiltration of CD8⁺ T cells, CD4⁺ T_{H} cells, and NK cells and a significant reduction in the level of macrophage and MDSC infiltration. Similarly, tumors from mice treated with combination KX147.AB&C and anti-IL-6 mAb (FIG. 13D and FIG. 14) displayed significantly increased CD8⁺ T cells, CD4⁺ T_{H} cells, and NK cells infiltration and significantly decreased macrophage and MDSC infiltration, although the percent of CD4⁺ T_{H} cells was doubled compared to KX147.AB&C alone or in combination with anti-TNFα mAb.

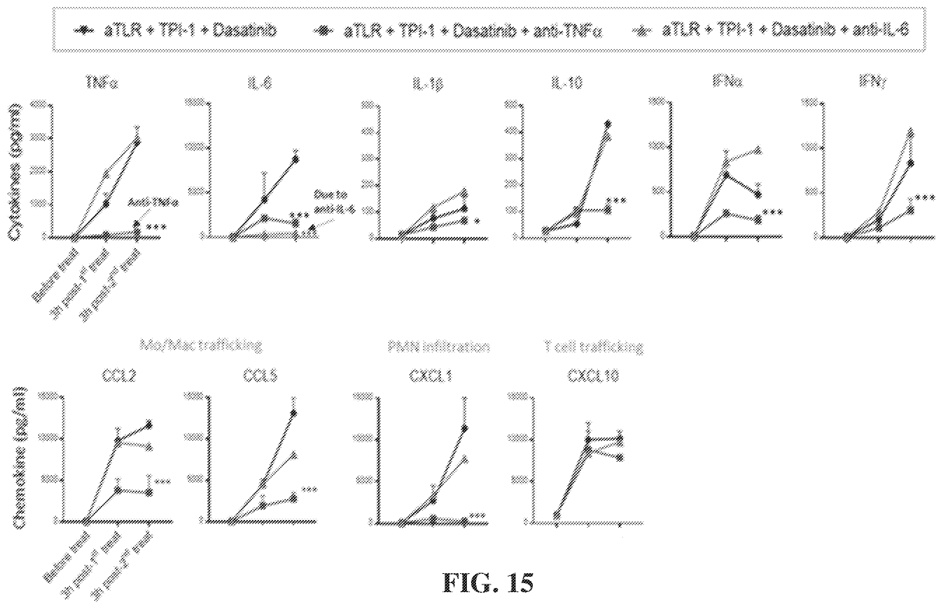

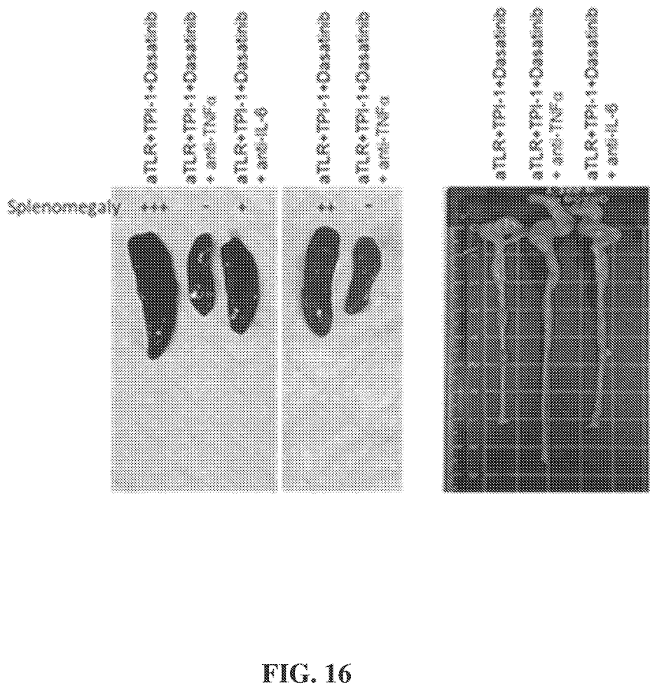

Mice were analyzed for CRS (FIG. 15) and organ dysfunction (FIG. 16). Across all cytokines tested, mice that were co-treated with anti-TNFα mAb showed reduced serum cytokine levels as shown in FIG. 15, as well as reduced CCL2, CCL5, and CXCL1 chemokine levels. Importantly, no change was observed in chemokine CXCL10 levels, which is critical for T cell trafficking to the TME. FIG. 16 demonstrates the splenomegaly and colitis that was found to occur in mice aggressively treated with KX147.AB&C was still present in mice that were co-treated with KX147.AB&C and anti-IL-6 mAb but not in mice co-treated with KX147.AB&C and anti-TNFα mAb.

These results demonstrate that the addition of anti-TNFα mAb treatment does not reduce, ameliorate, or eliminate the adaptive immune cell-promoting actions of the KX147.AB&C combination treatment. However, these results do confirm the results from the LLC murine model in Example 1 wherein anti-TNFα mAb treatment reduced systemic CRS and organ damage, which was an effect that surprisingly was not seen with anti-IL-6 mAb treatment. These results identify that anti-TNFα therapy is responsible for the reduction in KX147.AB&C toxicities and is not interchangeable with antibody depletion therapy of another inflammatory cytokine such as IL-6.

Moreover, although the above specific experiment involves using both TPI-1 and dasatinib and both polyI:C and R848, results from experiments using either one of TPI-1 and dasatinib and either one of polyI:C and R848 achieved similar effects (data not shown). It was also found that the proper time window for anti-TNFα antibody treatment can be from at least a week prior (as long as the antibody is stable for the time window) to immediately after (e.g., within 0.5-1 hour) the SHP-1 inhibitor/αTLR treatment. It is preferable that the anti-TNFα antibody is provided prior to or simultaneously with the SHP-1 inhibitor and/or αTLR so that it maximally blocks the TNFα induced after the treatment of SHP-1 inhibitor and the pro-inflammatory agent (i.e., myeloid cell activating agent or therapy).

### Example 4: KX147.AB&C Combination Therapy and Effects of ±Anti-TNFα to Promote the Treatment of Subcutaneous and Orthotopic Multi-Lesion KPC Pancreatic Ductal Adenocarcinoma

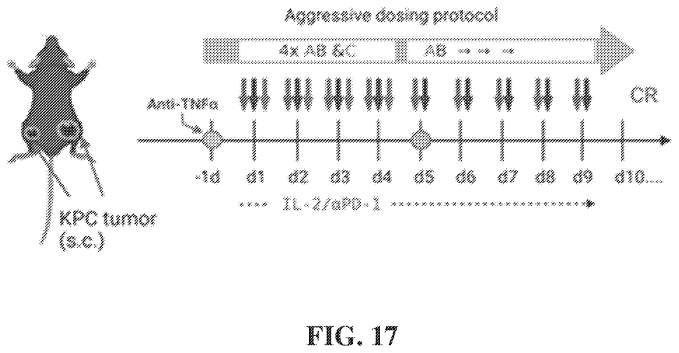

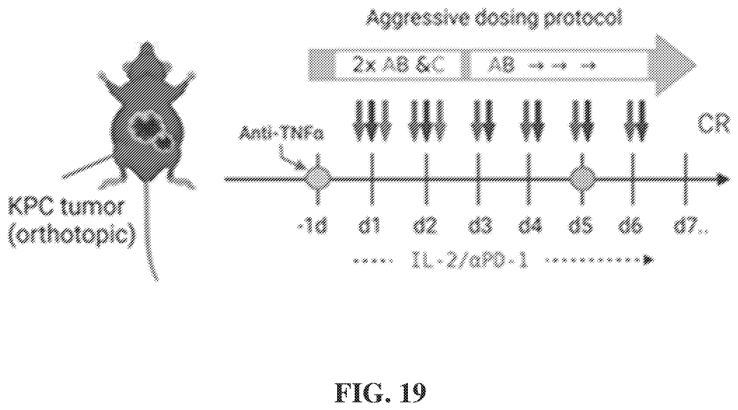

A third cancer murine model was tested to confirm and expand upon the results obtained above. As shown in FIG. 17 and FIG. 19, KPC pancreatic ductal adenocarcinoma was engrafted either s.c. (FIG. 17) or orthotopically (FIG. 19) into syngeneic C57Bl/6 mice. One day before KX147.AB&C combination treatment was to begin and again on Day 5, mice were administered either an anti-TNFα antibody at 100 μg or vehicle (i.p.). Then on each of Days 1-4 (FIG. 17) or on each of Days 1-2 (FIG. 19), mice were administered KX147.AB&C combination treatment (s.c.). Starting from Day 4 or Day 2 until tumor elimination, mice were then switched to KX147.AB combination treatment. KX147.AB&C treatment comprised: A: s.c. TPI-1 at 1 mg/kg; B: s.c. PolyI:C+R848, each at 20 μg; and C: s.c. Dasatinib at 2 mg/kg. KX147.AB treatment comprised: A: s.c. TPI-1 at 1 mg/kg; and B: s.c. PolyI:C+R848, each at 20 μg. Simultaneous administration of daily IL-2 administration at 10000 IU (i.p.) and anti-PD-1 administration every three days at 100 μg (i.p.) also occurred starting on Day 1 to promote T cell immunity.

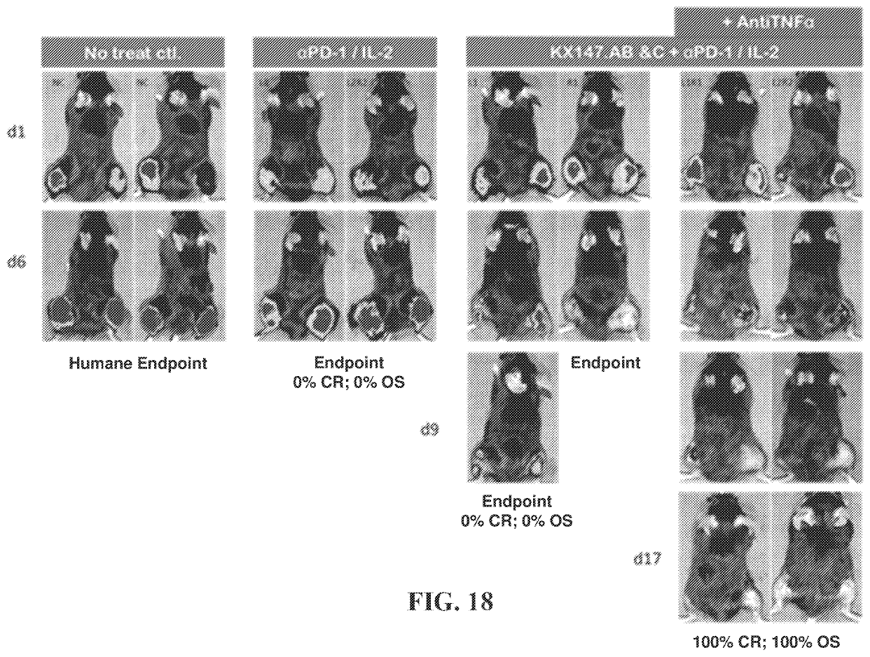

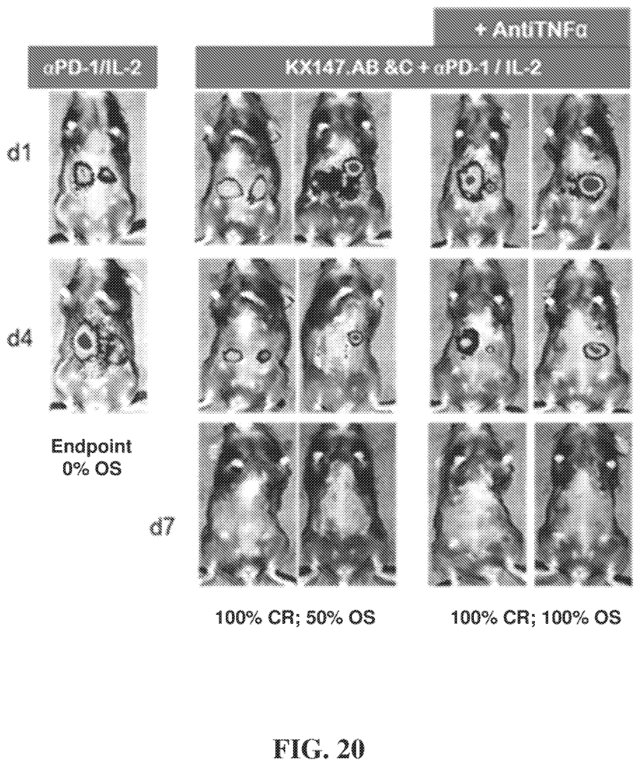

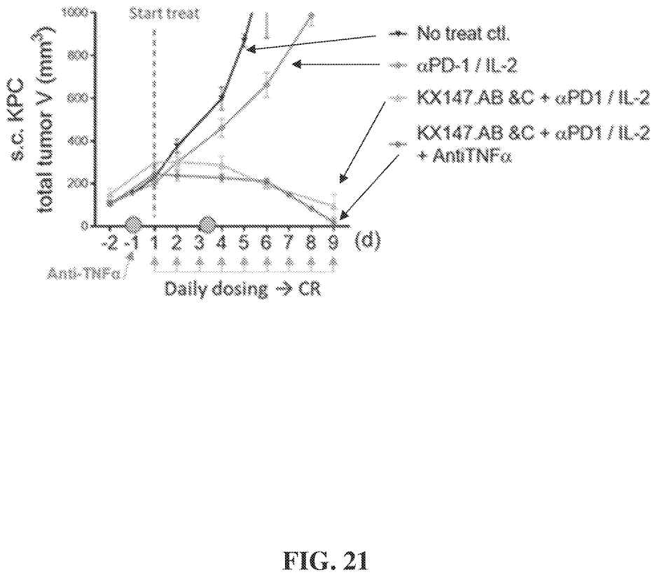

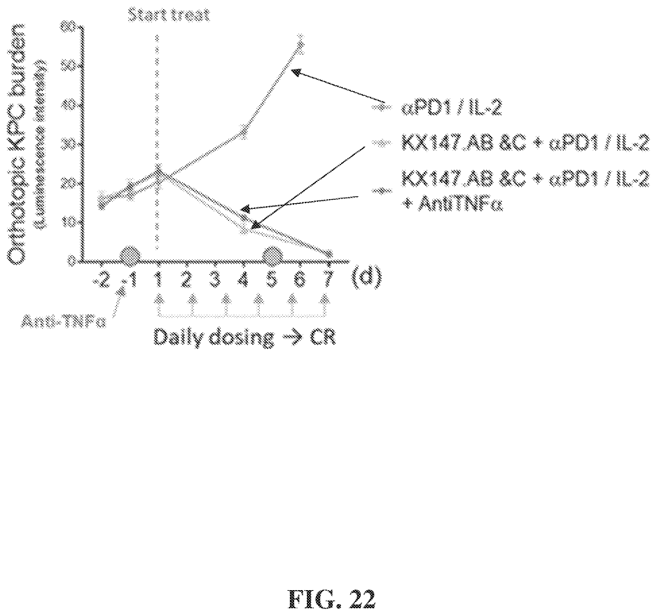

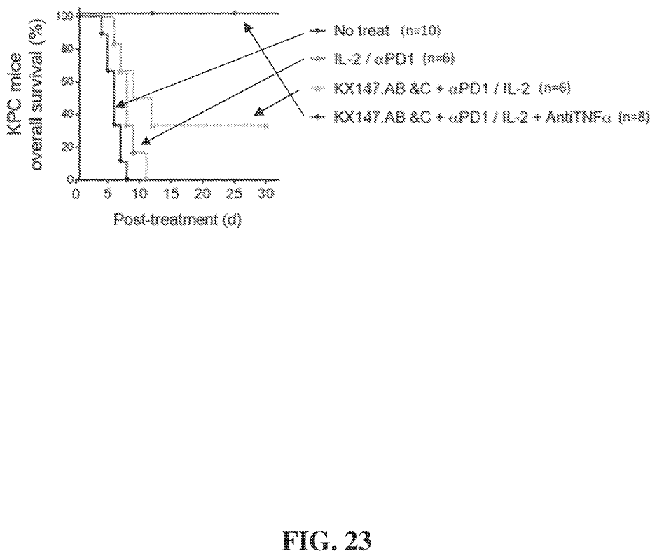

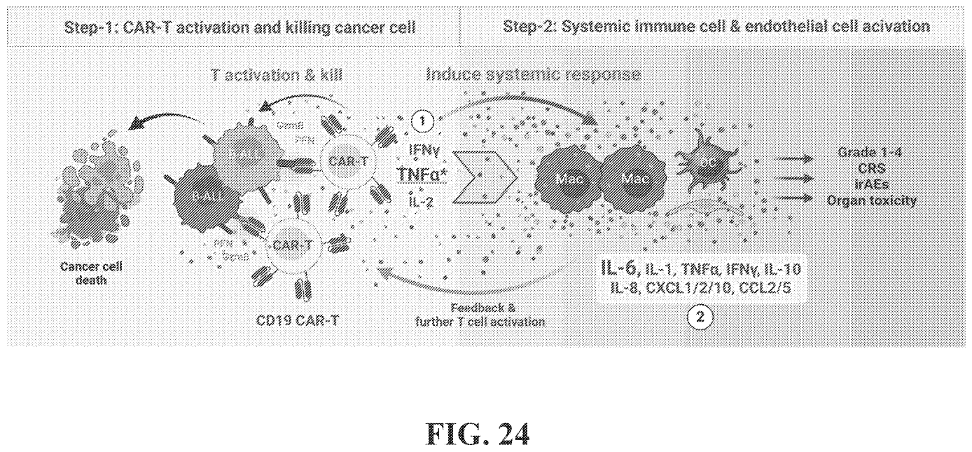

Combination therapy of KX147.AB&C for 2 days (FIG. 20) or for 4 (FIG. 18) days and switching to KX147.AB combination therapy rapidly reduced tumor burden, and anti-TNFα effectively controlled systemic inflammation and reduced adverse effects, thereby promoting animal survival (FIG. 18, FIG. 20, and FIG. 23). KX147.AB&C→KX147.AB combined with IL-2/αPD1 induced potent anti-cancer effects, resulting in rapid tumor volume/burden reduction in s.c. (FIG. 18 and FIG. 21) or orthotopic (FIG. 20 and FIG. 22) engrafted mice. These results also showed that treatment with anti-TNFα mAb did not impact the treatment efficacy.

Taken together, the results from the Examples described above show that despite the fact that anti-TNFα therapy did not contribute to KX147.AB&C-induced tumor elimination, anti-TNFα therapy was responsible for ameliorating the adverse effects associated with the KX147.AB&C therapy, thereby enabling 100% survival rates across all tested murine cancer models. In contrast, the KX147.AB&C-treatment group that was not administered anti-TNFα suffered high rates of post-treatment death even while displaying complete tumor regression.

### Example 5: Neutralization of TNFα Produced by Activated CAR-T Largely Curbed Subsequent Macrophage Activation and Production of Proinflammatory Cytokines and Chemokines

Described herein is an in vitro model system to test whether depletion of activated CAR-T-produced cytokines would prevent activation of macrophages and proinflammatory signaling associated with CRS in corresponding in vivo systems.

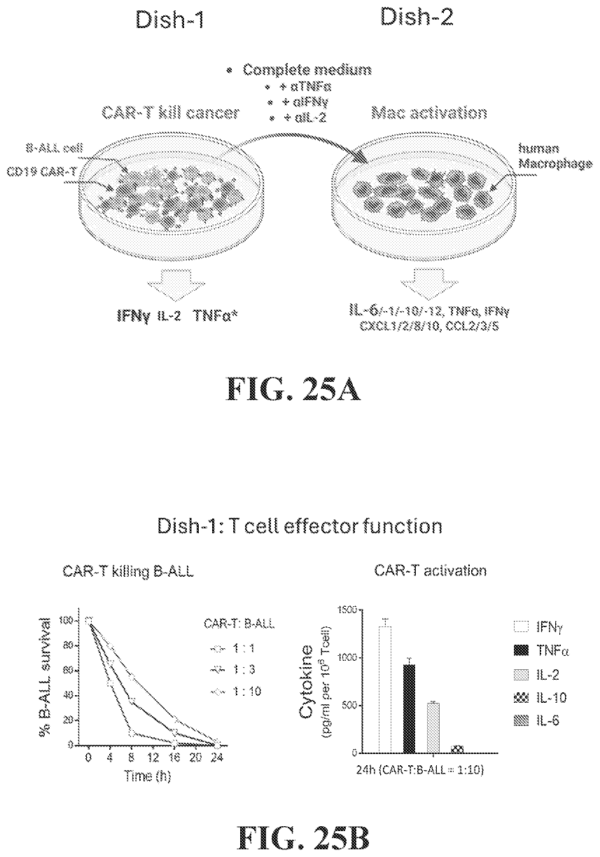

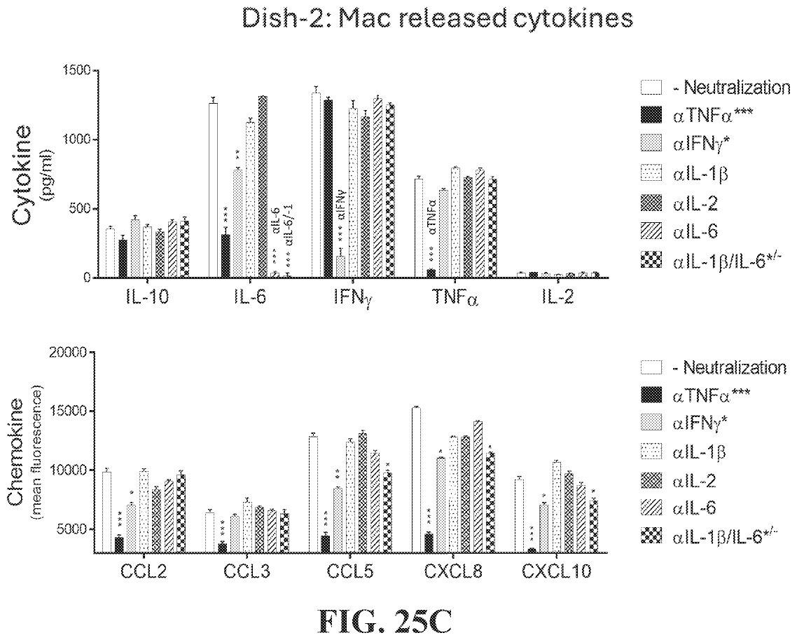

FIG. 25A shows the experimental model system comprising Dish 1 and Dish 2. In Dish-1, CD19 CAR-T and B-ALL leukemia cells were co-cultured for 24 h, during which CAR-T detected B-ALL cells and executed killing along with production of stimulating cytokines IFNg, TNFa and IL-2. The effector function of CD19 CAR-T against B-ALL cells in the co-culture of Dish 1 is shown in FIG. 25B. Dish 2 was seeded with human monocytes-derived macrophages prepared by treating freshly isolated peripheral monocytes with M-CSF for 5 days for macrophage differentiation. After 24 hr of co-culture, the cell culture medium was then collected from Dish 1, pretreated with neutralization mAb against IFNγ, TNFα, IL-2, IL-1β, IL-6, or IL-1β/IL-6^{+/-}. Or no neutralization control and provided to Dish 2 to stimulate human monocytes-derived macrophages. After 16 h, macrophage culture medium from Dish 2 was collected and cytokines in the medium were determined by ELISA.

The resulting effect of neutralization on macrophage activation is shown in FIG. 25C. ELISA was performed to measure the levels cytokines IL-10, IL-6, IFNγ, TNFα and IL-2, and chemokines CCL2, CCL3, CCL5, CXCL8, and CXCL10 after 16 hr of human macrophage culture in Dish 2 with the medium of Dish 1 with or without neutralization. Results showed that, without neutralization, CD19 CAR-T are capable of stimulating macrophage proinflammatory response in vitro, which, without being bound by theory, may help explain the occurrence of CRS in patients receiving CD19 CAR-T therapy.

Furthermore, as shown in FIG. 25C, neutralization of Dish 1 medium with anti-TNFα mAb treatment significantly abated macrophage production of the cytokine IL-6 and all measured chemokines relative to the control receiving no neutralization and neutralization with mAb against IFNγ and IL-2.

Taken together, these results show that TNFα is the prime mediator of macrophage activation by CD19 CAR-T cells. Furthermore, these results suggest, without being bound by theory, that anti-TNFα monoclonal antibody treatment may help prevent macrophage activation and associated CRS in patients receiving CD19 CAR-T therapy. This finding opens up new avenues for improving CAR-T patient outcomes by ameliorating adverse side-effects of the therapy.

### Example 6: Neutralization of TNFα by CAR-T Effectively Abated Surrounding Macrophages from Producing High Level IL-6 and Inflammatory Chemokines

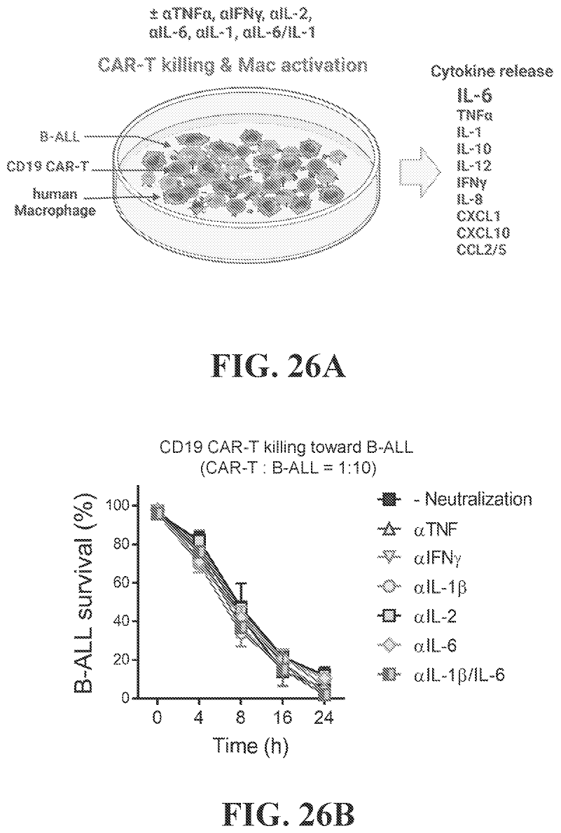

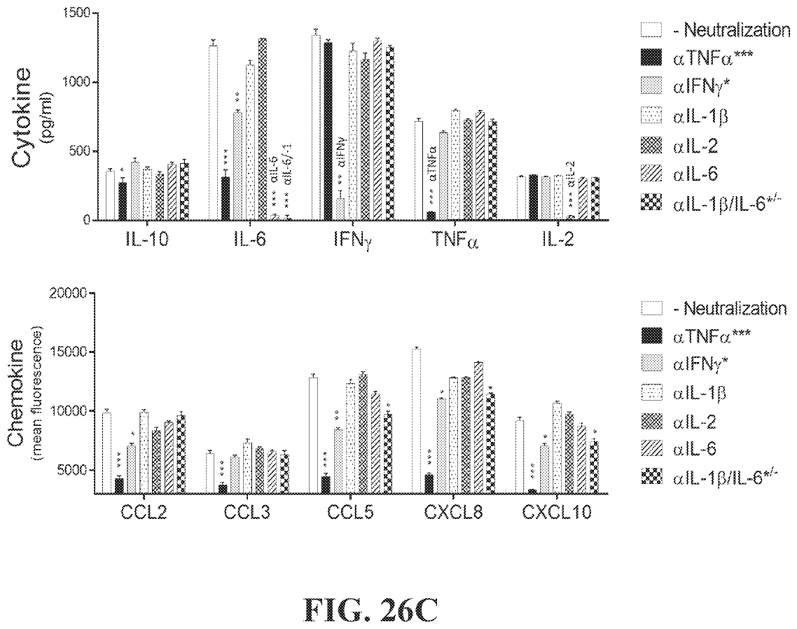

To examine further the nature of macrophage activation by CAR-T cells, a second in vitro assay system was employed. As shown in FIG. 26A, CD19 CAR-T and B-ALL were co-cultured with human monocytes-derived macrophages in a single dish with a cell ratio of CAR-T:B-ALL:hMac=1:10:3 (1×10⁶ T cells/ml). Each individual culture either received no neutralizing treatment, or neutralizing mAb against IFNγ, TNFα, IL-2, IL-1β, IL-6, or IL-1β/IL-6^{+/-}. FIG. 26B shows B-ALL cell death in response to CD19 CAR-T, and establishes that killing was not affected by the addition of cytokine neutralization mAbs. After 24 h, the cell-free medium was collected and cytokines and chemokines within the medium were determined by ELISA.

ELISA was performed to measure the levels cytokines IL-10, IL-6, IFNγ, TNFα and IL-2, and chemokines CCL2, CCL3, CCL5, CXCL8, and CXCL10. The resulting effect of neutralization on macrophage activation is shown in FIG. 26C. Results again confirmed that, without neutralization, CD19 CAR-T are capable of stimulating macrophage proinflammatory response in vitro, which, without being bound by theory, may help explain the occurrence of CRS in patients receiving CD19 CAR-T therapy.

As further shown in FIG. 26C, neutralization with anti-TNFα mAb treatment significantly abated macrophage production of the cytokine IL-6 and all measured chemokines relative to the control receiving no neutralization and neutralization with mAb against IFNγ and IL-2.

Taken together, these results further establish TNFα produced by CD19 CAR-T cells as a major activator of macrophage activation and suggest, without being bound by theory, that anti-TNFα monoclonal antibody treatment may help prevent macrophage activation and associated CRS in patients receiving CD19 CAR-T therapy. Furthermore, the CAR-T/B-ALL/macrophage co-culture model establishes the efficacy of anti-TNFα mAb treatment in an environment that more closely mirrors a physiologically-relevant tumor microenvironment (TME).

### Example 7: Prophylactic Anti-TNFα Prevented CRS without Compromising CAR-T Antitumor Efficacy

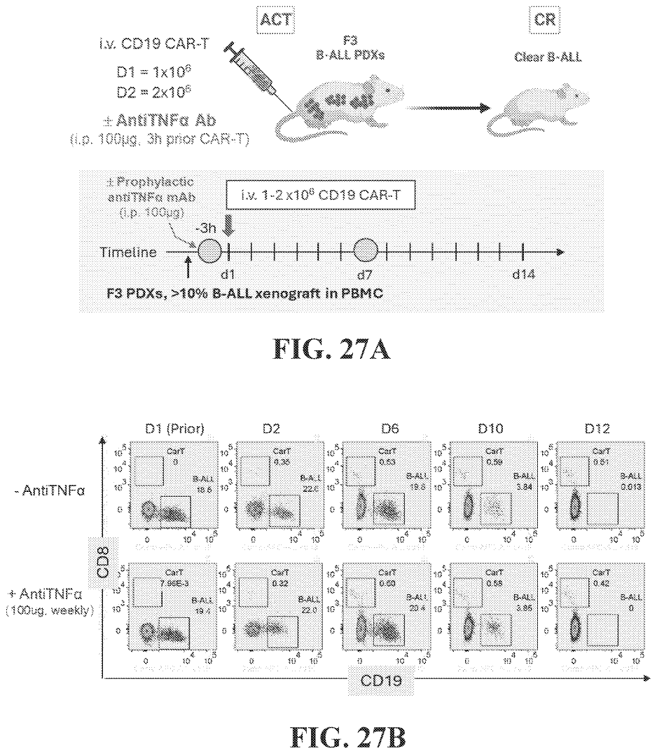

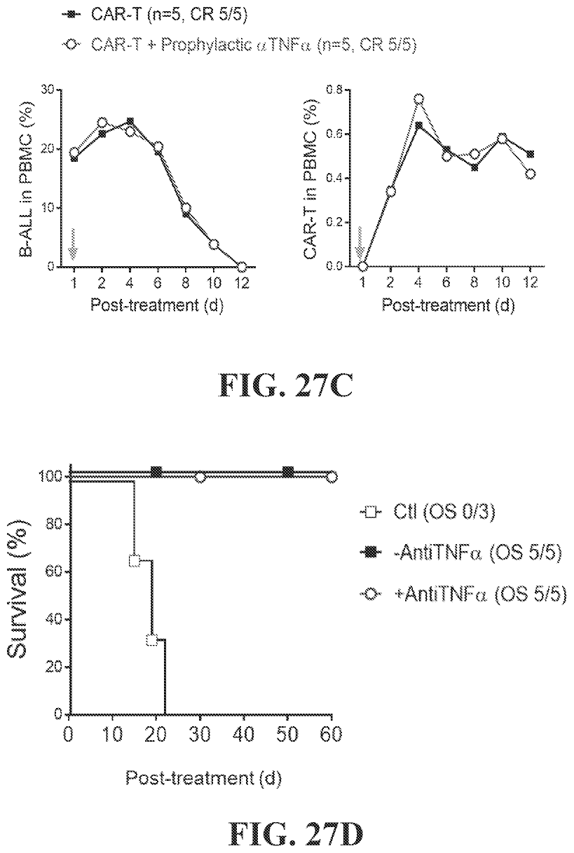

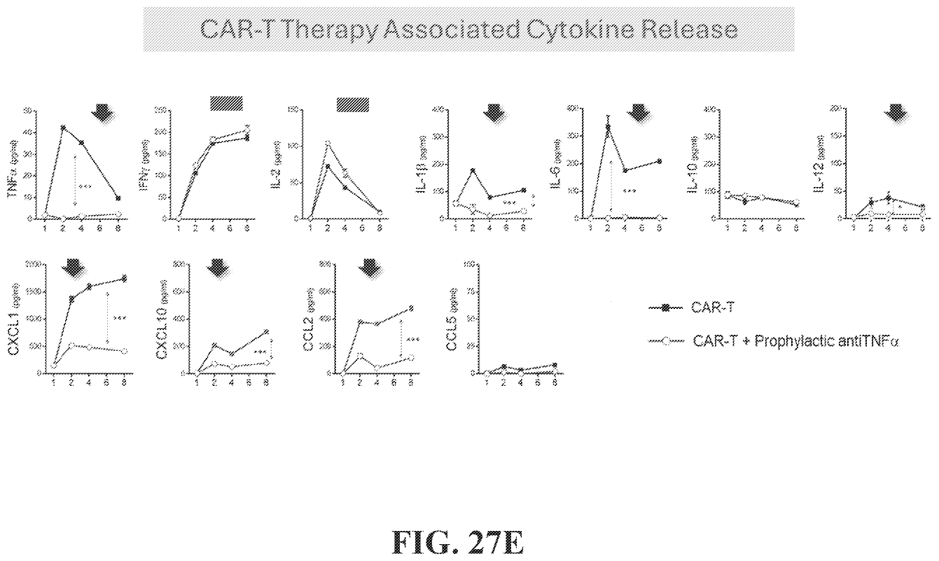

We then sought to determine whether anti-TNFα monoclonal antibody treatment may help prevent CRS occurrence during CAR-T therapy. In vivo CAR-T therapy to B-ALL was carried out in patient-derived xenografts (PDXs) mouse models. We first established the F3 generation of B-ALL PDXs in NSG mice. Once the disease was stable and B-ALL manifested in the peripheral blood of PDX mice, (>10% in PBMC), a single dose of CD19 CAR-T cells (1×106 or 2×106, both effectiveness of eliminating cancer) were infused into the mice without or with prophylactic anti-TNFα administration (100 μg Humira anti-TNFα mAb, i.p.). Prophylactic anti-TNFα administration comprised a single dose of anti-TNFα prior to CAR-T treatment, as well as a follow-on dose at 7-days after occurrence of B-ALL as >10% in PBMC in PDX mice. This experimental approach is outlined in FIG. 27A.

It is important to note in this model that Humira anti-TNFα mAb neutralizes human TNFα produced by CD19 CAR-T, not murine TNFα. Furthermore, CD19 CAR-T produced human TNFα is capable of cross binding to murine TNFR to induce signal transduction and functional effect in mice. Finally, CD19 CAR-T produced human IFNγ does not cross-bind to murine IFNγ receptor, and therefore does not exert a functional effect in mice.

We monitored the peripheral blood of PDX mice for 12 days following CD19 CAR-T treatment with or without prophylactic anti-TNFα mAb. FIG. 27B shows exemplary flow cytometric data from these mice. Sample PBMC analyses showed reduction of B-ALL following CAR-T therapy irrespective prophylactic anti-TNFα mAb administration until mice reached complete response (CR). Furthermore, time-course reduction of B-ALL and increases in CAR-T in PBMC following infusion did not change with anti-TNFα mAb administration as shown in FIG. 27C, and mice from both groups showed 100% survival at 60 days post-treatment (FIG. 27D).

We also measured cytokine and chemokine levels following CAR-T therapy with or without prophylactic anti-TNFα mAb administration. ELISA was performed on blood serum to measure levels of TNFα, IFNγ, IL-2, IL-1β, IL-6, IL-10, IL-12, CXCL1, CXCL10, CCL2, and CCL5 at days 1, 2, 4, 6, and 8 following administration of CD19 CAR-T. As shown in FIG. 27E, anti-TNFα mAb largely abated increased production of IL-6 and inflammatory chemokines associated with CAR-T therapy.

These findings show that prophylactic anti-TNFα is effective at depleting TNFα produced by activated CAR-T during CAR-T “on-target” effector activities in vivo while preserving the therapeutic efficacy of CAR-T. Furthermore, prophylactic anti-TNFα does not disrupt production of IFNγ and IL-2 from CAR-T cells in vivo, nor prevent the in vivo proliferation and persistency of CAR-T cells. Prophylactic anti-TNFα largely abated CAR-T therapy-associated CRS by reducing proinflammatory cytokines including TNFα, IL-6, IL-1β and IL-12, and chemokines such as CXCL1, CXCL10 and CCL2 that drive infiltration of polymorphonuclear leukocytes (PMN) and monocytes. Finally, prophylactic anti-TNFα may help prevent life-threatening immune-related adverse effects (irAEs) through its efficacy in abating CAR-T therapy-associated CRS.

### Example 8: Prophylactic Anti-TNFα Protected Mice from CD3/CD28 TCR Ligation-Induced CRS

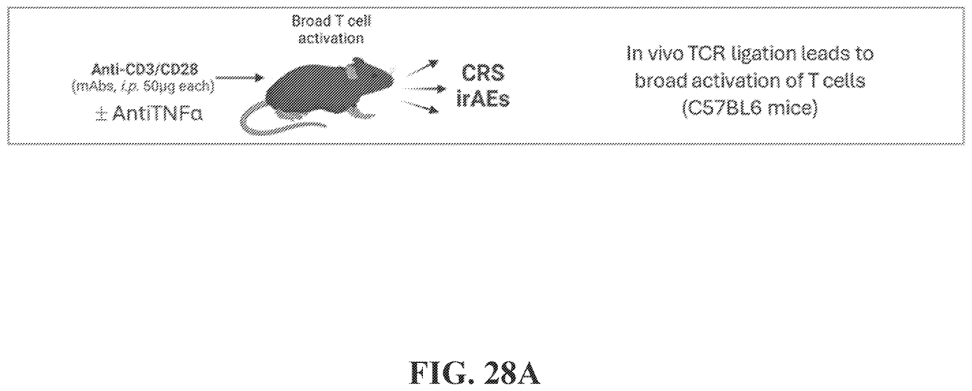

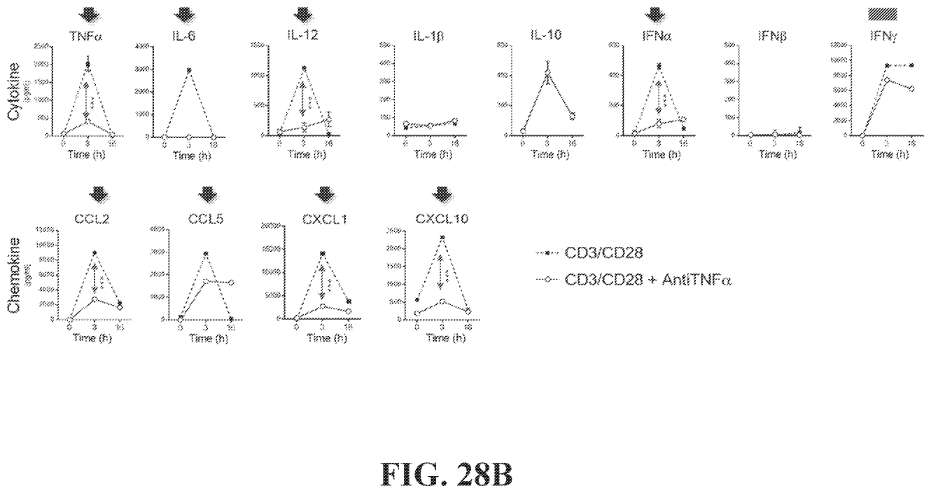

We next sought to determine whether prophylactic anti-TNFα mAb administration was sufficient to prevent CRS occurrence from broad antibody-induced T cell activation. Mice with or without prophylactic anti-TNFα mAb (100 μg i.p. prior to treatment) were given a mixture of CD3 and CD28 ligation mAbs (50 μg each, i.p.) and assayed for cytokine and chemokine release. Serum levels of TNFα, IL-1β, IL-6, IL-10, IL-12, IFNα, IFNβ, IFNγ, CXCL1, CXCL10, CCL2, and CCL5 were assayed by ELISA at 0 hr, 3 hr, and 16 hr post-CD3/CD28 ligation treatment. This experiment approach is shown in FIG. 28A.

As shown in FIG. 28B, prophylactic anti-TNFα mAb administration effectively neutralized TNFα while also abating the release of IL-6, IL-12 and IFNα levels. Furthermore, anti-TNFα did not affect the release of IFNγ a key cytokine for T cell effector function. Prophylactic anti-TNFα mAb also abated the release of proinflammatory chemokines that mobilize PMN (CXCL1), monocytes (CCL2) and T cells (CXCL10). These result establish the efficacy of prophylactic anti-TNFα mAb administration in preventing CRS in response to broad T cell activation.

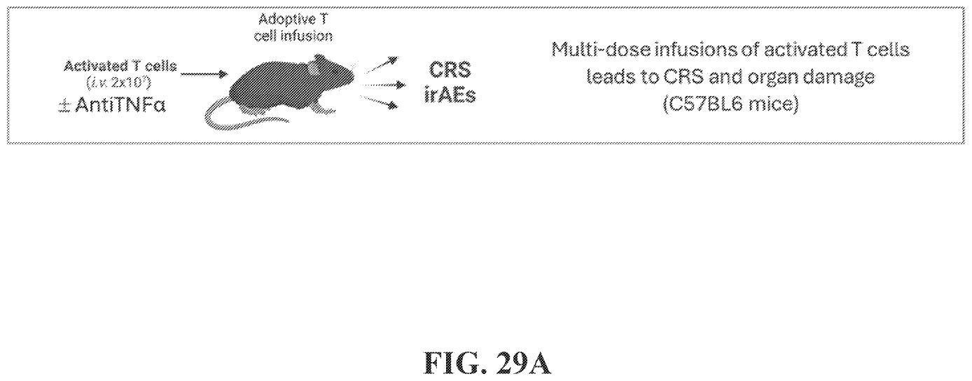

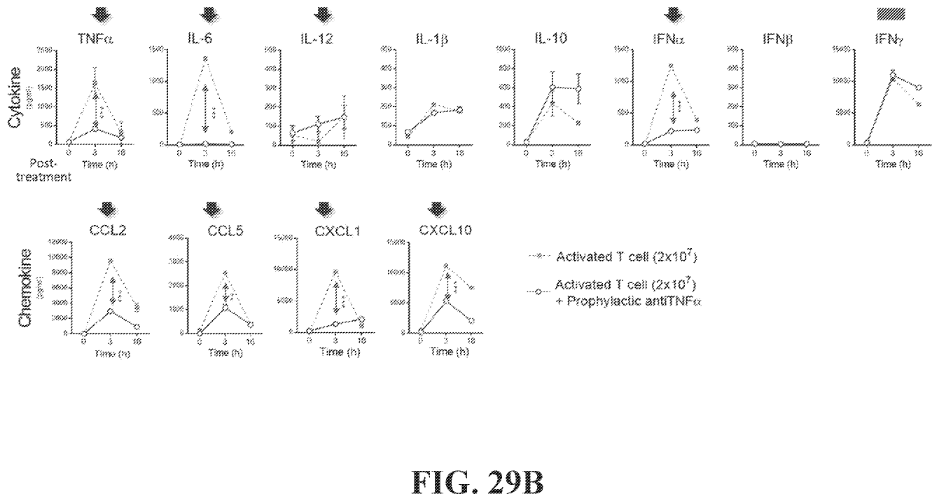

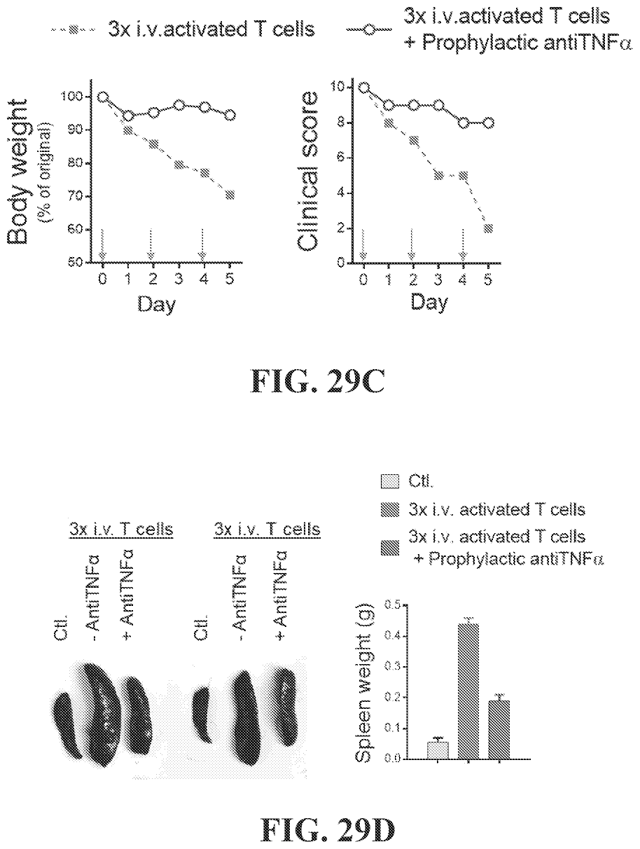

We further tested prophylactic anti-TNFα mAb in the context of activated T cell infusions in mouse models. As outlined in FIG. 29A, splenocytic T cells (2×10⁷ cells) were activated with TCR-ligating CD3 and CD28 antibodies, followed by i.v. infusion into syngeneic mice (C57BL6) with or without prophylactic anti-TNFα administration (100 μg i.p. prior to treatment). Multiplex ELISA was employed to measure cytokine and chemokine levels in blood serum at 0 hr, 3 hr, and 16 hr following a single infusion of splenocytic T cells. FIG. 29B shows that prophylactic anti-TNFα administration abolished proinflammatory cytokines TNFα, IL-6, IL-12, and IFNα in blood serum, and also substantially reduced the level of chemokines that mobilize PMN (CXCL1), monocytes (CCL2, CCL5) and T cells (CXCL10).

We then proceeded to test the effect of prophylactic anti-TNFα mAb administration on repeated infusion of activated T cells. Syngeneic mice (C57BL6) received three doses of splenocytic T cells activated with TCR-ligating CD3 and CD28 antibodies at day 0, day 2, and day 4 of treatment (each 2×10⁷ cells by i.v.). FIG. 29C shows that prophylactic anti-TNFα mAb treatment prevented loss of body weight and decreased clinical score associated with administration of activated T cells, while also preventing the formation of splenomegaly (FIG. 29D).

Overall, these results showed that prophylactic anti-TNFα mAb administration ameliorated CRS and irAEs associated with activated T cells. This suggests that prophylactic anti-TNFα has application in improved outcomes during bispecific monoclonal antibody (BsAb) and other antibody-induced T cell activation. Furthermore, prophylactic anti-TNFα treatment may be used for improved outcomes with adoptive T cell therapies, including tumor infiltrating lymphocyte (TIL) therapy, NeoT therapy, TCR-activated T cell therapy, and others.

### Example 9: Topical Treatment with a Combination of TLR Agonists and SHP-1 Inhibitor dTPI-1 Induces Profound Anti-Tumor Response Against 4T1 Cutaneous Breast Cancer Tumors in Balb C Mice

SHP-1 is abundantly expressed in macrophages. Proteomic studies examining expression of non-receptor protein tyrosine phosphatases (PTPs) in macrophages reveal that SHP-1 has the highest expression level. SHP-1 is the most abundant protein tyrosine phosphatase expressed in macrophages of both human and murine origins. Macrophages with high SHP-1 activity in a tumor milieu diminish production of proinflammatory cytokines but produce high IL-10 under stimulation of IFNγ/LPS, establishing a strong immunosuppressive state.

Immunosuppression by tumor associated macrophages (TAMs) results in a “Cold” tumor microenvironment (i.e., COLD TME) wherein T cells exist in small number and exhibit an exhausted phenotype. Within the COLD TME, inhibition of PD-1 with αPD-1 treatment only partially diminishes immunosuppression, thereby leading to a slight delay in tumor growth and progression.

Treatment of tumors with inhibitors of SHP-1 (e.g., TPI-1, dTPI-1, TPI derivatives, or other SHP-1 inhibitors) in combination with proinflammatory activators (e.g., TLR agonists, Sting activators, proinflammatory cytokines, or RT) reprograms TAMs, thereby inducing their proinflammatory phenotype, phagocytosis, and immunogenic antigen presentation that further activates cancer specific T cells for expansion and anti-cancer cytotoxicity. This treatment thereby turns the tumor microenvironment into a “HOT TME” wherein T cells can target and potently kill cancer cells. Addition of αPD-1 removes additional inhibition on T cells, thereby boosting T cells for anti-cancer activities within the HOT TME.

Topical treatments and transdermal therapies (e.g., microneedle devices) for the application of medications such as drugs and immune regulatory agents (e.g., imiquimod) to the skin are broadly used to treat cutaneous cancers including melanoma, other forms of skin cancer, and precancer skin lesions. The same topical/transdermal therapies have also been explored to treat cutaneous metastases, such as cutaneous metastases of breast cancer (CMOBC), which demonstrate synergistic effect with systemic therapies including immune checkpoint inhibitors and globally reduce cancer burden.

Since topical treatments serve both to locally treat cancer lesions in situ and to trigger a systemic anti-cancer response and achieve therapeutic efficacies, we designed topical immunotherapies against cancer comprising proinflammatory activators (e.g., TLR agonists, Sting activators, proinflammatory cytokines, or RT) and inhibitors of SHP-1 (e.g., TPI-1, dTPI-1, TPI derivatives, or other SHP-1 inhibitors).

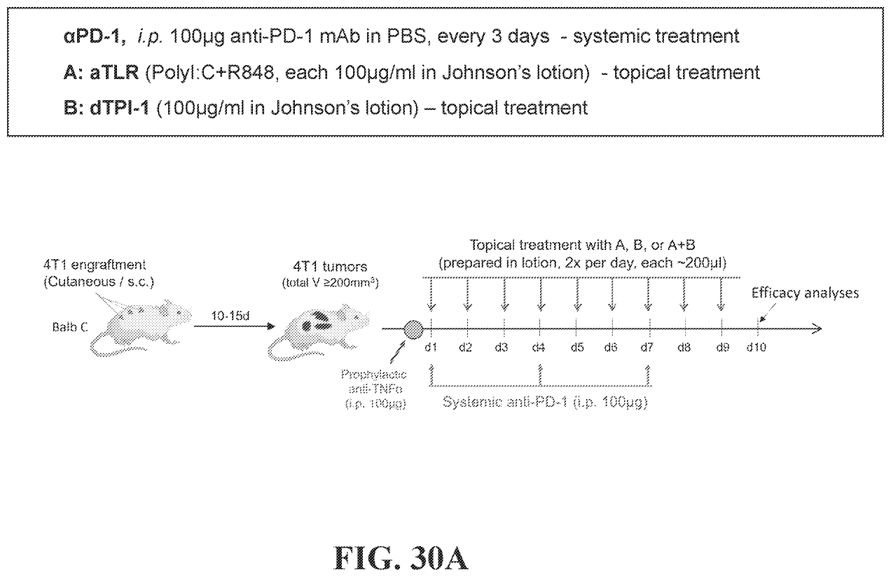

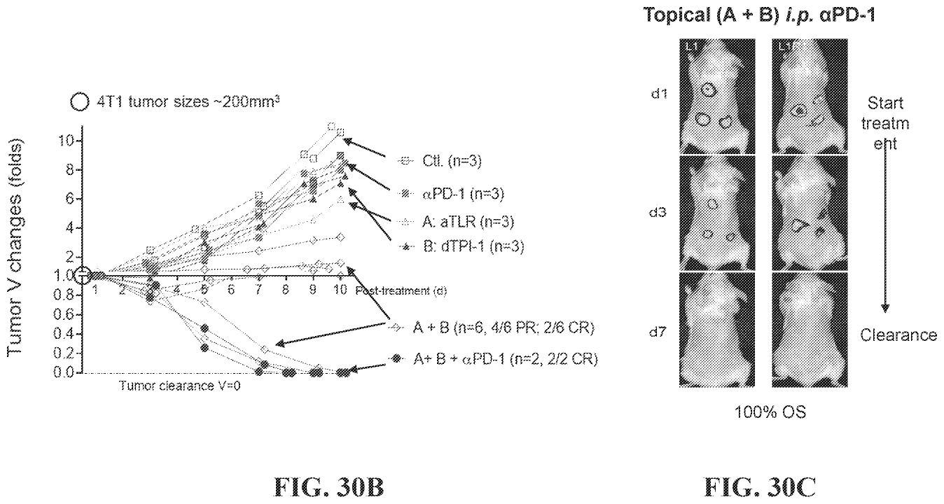

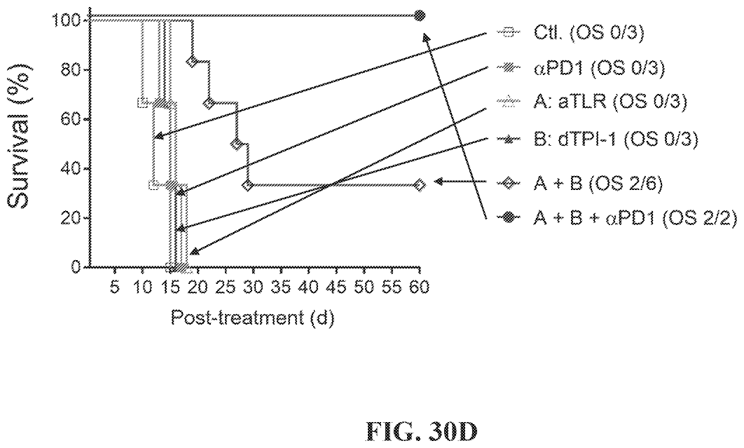

Efficacy of topical treatments against tumor growth and/or progression was tested using murine models of cutaneous/subcutaneous breast cancer. As shown in FIG. 30A, murine 4T1 (4T1-Luc) breast cancer was engrafted into genetically matched Balb C mice (syngeneic model) in a multi-point cutaneous/subcutaneous manner into the dorsal skinfold. Palpable tumors were formed in 10-15 days and were traced by bioluminescence imaging. Tumor sizes were measured manually, and the volume was calculated by the equation=[length (mm)×width (mm)×width (mm)]/2. Once the total tumor volume reached ≥200 mm³, treatments were started.

Treatment groups included: (i) Control treatment (ctl.), wherein mice received topical non-drug lotion (vehicle) only; (ii) αPD-1 treatment, wherein mice received topical non-drug lotion (vehicle) treatment along with 100 μg of anti-PD-1 monoclonal antibody once every three days through intraperitoneal injection; (iii) Topical treatment with TLR agonists only (i.e. Condition A), wherein mice received aTLR (Resiquimod (R848) and polyI:C) prepared in Johnson's lotion, each at 100 μg/ml in a final volume of 100-200 μL lotion: (iv) Topical treatment with SHP-1 inhibitors only (i.e. Condition B), wherein mice received deuterated TPI-1 (dTPI-1) at 100 μg/ml in a final volume of 100-200 μL lotion; (v) Topical treatment with Conditions A+B only in a final volume of 100-200 μL lotion; and (vi) Topical treatment with Conditions A+B in a final volume of 100-200 μL lotion and systemic αPD-1 treatment. All lotion treatments were applied twice a day for each day of the duration of the experiment. Furthermore, mice within all treatment groups received a dose of prophylactic anti-TNFα mAb (100 μg through intraperitoneal injection) three hours before the first dose of topical therapies for prophylactic neutralization of TNFα to ameliorate treatment-associated Cytokine Release Syndrome (CRS). See FIG. 30A. At 10 days after initiation of treatment, efficacy analysis was performed to determine tumor growth in each treatment group.

As shown in FIG. 30B, 4T1 cutaneous breast cancer showed strong resistance to immune checkpoint inhibition with anti-PD-1 treatment alone (treatment group (ii)), wherein tumor growth was only slightly delayed relative to control treatment group (i). Topical treatment of 4T1 tumors with TLR agonists alone (treatment group (iii)) or SHP-1 inhibitor dTPI-1 alone (treatment group (iv)) only slightly delayed tumor progression beyond treatment group (ii). The combination of TLR agonists and dTPI-1 (treatment group (v)) induced profound anti-tumor response, significantly inhibiting tumor growth and inducing tumor regression beyond either TLR agonists alone (treatment group (iii)) or SHP-1 inhibitors alone (treatment group (iv)). Finally, topical treatment with both TLR agonists and SHP-1 inhibitors alongside systemic anti-PD-1 therapy (treatment group (vi)) further enhanced tumor regression, inducing immune clearance of all 4T1 breast cancer lesions.

Mice in treatment group (vi), which received topical treatment comprising TLR agonists and SHP-1 inhibitors alongside systemic anti-PD-1 therapy reached complete response (CR) to therapy. See FIG. 30C. Furthermore, mice in treatment group (vi) exhibited 100% overall survival, which is markedly improved over all other tested treatment groups. See FIG. 30D. This study found that topical application of combined proinflammatory activators (TLR agonists) and SHP-1 inhibitors (dTPI-1), a combination previously shown to induce TAM proinflammatory signaling and T cell activation within a tumor microenvironment (i.e., a “HOT TME”), is capable of significantly inhibiting tumor growth and inducing tumor regression in mouse models of cutaneous breast cancer metastasis. This effect is enhanced through immune checkpoint inhibition with systemic anti-PD-1 therapy. Finally, prophylactic treatment with anti-TNFα mAb is effective in avoiding toxic side-effects to treatment such as cytokine release syndrome (CRS). Therefore, this study displays the efficacy of topical administration for SHP-1 inhibitor- and proinflammatory agonist-mediated cancer immunotherapy.

### Example 10: Topical Treatment with a Combination of TLR Agonists and SHP-1 Inhibitor dTPI-1 Induces Profound Anti-Tumor Response Against Multiple Forms of Cancer in Murine Models

Given the efficacy of topical treatments comprising SHP-1 inhibitors and proinflammatory activators against cutaneous/subcutaneous metastatic breast cancer, next we wanted to establish broad applicability of our topical treatment therapy across multiple forms of cutaneous or subcutaneous cancers.

We tested the efficacy of our topical treatment using murine models of cutaneous/subcutaneous lung cancer. Murine lung cancer (LLC-luc) was engrafted into syngeneic C57Bl/6 mice in a multi-point cutaneous/subcutaneous manner into the dorsal skinfold. Lung cancer lesions were formed in 10-15 days and was traced by bioluminescence imaging as described above. Tumor growth was monitored as outlined above, and treatments were initiated once total tumor volume reached ≥200 mm³.

Treatment groups included: (i) Control treatment (ctl.), wherein mice received topical non-drug lotion (vehicle) only: (ii) αPD-1 treatment, wherein mice received topical non-drug lotion (vehicle) treatment in combination with 100 μg intraperitoneal injection of αPD-1 once every three days; (iii) Topical treatment with aTLR and SHP-1 inhibitor, wherein mice received aTLR, (Resiquimod (R848), polyI:C, and LPS) (Condition A) and SHP-1 inhibitor (dTPI-1) each at 100 μg/ml prepared in Johnson's lotion in a final volume of 100-200 μL alongside systemic αPD-1 treatment as outlined in treatment group (ii); and (iv) Topical treatment with aSting and SHP-1 inhibitor, wherein mice received aSting (MSA-2, ADU-S100 and cGAMP, each 5 mg/ml) and SHP-1 inhibitor (dTPI-1) alongside systemic αPD-1 treatment as outlined in treatment group (ii). All lotion treatments were applied twice a day for each day of the duration of the experiment. Furthermore, mice within all treatment groups received a dose of anti-TNFα mAb (100 μg through intraperitoneal injection) three hours before the first dose of topical therapies for prophylactic neutralization of TNFα to ameliorate treatment-associated Cytokine Release Syndrome (CRS).

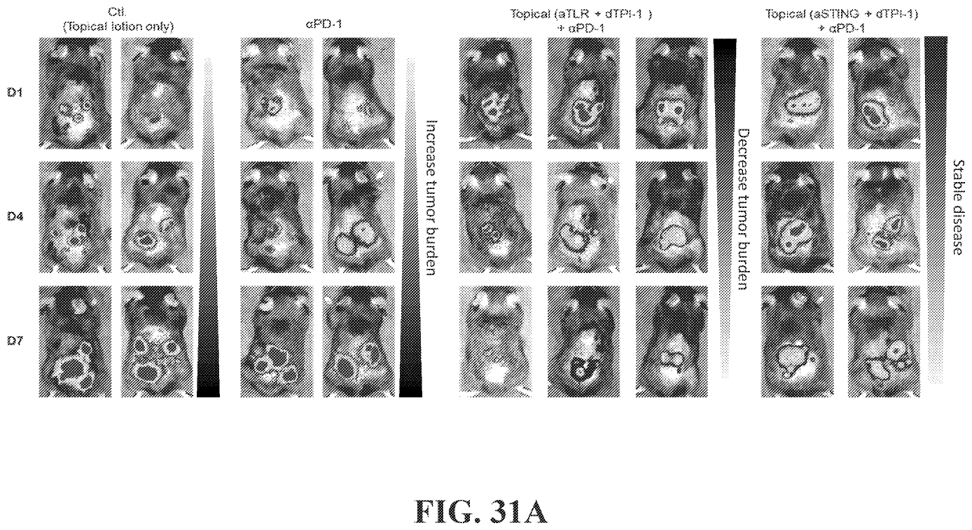

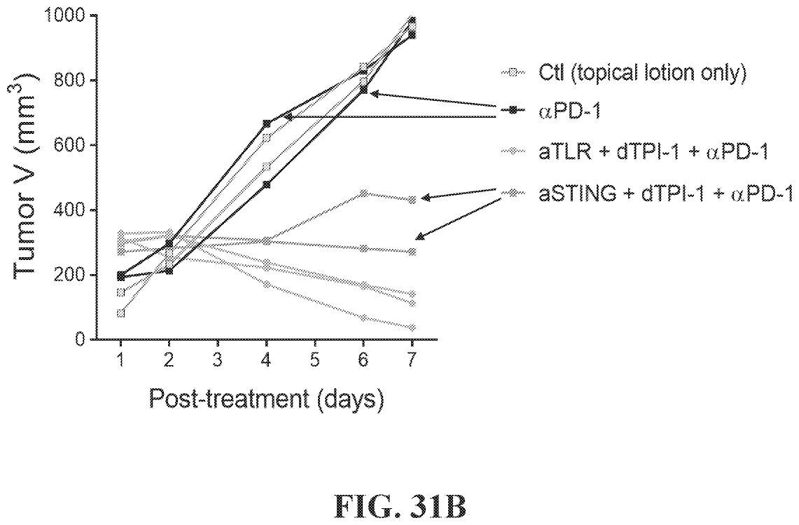

FIG. 31A shows tumor growth over seven days of treatment following anti-TNFα dosing. Representative mice from αPD-1 treatment group (ii) showed only minor reduction in tumor progression relative to the control group (i). Tumor growth and progression was strongly impeded by topical administration of aTLR and SHP-1 inhibitor alongside systemic anti-PD-1 therapy in treatment group (iii), to a greater extent than observed in mice receiving topical treatment with aSting and SHP-1 inhibitor alongside systemic anti-PD-1 therapy (treatment group (iv)). Changes in tumor volume are quantified in FIG. 31B.

Overall, our results display the efficacy of topical treatment comprising SHP-1 inhibitors and TLR activators against a broad range of cutaneous/subcutaneous cancers. Without being bound by theory, these results may also establish that topical administration of SHP-1 inhibitors activate TAMs within immunosuppressed tumor microenvironments (i.e., “COLD TMEs”), enabling macrophage proinflammatory response and antigen-presentation activity that can activate anti-tumor T cell immunity and induce a cytotoxic response against cancer cells. Finally, these results confirm the efficacy of prophylactic anti-TNFα mAb dosing in preventing treatment side-effects such as CRS without impeding anti-tumor activity of SHP-1 inhibitors and TLR agonists in the topical treatment.

### Example 11: Inhibition of SHP-1 Promotes ICI and IL-2 Mediated Anti-Cancer Efficacies

Inhibition of SHP-1 was tested in vivo for tumor therapies. In short, tumor cells (i.e., MC38) were engrafted into C57Bl/6 mice and the mice administered (i) TPI-1, (ii) αPD-1 antibody, (iii) IL-2, (iv) anti-CD3/CD28 antibodies, (v) TPI-1+αPD-1 antibody, (vi) TPI-1+IL-2, (vii) TPI-1+anti-CD3/CD28 antibodies, or (viii) TPI-1+IL-2+prophylactic anti-TNFα/anti-IL-6 antibodies. Tumor growth was measured over time until the tumors reached 2.0 cm in diameters. TPI-1 was administered at a dose of 1 mg/kg every 2 days. IL-2 was administered at 30,000 IU by intratumoral injection every 3 days. Anti-PD-1 antibody was 205dminister at 50 μg by intratumoral injection every 3 days. Anti-CD3 antibody and anti-CD28 antibody were each 205dminister at 50 μg by intratumoral injection in a single dose.

These studies found that treating established tumors, such as MC38, KPC, or LLC, with the SHP-1 inhibitor TPI-1, αPD-1, IL-2, or anti-CD3/CD28 alone had only minor effects and did not trigger strong intratumoral T cell response or tumor elimination. However, SHP-1 inhibition enabled the intratumoral injection of αPD-1, IL-2, or anti-CD3/CD28 to achieve strong anti-tumor effects, leading to elimination of previously treatment-refractory tumors.

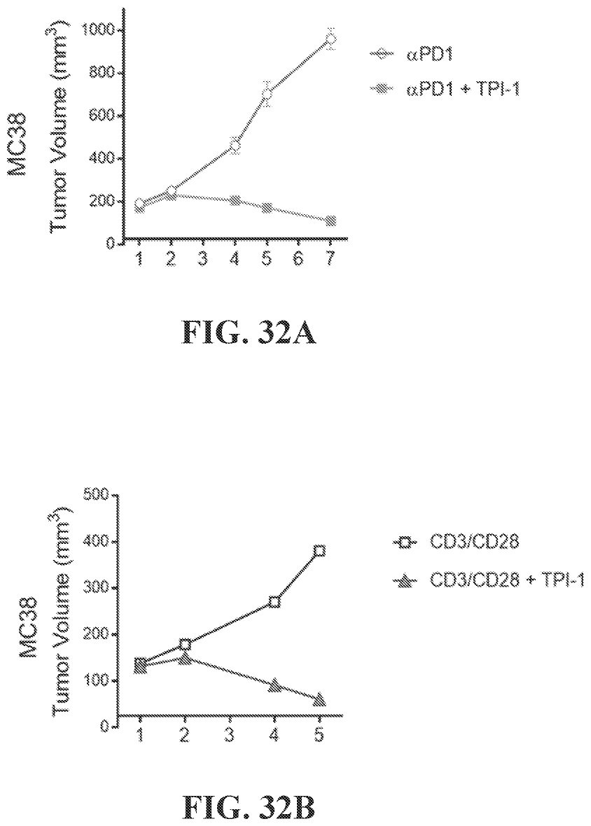

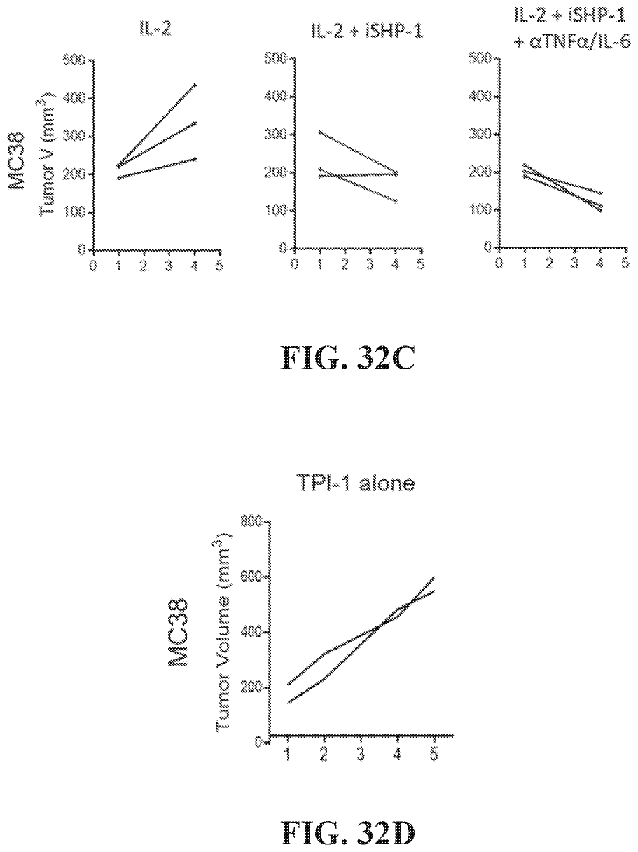

As shown in FIGS. 32A-32C, after establishment, MC38 tumors exhibited strong resistance to αPD-1 (FIG. 32A) or anti-CD3/CD28 antibodies (FIG. 32B) or IL-2 (FIG. 32C), or αPD-1+IL-2 combination (data not shown). Addition of the SHP-1 inhibitor, TPI-1, into the therapeutic regimens instantly overcame treatment resistance by increasing sensitivity to αPD-1, IL-2, or αPD-1+IL-2 combination, leading to the elimination of even large size, late-stage MC38 tumors. Importantly, TPI-1 alone did not attenuate tumor growth (FIG. 32D). Notably, prophylactic treatment with anti-TNFα/anti-IL-6 antibodies did not impede the ability of IL-2+iSHP-1 to eliminate MC38 tumors, showing that prophylactic treatment to avoid CRS is compatible with iSHP-1 mediated anti-tumor therapies.

### Example 12: Protective Effects of Anti-TNFα Treatment Against Adverse Events when Used in Combination with TPI-1 and/or a Myeloid Cell Activating Agent or Therapy to Treat KPC Pancreatic Ductal Adenocarcinoma

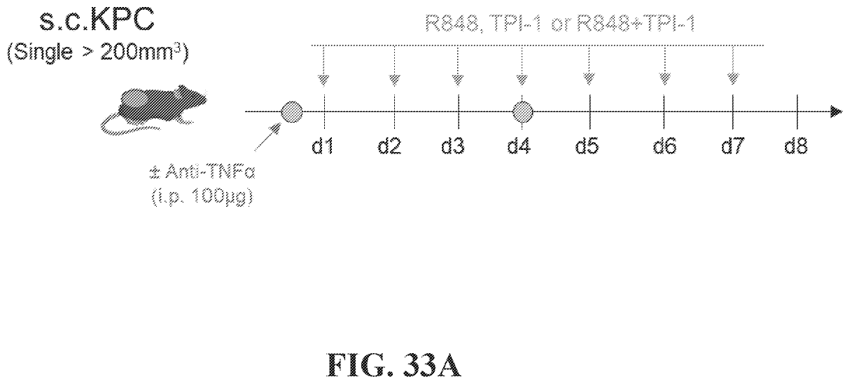

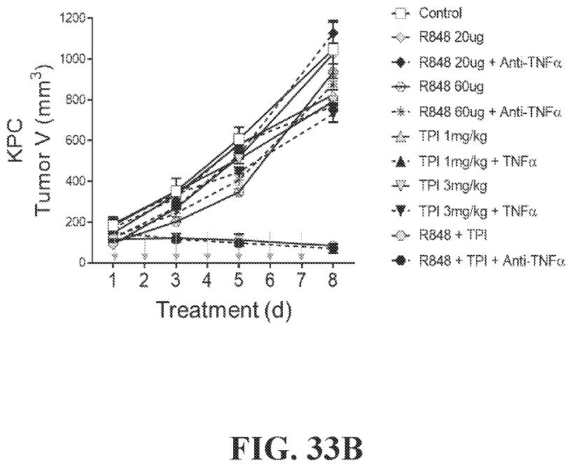

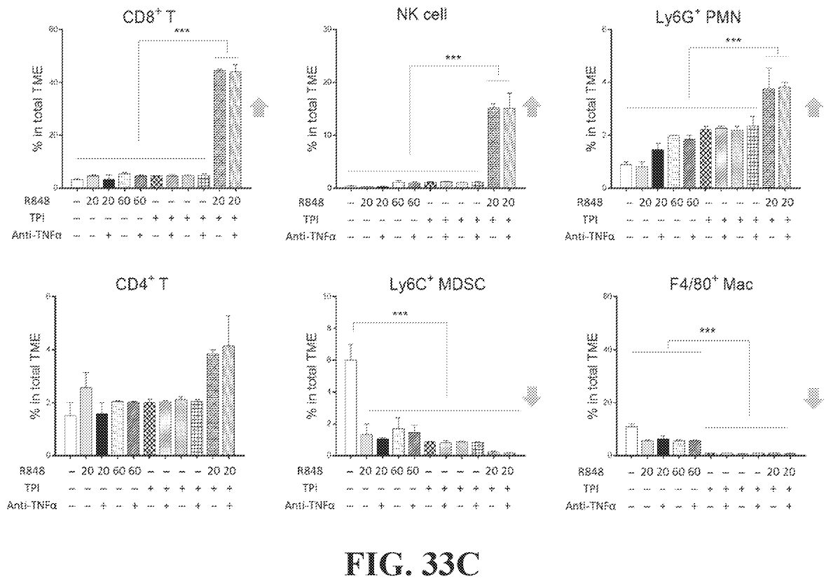

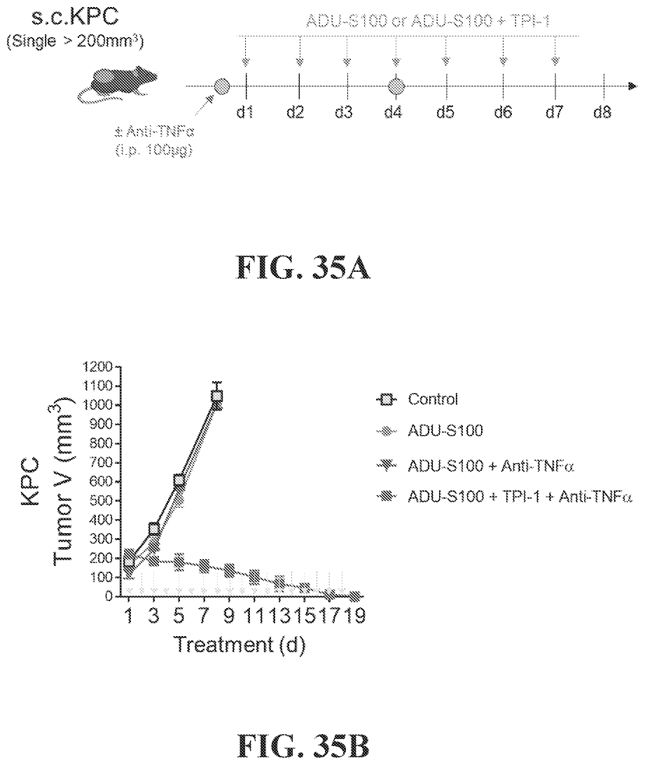

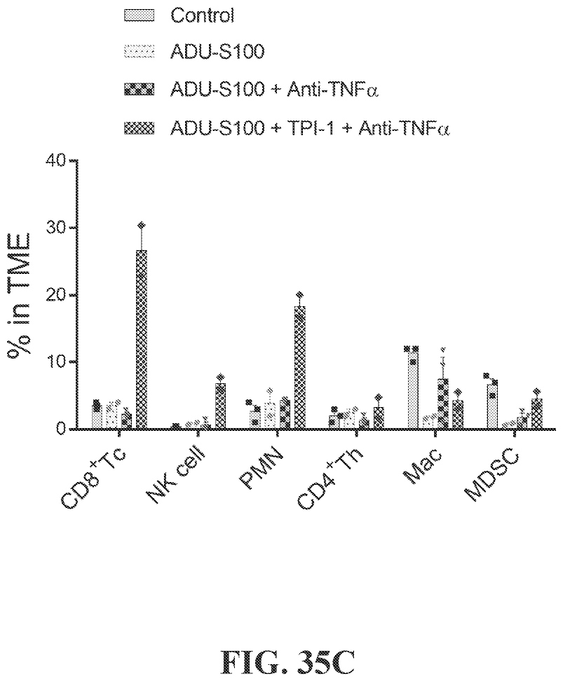

Mice with established KPC pancreatic ductal adenocarcinoma (~200 mm³) were treated with TPI-1 alone or in combination with a myeloid cell activating agent or therapy (R848, as shown in FIG. 33A, or the STING activator ADU-S100, as shown in FIG. 35A) with or without prophylactic anti-TNFα mAb (150 μg, i.p, 3 hr prior to treatment initiation). Treatment comprised: 1) TPI-1 at 1 mg/kg or 3 mg/kg s.c. and/or 2) R848 at 20 μg or 60 μg, s.c or ADU-S100 at 100 μg/mouse, s.c. The treatment was administered daily. Tumor volume changes were recorded daily, and tumor microenvironments (TMEs) were analyzed for immune cell infiltration on Day 8 after treatment administration was started. Immune cell lineages assessed by flow cytometry included: CD8⁺ T cells, CD4⁺ T_{H} cells, NK cells, PMNs, macrophages, and myeloid derived suppressor cells (MDSCs). CRS was assessed by collecting blood serum prior to and 3 hrs after each round of KX147.AB&C treatment. Serum cytokine and chemokine levels (TNFα, IL-6, IL-1β, IL-10, IFNα, IFNγ, CCL2, CCL5, CXCL1, etc.) were assayed. Organ examination was performed after euthanasia. Various organs such as spleen, liver, kidney, and colon were resected, examined, and weighed.

As shown in FIG. 33B, all mice treated with TPI-1 and RP848 and prophylactic anti-TNFα showed tumor elimination. Similarly, all mice treated with TPI-1 and ADU-S100 and prophylactic anti-TNFα showed tumor elimination (FIG. 35B). Tumors from control mice showed poor infiltration of anti-tumor-acting T cells, NK cells, and PMNs but high levels of tumor-promoting macrophages and MDSCs (FIG. 33C and FIG. 35C). Tumors from mice treated with TPI-1 and R848 with or without anti-TNFα mAb displayed significant increase in the infiltration of CD8⁺ T cells, CD4⁺ T_{H} cells, and NK cells and a significant reduction in the level of macrophage and MDSC infiltration (FIG. 33C). Similarly, tumors from mice treated with TPI-1 and ADU-S100 with or without anti-TNFα mAb displayed significant increase in the infiltration of CD8⁺ T cells, CD4⁺ T_{H} cells, and NK cells and a significant reduction in the level of macrophage and MDSC infiltration (FIG. 35C).

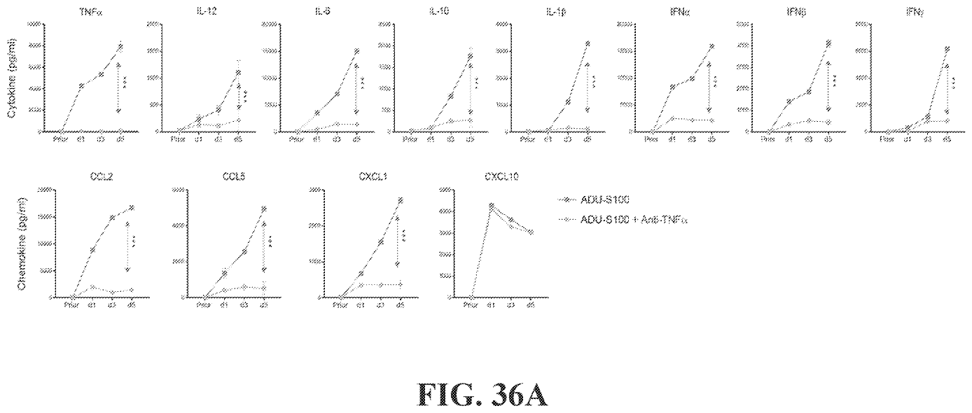

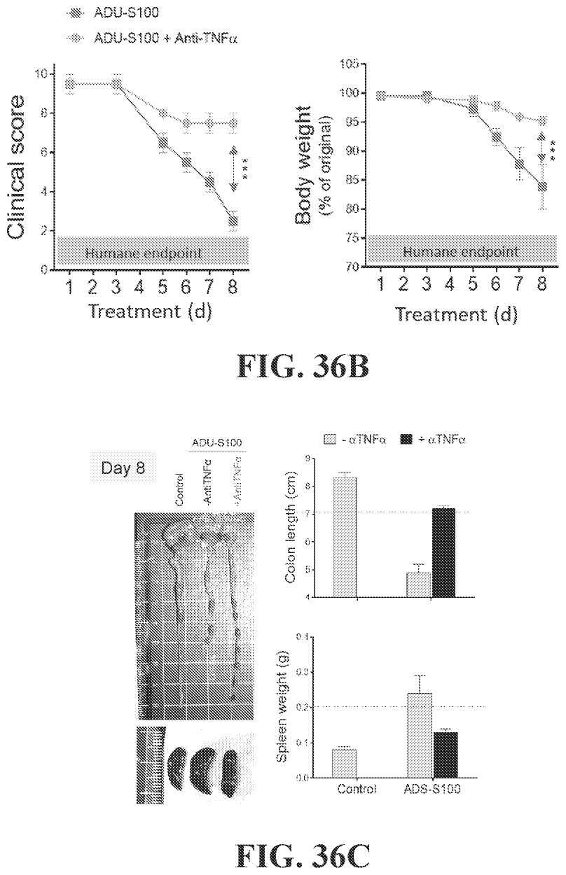

In all treatment models, mice were analyzed for CRS and organ dysfunction. Across all cytokines tested, mice that were co-treated with anti-TNFα mAb showed reduced serum cytokine levels as shown in FIG. 34A as well as FIG. 36A, as well as reduced CCL2, CCL5, and CXCL1 chemokine levels. Importantly, no significant changes were observed in chemokine CXCL10 levels, which is critical for T cell trafficking to the TME. Both FIG. 34B and FIG. 36B demonstrate that the addition of prophylactic anti-TNFα mAb treatment was able to rescue loss of body weight and decreased clinical scores associated with therapy-induced CRS. Finally, FIG. 34C and FIG. 36C demonstrate the splenomegaly and colitis that was found to occur in mice aggressively treated with TPI-1 and/or a myeloid cell activating agent or therapy (R848 or ADU-S100) was ameliorated with prophylactic anti-TNFα mAb treatment.

These results demonstrate that the addition of anti-TNFα mAb treatment does not reduce, ameliorate, or eliminate the adaptive immune cell-promoting actions of the TPI-1 and/or myeloid cell activating agent or therapy combination treatment. However, these results do confirm the results from the LLC murine model in Example 1 wherein anti-TNFα mAb treatment reduced systemic CRS and organ damage, which was an effect that surprisingly was not seen with anti-IL-6 mAb treatment. These results identify that anti-TNFα therapy is responsible for the reduction in therapy-related toxicities and is not interchangeable with antibody depletion therapy of another inflammatory cytokine such as IL-6.

Moreover, it was also found that the proper time window for anti-TNFα antibody treatment can be from at least a week prior (as long as the antibody is stable for the time window) to immediately after (e.g., within 0.5-1 hour) the SHP-1 inhibitor/αTLR treatment. It is preferable that the anti-TNFα antibody is provided prior to or simultaneously with therapy so that it maximally blocks the TNFα induced after the treatment with the myeloid cell activating agent or therapy.

All references mentioned in the present invention are incorporated herein by reference as if each of those references has been incorporated by reference individually. Although the description referred to particular embodiments, it will be clear to a person skilled in the art that the present invention may be practiced with variation of these specific details. Hence this invention should not be construed as limited to the embodiments set forth herein.

1. A method of treating a cancer in an individual, comprising administering to the individual a) a myeloid cell activating agent or therapy, and b) a TNFα inhibitor.

2. The method of claim 1, wherein the method further comprises administering to the individual an inhibitor of the SHP-1 pathway.

3. A method of treating a cancer in an individual, comprising administering to the individual a TNFα inhibitor and an inhibitor of the SHP-1 pathway, optionally wherein the individual is under an inflammation reaction, optionally wherein the inflammation reaction is characterized by
a) an acute inflammation,
b) a cytokine release syndrome, or
c) an increased level of 1) at least two or three of TNFa, IL-6, IFN-g, and IFN-α, and/or 2) at least two or three of CCL2, CCL5, CXCL1, and CXCL10, further optionally wherein the inflammation reaction is characterized by an increased level of IL-2, IL-12, IL1b, and/or IL-10.

4. The method of claim 2 or 3, wherein the inhibitor of the SHP-1 pathway comprises a SHP-1 inhibitor, optionally wherein the SHP-1 inhibitor is selected from the group consisting of: a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZFN, or TALENS systems), a peptide agent, a protein agent (e.g., an antibody agent that targets SHP-1), a protein degrading or destabilizing agent, a protein modified with an unnatural amino acid, an antibody directed therapy, an antibody drug conjugate, and any combination thereof.

5. The method of claim 4, wherein the SHP-1 inhibitor is selected from the group consisting of TPI-1 and analogs or derivatives thereof, PTP-I, NSC-87877, NSC-87877 disodium, sodium stibogluconate, phenylhydrazonopyrazolone (PHPS1) sulfonate, oxindole, NSC-117199, salicylic acid, diterpenoid quinone, cryptotanshinone, vitamin E derivative, tocofersolan (TPGS), α-tocopherol acetate (αTA), α-tocopheryl succinate (αTOS), phomoxanthone A (PXA), and a PKCθ activator.

6. The method of claim 4 or claim 5, wherein the SHP-1 inhibitor is TPI-1 or an analog or derivative thereof.

7. The method of any one of claims 1-6, wherein the myeloid cell activating agent or therapy activates a cell selected from any one of: macrophages having the M1 phenotype, intratumoral dendritic cells, intratumoral B cells, antigen presenting cells, and any combination thereof.

8. The method of any one of claims 1-7, wherein the myeloid cell activating agent or therapy is selected from the group consisting of: a STING activator, a Toll-like receptor (TLR) agonist, a PAMP/DAMP activator, a chemotherapy, a pro-inflammatory cytokine, a vaccine (e.g., a cancer vaccine), a bacteria or component thereof, a virus or component thereof, a fungus or component thereof, an immune cell, a sound treatment, a magnetic therapy, an electrical treatment, a cryotherapy, a surgery, a thermotherapy, a radiation treatment, a radiopharmaceutical treatment, an electrostatic treatment, an antibody drug conjugate, and any combination thereof.

9. The method of claim 8, wherein the myeloid cell activating agent or therapy is a STING activator or a Toll-like receptor (TLR) agonist.

10. The method of claim 8 or claim 9, wherein the myeloid cell activating agent or therapy comprises a TLR agonist, optionally wherein the TLR agonist activates TLR2, TLR3, TLR4, TLR5, TLR7, TLR8, TLR9, and/or zymosan, further optionally wherein the TLR agonist comprises CpG, polyI:C, and/or R848.

11. The method of claim 8, wherein the myeloid cell activating agent or therapy comprises a STING activator, optionally wherein the STING activator is selected from the group consisting of: 2′3′-cGAMP, ADU-s100, G10, SR-717, Vadimezan (DMXAA; ASA-404), Sting agonist-20, MSA-2, diABZI STING agonist-1, cGAMP (Cyclic GMP-AMPP), STING agonist-3, and c-di-AMP (Cyclic diadenylate) sodium.

12. The method of claim 8, wherein the myeloid cell activating agent or therapy comprises immune cells.

13. The method of claim 12, wherein the immune cells comprise T cells, optionally wherein the T cells express a chimeric antigen receptor (CAR) or an antigen-specific TCR, optionally the immune cells comprise at least about 10⁶, 2×10⁶, 5×10⁶, 10⁷, 2×10⁷, 5×10⁷, 10⁸, 2×10⁸, 5×10⁸ T cells, further optionally wherein the method comprises administering at least two or three doses of the immune cells.

14. The method of any one of claims 1-13, wherein the TNFα inhibitor is selected from the group consisting of: a small molecule inhibitor, a neutralizing antibody, a TNFα receptor blockade antibody, a soluble TNFα receptor, a TNFα-targeting short interfering RNA (siRNA), a chemical inhibitor of TNFα mRNA stability, an inhibitor of TNFα converting enzyme (TACE), and derivatives thereof.

15. The method of claim 14, wherein the TNFα inhibitor is a TNFα neutralizing antibody, further optionally wherein the antibody is selected from the group consisting of: infliximab, adalimumab, etanercept, golimumab, and certolizumab.

16. The method of any one of claims 1-15, wherein the method further comprises administering to the individual an effective amount of a lymphocyte activating agent.

17. The method of claim 16, wherein the lymphocyte is a T cell.

18. The method of claim 16 or claim 17, wherein the lymphocyte activating agent is selected from the group consisting of: a cytokine, a chemokine, a metabolism-modulating drug, a metabolite antagonist, an immune checkpoint inhibitor, an immune cell, a cancer vaccine, a bacteria or component thereof, a virus or component thereof, a fungus or component thereof, a bispecific T cell engager (BiTE), an antibody-drug conjugate, and any combination thereof.

19. The method of any one of claims 1-18, wherein the TNFα inhibitor is administered within two weeks prior to, concurrently, or within 3 hours after the administration of a) the myeloid cell activating agent or therapy and/or b) inhibitor of the SHP-1 pathway.

20. The method of any one of claims 2-19, wherein the TNFα inhibitor is administered within two weeks prior to, concurrently, or within 3 hours after the administration of inhibitor of the SHP-1 pathway.

21. The method of any one of claims 2-20, wherein the inhibitor of the SHP-1 signaling pathway is administered systemically, optionally wherein the inhibitor of the SHP-1 signaling pathway is administered orally, intravenously, subcutaneously, or intraperitoneally.

22. The method of any one of claims 2-20, wherein the inhibitor of the SHP-1 signaling pathway is administered locally, optionally wherein the inhibitor of the SHP-1 signaling pathway is administered intratumorally or topically.

23. The method of any one of claims 1-22, wherein the myeloid cell activating agent or therapy is administered systemically, optionally wherein the inhibitor of myeloid cell activating agent or therapy is administered orally, intravenously, subcutaneously, or intraperitoneally.

24. The method of any one of claims 1-22, wherein the myeloid cell activating agent or therapy is administered locally, optionally wherein the inhibitor of the myeloid cell activating agent or therapy is administered intratumorally or topically.

25. The method of any one of claims 1-24, wherein the myeloid cell activating agent or therapy is administered daily for at least 2, 3, 4, 5, 6, or 7 days.

26. The method of any one of claims 2-25, wherein the inhibitor of the SHP-1 signaling pathway is administered daily for at least 2, 3, 4, 5, 6, or 7 days.

27. The method of any one of claims 2-26, wherein the inhibitor of the SHP-1 pathway and the myeloid cell activating agent or therapy are administered within 24 hours of each other, optionally wherein the inhibitor of the SHP-1 pathway and the myeloid cell activating agent or therapy are administered to the individual simultaneously or concurrently.

28. The method of any one of claims 1-27, wherein the TNFα inhibitor is administered at least once a week, once every five days, once every three days, or daily.

29. The method of any one of claims 1-27, wherein the TNFα inhibitor is administered no more than about once a week.

30. The method of any one of claims 1-29, wherein the method further comprises assessing the level of TNFα level in the individual (e.g., serum or blood TNFα level).

31. The method of any one of claims 1-30, wherein the method further comprises administering an IL-6 inhibitor.

32. The method of any one of claims 1-31, wherein the method comprises administering at least two doses of the TNFα inhibitor, optionally wherein the two doses of the TNFα inhibitor is separated a) at least by 2, 3, 4, 5, 6, or 7 days, or b) at most by 4, 3, 2 or 1 week, 6, or 5 days.

33. The method of any one of claims 1-32, wherein the TNFα inhibitor is administered to the individual for at least two cycles, wherein each cycle has about three to about seven days.

34. The method of any one of claims 2-33, wherein the method comprises administering both a tyrosine kinase inhibitor and a SHP-1 inhibitor.

35. The method of any one of claims 1-34, wherein the method comprises administering a) a SHP-1 inhibitor, optionally the SHP-1 inhibitor is a TPI-1 or an analog or derivative thereof, b) a TLR agonist, optionally wherein the TLR agonist activates TLR2, TLR3, TLR4, TLR5, TLR7, TLR8, TLR9, and/or zymosan, and c) an TNFα inhibitor, optionally wherein the TNFα inhibitor is an anti-TNFα antibody.

36. The method of any one of claims 1-35, wherein the method comprises administering a) a SHP-1 inhibitor, optionally the SHP-1 inhibitor is a TPI-1 or an analog or derivative thereof, b) a STING activator, and c) an TNFα inhibitor, optionally wherein the TNFα inhibitor is an anti-TNFα antibody.

37. The method of any one of claims 1-36, wherein the method comprises administering a) a SHP-1 inhibitor, optionally the SHP-1 inhibitor is a TPI-1 or an analog or derivative thereof, b) a radiotherapy, and c) an TNFα inhibitor, optionally wherein the TNFα inhibitor is an anti-TNFα antibody.

38. The method of any one of claims 2-37, wherein the inhibitor of the SHP-1 pathway and the myeloid cell activating agent or therapy are administered to the individual until the individual undergoes tumor clearance.

39. The method of any one of claims 16-38, wherein the lymphocyte activating agent is a cytokine, wherein the cytokine comprises IL-2, IL-4, IL-7, IL-9, IL-21, or IL-15, or a biologically active derivative thereof, optionally the cytokine comprises IL-2 or a biologically active derivative thereof.

40. The method of any one of claims 16-39, wherein the lymphocyte activating agent is an immune checkpoint inhibitor, wherein the immune checkpoint inhibitor comprises an anti-PD-1 antibody.

41. The method of claim 39 or claim 40, wherein the cytokine and/or the anti-PD-1 antibody is administered to the individual daily for at least 2, 3, 4, 5, 6, or 7 days, optionally wherein the cytokine and/or the anti-PD-1 antibody is administered to the individual for at least two cycles, wherein each cycle has about three to about 20 days.

42. The method of any one of claims 1-41, wherein the individual does not develop Grade 2-4 cytokine release syndrome or pro-inflammatory organ damage.

43. The method of any one of claims 1-42, wherein administration of the TNFα inhibitor does not compromise or weakly compromises tumor clearance.

44. The method of any one of claims 1-43, wherein the cancer is
a) a solid tumor or a hematological cancer,
b) a late-stage cancer, and/or
c) resistant or refractory to a radiation therapy, a chemotherapeutic agent, and/or a checkpoint inhibitor.

45. The method of any one of claims 1-44, wherein the individual is a human.
